(19) United States
(12) Patent Application Publication
(10) Pub. No.: US 2022/0273312 A1
(43) Pub. Date: Sep. 1, 2022

(54) Vascular valves and servovalves - and prosthetic disorder response systems

(71) Applicant: David S. Goldsmith, Atlanta, GA (US)

(72) Inventor: David S. Goldsmith, Atlanta, GA (US)

(21) Appl. No.: 16/873,914

(22) Filed: Aug. 11, 2020

(51) Int. Cl. A61B 17/122; A61F 2/24

(52) U.S. Cl. CPC A61B 17/1227; A61F 2/2476; A61B 2017/00969

(57) ABSTRACT

Set forth are the structure, function, placement, and applications of vascular valves and servovalves. In the vascular tree, the diversion, shunting, and bypass of flow these provide allow solid organ transplantation which eliminates anoxia and graft organ degradation following harvesting and storage, likely including late term cardiac allograft vasculopathy. Along the lower urinary tract, the diversion of urine from damaged ureters to the native or an artificial bladder or collection bag alleviates problems of intractable urinary incontinence, nocturia, overactive bladder, and frequent urination. Where the lower tract is missing, the synthetics in a valve-based prosthesis preclude infection and degenerative metaplastic transition which can result in malignancy when gut is used to construct a neobladder and/or high maintenance stoma. Accessory channels in side-entry valves and servovalves allow the direct pipe-targeting of medication to sites of disease, anastomoses, or any other trouble spots.

### CROSS REFERENCE TO RELATED APPLICATION

This nonprovisional application follows provisional application 69/922,526, entitled Intravascular Valves and Servovalves and Prosthetic Disorder Response Systems, filed on 13 Aug. 2019 under 35 U.S.C. 119(e), herewith retitled Vascular Valves and Servovalves and Prosthetic Disorder Response Systems, in describing ductus side-entry jackets adapted to function as vascular valves and the capabilities such valves make possible. The inventive aspects pertaining to related devices—stent and impasse jackets, described in copending application Ser. No. 15/932,172; ductus side-entry jackets described in copending application Ser. No. 15/998,002; and nonjacketing side-entry connectors, described in copending application Ser. No. 14/998,495—applicable to side-entry flow diversion jackets and vascular servovalves are incorporated by reference.

### FEDERALLY SPONSORED RESEARCH OR DEVELOPMENT

None

### PARTIES TO A JOINT RESEARCH AGREEMENT

None.

### SEQUENCE LISTING

None.

### PRIOR DISCLOSURE

Provisional application 69/922,526.

### SUMMARY TABLE OF CONTENTS

1. Field of the invention
2. Background of the invention
3. Summary of the invention
4. Objects of the invention
5. Description of the drawing figures
6. Description of the preferred embodiments of the invention

### Claims

### Abstract

### 1. FIELD OF THE INVENTION

The apparatus and methods to be described are intended for use by pediatric and adult cardiovascular and cardiothoracic surgeons, interventional cardiologists and radiologists, general surgeons, neurosurgeons, and veterinary surgeons to make possible the diversion of blood through shunts or bypasses, and by urologists and endourologists to allow the diversion of urine from one or both ureters to a damage and disease-free distal, or inferior, part of the lower urinary tract, or into a prosthetic orthotopic ‘neobladder’ made of synthetic materials or one surgically constructed using autologous or allogeneic transplanted or engineered tissue, whence either direct voiding into a bathroom receptacle or facilities-intermediate external collection bag is made possible.

Blood diversion valves make possible the direct transfer of a solid organ by anoxia-free bypass, switch-to-bypass, or switch, transplantation, and the mid- and/or postprocedural bypass of pathological or surgical wounds, resected vessels, an aneurysm, or coarctation, for example, in the vascular tree. Medical conditions spectral in comprehending numberless variables requiring treatment, the means described are intended to serve as alternatives to supplant or as adjuvants to be used with established measures to treat intractable disease that is serious and chronic, never acute and responsive to established conventional treatment.

### 2. BACKGROUND OF THE INVENTION

This nonprovisional application describes ductus side-entry jackets modified to serve as vascular valves or servovalves to adjustably divert urine from a ureter in order to bypass the lower urinary tract or an intervening portion thereof, or to divert blood from a vessel through a catheter to an insufficiently oxygenated organ or region through a shunt or bypass, or to divert blood to a transplant organ of a donor from the corresponding blood supply and drainage vessels of the recipient. Also described are related devices such as vascular servochokes and sphincters suitable for adjusting a local blood pressure, for example, which also represent types of valves.

Ductus side-entry jackets and prosthetic disorder response systems are described in copending nonprovisional application Ser. No. 15/998,002, entitled Ductus Side-entry Jackets and Prosthetic Disorder Response Systems, filed on 8 Jun. 2018 and in its parent, Ser. No. 14/121,365, filed on 25 Aug. 2014 and published as US2016/0051806 on 25 Feb. 2016, as well as in the continuation in part thereto, Ser. No. 15/998,002 published on 12 Dec. 19 as US20190374213. Stent jackets and impasse jackets are described in copending application Ser. No. 13/694,835 filed on Jan. 9, 2013 and published as US 2014/0163664 on Jun. 12, 2014, and Ser. Nos. 14/121,365 and 15/998,002, the provisions of the foregoing applicable to side-entry flow diversion jackets and incorporated herein by reference.

Previous applications belonging to the series of which the titles include the appellation ‘ . . . and Prosthetic Disorder Response Systems’ comprise Ser. No. 15/998,002, entitled Ductus Side-entry Jackets and Prosthetic Disorder Response Systems and Ser. No. 14/998,495 entitled Nonjacketing Side-entry Connectors and Prosthetic Disorder Response Systems. These and the application presaging these, Ser. No. 15/932,172, entitled Integrated System for the Infixion and Retrieval of Implants, seek to implement the treatment of serious chronic disease by means of a fully and when necessary, permanently implanted system.

No measures described are intended to treat disease that is acute, not serious, or better treated by conventional medical means. A prosthetic disorder response system comprises small drug reservoirs and direct-to-target drug delivering fluid and implanted electrical stimulation wired or wireless lines to allow the direct application to the nidi or sites of disease of drugs and/or electrostimulation, imaging probes, and other diagnostic and therapeutic end-devices as necessary under the control of an implanted microcontroller or microprocessor as master controller responsive to implanted sensors and executing a prescription-program.

In complex comorbid disease, an implanted hierarchical control system with overall control over the direct pipe-targeting to a nidus, for example, of medication, for example, coordinates sensor inputs received at the local processor level, whereupon the sum thereof is passed up through intervening levels of integration and coordination to be acted upon by the master control microprocessor. The channels or arms of the control system can represent different organ systems or less intimately related comorbidities.

This application expands upon the provisions of copending application Ser. Nos. 14/121,365 and 15/998,002 by describing side-entry jackets which incorporate flow diversion chutes. Such constitute valves which can be used to redirect all or a controllable portion of the flow through a native ductus through a shunt or bypass into another native ductus. The incorporation into such valves of one or more accessory channels allows the direct targeting of drugs such as anticoagulants, antimicrobials, anti-inflammatories, anesthetics, anticrystallization agents, and others into the valve and the synthetic line into which the valve diverts flow.

One reason for cross-references among the applications is that drug delivery through a line from a small drug reservoir implanted subdermally in the pectoral region can be dynamically adjusted responsive to implanted sensor feedback either or both at the reservoir outlet pump or by diversion of a proportion of the blood flow carrying the drug to the target into another, larger, vessel, with an equal volume of blood returned to the vessel of origin from that or another vessel. Different aspects of this action are developed in different applications. The drug having previously been confirmed efficacious, adjustment in the dose might be necessitated due to a feedback-indicated lack of response requiring an increase in the dose or a response indicative of an overdose, requiring the reduction or stoppage of continued delivery.

In general, a single chronic morbidity is administered by an implant microcontroller, whereas comorbid disease is by a microprocessor. In more complex comorbid disease, administration is by an implanted microprocessor as the master controller in a hierarchical control system of which each arm is assigned to a single morbidity or organ system. The master controller continuously coordinates these inputs to command the best overall resolution in terms of directly pipe-targeted medication or other treatment when applicable across the set of morbidities in accordance with its prescription-program.

To allow the internal structures to be shown clearly, valves have been drawn disproportionately large, prompting a misleading impression of excessive size and weight. Each type valve has also been shown with every element necessary to deal with any potential application. In reality, the simplest valve for a given purpose is used; not all features are likely to be needed for a specific application, so that the cost for a specific valve will be less than the drawings suggest.

To prevent backfilling with blood and the possibility of obstruction by clot, accessory channels along or emptying into blood flow passageways are filled with water and/or provided with an anticoagulant drip. The implanted controller adjusting the diversion chutes, water fill and the release of an anticoagulant are coordinated automatically.

Adjustment at the outlet pump allows increasing, reducing, or stopping the dose at the pump outlet. However, adjustment at the pump does not allow recovery of the drug or any portion thereof once it has been released into the drug delivery line, or drugline, which passes through an accessory channel in the jacket or valve. To reverse the pump would still leave a coating of the drug along the surface of the drugline, or sideline, lumen. Reservoirs are usually used for a single drug; however, if needed for another drug, the reservoir can be flushed out with a double lumen syringe that allows water to be circulated through it.

Blood 92 percent water, a coating along the inner walls of the delivery line which represents a negligible dose can be rinsed forward into the target vessel with water. The reservoir can then be refilled with another drug as usual, by insertion of a hypodermic syringe through the reservoir opening in the subdermal port. Once delivered into the blood supply of the target organ or tissue, reduction or the prevention of its delivery to the target organ or tissue requires the use of 2-way, or bidirectional, diversion servovalves, which draw off flow and divert it into another vessel. Bidirectional servovalves are described below.

The requirements imposed by lifelong implantation in minimizing if not eliminating adverse tissue reactions, sharp corners or edges, and avoidable mass and size, along with the medical requirements then drive the design of the components. Other requirements imposed for lifelong implantation include the ability to deliver antifouling agents to counteract the accumulation if not formation of clot and biofilm in vessels, crystal in the urinary tract, and if necessary, the ability to handle radioactive substances. The timely automatic direct targeting of drugs in an ambulatory patient according to the program and/or responsive to data fed from implanted sensors to the implanted microcontroller in monomorbidity or microprocessor in multimorbidity eliminates delay in treatment and dependency upon the patient to adhere to the prescription regimen.

The placement of such a system responds to the conviction that chronic and especially progressive disease warrant decisive treatment upon initial diagnosis, not ‘half-way’ measures likely to lead to surgical or radiological treatment anyway. The application of more decisive measures capable of providing a decisive relieving of symptoms ab initio is to be preferred. Provided the benefits to be gained from the small incisions and endoscopic manipulations required to implant an automatic prosthetic disorder response system stand to materially improve the quality of the patient's life and avert the eventual need for more radical treatment, then compared to conventional medical management, the outcome will have been worth the brief episode of invasiveness it took to provide.

The applications for such a system range from the relative simplicity of a central line to treat a chronic condition which once placed must remain permanently, to a permanent line for the direct release into the portal vein of insulin responsive to the data supplied by glucose sensors, to bilateral lines for the direct targeting through the internal carotids of antipsychotics, anticonvulsants, dementia counteractants, and antidepressants, for example, to the brain for release on a scheduled basis, to multiple lines to treat comorbid disease as channels in a hierarchical control system under the coordinated control of a master microprocessor. Variation in the dose fed to either side of the brain may yield therapeutic information for use in patients and neurophysiological information through translational experimentation.

Vascular jackets and servovalves fall into four types or families, of which the different embodiments serve different medical functions. To permit a reduction in the cost of manufacture, the embodiments within each type are based upon a common platform. The types are 1. Diversion jackets without a powered driver for continuously variable direct manual control along the lower urinary tract; 2. Plunger solenoid diversion jackets which allow two state, or bistable fully open/fully closed function, for application along the lower urinary tract and to implement sudden switch, or bypass, transplantation to be described; 3. Servomotor-driven valves used to implement metered switch, or bypass, transplantation to be described; and 4. Servomotor-driven valves with direct manual adjustability to allow bypass of the carotid bifurcation during and following a carotid endarterectomy.

The term ductus denotes a tubular anatomical structure, regardless of type. Ductus side-entry jackets and the procedure for placing these are described in copending Ser. No. 14/121,365 published as US 2016/0051806, or Ser. No. 15/998,002, entitled Ductus Side-entry Jackets and Prosthetic Disorder Response Systems, and the term ‘body surface port’ denotes a port whether on or beneath the skin. As shown in copending application Ser. No. 14/998,495, entitled Nonjacketing Side-entry Connectors and Prosthetic Disorder Response Systems, the targeted release of medication into a cavity, such as the urinary bladder, or into tissue, under the control of a fully implanted automatic disorder response system executing a prescription-program is through nonjacketing side-entry connectors, while ductus side-entry jackets, described in copending application Ser. No. 15/998,002, entitled Ductus Side-entry Jackets and Prosthetic Disorder Response Systems, are used to communicate with the lumina of tubular anatomical structures. (‘Injection’ denotes delivery through a syringe at a pressure greater than gravity; ‘infusion’ is the passive delivery by a drip.

The removal of a degraded organ for replacement is neither ‘harvested,’ which term denotes the procurement of tissue needed for transplantation, nor, having never been implanted, is it ‘explanted.’ Ductus side-entry jackets are positioned to encircle a tubular anatomical structure, or bodily conduit, to create a continuous passageway between the lumen of the conduit and the line leading to the jacket, the jacket itself, and the native lumen beyond the jacket. This line can support movement inward or outward; that is, conduct either incurrent flow to deliver medication and provide a passageway to insert a cabled device, or excurrent flow to take a needle aspiration or a core or incisional biopsy tissue test sample, or to withdraw a cabled device, such as a fine fiber endoscope, intravascular ultrasound probe, or laser.

In an implanted automatic disorder response system, basic side-entry jackets, nonjacketng side-entry connectors, and intravascular servovalves, to include flow diversion valves and chokevalves, can all be used in a coordinated manner to optimize circulation, for example. Such a system is intended to remain in place over the life of the patient. Means incorporated to allow for considerable growth, placement even in a small child should not require reentry to replace parts more than once.

Existing means for connecting a synthetic to an anatomical tube such as a ‘butterfly needle’ (scalp vein set, winged infusion set) an indwelling catheter cannot begin to meet a requirement for lifelong function. The connections essential to sustain such an implanted system cannot otherwise be produced. All are limited to short term use; neglecting to withdraw the devise within the recommended time will result in irritation, tissue damage infection, leakage, and/or obstruction.

The leading edge of the blood-conveying bloodline, or mainline connector is a stainless steel (inox) trepan (trephine, tissue circle cutter, circular blade, punch) mainline end-piece, which placed against the outer surface of the ductus and connected to a vacuum pump, draws the wall of the ductus past it to cut a circular hole (ostium, aperture) in the side of the ductus, the plug, or cylindrical core, of tissue cut then removed by continued suction. The term ‘mainline’ applies when the fluid conducted is urine, as when drawn directly from a ureter through such a line into a collection bag. An adverse allergic reaction to the nickel in stainless steel common, an alternative or coating material for the trepan is polyetherether ketone with a razor sharp cutting edge.

In most applications, ductus side-entry jackets, vascular valves, servovalves, and nonjacketing side-entry connectors are not removed once the procedure has been completed but rather left in place to allow continued use of their accessory channels through the connected sidelines, or druglines, to implement automatic sensor input-driven follow-up drug treatment.

The fully implanted automatic disorder response system can also incorporate diagnostic sensors, such as a tiny oximeter, signaling means for alerting the clinic to a cerebrovascular accident, electrical effectors such as angioscopes, and electrical effectors such as neurostimulatory. Usually more than one incorporated into a diversion jacket or valve, an accessory channel, or drugline, remains usable whether the accompanying mainline is removed or left in place as a prosthesis, as might, for example, be decided following an ischemia-free carotid endarterectomy where the postprocedural condition of the common, internal, and external carotids recommended this rather than depending upon the native anatomy.

Citing the extracranial carotids as exemplary of comorbid vascular disease, when these vessels are observed to not only have become atherosed (atheromatous, atherosclerosed), but whether aneurysmal, dissected, or flow-mediated vasodilated due to Marfan's syndrome, for example, or malacotic due to a connective tissue disorder, malignant invasion, trauma, or otherwise additionally diseased in such manner as to have become structurally weakened, and/or extensively deformed, or subject to recurrent stenosis, so that—especially following earlier stenting or endarterectomy—a stent graft or saphenous or femoral vein interposition graft are felt inadequate, the synthetic common carotid with branches bypass is applied ab initio as a prosthesis and the native carotid with branches removed.

The terms ‘bypass,’ ‘compound bypass,’ and ‘switch’ transplantation’ denote the transfer of circulation from the donor to the recipient organ. Providing major histocompatibility complex, organ size matching, and tolerance induction have been accomplished, this transfer can be sudden, with removal of the innate organ and its replacement with the graft organ in immediate succession. While the transfer requires that donor and recipient be positioned beside one another, preliminary match testing can be remote. Only in the most exigent circumstances would a sudden switch transplantation be performed without such preparation. Both ‘brain-dead’ and live donor bypass, or switch, transplantation requiring the compresence of both donor or donors and recipient or recipients, absent special planning, smaller centers without heliports or quick access to a regional airport are limited to less urgent cases.

The reason that life support is currently stopped following the death of a prospective organ donor is precisely the lack of a means for directly and seamlessly transferring the graft organ from the circulatory system of the donor into the circulatory system of the recipient with no interruption in the delivery of oxygenated blood, and in so doing, avert the cytokine-generating havoc associated with death, which at least in half of all heart transplants, is likely to prove a key factor in the eventual failure of the graft organ. The removal of tissue, much less an intact organ, with hormonal and neurological connections that eventually extend throughout the body, to suddenly sever it from its unique situation within this milieu, represents a stunning insult with a long memory.

Nowhere is the benefit of an anoxia-free heart transplant more propitious than at the outset of life, in a neonate with a heart which due to developmental factors grew too malformed in utero to allow normal circulation despite the best of reconstructions or a conventional transplant, whether alone or with the addition of a mechanical assist device. Every tissue and organ would be spared the secondary maldevelopment due to hypoxia that would continue to the end of a sick life shortened for that reason in itself as well as the stress of retransplantations and other surgical procedures. Such contemplates that a new heart without the experience of having died is more likely to survive.

Solenoid driven valves accomplish this suddenly, while servovalves allow the gradual transition from the donor to the recipient, which affords an interval during which immune tolerogenic induction can be accomplished. In the present context, the terms valve, vascular valve, vascular jacket, diversion valve, valve jacket, and diversion jacket are synonymous and can refer to either solenoid or servomotor driven valves. Essentially, sudden switch, or compound bypass, transplantation responds to urgency in end-stage disease with death imminent as to require that the affected organ be replaced as soon as possible.

The term ‘metered switch’ transplantation denotes a tightly controlled transfer of circulation from the recipient to the donor, or graft, organ at a rate directly responsive to the detection of rejection analytes by implant sensors. Demanding the continuous and immediate detection and coordination of multiple data streams and immediate response to any adverse indicants, this process is entrusted to a master controller to execute automatically in accordance with a prescription-program. Such programs are prepared and archived to administer a type procedure, such as a heart, kidney, or heart and kidney transplant, allowing for the variables encountered with each.

An emergency condition that justifies use of the sudden rather than the metered switch technique is encountered when the patient arrives without having been stabilized, or even if an inpatient, experiences a potentially fatal cardiac accident, for example, which if conventionally treated would still leave the patient with end-stage heart failure intractable to medical treatment and posing the risk of death. Barring such an exigent circumstance, metered switch transplantation is preferred. This is especially true of the heart with its multiple points for the in- and outflow of blood. Due to the abruptness of sudden bypass, or switch, transplantation, confirmation of compatibility before initiating the process is crucial.

When urgency does not allow sufficient time to adequately evaluate compatibility, sudden switch transplantation can be preceded by supplementary manual infusion of tolerance inducing, or cross chimerizing, cells and drugs. However, the valves used to perform a metered switch transplantation instantly adaptable to the reaction to this infusion as reported to the implanted disorder response system controller by implanted sensors, initiation of the process by metered switching is preferable as allowing immediate cessation, or ‘bailout,’ or if the reaction is nonacute, temporary ‘watchful waiting’ until the adverse reaction subsides. Homozygous, or identical twins, or in experimental work, cloned replicate specimens of the source or index specimen, can be sudden switch transplanted without concern.

The number of variables involved in an orthotopic or double switch heart transplant prohibits a reduction to and straightforward delineation of a simple procedure. In both single and double heart transplants, the detailed condition of the heart or hearts, comorbidity, organ system interconnections, electrolyte disturbances, available drugs, and alternative means of therapy must all be taken into account. To this, double heart transplantation adds the need to coordinate the relative ejection fraction, or absolute stroke volume, of either heart as well as the timing of each.

Following a bypass, or switch, heart transplant, whether donor and recipient stumps are anastomosed, the vascular servovalves used to connect all of the bloodlines are retained in place where these remain capable of automatic sensor-driven adjustments in flow rate and direct drug delivery through their accessory channels. That anastomosed, the implanted controller can switch between the bypass and native route in coordination with the release of medication directly pipe-targeted to the treatment site is a significant factor in facilitating healing.

FIG. 22 shows that rather than to cover over the anastomoses, the valves straddle these at a distance, so that when donor and recipient stumps are sutured, the valves are positioned to deliver medication directly to the site in support of healing. More generally, because it eliminates the exposure of nontargeted tissue, the direct pipe-targeting of drugs such as immunosuppressive or chemotherapeutic to a nidus eliminates such exposure as a cause for increased susceptibility to infection and malignancy. Moreover, when the components used to deliver the drugs are radiation shielded, the drugs can include radioactive chemotherapeutics, for example.

Both single and double heart transplantation, for example, can do much to alleviate the shortage in hearts available for transplantation, which due to the current means for preservation, are invariably impaired upon placement and likely to fail due to cardiac allograft vasculopathy, for example. Current methods guarantee that the misery of a defective heart will not have been obliterated at the outset but instead persist throughout a much shortened and miserable life, wherein a mind capable of high accomplishment is instead diverted to obsessive anxiety over what will certainly be an early death.

In children born with materially defective if not unsurvivably deformed hearts, replacement with one or two hearts capable of normal, pulsatant, or non-Fontan circulation, which pumps able to do so having posed problems of clotting and a lack of dependability, only a transplant can provide, is able to overcome the inevitable deterioration in all other organs that will inevitably ensue following the most adept surgical repair. Double heart transplantation is also useful as a bridging strategy that provides normal circulation pending the availability of a good heart. End stage heart failure in the elderly is usually controllable with drugs and mechanical assist devices, but this is not so in a prenate or neonate with a heart that is unsurvivably malformed and will continue to prevent normal development anywhere else in the body.

Not a bridging strategy to transplantation as is often the object in placing a totally artificial heart or ventricular assist device, a double heart transplant provides pulsatant circulation with the placement of a second heart to cooperate with that native in a relation of mutual support intended to serve to the end of life. Sensors incorporated into the vascular servovalves or positioned separately continuously monitor the hearts so that upon detection of a dysrhythmia, or of one or more analytes indicative of rejection, for example, the implanted controller responds in accordance with its prescription-program.

The response can consist of the direct pipe-targeting of drugs to the site, adjustments to the vascular servovalves, and/or the delivery through prepositioned electrostimulators of synchronization discharges to ameliorate if not reverse the problem. These measures, effectuated immediately and automatically while the patient is ambulatory stand to avert more serious consequences that would ensue were attention deferred pending arrival at a clinic where to perform a diagnosis would itself take time. Continuous protection supported by coordinated means for immediate response medicinal and electrical to an exigency should extend the durability of the graft organ well beyond that associated with conventional transplantation. Mechanical assist devices are not only characterized by limited life but an expense that makes these prohibitive in much of the world.

Any means for imparting normal circulation which depends in whole or part upon mechanical components must answer to the reliability of the components employed. Sudden and metered switch heart transplantation is made possible by vascular valves. These are less complex than are alternative devices such as ventricular assist devices and totally artificial hearts, which do not provide normal blood flow in any event, making these properly unacceptable for use in children.

By contrast, the bypasses used in switch transplantation can back up anastomoses of the donor to the recipient vessels and the implanted automatic disorder control system can switch between the direct and bypass pathways in response to sensor inputs that indicate a malfunction or as specified in the prescription-program of the implanted controller. This alternation can not only be used to occasionally relieve stress during healing, but the tiny servovalves used can be left in place to directly pipe-target drugs to the locus of inflammation or the appearance of a lesion, for example, wherever it arises. Suffice it to say, vascular valves can be made to a level of reliability that meets and exceeds that essential.

Much the same as switching between the bypass and native ductus following a carotid endarterectomy as shown in FIGS. 25A and 25B where transection and anastomosis of supply and drainage vessels are uninvolved, following a transplant such as shown in FIGS. 22A and 22B, where transection and anastomosis of supply and drainage vessels are involved, switching thus affords additional advantages. Accessory channels to deliver immunosuppressants to the severed ends of the donor and recipient vessels can also continue to induce immune tolerance until anastomosis is accomplished endoscopically through ‘keyhole’ incisions.

As depicted in FIG. 22B where only those outside the vessels are shown, accessory channels 154 and 154′ can drip drugs to wet the cut ends of the vessels both inside and outside. Retention of the bypass thereafter allows switching between the bypass and native ductus. Switching thus can be accomplished automatically by the implanted controller in response to sensor feedback indicative of rejection requiring immunosuppression or disease requiring the appropriate drugs.

The direct pipe-targeting of drugs and other fluid agents directly to the site of disease whether an organ, circumscribed tissue, or nidus bypasses untargeted tissue and allows doses to be increased as appropriate to the therapy without concern for adverse side effects elsewhere in the body. Such applications are not without caveats—prodrugs, for example, or agents activated by passing through the liver must first be converted into their form following processing by the liver to function when directly targeted for local or topical use. That the benefit gained from chemotherapeutics and steroids would be significantly amplified were untargeted tissue bypassed is clear. Still greater benefit will be had from these and other drugs when radioactive and directly pipe-targeted at dose rates higher than tracer levels. The use of radioactive substances when directly targeted to the site of disease has gone unexplored due to the lack of means for controlling the targeting of radioactive radionuclides, for example, while containing these to protect other tissue. Copending application Ser. No. 14/998,495, entitled Nonjacketing Side-entry Connectors and Prosthetic Disorder Response Systems, illustrates and describes absorbable and permanent radiation shielding for tissue connectors.

Temporary or permanent radiation protective encasement thus applies to every component along the drug delivery path, to include implanted drug reservoirs, reservoir outlet pumps, the druglines leading from the reservoir outlet to the targeted connector, as well as the targeted connector, itself. Where uptake within the target is not complete, three methods for preventing the release of a residue from the targeted site, organ, or gland into the circulation to affect nontargeted tissue are delineated below as well as in the other applications in this related series.

Given this circumstance, the response is to leave in place the servovalves and druglines used to perform the transplant and to have these automatically adjusted as necessary in response to sensor data fed to the automatic response system controller programmed to initiate an optimizing routine should a dysrhythmia, for example, be detected. When the donor organ is removed, the highly elastic bloodlines, or mainlines, relieved of the pull exerted by traversing the distance between recipient and donor organs, contract in length. Other than using highly elastic accordioned lines, additional methods for shortening lines are provided under the section below entitled Description of the Preferred Embodiments.

While the fluid line delivering drugs into the accessory channels of the jackets or valves are separable from the accessory channels per se, as providing one continuous passageway from the small flat drug reservoir implanted subdermally, or subcutaneously, in the pectoral region, to the native lumen, the two are usually referred to collectively as accessory channels, the distinction between the lines and passageway through the jacket or valve taken for granted. The retention of a servovalve with druglines at the connections of the bloodlines allows control over the flow past each. Before and after views of the disposition of the servovalves before harvesting and implantation of the graft organ are shown in the accompanying figure of a left lung transplant.

As seen in FIG. 21 showing the doubling of valves following placement of the graft lung in the recipient, upon completion of a switch transplantation, the valves on the stumps of the graft organ and the recipient organ since removed will be brought into proximity to either side of the stump junctions where these would normally be anastomosed in accordance with the distance from the transection at which the valve is spaced. Where conditions favor retaining the mainline or mainlines connecting the jackets or valves, the stumps would generally not be anastomosed but closed off with suture and a fibrin sealant, for example, flow then passing through the bypass permanently as a prosthesis where, for example, the vessels are malacotic or otherwise impaired.

If so, the drugline (sideline, accessory channel feeding line) of the donor jacket is attached to the drug reservoir implanted in the recipient. If anastomosed, leaving a recipient arterial valve jacket with accessory channel in place allows targeting medication to the anastomosis and the graft organ. A retained venous donor jacket allows targeting the venous anastomosis, and the recipient venous jacket the vein. Pending the development of telescoping tubing suitable for use as bloodlines, or mainlines, temporary anastomosis with the aid of a surgical cyanoacrylate cement, for example, can also be used to allow the mainlines, or bloodlines, to be trimmed of excess length no longer needed with the graft organ in its new position.

Thus, after transplantation, the servovalves straddle the free ends of the donor and recipient stumps so that flow is through the bypass which crosses over the ends. At this point, with the valves fully extended to divert all flow, flow is through the bypass. In some cases where, for example, the recipient vessel is diseased and not trusted to resist dehiscence of the anastomosis, the bypass is left in place as a prosthesis with the free ends sutured closed and sealed with a surgical cement or sealant. Alternatively, the valves can be fully retracted for zero diversion through the bypass, and the free ends anastomosed, one of the valves with drugline retained for follow-up drug targeting by the disorder response system.

Which valve is retained depends upon the direction of flow over the anastomosis as more likely to require treatment. Removal of the other valve is by brief upstream clamping, snipping off the clamp—which will remove the side-entry hole or ostium—and anastomosing the free ends, preferably, with surgical cyanoacrylate cement kept away from the lumen and microsuture, typically 10-0 nylon with a 140 micron needle. Combined with the targeted release of drugs and/or collateral treatment such as electrical, the control system is able to apply such adjustments as are most likely to achieve functional optimization.

The denervation in conventional heart transplantation that would increase the heartrate in response to baroceptor-detected exertion, for example, is compensated for by blood pressure sensors, especially those positioned at critical points, such as along the pulmonary arteries. Transplantation of the heart is more complex than that of other organs where using the metered switch technique, a vascular servovalve may actually be needed only for the blood supplies to the donor and recipient organs. Additional valves, then, may be used to direct flow to avoid shear stress, or to gain immediate proximity to the substrate ductus for the release of drugs through the valve accessory channel or channels, for example.

In a single or double heart transplant, high and low blood pressure detected by implanted pressure sensors can be automatically adjusted to normotensive with the aid of diversion servovalve-connected shunts leading to or from larger extracardiac vessels. The heart is unique in that it receives desaturated blood through the venae cavae, and in that this venous rather than arterial blood ‘supply’ arrives through two great veins rather than a single vessel, or as in the lung, branches thereof. Thus, orthotopic switch transplantation of a replacement heart or heterotopic addition of a second heart requires servovalves on both the superior and inferior venae cavae.

Other than with the heart, such as in a switch heterotopic transplantation of a kidney, a single servovalve on the donor supply bloodline will usually be sufficient to apportion flow between the donor and recipient organs or nearly all flow to either, zero blood supply to be avoided as injurious. When the native organ of the recipient is retained and the donor organ added to create a doublet, the valve can be used to direct all blood to the one or to the other, facilitating the removal of either. In switch transplantation of other organs and glands, such as the kidneys, where multiple major in- and central outflow orifices are not involved in ejection and venous return, the valving required is much simpler.

One servovalve on the donor renal artery is needed. Connection of the venous bloodline—the line connecting donor and recipient renal veins—is ordinarily with basic ductus side-entry jackets incorporating a diversion chute inside the jacket side-stems molded in a suitable material for making jacket and valve parts such as polyether ether ketone. Unless routing the lines to a valve jacket is problematic, druglines to the jacket accessory channel or channels are best run alongside the corresponding bloodlines, or mainlines.

When the distance between the valves is larger so that the weight of both would cause no discomfort, and the pair would achieve more extended coverage of the tissue to be monitored and treated as necessary postprocedurally, then both are left in place. If not, then one of the valves with its mainline and one or more druglines is removed and the stumps anastomosed. A partial ureter cannot be successfully continued by inserting a catheter inside its distal end—where the encircling diversion jacket is strongly resistant to migration and provided with one or more accessory channels, peristalsis will quickly dislodge a catheter.

Once the graft organ is orthotopically positioned, if the bypass is to remain as a prosthesis, the free ends of the donor and recipient stumps are sealed; if not these are anastomosed. When proximity between donor and recipient jackets or valves results in needless bulk and/or encroaches upon neighboring tissue as to either demand adjustment to allow use of the bypass as a prosthesis or the removal of either or both jackets disallowing such use. Jacket removal and resituation along the substrate ductus is accomplished expeditiously not by detachment and reattachment but by clamping, excision, anastomosis, then stitching the jacket with attached lines into the new level, the same remedy used to rotate a valve jacket. Most adjustments and revisions can be accomplished endoscopically or through a combination of endoscopic and transluminal techniques.

Accordingly, the need to remove a jacket or valve by separating it from the substrate ductus should seldom if ever be needed. If some rare eventuality were to override this, removal with minimal loss of blood is more expeditiously accomplished by brief clamping, or in an open field, manual compression, and if the jacket or valve jacket is just short of a transection, the jacket or valve jacket and the mainline entry opening, or surgical ostium created by the trepan (trepine, trephine), simply snipped off and the free end anastomosed or sealed. The least invasive option is the use a vascular closure device.

The rerouting of lines is best by brief detachment, that of mainlines conducting blood requiring brief clamping. The practical absolute distance for such a move negligible, for this to necessitate replacement of the highly stretchable lines as too short should not arise. The removal of a ductus side-entry or valve jacket and mainline, which are essential to the bypass and to allowing it to remain in place as a prosthesis, requires that the free ends of the stumps be anastomosed. If the catheter could be stabilized, it would eventually fail anyway, if not due to an adverse tissue reaction for which an unsupported catheter offers no defense, then due to encrustation and/or the formation of a biofilm. That fresh urine is sterile is mistaken—the bladder of a healthy person harbors uropathogenic microbiota.

By contrast, the accessory channel associated with a side-entry jacket affords a direct path to eradicate any cause of obstruction whether the agent called for is an anticoagulant, antibiotic, antimycotic, and/or an anti-inflammatory. The implanted automatic disorder response system that ductus side-entry jackets, nonjacketing side-entry connectors, and the various valves and servovalves described make possible can directly pipe-target antimicrobials, for example, to the ureters or bladder, for example. The drug is stored in a small flat reservoir placed subdermally in the pectoral region, and an implant microcontroller executing its prescription-program automatically triggers the reservoir outlet pump to release the drug. The advantages of immediate access to a nidus of disease for diagnostic and therapeutic purposes cannot be overstated. Also important is that in a regimen adherent-undependable patient, an implanted disorder response system can automatically deliver medication directly to any such site.

That the drug, an antibiotic or antibiotics, for example, is tightly targeted—and if necessary, can be eliminated from continuing through the circulation in several ways—reduces if not eliminates the development of resistance to it, safeguards the gut microbiota, preventing disruption to the microbiome-gut-brain axis and the numerous neuropsychological, hepatic, and other consequences to which this can give rise. Moreover, that administration is automatic protects regimen noncompliant patients, whether very young, of diminished cognitive capacity, psychopathological, or physically disabled. For example, methicillin resistant Staphylococcus aureus and Staphylococcus epidermidis and the biofilms these produce have been found to yield to thiazolidione derivatives at nonhemolytic concentrations.

Microbial intrusion is dispelled not only through the use of antimicrobial material, but if necessary, the directly targeted delivery of an antimicrobial through the valves accessory channels. The synthetics used in the catheters can be prepared to resist intrusion and colonization by many biofilm-producing bacteria. Materials that release the antibacterial agent or agents only when actually in contact with a pathogen, or infectious agent, the release thereof is not likely to continue over an indefinite period as required for the permanent parts of the valves. For implantation on a long-term basis, thromboresistance based upon the intrinsic properties of the materials used is preferable; however, for long term use, it is possible to include a small replenishable or replaceable canister in a surface port to release nitric oxide when commanded by the control microprocessor. Such could, however, prove adequate for catheters and end-tools to be inserted into or through the valves for diagnostic purposes or for temporary use.

Recent advances at incorporating antimicrobial agents into the material of catheters addressed here complement the ability to pipe antimicrobials to the treatment site through the accessory channels, coating the internal surfaces of the channels and wetting the tissue at the terminus, enhancing the ability to implant catheters for long-term use. References pertaining to some of these recent developments are cited above. Tissue engineered vascular tissue will eventually supplant the materials currently used for prosthetic ductus. The lines are made of debris accumulation-resistant materials, and whether synthetic or tissue engineered, the use of ductus side-entry jackets to join such lines to native ductus allows the lines, valves, and graft to be directly pipe-targeted for maintenance agents to prevent clotting, crystallization, and biofilm.

The main complication in the use of small valves to steer the bloodstream—the risk of occlusion due to a buildup of clot—is dispelled through the delivery of a fractionated heparin drip through the valve accessory channels and by making the lines and valves of clot-resistant materials. Rare instances to the contrary, in limited and localized doses, the risk of inducing thrombocytopenia is improbable.

Moreover, heparin-coated lines are also positively endorsed (see, for example, Lumsden, A. B. and Morrissey, N.J. with 25 collaborators 2015. “Randomized Controlled Trial Comparing the Safety and Efficacy between the FUSION BIOLINE [Getinge Group, Gothenburg, Sweden] Heparin-coated Vascular Graft and the Standard Expanded Polytetrafluoroethylene Graft for Femoropopliteal Bypass,” Journal of Vascular Surgery 61(3):703-712.e1). An alternative to the use of synthetic vessels incorporating heparin is the release of another anticoagulant of which there are many, such as warfarin. Seen thus, heparin bonding is merely a convenience. Antimicrobial polymers for biomedical applications are addressed just above (see, for example, Wo, Y., Brisbois, E., Bartlett, R. H., and Meyerhoff, M. E. 2016, Op cit.). Along the urinary tract, the risk of occlusion due to a buildup of crystal is dispelled through the delivery of a crystal solvent.

Materials that release thromboresistant substances have a limited life, while those that consist of or are coated by highly slippery materials, especially when supplemented by the direct pipe-targeting of a heparin, for example, can remain in place for years if not life. This does, however, presuppose that targeted rather than systemic agents are preferable and that the placement of a subdermal port just beneath the dermis is justified. Untenably susceptible to occlusion without the ability to directly target antithrombotic, antimicrobial, and crystal dissolving agents to the sites of fouling, synthetics are not used to replace small caliber ductus. For this reason, the conventional use of synthetics is limited to large caliber ductus, specifically, in aortofemoral (aortobifemoral), aortoiliac, iliofemoral, and femoropopliteal bypass grafts.

The incentive to use synthetic tubing harbored the implicit desire for full implantation whereby no piping of medication from a port at the body surface to the graft would be needed. However, it is now possible to establish an automatically administered internal drip of essential agents where periodic replenishment is by needle free disposable cartridge jet injection through a subdermally or subcutaneously implanted port or subdermally implanted accessory channel hole in the surface port.

When the same type diversion jacket is applied to the diversion of blood from an artery too small for end-to-end anastomosis with a length of synthetic tubing to terminate just beneath the affected surface, such as with a crus or foot ulcer, the accessory channel with a small pump under the control of a timing module or microcontroller is used to drip an anticoagulant into the line, typically low molecular weight or unfractionated heparin or vitamin K antagonists with careful monitoring of any patient with impaired kidney function, who would then be given a lower dose of low molecular weight heparin.

As described in copending application Ser. Nos. 14/998,495 and 15/998,002, the distal terminus at the hypoxic site is configured to effect reperfusion after Vineberg's method for treatment of the under-oxygenated heart. Such a service channel can also be positioned proximal to a smaller caliber synthetic catheter placed along a native blood vessel or in the urinary tract, or a synthetic (silicone) neobladder to prevent rapid incrustation (encrustation) (see Stein, et al., 2012 cited below) to directly target the counteractant to the synthetic component.

Combining current split thickness skin grafting procedure to close over the ulcerated surface with direct perfusion by the diversion of oxygenated blood as indicated, facilitates the healing of a venous stasis ulcer, for example, and does so with results esthetically superior to older pinch graft methods. Skin grafting aside, the diversion of blood using a synthetic line with heparin support as specified is no less applicable to under-oxygenated tissue, regardless of region.

As will be made clear, when a prospective organ donor dies, is sustained past death in virtually living condition, and a graft organ taken from that donor is transferred into a matched recipient, the infusion reactions, or ‘cytokine storms’ (references cited below), and systemic inflammatory response syndrome associated with organ failure and death will have been considerably prevented from degrading the prospective graft. Broadly, eliminating this deterioration reduces the multiple obstacles of transplantation to one of immune tolerance, and the metered switch, or compound bypass, method to be described reduces this remaining problem as well.

“We have recently demonstrated that extracellular mitochondria are abundant in the circulation of deceased organ donors and that their presence correlates with early allograft dysfunction” (Lin, L., Xu, H., Bishawi, M., Feng, F., Samy, K., and 4 others 2019. “Circulating Mitochondria in Organ Donors Promote Allograft Rejection,” American Journal of Transplantation 19(7):1917-1929). Accordingly, initiating switch transplantation immediately upon death avoids an increase in donor blood factors inimical to success.

In apparent upon gross inspection, this microscopic level of degeneration will not be avoided much less reversed by chilled storage, and whether ‘normal’ reperfusion and revival of living cells will allow clearance of this microdebris likely depends upon its prevalence and the relative proportion of cells capable of recovering to initiate its clearup, which argues in favor of hematopoietic cell therapy. Once this has been allowed to happen, the organ has already become severely degraded for transplantation, already broken down before the final insult—placement in an alien, if allogeneic milieu. Often the donor is discovered having already died and a long waiting list forces the staff to make the most of the organs available. Then, improvements in preservation strategies are of incontestable value. Ordinarily, when a prospective donor dies, blood is cut off from all of the donor's organs.

While anatomically distinguishable, solid organs are not properly viewed as separate from the rest of the body. Ex vivo perfusion machines facilitate targeted therapy of the graft organ through the crude method of having excised and thus isolated the organ from its physiological context. When intact in its normal situation, the chemical and mechanical interactions of the organ with the rest of the body are multifarious and many, completely interconnected as would interact with the therapy in ways numerous and complex. To then transplant the organ resituates it in the equivalent milieu with no reduction in the complexity of contextual function but with the added complication of immunological alteration.

In contrast to therapy with a perfusion machine, metered switch transplantation allows the incorporation of graft organ-targeted therapy while the organ remains in situ within the donor sustained on life support and during transfer from the circulatory system of the donor to that of the recipient. It accomplishes this, moreover, with no breach in bodily connections throughout this unprecedented transition—that is, with no interruption in perfusion, respiration, or other life functions despite having been resituated from one body to another. By continuously sustaining all functions of the graft organ, first while remaining in the donor, then, albeit partially denervated (Choudhury, M. 2017. “Post-cardiac Transplant Recipient: Implications for Anaesthesia,” Indian J Anaesthesia 61(9):768-774) but with intact vascular connections in the recipient, the considerable trauma associated with death, organ preservation, and conventional transplantation are avoided.

In addition to eliminating numerous insults associated with conventional transplantation that contribute to graft failure, metered switch transplantation inherently accomplishes a measure of immune tolerance induction in the interval during which the organ is transferred from the donor to the recipient. Furthermore, because the accessory channels of the diversion chute servovalves used to connect the native organ of the recipient to the graft organ while it remains in vivo in the donor allow the direct targeting of agents to either or both hearts during the transfer, the application of concurrent therapy, pharmaceutical or genetic can be used to restore the graft organ while it is being resituated in the recipient.

The immune tolerance induction accomplished concurrently with transfer to the recipient of the graft organ may allow a reduction in the dosages of tolerance induction drugs such as antithymocyte globulin and dendritic cells, which risk numerous disturbing side effects, to include fever, chills, aphthous ulcers, malaise, tachycardia, and dyspnea. Basiliximab risks tremor, fever, chills, diarrhea, and vomiting. While drugs, regulatory T cells, donor bone marrow, and stem cells can also be integrated into the process, organ transplantation using the metered switch method should accomplish a measure of blood-induced immune tolerance induction as an inherent byproduct of the reciprocal cross-circulation that mediates transfer of the organ from the donor to the recipient.

This factor likely improves the prospects for kidneys and livers more than lungs and hearts, the latter tending to resist tolerance induction. However, with the incorporation of gene therapy (see Bishawi, M., Roan, J. N., Milano, C. A., Daneshmand, M. A., Schroder, J. N., and 10 others 2019. “A Normothermic ex Vivo Organ Perfusion Delivery Method for Cardiac Transplantation Gene Therapy,” Scientific Reports 9(1):8029) both to augment microchimerization and aid restoration, and with the further addition of the following and agents specified below, the prospects for lungs and hearts should also improve. Additives to the perfusate using conventional means for graft organ preservation, such as ex vivo perfusion, here pertain to addition of the agent into either bloodstream in the shared circulation that mediates the metered switch.

a. Chimerizing hematopoietic stem cell therapy.
b. Restorative stem cell therapy, to restore a donor heart if necessary.
c. Gene silencing. Another approach to maximize tolerance induction before sudden switch and during and after metered switch transplantation is gene silencing with siRNA. That the conventional object is to reduce ischemia-reperfusion injury following organ storage, and this has already been eliminated in switch transplantation, treatment thus may also improve the odds for avoiding the development of post-transplantation heart vasculopathy.

What is true for a heart transplant is likely no less true for other organ transplants: the avoidance of oxygen deprivation and an adverse immune response before and during transplantation, which switch transplantation provides, and controlling comorbidities after transplantation, which an implanted disorder response system provides, will improve the prospects for longer eventual survival of any organ transplant (see, for example, Braun, A. T. and Merlo, C. A. 2011. “Cystic Fibrosis Lung Transplantation,” Current Opinion in Pulmonary Medicine 17(6):467-472).

The complete avoidance of preservation, hence, preservation injury, obtained by directly transferring the graft organ from the circulatory system of the donor into that of the recipient materially if not entirely eliminates breakdown products of injured and dying donor cells the result of prolonged ischemia during storage, such cells a prime target of recipient immune cells. Moreover, that the donor is supported in oxygen delivery while still alive means that fewer such cells result from an hypoxia of the donor prior to death.

Pretransplantation cellular breakdown contributes to graft rejection both endothelial to include even late term cardiac allograft vasculopathy and “In the parenchyma, complement fixation initiates the complement cascade, culminating in the formation of a membrane attack complex,” and this breakdown in the cell membranes opens the cells to osmotic injury (Rohrer, R. J. 2002. Basic Immunology for Surgeons,” in O'Leary, J. P. (ed.), The Physiologic Basis of Surgery, Philadelphia, Pa.: Lippincott Williams and Wilkins).

d. Supplemental hydrogen sulfide (sulphide).

The main objective in tolerance induction is to reduce if not eliminate the need for lifelong immunosuppression. Another factor in switch transplantation, the use of an implanted automatic disorder response system to directly pipe-target immunosuppressants to the transplant, sparing exposure of the rest of the body, can compensate for a shortcoming in tolerance induction and increased susceptibility to infection. Addressed below, another implicated sequela, cardiac allograft vasculopathy, responsible for half of failed heart transplants, is likely due, at least in part, to ischemia during preservation.

Both sudden and metered switch heart transplantation are extracardiac, eliminating the risk of problem bleeding and the extensive dissection of both donor and recipient hearts which is an integral part of conventional methods of heart transplantation. While the benefits of avoiding surgical trauma in the transition from the original biatrial to the bicaval technique are well documented, the additional benefits in continuing the pattern of retreating from the heart entirely during heart transplantation has no precedent in human medical practice, and while animal experiments are documented, no such operations are recorded in the literature.

That neither donor nor recipient hearts are entered eliminates the risks associated with cardioplegia, cross-clamping, general anesthesia, cardiopulmonary bypass, ischemia-reperfusion injury, and infection, should reduce the incidence of post-transplantation vasculopathy, and as addressed herein, given that immunosuppressants and tolerance induction drugs are directly pipe-targeted to the transplant, should reduce de novo post-transplantation cancer—that is, reduce or eliminate the complications known to limit transplant and recipient longevity. The elimination of cardiopulmonary bypass may be more significant than just the avoidance of postoperative cognitive deficits.

The avoidance of these complications depends upon the avoidance of the biochemical upheaval associated with death, the abrupt loss of perfusion, anoxia, organ harvesting, preservation, and insertion into an alien milieu. The initiation before and sustainment through and following brainstem death and the switch method allow these eventualities, ruinous soon and/or posing the risk of vasculopathy and/or cancer. Except where an interminably comatose patient must be removed from life support to effectuate death, it is a key error to turn off life support and then harvest the organs needed. In fact, as will be described and made clear, so that there is no interruption in oxygenation and metabolic function, organs for transplant should not be harvested until having been already been placed in the recipient. Transplantation without interruption in blood flow requires and is dependent upon access to vascular valves and servovalves.

The metered switch method effectively achieves intrinsic and concurrent in vivo graft organ/recipient immunosuppression, immune tolerance induction, and a measure of cross-microchimerization through reciprocal cross-circulation during organ transfer as well as the transfer of donor-derived passenger leukocytes to the recipient, and to the cross-chimerization inherent in reciprocal cross-circulation, allows the incorporation of gene and stem cell therapy for both increased tolerance induction and graft organ restoration superior to that obtained with ex vivo perfusion with its numerous detractions in exchange for this one advantage.

Always a ‘two-edged sword,’ the introduction of allogeneic protein can provoke an immune reaction, even intense, or effect a measure of immune tolerance, even chimerization, thus facilitating the acceptance of an allogeneic graft organ. As with immunosuppression by any means, an object is to blunt if not avoid an initial reaction of outright and immediate recoil and educe instead an initial accommodation. In mollifying an adverse reaction, eventual acceptance should be facilitated, and in a heart transplant, the odds favoring eventual failure due to allograft vasculopathy should be reduced.

In a metered switch transplantation, the transfer of the graft organ from the donor to the recipient is initiated by admitting a minute volume of recipient blood into the circulation of the donor. Implanted sensors detect signs or biomarkers of rejection by the recipient or an adverse immune reaction by the donor that would negatively affect the other organs. If none, then the process is continued, very gradually increasing the relative proportion of recipient over donor blood until the blood circulating through both is half that of the other. At any point in the process, the valves can are automatically adjusted to cut off entirely, or gradually reduce, or increase the relative volume of blood exchanged.

An adverse reaction is responded to by infusing immunosuppressants, and once suppressed, the process is continued. The treatment of problem bleeding if any following the procedure is dealt with by conventional means (Kunadian, V., Zorkun, C., Gibson, W. J., Nethala, N., Harrigan, C., and 10 others 2009. “Transfusion Associated Microchimerism: A Heretofore Little-recognized Complication following Transfusion,” Journal of Thrombosis and Thrombolysis 27(1):57-67).

Transfusion associated microchimerization has been long recognized, and the cross-chimerization inherent in reciprocal cross-circulation, allows the incorporation of gene and stem cell therapy for both increased tolerance induction and graft organ restoration superior to that obtained with ex vivo perfusion with its numerous detractions in exchange for this one advantage. Rather than isolated from the rest of the body in a perfusion machine, the organ remains in its physiological situation, and following this procedure, the implanted automatic response system can continue this treatment. If uptake of the agent by the target organ is incomplete and the agent would best be kept from nontargeted tissue, the accessory channels in the valve jackets at the venous drainage of the organ can be used to release a reversal agent or if necessary, the agent can be formulated for magnetic extraction.

Radioactive agents can be targeted to the organ and if necessary, extracted, through shielded lines and connectors. As shown in copending application Ser. No. 15/998,002 entitled Ductus Side-entry Jackets and Prosthetic Disorder Response Systems, such a side-entry jacket modified to serve as a vascular valve can incorporate a layer of permanent, preferably neodymium, magnets magnetized to project an attractive field radially toward the central axis of the substrate ductus, one object in incorporating a magnetized layer being to facilitate the uptake of superparamagnetic micro- and nanoparticle carrier bound drugs into the intima or comparable layer in ductus other than an artery. Such valves can also incorporate different type sensors to detect various physiological indicia, the positioning of these dependent upon the index to be measured.

This cutoff from oxygen, nutrients, electrolytes, enzymes, hormones, neurochemical and nervous connections, and metabolites known and possibly remaining to be discovered initiates a cascade of degenerative changes that will culminate in autophagy. The graft organ is transplanted not just with its allogeneic genome and its individual differences but also with immune system-produced irreversible degradation products, dead cells, cells losing or having lost membrane permeability, and likely, other cellular level toxins associated with the death of the organism and cell death. The degrees to which post-death measures can revive this tissue and to which harvesting adds further damage depend upon the time elapsed since death.

Recipient-donor cross chimerization is a central factor in metered switch transplantation. It warrants emphasis that, acknowledging the uncontrollability of donor organ procurement in most cases, whenever possible, the direct interconnection of the circulatory systems of recipient and donor through the native and graft organs prior to harvesting is fundamentally superior to any alternative, whether machine perfusion, cold storage, special organ transport support machines, normothermal, cryothermal, or any other alternative.

When refrigerated and/or preserved in a static perfusate, the deterioration of the graft organ can be slowed down if not stabilized at the level to which it had progressed, and unless the time of this stasis is relatively brief and aggressive measures are applied and succeed, it is in this condition that the graft organ is placed in a recipient. Ex vivo perfusion machines afford an opportunity to apply therapy and ameliorate but not eliminate the problem. Metered switch transplantation can continue long enough to achieve a measure of immune tolerance induction as well as meet or exceed the repair obtained using ex vivo lung perfusion. Caustically, to the molecular and cellular level degradation products of death, reperfusion injury then adds further obstacles to the success of transplantation.

As currently practiced, the two major obstacles to successful transplantation are, first, immune tolerance aside, the need to optimize the condition of the graft organ, usually taken from a recently deceased patient, and second, the need to optimize the genetic compatibility mutuating between graft and recipient. While the ability to avoid rather than have to secondarily ameliorate this posthumous deterioration would fundamentally elevate the odds for successful outcomes, the standard practice remains harvesting the organ and then preserving it with or without remediation of both deteriorative and immunological obstacles. Provided the time since the terminal event will allow, new strategies currently under study, to include exogenous stem cell therapy, do appear to offer some degree of recoverability. Were these methods fully successful, the problem of optimizing organs that become available only following death—currently the rule—would be substantially reduced.

Emerging practices, notably stem cell therapy, readily integrated into switch transplantation along with immunosuppressants, do appear to have the potential both to reverse the cellular damage of death as well as initiate immune tolerance, so that the pursuit of both objectives proceeds simultaneously in parallel, and moreover, appears to protect against cold ischemia-reperfusion injury. Autologous stem cells aid healing, while allogeneic stem cells can induce immune tolerance as well as aid healing.

However, when the patient dies having consented, ex vivo preservation should be replaced with in vivo sustainment. Death and preservation severely degrading the viability and transplantabity of graft organs, when a patient is terminal in the center, the body can be sustained in a nearly optimal condition for the organs to be transplanted before the patient finally dies (see, for example, (Shetty, V. L., Mali, S. S., Shetty, S. V., and Shinde, P. D. 2017. “The Brain-dead Donor: An Anaesthesiologist's Perspective,” Indian Journal of Anaesthesia 61(12):952-956). Once the patient on life support has descended into an irrecoverable depth of coma at which death is certain, the graft organs should be transferred into the recipients.

Life support should never be shut off and the patient allowed to finally die before the organs are taken. That the switch method continuously sustains perfusion and oxygenation throughout the transfer of the graft organ from the donor to the recipient—with a gradual transition to the blood of the recipient in a metered switch—is of central benefit in the avoidance of ischemia-reperfusion injury and most likely post-transplantation vasculopathy, which is the cause for the failure in large percentage of heart transplants.

Furthermore, the switch methods leave intact and therefore impose much less trauma on graft organs than do conventional procedures. The objective sought are to isolate graft organs from the chemical consequences of a cessation in perfusion and oxygenation associated with death, and transfer the organs into recipients using a continuous blood exchange process that gradually transitions the organ from the circulatory system of the donor into that of the recipient. Simply stated, an effort is made to conceal from graft organs the death of the host and transfer into the circulatory system of another. This is enhanced in metered switch transplantation which seeks to accomplish immune tolerance induction as an integral component of the continuous blood transfer.

By suddenly transferring the donor, or graft, organ from the circulatory system of the donor into that of the recipient without interruption in perfusion, and if a lung, without interruption in spontaneous or natural breathing, this technique reduces the causes for graft failure other than immune intolerance. Metered switch transplantation affects the transfer of the graft organ between the circulatory systems gradually, so that the interval over which recipient and donor are exposed to one another's blood is extended. Perfusion of the graft organ with the blood of the recipient gradually increases in relative proportion until the organ is transferred to the recipient.

This process of reciprocal cross-circulation where the blood of the recipient and donor gradually blend provides a measure of immune tolerance induction, thus addressing the remaining obstacle to success in transplantation. After transplantation, the implanted automatic disorder response system will continue the administration of immunosuppressants, but in a directly targeted manner that does not immunocompromise the patient. Minimization in the ability of the graft organ to recognize its alien new milieu and of the recipient to recognize the fact of an alien intrusion allows transplantation which if not completely adverse reaction-free, is at least as unprovocative and uneventful as might reasonably be accomplished.

While delivered to the donor organ as a constituent in the blood of the recipient—which does necessitate larger dosing to compensate for the dilution factor—the recipient-donor reciprocal cross-chimerization achieved by reciprocal cross-circulation in metered switch transplantation inherently targets the gene transfer vector directly to the graft organ of the recipient, augmenting the chimerization provided by reciprocal cross-circulation alone. The removal of plasma or serum from the blood of the donor, which has been found to interfere with transgene uptake is easily accomplished along the mainlines or bloodlines joining donor and recipient (Bishawi, M., Roan, J. N., Milano, C. A., Daneshmand, M. A., Schroder, J. N., and 10 others 2019, Op cit., in this section).

Delivery essentially tracks Bishawi et al. but is adapted to the in vivo arrangement used in metered switch transplantation rather than the ex vivo arrangement imposed by use of the TransMedics Incorporated, Andover, Mass., Organ Care System.™ Where ex vivo heart and lung perfusion machines seek to minimize ischemia, switch transplantation avoids ischemia altogether, but does require that the bodily systems of the donor be sustained past death and the recipient be positioned alongside the donor.

The donor and recipient are positioned to afford the smallest distance between the greatest number of vessels to be connected, the organs involved aligned in adjacent relation. Except when to connect all the vessels would not necessitate eventually having to rotate either the donor or recipient, the relatively small differences in distance between like vessels of paired and unpaired organs due to chirality with donor and recipient uninverted, with both heads up or inverted, with one head up and the other down are discounted. Unless significant, differences in organ to organ distance should be disregarded. Where ex vivo perfusion machines require that the donor heart be removed for pretransplantation gene therapy, metered switch transplantation effects gene therapy as integrated into the reciprocal cross-circulation and reciprocal preliminary cross-chimerization between donor and recipient.

Ex vivo graft hearts may be maintained in an anoxic state for hours prior to machine perfusion, all the while accumulating constituents in the blood inimical to a successful outcome. Metered switch transplantation can progress for a period considerably longer than the two hours used by Bishawi, et al. As well as to supplement the chimerizing effect of reciprocal cross-circulation, gene therapy incorporated into metered switch transplantation can also be used, alone or with stem cells and drugs, to restore a subcritically failing donor heart, lung, or other solid organ, thus further increasing the pool of organs available for transplant, and while ex vivo perfusion machines also allows this, the numerous detractions attendant upon such use, of which none pertain to metered switch transplantation, make the latter preferable for such treatment.

Outside of transplantation, in vivo organ isolation and perfusion as in isolated hepatic perfusion is likewise superior to systemic treatment. The use of ductus side-entry diversion chute servovalves and nonjacketing side-entry connectors in a totally implanted system as described in copending application Ser. No. 15/998,002 entitled Ductus Side-entry Jackets and Prosthetic Disorder Response Systems would, however, provide more dependable balloon catheter and leak complication-free outcomes. Flowrate controllable connections for the catheters, controllable pressures, and the targeting of supplemental or adjuvant agents to the same organ through direct control by the implanted controller of source-outlet pump settings, or through diversion servovalves or through upstream basic side-entry jacket accessory channels provides an additional layer of control.

The use of these components would allow isolated perfusion of the liver, substantially if not entirely in isolation, conventionally used to target stem cell and chemotherapy to the liver but readily adaptable to the targeting of restorative, and in transplantation, chimerizing gene therapy, as well as the targeted administration of tumor necrosis factor, to continue over an indefinite period. Thus, taking these various factors into consideration, application to metered switch transplantation involves a fundamental improvement in changing the process from ex- to in vivo, of which the advantages include the elimination of pretransplantation organ harvesting and placement, eliminating ischemia and reperfusion injury, along with the cardioplegia, general anesthesia, extensive incision into both donor and recipient hearts, and so on, all potentially injurious and central in conventional heart transplantation.

This will prove advantageous in the ability to not only augment the reciprocal cross-chimerization of the donor and recipient to facilitate solid organ acceptance but allow the transplantation preliminary and/or concurrent application of gene therapy as well as chimerization to increase the pool of available hearts by allowing the use of those subcritically failing for double heart transplantation, primarily in the elderly. The process of gene transfer as incorporated into and concurrent with donor-recipient reciprocal cross-chimerization and physical transfer of the graft organ during reciprocal cross-circulation with the added advantage of gene therapy is no less applicable to the transplantation using the switch method of other solid organs. Also integrable is the introduction of agents such as thymosin β4, or timbetasin, which can be added to increase the plasma concentration of the donor-recipient shared bloodstream during switch transplantation to reduce inflammation and facilitate graft survival. Accordingly, the subject is addressed below.

Furtherance in the dependability and efficacy as well as a reduction in the complexity of organ transplantation can be expected to encourage the abandonment of less effective, or ‘half-way,’ medical procedures. For example, perivascular stent jackets and stay based stents as described in copending application Ser. No. 15/932,172 require placement through a small incision, making this an invasive procedure, while the transcatheteric placement of conventional stents is minimally invasive; however, the invasive factor is more than compensated for by certain distinct advantages. For another example pertinent to organ transplantation, transcatheteric repair of the severely malformed heart in utero, open surgical repair in the neonate or infant, and conventional replacement of the severely malformed heart usually save the life of the baby, but unless normal circulation ensues, results of the procedure over the long term prove disappointing.

By contrast, orthotopic heart transplantation whether conventional or by means of the switch technique described herein can provide pulsatant rather than nonpulsatant or passive Fontan pulmonary arterial flow with sufficient oxygenation, thereby allowing normal development. Absent pulmonary vascular resistance, some transient left ventricular weakness should not occur. If it does, then the weakness should respond to the direct targeting to the ventricular myocardia of one or more positive inotropic agents, such as dopexamine, angiotensin II, a prostaglandin, or enoxomone, alone or in a special formulation, over a duration sufficient to allow it to strengthen.

If positive inotropes would aggravate the underlying disease process, then vasoactive and neurohumoral agents are directly pipe-targeted through a basic ductus side-entry jacket placed about the left anterior descending coronary artery in the manner described in copending application Ser. No. 14/998,495, entitled Ductus Side-entry Jackets and Prosthetic Disorder Response Systems. The term ‘ejection fraction’ rather than ‘absolute stroke volume’ has been criticized as unindicative of the type of myopathy (Konstam, M. A. and Abboud, F. M. 2017. “Ejection Fraction: Misunderstood and Overrated (Changing the Paradigm in Categorizing Heart Failure)” Circulation 135(8):717-719; Cikes, M and Solomon, S. D. 2016. “Beyond Ejection Fraction: An Integrative Approach for Assessment of Cardiac Structure and Function in Heart Failure,” European Heart Journal 37(21):1642-1650).

Accordingly, a new dependability, facility, and relative simplicity should result in an increase in the number of individuals amenable to receiving a transplant and the number of medical professionals able to perform such operations. It follows that the availability of transplantation would reasonably be distributed from the largest centers to community hospitals. Here solid organ transplantation with zero ischemia time is implemented by means of a compound bypass of the native organ through intravascular diversion servovalves and bloodlines the same in caliber as the vessels to which each is attached. The bypass transfers the organ from the circulatory system of the donor to that of the recipient. When the condition of the patient is urgent, the switch from recipient to donor heart is immediate, supporting therapy then accomplished following transplantation.

When carried out gradually as described below under 1b(1)(b), the procedure affords a measure of immune tolerance induction which also allows gene and stem cell therapy to initiate the restoration of a less than ideal replacement heart. Implanted suddenly, a replacement heart for sudden switch transplantation must be less impaired than one meter switched, where time can be taken to treat nondisqualifying deficits. In sudden switch orthotopic heart transplantation, solenoid operated diversion valves are switched simultaneously from full flow through the native to full flow through the graft organ by actuation from the same plunger switch by the operator or an assistant. In metered switch orthotopic heart transplantation, this transfer of flow is also complete but accomplished gradually.

In metered switch transplantation, small diversion servovalves driven by tiny servomotors are coordinated by the implanted microcontroller in substantially single morbidity disease or master control microprocessor in comorbid disease. Until adequate remedial measures have been developed, hearts for switch transplantation with marginal heart failure must be of the ejection fraction reduced, not preserved kind. The microprocessor executes a prescription-program that is written to respond to the various contingencies that might arise during the operation.

Addressed below, double heart switch transplantation is always metered, and the end-point requiring flow through either heart in proportion to its ejection fraction, the valves are stopped around the half way point. As in conventional orthotopic heart transplantation, the replacement heart will usually be left partially denervated; however, where pain sensation is desired, the positioning of a ductus side-entry jacket or diversion servovalve at or about the anastomosis between severed ends of the vagus will allow the direct delivery to the site of immunosuppressive and other medication, as well as stem cells, to facilitate knitting, or coaptation, between donor and recipient stumps.

In almost half of cases, the process of reinnervation can be confirmed as having begun by a year post-transplantation. “In conclusion, partial sympathetic reinnervation increases with time after HT [heart transplantation]; it was seen in 40% of patients at 1 year after the operation.” (Buendia-Fuentes, F., Almenar, L., Ruiz, C., Vercher, J. L., Sánchez-Lázaro, I., and 4 others 2011. “Sympathetic Reinnervation 1 Year after Heart Transplantation, Assessed Using Iodine-123 Metaiodobenzylguanidine Imaging,” Transplantation Proceedings 43(6):2247-2248) (but see also Lee, S. R., Kang, D. Y., Cho, Y., Cho, H. J., Lee, H. Y., Choi, E. K., and Oh, S. 2016. “Early Parasympathetic Reinnervation is Not Related to Reconnection of Major Branches of the Vagus Nerve after Heart Transplantation,” Korean Circulation Journal 46(2):197-206).

The vagus and superior cervical and inferior or thoracic cardiac branches of the vagus nerve, technically cranial, have been observed to eventually fuse following heart transplantation. The modulation of the heartbeat, much less its communication with the rest of the body through the bloodstream, however, is not exclusively confined to the parasympathetic fibers of the vagus and the sympathetic fibers of the accelerans nerve. Nevertheless, by definition, the reinstatement of normal sensation that would allow the patient to become aware of a cardiac or coronary malfunction is to be preferred. Adjustment of cardiac function responsive to orthostatic hypotension upon rising from a reclined to an upright position is less serious but can result in syncope, or the loss of consciousness.

For now, fusion, or coaptation, of peripheral nerves is possible, although with the exception of the long term fusion of the vagus following heart transplantation, central nervous tissue, in particular, the optic cranial nerves to allow whole-eye transplantation, remains elusive (Canavero, S. and Ren, X. 2019. “Advancing the Technology for Head Transplants: From Immunology to Peripheral Nerve Fusion,” Surgical Neurology International 10:240). For a double heart transplant, there is no normal precedent. In double heart switch transplantation to add a second heart, that native will remain fully innervated while that added will be fully denervated in relation to the new body.

In double heart switch transplantation that involves replacing the native as well as adding a second heart, the orthotopically positioned heart will be partially, and that heterotopically positioned, entirely denervated with no corresponding nerve to allow reconnection in the recipient. Nervous recovery of the orthotopically transplanted heart takes about five years, but a second heart must be positioned heterotopically (see, for example, Stover, E. P. and Siegel, L. C. 1995. “Physiology of the Transplanted Heart,” International Anesthesiology Clinics 33(2):11-20; Gaer, J. 1992. “Physiological Consequences of Complete Cardiac Denervation,” British Journal of Hospital Medicine 48(5):220-225). Whether a second heart placed to function much as a living assist device need respond to stimuli such as exertional or emotional when the native heart remains neurologically and endocrinologically integrated for such response warrants study.

The unclear status in neurological recovery of even a patient with one heart conventionally transplanted orthotopically complicates the application of anesthesia in various surgical procedures before the question of a second heart even arises. For double heart transplantation, the answers must await the accumulation of experience.

“Mechanical ventilation can initiate ventilator-associated lung injury (VALI) and contribute to the development of multiple organ dysfunction. Although a lung protective strategy limiting both tidal volume and plateau pressure reduces VALI, uneven intrapulmonary gas distribution is still capable of increasing regional stress and strain, especially in non-homogeneous lungs, such as during acute respiratory distress syndrome. Real-time monitoring of regional ventilation may prevent inhomogeneous ventilation, leading to a reduction in VALI.” (Shono, A. and Kotani, T. 2019. “Clinical Implication of Monitoring Regional Ventilation Using Electrical Impedance Tomography,” Journal of Intensive Care (BioMed Central, London, England) 7:4).

Used inconsistently in the literature, here the terms ‘spontaneous ventilation,’ ‘spontaneous respiration,’ and spontaneous breathing are used to denote normal unassisted breathing. Usually ‘spontaneous ventilation’ under general anesthesia does involve mechanical assistance, while the same absent general anesthesia does not. Switch transplantation, for example, usually eliminates the need for general anesthesia, and use of a surgical chestdome as described herein is intended to enable spontaneous breathing with general anesthesia eliminated.

A valid need for mechanical ventilatory assistance independently of—that is, during or not during—general anesthesia however, is not discounted (Ball, L., Costantino, F., Fiorito, M., Amodio, S., and Pelosi, P. 2018. “Respiratory Mechanics during General Anaesthesia,” Annals of Translational Medicine 6(19):379). Although seldom allowed, “Spontaneous ventilation [here denoting self-initiated breathing with mechanical assistance] during general anesthesia has been shown to favour atelectasis formation and decreased functional residual capacity,” more recent developments having dispelled this problem (Magnusson, L. 2010. “Role of Spontaneous and Assisted Ventilation during General Anaesthesia,” Best Practice and Research. Clinical Anaesthesiology 24(2):243-252). Most often, genuinely spontaneous, or normal, breathing during surgery is possible and is associated with the avoidance of adverse complications. Normal breathing is less susceptible to an adverse distribution of ventilation within the lungs.

Similar questions arise in relation to perioperative anesthetic management following transplantation of other organs, all transplantable using the switch method, often with the aid of a surgical chestdome as described below to avoid a remaining source of complications in the form of mechanical rather than spontaneous respiration (normal breathing, normal ventilation).

In double heart switch transplantation, the prescription-program will continue the advancement of the diversion servovalves until the implanted cardiac rhythm sensors indicate the inception of a dysrhythmia, which the program will have been written to reverse through electrical, temperature, pharmaceutical, and it can be predicted, optogenetic means (Guru, A., Post, R. I., Ho, Y. Y., and Warden, M. R. 2015. “Making Sense of Optogenetics,” International Journal of Neuropsychopharmacology 18(11):pyv079).

Once the native organ is removed, the graft organ is placed in the recipient with the valves and bloodlines connected to the graft organ and to the implanted microcontroller which had administered the operation. Thereafter, the controller, provided with data from implanted sensors, monitors and is able to target medication to the transplant from implanted small flat drug reservoirs connected to accessory channels in each valve jacket, can adjust the valves to regulate or fine tune the blood pressure, and can apply electrostimulation in accordance with the controller prescription-program. Without accessory channels, common catheteric materials used as mainlines or bloodlines would be susceptible to the buildup of thrombus and/or biofilm to pose the risk of infection.

This automatic post-transplantation maintenance, which can instantly provide an immunosuppressive or anti-inflammatory, for example, can not only prevent graft organ failure but make possible organ transplants not previously considered, alleviating the shortage of replacement organs as but one of several benefits. With respect to long term synthetic-to-native tissue ductus junctions whether end-to-end, end-to-side anastomotic, or those involving synthetic grafts such as a Dacron® patch on the bladder, in a Dor endoventricular circular patch plasty on the left ventricle, or in a vascular bypass such as femorofemoral shunt, for example, absent immediately responsive protective measures, adverse tissue reactions, infection, and the accumulation of clot often prove problematic.

The ability afforded by the accessory channels in ductus side-entry jackets or nonjacketing side-entry connectors to target anti-inflammatory, antithrombotic, and antimicrobial medication to these junctions is a powerful deterrent to junction failure. The thrombotic, infection, and adverse tissue reaction problems associated with Dacron® and similar synthetic textiles in direct contact with tissue can be significantly ameliorated if not eliminated. Patches and shunts, especially if supported as indicated, are effective for repairing defects in the circulatory system that would otherwise be fatal for at least a few years, making use in the elderly satisfactory.

Prevention of another junction-related complication, perigraft seroma, is by conventional methods for eliminating spaces where fluid can accumulate (Halloul, Z., Rimpler, H., Waliszewski, M., Beier, N., Meyer, F., ad 3 others 2014. “First-in-Man Results of a Novel Vascular Graft Coated with Resorbable Polymer for Aortic Reconstructions—a Multicenter, Non-randomized Safety Study,” Langenbeck's Archives of Surgery 399(5):629-638). Yet other junctional or interface contact-related problems result from the use of polymer surface coatings on catheters and other implant devices; such problems are identified with intracranial procedures where access is difficult.

In some cases, particles of the coating shed due to sliding contact during the insertion process result in an inflammatory response, arterial pathology, or embolism, for example, long after the procedure when the coated insertion device had been withdrawn. While in extracranial applications, immunomodulatory therapy with mycophenolate, azathioprine, and steroids can be targeted to the site through the accessory channels, these too can produce serious side effects, leaving the problem best avoided by not using polymer coatings such as polyvinylpyrrolidone, mostly if not all hydrophilic, implicated thus in the first place.

No such coatings, which rub off the substrate during insertion to remain behind as an irritant, are applied to side-entry jackets, nonjacketing side-entry connectors, side-entry diversion valves, diversion servovalves, chokevalves, or servo chokevalves. The coatings, applied to obtain a slippery surface to avoid intravascular clinging, would best be replaced with a sputter deposited fluoropolymer, for example. However, because the nonliving, static material is incapable of the functional, restorative, and adaptive attributes of living vascular or bladder tissue, it is unable to reverse a loss in tautness, for example, and is subject to deterioration over the long term, so that problems unrelated to the synthetic-native junction continue to interfere with its sufficiency for use in the young (see, for example, Singh, C., Wong, C. S., and Wang, X. 2015. “Medical Textiles as Vascular Implants and Their Success to Mimic Natural Arteries,” Journal of Functional Biomaterials 6(3):500-525;

Ductus side-entry jackets, nonjacketing side-entry connectors, diversion jackets used as intravascular servovalves, and servochokes incorporate features specifically intended to overcome the factors that historically precluded the permanent implantation of catheters and smaller gauge tubing of a material such as Dacron® to move blood or urine. Accessory channels giving access through a small port at the body surface are provided not only to allow the direct targeting to the treatment site of medication, but to wet the interior surface of lines with maintenance materials to counteract thrombus in the vascular tree, crystallization in the urinary tract, and the risk of biofilm and infection.

Whereas a diversion jacket redirects all flow through a vessel, ureter, spermatic duct, fallopian tube, glandular duct, or other tubular anatomical structure, the diversion of arterial blood usually seeks to redirect only a fraction of the blood moving through the artery to perfuse an under-oxygenated region or organ. Partial diversion is accomplished with a conventional side-entry jacket used to create a branch of the main vessel. To prevent the buildup of thrombus, crystallization, and/or biofilm as deterrents to the use on a permanent, fully implanted, basis of synthetic materials and smaller caliber catheters in particular, a special port at the body surface having openings to allow the direct delivery through the jacket or connector accessory channel or channels into the synthetic lumen, side-entry jacket, and native lumen of coagulation, crystallization, and biofilm forming counteractants as necessary are injected into the small flat drug reservoir or reservoirs implanted subcutaneously in the pectoral region.

The permanent implantation of catheters by the means described herein with subcutaneously positioned body surface port which can be made unnoticeable, allows the use of larger caliber catheters than does brachial, jugular, subclavian, and even vena cava insertion, for example (Fonseca, I. Y., Krutman, M., Nishinari, K., Yazbek, G., Teivelis, M. P., Bomfim, G. A., Cavalcante, R. N., and Wolosker, N. 2016. “Brachial Insertion of Fully Implantable Venous Catheters for Chemotherapy: Complications and Quality of Life Assessment in 35 Patients,” Einstein (Sao Paulo, Brazil) 14(4):473-479).

Furthermore, the connection to the ductus of the catheter through the side-entry jacket is accomplished without endoluminal, or intravascular, entry, and the type of port described here is less susceptible to complications, to include infection, fracture, or migration. The minuscule side plug extraction method for gaining endoluminal entry into the substrate ductus is less susceptible to complications such as the inducement of clotting or infection, than alternative methods.

For example, two hearts with marginal ventricular absolute stroke volumes (ejection fractions) were these measured while the donor heart also remained in its respective circulatory system (Konstam, M. A. and Abboud, F. M. 2017, Op cit.) would fail to meet the current criteria for transplantation. However, using metered double heart transplantation as addressed below can be coordinated to approximate the function of a normal heart, and if the need is detected by the implanted response system sensors, either or both hearts can be directly pipe-targeted supportive medication and/or electrostimulation postoperatively for as long as necessary.

Evaluating the potential for reverse remodeling and recovery of a marginally impaired replacement heart once transferred from the circulatory system of the deceased prospective donor on life support into that of the recipient must be based upon the detailed pathology of both and the graft organ. Such an evaluation draws upon all aspects of cardiology and much of internal medicine. To this must be added the complexity of weighing these factors against the relative merits and risks posed by conventional and metered switch orthotopic transplantation or a double heart transplantation whereby the graft is implanted as a second heart.

The donor, graft organ, recipient, and probable consequences of different courses of action must be taken into account. Then post-transplantation care requires detailed familiarity with cardiology and cardiological pharmacotherapeutics, and if a need therefor had not been realized in advance, this must include the use of various ventricular assist devices and/or implanted direct stimulatory means both as mentioned above and/or in the form of conventional pacemaker/cardioverter defibrillators in relation to the detailed condition of the recipient. Comorbidity if not intercurrent disease is likely to further complicate matters, potentially demanding a knowledge of almost any aspect of internal medicine.

Cardiac remodeling following a myocardial infarction is not unique to reduced ejection fraction; remodeling can lead to reduced, midrange, or preserved ejection fraction. Neither is pulmonary hypertension exclusively associated with remodeling of the right or left ventricle or are cause or effect necessarily clear as to right or left heart dysfunction More generally, in heart failure, the major features—premonitory remodeling, right or left heart dysfunction, reduced, midrange, or preserved ejection fraction, right or left ventricular hypertension, and so on, present in all combinations to present as independent variables. Furthermore, the interrelations of each type disorder to the other organs comprises a considerable profusion of combinations and permutations. Hearts that had undergone a myocardial insult, typically, a myocardial infarction, likely to induce remodeling leading to heart failure can be evaluated for such a prognosis. Means available and under development exist to monitor and suppress adverse remodeling in heart failure with reduced ejection fraction.

Ventricular assist devices, conventional, and double heart heterotopic transplantation each have advantages and disadvantages. Vascular assist devices risk coagulopathy but involve the least surgical trauma to place. Conventional and double heart heterotopic transplantation should not cause a coagulophathy, but both require open chest surgery, will never be endoscopic, and the conventional heterotopic technique inflicts considerable surgical trauma upon both the recipient and donor hearts. The reversal of pathology-adaptive remodeling with the use of a ventricular assist device and conventional heterotopic transplantation is infrequent and anticipated to be greater following a double heart transplant. The assist device is adjustable where the organic alternatives prompt adjustment by spontaneous adaptation over time.

By allowing adjustment in the frequency of switching to every second or third successive systole of either heart, for example, double heart switch transplantation permits the effective ejection fraction to be controlled. This factor allows compensating for the hypertension experienced by all transplant recipient within ten years following transplantation, as well as the renal insufficiency probably secondary thereto Moreover, by 1. Eliminating ischemia and 2. Accomplishing initial immune tolerance induction integral to the transferring of the transplant from the donor to the recipient, the switch procedure should reduce the severity if not the incidence of cardiac allograft vasculopathy

While in double heart transplantation the work of driving the blood is divided between two hearts assisted only by one another and drugs as appropriate in a reciprocal mutual assistance arrangement, a ventricular assist device supplements ventricular contractile function, partially if not entirely relieving the ventricular myocardium of exertion. The physiological indicia surrounding each include similarities and differences. Requiring to be substantiated through physiological testing, that following a double heart transplant, the left ventricles continue functioning but under a reduced load would seem equally conducive to recovery and reverse remodeling than the use of an optimally adjusted assist device and superior were the device adjusted to assume more of the work than necessary.

Double heart transplantation makes available an alternative to a ventricular assist device when the latter is contraindicated, especially due to its cumulative adverse effects in a child, or an orthotopic replacement is precluded due to the nonavailability in exigent circumstances of an adequate heart, or because the cost therefor in that part of the world is not prohibitively expensive. Unless the need therefor could not have been known in advance, the intraoperative addition of an assist device to a double heart transplant will in some cases be discounted as an alternative to the need for a second heart. In parts of the world where surgery is less costly, a second heart that would otherwise be rejected is free and far more readily available than a healthy heart, so that double heart transplantation may be considered a ‘poor man's ventricular assist device.’

Assist devices for use in neonates and infants, to include those most recent, such as the Berlin Heart North America EXCOR® Pediatric, The Woodlands, Tex., are paracorporeal, that is, located outside the body, and an impediment to freedom of movement and the wearing of infant clothing, but in the absence of a viable alternative, life-saving (Shin, Y. R., Park, Y. H., and Park, H. K. 2019. “Pediatric Ventricular Assist Device,” Korean Circulation Journal 49(8):678-690)—important due to the limited availability of infant hearts (see, for example, Dipchand, A. I. 2018. “Current State of Pediatric Cardiac Transplantation,” Annals of Cardiothoracic Surgery 7(1):31-55; John, M, and Bailey, L. L. 2018. “Neonatal Heart Transplantation,” Annals of Cardiothoracic Surgery 7(1):118-125).

“We conclude that heterotopic transplantation of a marginal donor heart can save an otherwise-dying orthotopic transplant recipient.” “Ejection fraction as indicated by echocardiography showed that the left ventricular function of the donor 1 heart was poor (LVEF, 0.30) at the beginning, but that it gradually recovered—under the support of the donor 2 heart—to normal function (LVEF, 0.62) 3 weeks later.” “This experience shows that the additional transplantation of a marginally acceptable donor heart in heterotopic position can save a patient who is dying of graft failure immediately after orthotopic heart transplantation.” (Chu, S. H., Chiu, K. M., Lin, T. Y., Chu, T. W., Yeih, D. F., and Li, A. H. 2007. “Two Donor Hearts Beat in One Chest,” Texas Heart Institute Journal 34(2):230-232). The cost for an assist device, surgery, and follow-up care in developed countries can amount to several hundred thousand dollars.

In countries with socialized medicine, making double heart transplantation—which tolerates hearts less robust than those acceptable for an orthotopic transplant—an effective alternative to assist devices would likely allow needy patients to receive care more promptly Unless having been confirmed to respond to therapy, hearts previously described as presenting diastolic heart failure, or informally, as showing normal contractility, but now referred to as heart failure with preserved ejection fraction, are unlikely to provide favorable transplantation outcomes, even in double heart transplantation, so that representing more than half of all failing hearts notwithstanding, such hearts should be avoided. Confirmed effective in the treatment of heart failure with reduced ejection fraction, sacubitril-valsartan has not offered comparable results in the treatment of heart failure with preserved ejection fraction.

The transplantability of hearts with preserved or mid-range ejection fraction will increase as more effective treatments become available. Able to release agents into the general circulation and/or targeted to the site or sites of disease automatically, apply warmth, deliver electrical, and prospectively, leadless pacemaker or direct optogenetic or electrostimulation at nidi, the implant disorder response system will facilitate the application of new therapy both mid- and postprocedurally. “ . . . neprilysin inhibitor-angiotensin receptor blocker-combination (LCZ 696), ranolazine, or ivabradine . . . ,” for example, has shown promise (Edelmann, F. 2015. “Facts and Numbers on Epidemiology and Pharmacological Treatment of Heart Failure with Preserved Ejection Fraction,” European Society of Cardiology Heart Failure 2(2):41-45). Restorative therapy for hearts affected thus is an area of active research. Hearts with mid-range ejection fraction are those most likely to prove remediable.

Other areas where direct electrical or optogenetic stimulation can be used to assist motile function while avoiding the shortcomings of mechanical devices are in deceased prospective organ donors on life support and as implants in patients. The electrical discharge and light emitting sources are best secured in place by nonjacketing side-entry connectors as described in copending application Ser. No. 14/998,495, which can be made with hollow injectable anchoring needles able to deliver fluid agents to work with the stimulatory energy. Devices such as heart-lung and extracorporeal membrane oxygenation machines are confined to the clinic and limited in duration of use.

Such means do not apply to critically weakened hearts, that is, in hearts lacking the strength to generate an adequate ejection fraction without added power, inotropes, a second heart to divide the load, and/or direct stimulation; however, end-stage impaired hearts requiring placement of an assist device are unacceptable for transplantation in any event. Hearts suitable for transplantation with the support of an implanted automatic disorder response system should retain sufficient strength to function with no more than the aid of pharmaceutical, and/or electrical, and prospectively, optogenetic stimulation.

Electrical or optogenetic stimulation should, however, allow hearts with sufficient viable but dysfunctional left ventricular muscle but rather neurological impairment, for example, important in ascertaining the restorative consequences of coronary artery bypass grafting, angioplasty, or the implantation of a ventricular assist device. Using the methods delineated herein, a usable heart must not be so impaired as to necessitate a ventricular assist device. These are neither applied to the heart of a deceased prospective heart donor while remaining on life support for the harvesting of other organs nor applied during or after transfer to the recipient. Hearts this impaired should be rejected. When the need is dire, hearts which have recovered with the aid of an assist device on a short term basis should be considered.

Ventricular assist devices are effective for bridging to transplantation, for example, but often produce adverse sequelae in proportion to the duration of use. With the relief in load, some patients do experience reversal of the remodeling that had taken place to compensate for the weakened left ventricle; however, such cases are exceptional. Double heart metered switch transplantation is intended for use in prospective heart recipients, not donors. For weaker native hearts, the second heart can be thought of as a form of assist device. Severely impaired hearts in prospective recipients are well served by an assist device. For reasons of surgical trauma, marginal hearts in deceased donors would best be treated by direct stimulation and drugs.

Use along the urinary and digestive tracts of commercially available neuromodulators is of long standing. While application to patients with heart failure and recipients of marginal hearts are contemplated, the main objective here is to sustain or assist motile function in a deceased organ donor with a marginal heart that would otherwise be rejected for transplantation. For these purposes, a means for assisting ventricular function without the surgical trauma associated with the implantation of an assist device would both allow the use, and less impose additional impairment upon, less competent hearts. Even miniaturized, assist devices are larger than and no more amenable to endoscopic and robotic implantation than are stimulatory probes.

Involving greater complexity, mechanical assist devices include more parts with different distribution and are more susceptible to complications with increased probability overtime. The surgical procedure to place these and totally artificial hearts poses numerous serious risks, and when successful, the patient is strapped with the need to wear outside components such as a power pack and controller that must be worn at all times to support a complex device with many parts any of which might fail at any moment with grave consequences.

The shortcomings of these devices in terms of hindrance, risk upon implantation, power consumption, and risk of mechanical failure compared to a double heart transplant are well documented in the literature. An implanted disorder response system can support, or in a double heart transplant, administer the operation as well as replace such a device. This leaves the surgery of placement and the immunological challenges associated with any transplant as risks; however, the level of contingency the patient will face should be much reduced. Percutaneously inserted intraaortic balloon pumps and ventricular assist devices impose a considerable demand for power with the need to wear a power pack, include numerous mechanical parts, and are generally placed for short term use.

In contrast, the power requirement to sustain the mechanical support of a double heart transplant—when even necessary—is much less, requires a much smaller and lighter power pack, and the intravascular servovalves used are mechanically simple and directly targetable for the release of an anticoagulant or antimicrobial as the need arises. Where double heart transplantation is intended as destination therapy with removal should the native heart recover possible, mechanical devices are usually not meant to remain permanently as destination therapy but rather bridge to transplantation pending availability of a replacement heart, or to allow time during which the patient can be stabilized for transplantation, or to allow recovery with device removal.

Significantly, the contraindications to assist device placement—right ventricular dysfunction; acute cardiogenic shock with a neurological compromise; coexisting severe terminal comorbidity; active bleeding or thrombocytopenia; hypertrophic cardiomyopathy or a large ventricular septal defect; body surface area less than 1½ meters; and a patient who cannot be depended upon for prescription and maintenance compliance (Vaidya, Y., Riaz, S. and Dhamoon, A. S. 2020. “Left Ventricular Assist Devices (LVAD),” StatPearls online at https://www.ncbi.nlm.nih.gov/books/NBK 499841/) do not apply to double heart transplantation. Except that a relative or attendant must assure that drug reservoirs are replenished, the implanted automatic disorder response system essentially eliminates the requirement for compliance.

As with a mechanical device, recovery of the native heart following a double heart transplant can allow the implant to be removed. Disorders of coagulation a frequent complication with ventricular assist devices, the ability of the implanted disorder response system to directly target drugs whether coagulants (hemostats) or anticoagulants to the device—or to any other target—with means for preventing continued travel through the circulatory system should avert bleeding of the upper gastrointestinal tract as a common complication.

However, coagulopathy can occur in cardiovascular disease with no involvement of a continuous flow ventricular assist device. “Various cardiovascular diseases, such as aortic stenosis, hypertrophic obstructive cardiomyopathy (HOCM), and several congenital structural diseases, as well as mechanical circulatory support systems, generate excessive high shear stress in the bloodstream. These cause excessive cleavage of VWF [von Willebrand factor] multimers resulting in a loss of HMW [high molecular weight] multimers, known as acquired von Willebrand syndrome . . . ” (Horiuchi, H., Doman, T., Kokame, K., Saiki, Y., and Matsumoto, M. 2019. “Acquired von Willebrand Syndrome Associated with Cardiovascular Diseases,” Journal of Atherosclerosis and Thrombosis 26(4):303-314) (see also, for example, Mehta, R., Athar, M., Girgis, S., Hassan, A., and Becker, R. C. 2019. “Acquired von Willebrand Syndrome (AVWS) in Cardiovascular Disease: A State of the Art Review for Clinicians,” Journal of Thrombosis and Thrombolysis 48(1):14-26).

Regardless of the cause or causes, adverse sequelae may necessitate device replacement, or exchange, and as does any surgical procedure, this too poses the risk of complications. “Device exchange is associated with significant risk, including hemorrhage, stroke, air embolism, and death” (Bhama, J. K. and Bansal, A. 2018. “Left Ventricular Assist Device Inflow Cannula Position May Contribute to the Development of HeartMate II Left Ventricular Assist Device Pump Thrombosis,” Ochsner Journal 18(2):131-135). The contrary view has also been expressed (Agarwal, R., Kyvernitakis, A., Soleimani, B., Milano, C. A., Davis, R. P., and 5 others 2019. “Clinical Experience of HeartMate II to HeartWare Left Ventricular Assist Device Exchange: A Multicenter Experience,” Annals of Thoracic Surgery 108(4):1178-1182; Exchange for upgrade to an improved device, however, usually when the original requires removal in any event, appears to present an acceptable risk to benefit ratio.

The intensive and immediate support of a fully implanted automatic ambulatory automatic disorder response system as described in copending application Ser. No. 15/998,002 and double heart metered switch heart transplantation have as a primary object the provision of alternatives to conventional treatment and liberalization in the criteria governing, and therewith, expansion in the pool of usable hearts. Harvesting graft organs upon death of the donor having remained on life support without the loss of perfusion and continuous transfer into the circulatory system of the recipient also protect graft organs against terminal damage that could disqualify these for transplantation, thus also increasing the number of suitable organs. When reverse remodeling indicative of recovery justifies explantation, removal likewise poses the risk of trauma and complications, sometimes due to the nonremoval of all parts.

Following a double heart switch transplantation, both hearts are relieved from their previous loads and supported by the implanted automatic disorder response system. Because double heart transplantation allows adjustment in the combined or net ejection fraction, there is no concern that the one or two donor hearts working together would initially present absolute stroke volumes so high as to precipitate coma or convulsions as experienced in pediatric cases. Thus, impaired, either or both hearts by itself might not present normal ejective strength at first, but by sequencing every third or so systole of either in close succession—even without the use of inotropes—an ejection fraction falling within the normal range can be achieved.

Moreover, adjustment in the frequency of systole switching can be used to positively encourage recovery. Even without this capability, given time, such a new combination to include an additional heart would adapt to the changed physiological setting. Such a circumstance is presaged, for example, during the period following transplantation of an oversized heart. The overly large and powerful replacement heart generates a cardiac output that initially and for a number of days produces hypertension. However, this condition spontaneously resolves so that medical support is essential only during the adaptive period.

In the brain, this may cause reactive vasoconstriction that precipitates convulsions or coma. However, this condition is dispelled within days during which the new heart adapts to the lesser load placed upon it (Shin, H. J., Jhang, W. K., Park, J. J., Yun, T. J., Kim, Y. H., and 3 others 2011. “Heart Transplantation in Pediatric Patients: Twelve-year Experience of the Asan Medical Center,” Journal of Korean Medical Science 26(5):593-598). In the situation cited, the oversized heart adapts to the abrupt reduction in load while the patient is still in the hospital with clinicians to monitor and treat the condition. In a double heart switch transplant, the effective ejection fraction of both hearts working together in alternation is adjustable. This confers more than the selection of a second heart widely different in size; in fact, for placement in the chest as preferred, the choice of a smaller, healthier heart should be favored.

Adaptation consists first of relaxation—cessation in the expenditure of energy needlessly; however, if the reduction in load continues, the excess ventricular power will eventually decline. Since the load on either heart is controllable, it is prudent to maintain each heart at a level of strength somewhat in excess of that needed ordinarily. Under constant monitoring by implanted sensors, beat timing circuitry, and a control processor able to immediately target any fluid drug to either or both hearts, the likelihood of a cardiac or vascular accident is much reduced. Nevertheless, should either heart begin to malfunction, essentially to fibrillate, then having provided a safety margin will allow the unaffected heart to take over circulation on its own.

In so doing, not all the blood is passed through the unaffected heart, since—azygos factor, or azygos flow principle, notwithstanding—to eliminate all venous return to the affected heart if continued beyond a half hour would likely prove injurious. The level of immediate medical support provided much reduces the risk that either, much less both hearts would desynchronize. In fact, if the generally accepted explanation for the azygos factor is correct, that it evolved as a collateral or backup path to sustain venous return to the right atrium were either of the venae cavae to become obstructed, then azygos return added to the reduced venous return with one vena cava obstructed should sustain a patient indefinitely, or at least much longer than the time required to perform a less complex intracardiac repair under hypothermic conditions. In nature, however, the prospect for both venae cavae to be clampled with venous return completely closed off, as in open heart surgery, is remote.

With a significant reduction in load and the support of one another as well as the implant system, both hearts, initially impaired, would be strongly encouraged to recuperate, and without causing hyperperfusion, of which the added volume could immediately be shunted back into another large vessel through a diversion servovalve, for example. Even without adjustability, that two hearts allowed to recuperate would do so without adapting to the new condition, that is, relax in response to the reduction in load but instead persist in exerting the greater ejective force allowed by having recovered, and do so beyond the strength needed to drive the load, is contrary to experience of the heart as a component in an integrated system.

Where in vivo injury to a graft heart or other transplant organ had been secondary to a primary disorder, such as pulmonary hypertension, removal of the organ and its resituation in a body not so affected would reasonably be expected to allow for some recovery on that score alone, confrontation with new and different challenges, primarily immune intolerance, notwithstanding. Based upon the oversized heart as precedent, having been made integral in the circulatory system, the pair would respond to the load presented, not continue to develop needless strength as other than components in a integrated internally balanced system. It is, therefore, probable that the hearts would gain in fitness but not overdrive their loads.

While adherence to the setpoint established on the basis of the absolute stroke volume of either heart conserves energy, to apportion a close to normal venous return volume to each heart, the servovalves on the venae cavae of both hearts are intermittently shifted in alternation to allow the one or the other to intake 80-90 percent of the total venous return. That double heart switch transplantation allows adjustment in the effective ejection fraction of the two hearts working together liberalizes the selection of donor hearts based upon size, thus considerably increasing the pool of acceptable donor hearts. Moreover, adjustability makes it possible to positively encourage recovery. Ventricular assist devices are also adjustable but when used

Where merged atria, or conventional heterotopic heart transplantation, was performed prior to the discovery of the first significant immunosuppressive cyclosporin to sustain circulation in the event of rejection, more recently, the procedure is performed in lieu of a heart-lung transplant to attenuate fixed or intractably elevated pulmonary hypertension resistant to medicinal reversal, and/or to overcome a significant size mismatch, as well as to expand the zone of usable hearts. In comparison, a double heart transplant offers superior therapeutic versatility, survivability, and applicability, as well as further expands the zone of marginal hearts, that is, the pool of extended criteria donor hearts that can be accepted for transplant.

The transplantation of one or two hearts in less than ideal condition where neither has undergone the trauma of ischemia and extensive dissection is fundamentally superior to the placement of a heart, however intact from an anatomical standpoint, which had undergone circulatory death, or cardiorespiratory arrest, with ensuing asystole. Improved ex vivo reperfusion measures arose against a background of compulsory rejection of hearts that gave the outward appearance of adequacy despite having undergone severe endothelial injury. In relation to the preceding situation, during which such hearts were rejected, means for secondarily beneficiating hearts previously discarded was advantageous.

However, the use of hearts from the dead where the heart itself had already died is inferior to a heart that had never experienced ischemia and nonperfusion, not the least due to reperfusion injury when artificially reperfused prior to transplantation, but then extensively traumatized by having to undergo a bicaval operation. “Even though DCD [donated after cardiac death] hearts and lungs have been successfully transplanted, the risks associated with using these lower quality grafts is very high” (Bezinover, D. and Saner, F. 2019. “Organ Transplantation in the Modern Era,” BioMed Central Anesthesiology 19(1):32).

While an implanted sensor-driven automatic response system with direct fluid and electrical lines can be applied to any transplant to immediately and selectively pipe-target a fluid drug or deliver electrical discharges to it, in a conventinal heterotopic heart transplant, the hearts are merged, precluding this capability. By contrast, in a double heart transplant, each heart remains intact as to be separately targetable—the donor heart with an immunosuppressive, for example—sparing the native heart and all other tissues in the body from exposure to the drug or drugs. Not inextricably united as in a heterotopic transplant, each heart can be separately diagnosed, adjusted, treated, and should it become necessary, removed while the other heart sustained the recipient.

All immunosuppressants increase susceptibility to infection and produce adverse side effects. Cyclosporin, for example, is not only a carcinogen, but often causes hypertension, convulsions, nausea, kidney and/or liver dysfunction, dyspnea, paresthesia, gastrointestinal ulcers, hypercholesterolemia, along with several others which limit its dose when not targeted. Tightly targeted, steroids, radionuclides, chemotherapeutics, and immunosuppressants can be delivered to a specific organ in the optimal dose without regard to side effects. When necessary, such as to destroy malignant cells shed by a ‘mother’ neoplasm, the systemically dispersed dose can be reduced while that directly targeted increased. The hearts are therefore separately treatable, with any deficit or malfunction as might affect the one not inextricably tied to the other.

For physiological reasons, the donor heart is preferably positioned adjacent to that native in the chest. Separate, the implanted sensor-driven automatic response system with direct fluid and electrical lines can target either heart for medication and/or electrostimulation. With the native heart nearing end stage failure, that is, at American College of Cardiology Stage D or New York Heart Association Class IV, adding a second heart not only assists that native by alleviating most of the load but in so doing, affords the native heart the opportunity to recover in situ with the aid of medication automatically targeted to it as needed.

Should the reduction in intracardiac blood volume precipitate a dysrhythmia in either or both hearts, the implant microcontroller or microprocessor, based upon the input data stream received from implantable heart beat sensor circuits, or pacemaker/cardioverter defibrillator detection and control timing circuits as sensors, can alternate the volume of blood processed by either heart. That is, rather than to sustain the diversion chute settings at the apportionment set on the basis of relative absolute stroke volumes of either heart, the chutes are moved to alternately apportion almost all of the blood volume to either in turn, the rate and extent of alternate volume apportionment determined by the controller executing an algorithm that locates the pattern of apportionment which best yields rhythmical durability.

The controller is also able to adjust the dose of dysrhythmic medication and deliver resynchronizing optogenetic or electrostimulation. Thus, should cardiac allograft coronary artery vasculopathy impair the transplanted heart or hearts, the unaffected native or less affected cardiac allograft would continue circulation, and given that vasculopathy does not ensue for years following implantation, the unaffected native if not the allograft would have had time to recover function it had lost prior to implantation, much reducing the prospect of a sudden cardiac arrest.

Were either heart to become infected, rejected, or otherwise impaired, the backup would sustain the patient long enough for transport to a hospital. The best case outcome is that the native heart would have recovered sufficiently to sustain the patient indefinitely. If neither heart were able to serve the patient, then the time gained would allow a medical response. Post-transplantation development of vasculopathy is the cause of eventual graft failure in some half of all heart transplants. In comparison with the causes for cardiac allograft vasculopathy, the risk factors that promote its development—advanced age, smoking, obesity, hypertension, dyslipidemia, and so on are well established and mostly preventable.

A probable—and if so, critical—benefit of switch heart transplantation is that to the extent vasculopathy as the cause of failure in half of all heart transplants—appears at least in part as a late result of the cytokine storms associated with the death of the donor, cessation of function, excision, and ischemia usually extended throughout preservation in cold storage, followed by the ischemia and trauma of conventional transplantation, the elimination of these factors should result in an attenuation in if not complete avoidance of vasculopathy, and therewith the prelimitation in service life of the new heart it imposes. The literature on cardiac allograft vasculopathy is directed to its treatment, not prevention.

Given that a heart transplant supported with statins, platelet blockers, antiproliferatives such as sirolimus and everolimus, with other drugs responsive to particulars such as antihypertensives, anti-inflammatories, and antimicrobials, the elimination of ischemia-associated trauma attendant upon procurement and handling of the graft should allow a significant retardation if not elimination in cardiac allograft vasculopathy to allow the recipient a longer and better quality of life. “Cardiac allograft vasculopathy prevention may involve therapy that provides protection against endothelial injury implemented just before transplantation, during storage and transplantation as well as after transplantation” (Volpe, M., Rubattu, S., and Battistoni, A. 2019. “ARNi [angiotensin receptor-neprilysin inhibitors]: A Novel Approach to Counteract Cardiovascular Diseases,” International Journal of Molecular Sciences 20(9). pii: E2092).

In fact, the injury imposed upon the graft organ is considerably greater than that done to its endothelium. In eliminating the trauma associated with death, excision from the body from which, while remaining under life support, it is properly inseparable, ischemia, cold storage, extensive dissection, reperfusion injury, then fixation within an alien repellent milieu, the combination of life support and switch methodology should materially improve outcomes in solid organ transplantation Other causes are the immune response to the allograft and immunosuppressants, in addition to hyperlipidemia, insulin resistance, hypertension, infection, and/or hypertension if present.

Where atherosclerosis is largely shear stress-driven mechanically as to affect larger arteries of high flow rate especially at bifurcations while sparing arterioles and capillaries, cardiac allograft vasculopathy, albeit differently, affects all segments. “A lack of correlation between microvascular and epicardial vessel disease suggests discordant manifestations and progression of CAV” (Weis, M. and von Scheidt, W. 1997. “Cardiac Allograft Vasculopathy: A Review,” Circulation 96(6):2069-2077), even resulting in atrophy and loss of terminal microvasculature, or “pruning” (Lee, M. S., Tadwalkar, R. V., Fearon, W. F., Kirtane, A. J., Patel, A. J., and 3 others 2018. “Cardiac Allograft Vasculopathy: A Review,” Catheterization and Cardiovascular Interventions 92(7):E527-E536).

While the effect can take as long as ten years, cardiac allograft vasculopathy eventually necessitates the retransplantation of about half of conventional, or caval orthotopic, heart transplants. However, with the tolerance induction of metered switch heart transplantation and follow-up provided by the implant automatic response system to include the direct targeting of drugs to any part of the heart, the incidence of vasculopathy as a late term cause of sudden death should be considerably reduced.

The support of another heart sustains the patient past failure of either heart and likely both allows the native heart to recover as well as reduces the severity of sequelae. When a second heart is present, in addition to late term vasculopathy, a sudden cardiac arrest, myocardial infarction, graft failure, dysrhythmia, or infection, for example, in either the native or earlier orthotopically transplanted, or in the added heart need not equate to imminent or sudden cardiac death.

The advantage in switch transplantation in not requiring an interruption in functioning of the heart to be transplanted and therefore not requiring the heart to be bypassed so that cardiopulmonary support under general anesthesia is necessary may be discerned in coronary artery bypass grafting. When compared to machine supported coronary artery bypass grafting, off-pump, or beating-heart coronary bypass has long been known to yield a better outcome. “Off-pump coronary artery bypass was associated with a significant reduction in risk of death, stroke, acute renal failure, mortality or morbidity, and postoperative length of stay compared with on-pump coronary artery bypass after adjustment for 30 patient risk factors in the overall sample.” (Polomsky, M., He, X., O'Brien, S. M., and Puskas, J. D. 2013. “Outcomes of Off-pump versus On-pump Coronary Artery Bypass Grafting: Impact of Preoperative Risk,” Journal of Thoracic and Cardiovascular Surgery 145(5):1193-1198).

More favorable outcomes with off-pump technique are experienced no less with intracardiac and combination coronary-intracardiac procedures. Improvements in equipment, surgical technique, and patient selection have ameliorated some of the adverse consequences and elevated the value of cardiopulmonary support with respect to coronary artery bypass grafting; however, pumpless heart transplantation has not been an option. Given teams in large centers with much experience using both techniques, off-pump coronary artery bypass grafting yields better outcomes. The same guidelines as pertain to the qualifying conditions and preparation for the recipient and donor and follow-up for the recipient apply as usual. Postoperative care includes immunosuppression and the retarding of cardiac allograft vasculopathy—a factor that uninterrupted perfusion as well as periprocedural tolerance induction might reduce even with the metabolic syndrome with its hyperlipidemia as a mechanism of rejection.

FIG. 10A shows a servovalve suitable for bypass, or switch organ transplantation, and FIGS. 18 thru 21 depict the use of vascular servovalves for performing a perfusion interruption free solid organ transplant reduced to simplest terms. Because transplantation of the heart—the pump itself—is the most challenging procedure for off-pump heart surgery, one always distinguished from other transplant organs in a special intolerance for nonperfusion the one that therefore subsumes the feasibility of other organ transplants, to include the kidney, liver, gut, or lung, for example, that shown here is that of an off-pump heart transplant. In FIGS. 15 thru 17, the recipient heart is on the right and the donor on the left. Both donor and recipient are systemically medicated to reduce the risk of thrombus or infection.

The donor alone is kept on machine bypass following harvesting and positioned as closely parallel or side by side to the donor as does not interfere with the operation. In any heart transplant, the object is to employ the best technique for the specific recipient. Ultimately, any technique carries a risk for residual synchronization disturbances. Current techniques not taking the means described herein into account, using the method described, dissection that least alters the morphology of the donor heart and minimizes if not eliminates the need for anastomoses is preferred. Sufficiency of function and freedom from complications are the appropriate criteria, not mere survival.

The biatrial technique least preferred, depending upon the degree of structural matching between the hearts of the donor and recipient, the cardiectomy and replacement technique preferred is the total orthotopic. It is believed that using the procedure set forth herein, the advantages over the total orthotopic of the bicaval technique will no longer apply. The total orthotopic technique removes the donor heart with full excision of the atria, preserving intact the structure of these, the four main pulmonary veins, venae cavae, and other vessels. From the present standpoint, this has the advantage of avoiding the direct suturing in juxtaposed relation of allogeneic tissues at the anastomoses—the present approach seeks to reduce if not eliminate the need for direct anastomoses.

The bicaval technique improved upon the biatrial technique in eliminating the distortion in atrial conformation when the donor heart was anastomosed to the atria of the recipient. However, this consideration does not arise with the total orthotopic technique, and is advantageous only in the absence of the concept and means provided here. These techniques represent only partial transplants, the front of the defective original heart replaced with another stitched onto the original posterior portions. To allow sufficient space to position the jackets, preserve the morphology of the heart, and prevent leakage, notably from the venae cavae, these vessels are trimmed back leaving the perivascular fat which has physiological function and transected as long as possible, which does not involve resection through the posterior right atrial wall, with the donor heart implanted orthotopically.

If as preferred, the jackets are to replace anastomoses, the vascular stumps of the recipient, to include the ascending aorta, pulmonary trunk, or main pulmonary artery, and pulmonary veins, and venae cavae, are transected so that these will extend out of the jackets to be filled with a nontoxic and nonallergenic monomer or residual monomer and antibiotic-releasing bone cement or an acrylate-based dental cement, and sutured shut. The absolute amount and substantial isolation of the cement should preclude the development of adverse side effects. The avoidance of direct donor-recipient anastomoses liberalizes the matching in size of the vessels respective of each and bypasses the interfaces where the immunologically mismatched tissues are in direct contact. The universal lack of sufficient donor hearts has forced increased tolerance for a lack of suitable donor-recipient matching as to heart size.

Another set of problems resolved by allowing the blood to flow through the jackets and mainlines rather than anastomosing the stumps of donor and recipient is the liberalization in juxtaposing, that is, bringing the cut ends of the donor and recipient stumps into apposed, or juxtaposed, relation, either when transplantation can be avoided through the Jatene arterial switch procedure, or substantial reconstruction, or when transplantation is resorted to in order to correct anomalies of orientation that are irreparable where there is an urgent need for a heart transplant.

Provided the left atrium is completely detached from the thoracic wall, an additional degree of movement allowed when piping rather than direct anastomosis is employed is some liberalization in the exact rotational disposition of the donor heart, significant when the thorax is malformed, as presents with numerous congenital conditions. While unlikely, anastomosing some while bypassing other vessels must remain a clinical judgement based upon the specific condition of the donor heart and any anatomical peculiarity of the recipient. For example, unless the need for a heart transplant is not exigent so that time permits reconstruction of the anterior thoracic wall, the heart in a patient with pectus excavatum may be considerably displaced and even rotated.

If severe, the condition may have caused mitral valve prolapse, and therewith, the cardiopulmonary dysfunction that led to the need for a heart transplant. The abnormal mechanical forces which a severe thoracic malformity such as heterotaxy (heterotaxia, situs ambiguous, situs ambiguus, isomerism), Scheuermann's kyphosis, scoliosis, kyphoscoliosis, kyphoscoliotic Ehlers-Danlos Syndrome, straight back syndrome (thoracic lordosis), Parkinson's disease, or camptocormia (bent spine syndrome) places on the heart can also result in cardiac damage.

Should complete ligation or embolization nevertheless result in atrophy, then the ligation is subcomplete, allowing some flow from each vessel to pass and mix with the greater volume of blood crossed over. To accomplish this with woven reinforced polyethylene terephthalate (Dacron®, Terylene®, woven Dacron® or Terylene® tubing requires crossclamping under cardiopulmonary support and affords no way to target drugs directly to the treatment site. While side-entry diversion jackets connected by tubes connecting their mainlines as a bypass to instantly switch the flow through each vessel to the other to be followed by conventional anastomosis is possible, it is preferable to leave the bypass as a permanent prosthesis with the ability to target medication to the treatment site.

As the pressure is greater in the larger vessel and abruptly drops moving past the suture line leading into the smaller vessel, anastomoses of vessels differing in luminal diameter produce constant stress on the suture line, already challenged as the interface between immunologically incompatible tissues placed in direct contact. By contrast, interpositioning a length of gradually tapered tubing between the two removes the abrupt pressure drop, and the jackets coupling the tubing to the vessel at either end have accessory channels to target medication directly to the jacket-tissue junctions, which feature is even more beneficial when used in conjunction with conventional anastomoses in allowing the direct targeting to the site of immunosuppressive medication, allowing a background systemic dose and the side effects it causes to be reduced.

Such distinctions in vessel caliber do not equate to or even correlate with differences in body weight or surface area. The suture lines, that is, not the suture itself, but rather the train of stitches, of anastomoses the sites most susceptible to thrombosis, stenosis, or stricture, aneurysm, pseudoaneurysm, infection, and the generation of stray discharges that cause arrhythmias even when autologous, these risks are intensified when allogeneic tubular stumps are stitched together in direct contact over the incised surfaces. The immune system implemented primarily through the blood and endothelium, the transplant as a whole is attacked, but thinner, punctured, cut edges in direct contact are more vulnerable to breakdown. This in a context already fraught with impediments.

The risk of stump atrophy, which could result in jacket migration and failure, is additionally counteracted by means addressed elsewhere herein, and if necessary, the direct targeting to these blind pockets of medication through the accessory channel coursing along the bottom of the chute, which is connected to the port at the body surface, direct drug targeting on a permanent basis thus enabled from outside the body. Alternatively, the stumps are anastomosed, each to its counterpart, the jackets left in place to allow targeting drugs at the suture lines and pass fine cabled devices to each junction thereby providing a passageway for directly targeted treatment and viewing with entry at the surface port.

Before its advancement or deployment when the diversion jacket is placed, the ostium obturator is withdrawn inside the trepan tube where it blocks the way for passage of a miniature cabled device. Antegrade upstream and retrograde downstream, when advanced into the substrate native lumen, the ostium obturator will continue to block passage until it is sufficiently advanced to provide the clearance beyond its distal face (its front side closest to the far wall of the lumen) needed to access the vessel in the downstream direction and accommodate the steerability and size of the cabled device in relation to the size of the substrate native ductus and whether the ductus is straight or tortuous at the level sought to be entered. Passage in the upstream direction where the cabled device would pass along the upper side of the chute to be turned by the obturator and not restricted thus.

That is, with a side-entry servovalve, the ability to pass a miniature cabled device past the ostium obturator and diversion chute in the antegrade direction, meaning through the space separating the ostium obturator from the far wall, depends upon the size of the valves, the extension of the diversion chute into the native lumen, and whether the cabled device can negotiate a space of this size. With adequate clearance to accommodate the steerability and size of the cabled device, passage can be through the mainline and trepan tube. If tortuous at the level to be reached, access is by Seldinger cutdown upstream to the vessel jacketed or further upstream to the vessel of which it is a branch.

The use of side-entry jackets offers considerable advantages in maintaining autografts regardless of the transplantation technique employed; the ability to either avoid suture lines altogether or to deliver drugs to suture lines argues for the use of side-entry or side-entry diversion jackets. Cardiac allograft vasculopathy does not spare children for whom some form of replacement of a congenitally malformed heart is their only hope for life Antepartum metered switch heart transplantation). Metered switch transplantation does not interfere with the collateral application of apheresis or the simultaneous infusion or injection of agents not stored in and dispensed by the implanted system during its administration of the organ transfer from donor to recipient.

The current practice of harvesting donor organs only after shutting off life support and confirming death is injurious to the organs and a likely cause for failure, even long term, as in the case of cardiac allograft vasculopathy. The cessation of life support upon brainstem death, excision, and abrupt removal of the graft organ from its physiological and metabolic situation wherein it had been an integral, interdependent, and cooperating component of an intact unitary body represents a key basis of rejection no less injurious than does that immune. While harvesting is inevitable, functional replacement by such means as the placement of an artificial heart, renal and/or hepatic dialysis, total parenteral nutrition, ventilator, and the exogenous provision of hormones would better preserve the prospective graft organs.

The interdependence of the organs is well established, and transplantation of mutually dependent organs together might not only avert the need for later retransplantation of either based upon 1. Physiological interdependence, but because 2. One organ bolsters immune tolerance induction in the other, and especially if taken from the same donor, 3. Contributes to the ability of the other to avoid rejection. Diabetes, high blood pressure, and end-stage kidney disease are closely interrelated, cardiorenal, hepatorenal, cardiohepatic, and similar conjoint disorders common. Due to this mutual dependency, substantially interdependent organs tend to fare better when transplanted together or the one shortly after the other, and the same is true of retransplantation, which more durable, is growing in support.

While the switch methods are preferred, conventional transplantation is no less served by automatic intracorporeal support; the implantation of an automatic response system would reduce the incidence of fatal cardiac allograft vasculopathy whether the transplantation had been conventional or through the metered switch method. It is noteworthy that the continued support of the patient is carried out by the same system that administered the transplantation from the outset. One advantage of an implanted automatic response system is the ability to directly pipe-target medication to the coronaries to quell the cardiac allograft vasculopathy that develops to jeopardize late term graft survival.

Often leading to death as its cause within one year following a conventional heart transplant, the avoidance, or at least the minimization if not the elimination, of cardiac allograft vasculopathy is one advantage of the tolerance induction incorporated as integral in metered switch heart transplantation and similar adverse sequelae that follow the transplantation of other solid organs. Generally taking about a year to become a serious risk, that the same implanted automatic system used to perform the transplantation can periodically target a statin and immunosuppressant such as basiliximab monoclonal antibody, for example to the transplant further suppresses the emergence of frank vasculopathy.

The immune response to a pathogen involves pathogen-associated molecular pattern recognition receptors, such as Toll-like and C-type lectin receptors, and is distinguishable from the response involved in the rejection of a graft organ by damage- or danger-associated pattern biomolecules, or alarmins, which may be involved in vasculopathy. Increased heat alone signals inflammation, but not the cause or the specific type effector mechanism or mechanisms. The specification of sensors to be implanted as part of an automatic disorder response system depends upon the condition to be treated and the distinctive chemistry and cellular participants involved in the immune response and symptoms.

The potential applications for implanted automatic disorder response systems pertain to transplant recipients and dying prospective organ donors, as well as to living patients with one or more chronic diseases, but not self-limited acute diseases, wherewith it is essentially coextensive with the pathology of serious disease. The field of sensor development undergoing rapid expansion, with few exceptions, the diverse types of sensors required and operational mechanism of each exceeds in scope and level of detail necessary here. For patients undergoing solid organ transplantation, the implanted disorder response system must incorporate sensors to monitor rejection as well as infection in order to respond appropriately to these contingencies. At the same time, many diseases, notably the dementias, often comorbid in the elderly, require sensors to detect the presence and levels of disease-associated analytes.

The coincidence of carotid disease and dementia comorbidity high in the elderly, the benefit in releasing various substances directly into the internal carotids through one or more accessory channels in the vascular side-entry servovalves used to temporarily, intermittently, or permanently bypass flow from the common to the internal and usually, the external carotid is compelling, especially as the adverse side effects such as diarrhea caused by resveratrol would be avoided. For this purpose, the vascular side-entry jackets used can be of any type from those simplest, and example of which is shown in FIG. 16, copending application Ser. No. 15/998,002 entitled Ductus Side-entry Jackets and Prosthetic Disorder Response Systems to more complex vascular servovalves such as those shown herein in FIGS. 10A thru 10E, 25A, and 25B.

To function neuroprotectively, these substances, to include oxidized nicotinamide adenine dinucleotide (NAD⁺), reduced nicotinamide adenine dinucleotide (NADH), and resveratrol, would have first to establish the as yet unproven benefit these have been claimed to provide, and metabolism in the liver, lungs, and elsewhere in the body having been circumvented, the beneficial effect would have to result through direct, or topical, application in humans. More specifically, the advantage in directly pipe-targeting drugs through the carotids to the brain is that the dosage level need not be limited to by the need to avert adverse side effects elsewhere in the body. The constraint of drugs delivered thus to the target, for which several methods are available, is addressed below.

Sensors to detect analytes diagnostic for disease and adverse reactions, and the drugs to suppress these are identified on the basis of immunological and biochemical studies. The development of engineered sensors is expedited through the study and emulation of biological sensors, or biosensors. Sensors that must be supplied with living cells can be replenished as needed through an implanted drug reservoir as would any other agent. An exemplary citation of references here on metered switch transplantation can no more than indicate the scope of the subject. The progression of vasculopathy more frequent in hearts with donor-transmitted atherosclerosis, the preconditioning of metered bypass, or switch, transplantation and maintenance on a directly pipe-targeted statin and immunosuppressant reduces the undesirability of such hearts when available.

Whether for switch orthotopic or double heart transplant, donor transmitted atherosclerotic lesions are angioplastied in the donor before the donor heart is implanted. That the damage done to a graft organ is directly proportional to ischemia during preservation and cardiopulmonary bypass times with cardioplegia, and cross clamping, and harm to the recipient often done as a result of the need for general rather than regional anesthesia is universally acknowledged: “ . . . it is now important to be familiar with multiple new technical challenges associated with the surgical techniques of heart transplantation with an ultimate goal in reducing donor heart ischemic time, recipient cardiopulmonary bypass time and post-operative complications.” (Cheng, A. and Slaughter, M. S. 2014. “Heart Transplantation,” Journal of Thoracic Disease 6(8):1105-1109.

Numerous conditions can affect the electrocardiogram, to include chronic kidney disease, sarcoidosis, hypothyroidism, diabetes, heavy smoking, whether the affected heart is a transplant, obesity, and weight loss, for which the prescription program of the master control microcontroller must be adjusted (see, for example, Fernandes, F. M., Silva, E. P., Martins, R. R., and Oliveira, A. G. 2018. “QTc [corrected start of electrocardiogram Q to end of T wave duration] Interval Prolongation in Critically Ill Patients: Prevalence, Risk Factors, and Associated Medications,” Public Library of Science One 13(6):e0199028).

If either heart experienced a sudden arrest or ventricular fibrillation not reversible for more than a few seconds, more likely in patients with chronic kidney disease on dialysis, then rather than permitting the functioning heart to pull or push the setpoint adjusted proportional volume of venous return blood based upon the absolute stroke volumes of either heart through the abnormally functioning heart, risking malfunction of the functioning heart as well as the cessation of function of the lungs and brain, and pulseless electrical activity or asystole if not the cessation of circulation, the control microprocessor adjusts the valves to pass only so much blood through the nonfunctioning heart to supply it with sufficient oxygen to avert a sudden arrest and death-associated ‘cytokine storm’ (cytokine, or polypeptide mediator, release syndrome, or severe reaction systemic inflammatory response).

This continues until a detailed analysis and reversal can be conducted in the clinic (see, for example, Burak, C., Baysal, E., Süleymanoglu, M., Yayla, Ç., Cay, S., and Kervan, Ü. 2019. “Evaluation of Myocardial Dispersion of Repolarization in Patients with Heart Transplantation,” Turkish Journal of Medical Sciences 49(1):212-216). Whether due to an excessive increase in absolute stroke volume of either or both hearts, any such deviation in performance due to maladaptation or disease would originate past discharge from the hospital only to be counteracted upon detection by the implanted automatic response system. These safeguards and the usability of hearts that would otherwise be rejected for transplantation compensate for the extra space required to accommodate two hearts, and make a double heart transplant considerably preferable to a conventional heterotopic transplant, for example.

Then the implant system would detect and automatically adjust the output pressure to fall within the normal range by electromechanical means addressed in copending application Ser. No. 15/998,002 as will as herein, and/or pharmaceutical. Prior to any surgical procedure, an effort is made to predict and account for potential complications. Here this includes placement of the different type valves and shunts that might become necessary to adjust the pressure of inflow and ejection of the two hearts, determination of the drugs likely to be needed, and placement of the small flat reservoirs in the pectoral region from which the controller can command the release of the drugs.

Whereas in conventional orthotopic placement of an oversized heart and in heterotopic, or ‘piggyback’ transplantation, a right and possibly a left pericardectomy is needed, to place a second intact heart within an unencroached space as preferred in the chest will likely require a right pericardectomy and a partial pneumonectomy (partial pneumectomy, partial pulmonectomy) of the lower right lung well inferior or caudal to the pulmonary arteries, pulmonary veins, and the diaphragm. Necessitating a clamshell thoracotomy or a bilateral thoracosternotomy, the resection is accomplished at the same time as the transplant.

To allow free access to the vessels at either side of the heart for attachment of the servovalves, both orthotopic and double heart transplants require a clamshell thoracotomy or bilateral thoracosternotomy (see, for example, Pfannschmidt J. 2015. “Thoracotomy and Sternotomy,” in Dienemann H., Hoffmann H., and Detterbeck F. (eds,), Chest Surgery, Springer Surgery Atlas Series, Berlin, Germany: Springer, pages 9-14) to allow. Access thus is also needed in a double heart transplant when the second heart is to be positioned alongside to the left of the native heart, where a partial pneumonectrmy is needed to gain adequate space.

Since with the apportionment of the workload between the two hearts is automatically adjusted by the implanted automatic response system, which can also directly target medication to either heart, a second heart positioned in the chest can be smaller, minimizing any postoperative outward protrusion or bulging. Protection of the heart following placement of a second heart in the chest demands reinstatement of rib continuity. Relief of the severe pain that follows a thoracotomy allows the patient to cough and thus expel secretions, with the result that infection and atelectasis, for example, which might otherwise arise as significant sequelae, are avoided (see, for example, Gerner P. 2008. “Postthoracotomy Pain Management Problems,” Anesthesiology Clinics 26(2):355-367, vii.).

Management of the severe chest and often shoulder pain that ensues after a thoracotomy, or post-thoracotomy pain syndrome, demands minimizing nerve damage, avoiding incision into a bronchus, displacement of a scapula, and irritation from the thoracostomy tube or to the pericardium or mediastinal and diaphragmatic pleura. Chest pain is best ameliorated by thoracic epidural analgesia and/or paravertebral nerve block, serratus anterior plane block, pectoral nerve II block, erector spinae plane block and intercostal nerve block.

That a heart transplant using the switch technique, or any other extracardiac procedure, such as a coronary artery bypass graft, or a partial pneumonectomy can be accomplished without general anesthesia or cardiopulmonary bypass, and without collapse of the lungs calls for an explanation. Entry into the chest results in the admission of air into the pleural cavity causing lung collapse, or pneumothorax. Often a lung operated upon is allowed to collapse, the patient continuing to breathe with the isolated contralateral lung, with mechanical ventilation assistance as necessary.

While single-lung breathing eliminates the need for direct mechanical oxygenation of the blood—which does not, as does direct blood oxygenation, damage blood cells but nevertheless may lead to further complications, here, where double heart metered switch transplantation and heart-lung transplantation are best performed without, or if not, then minimal artificial support; collapsing both lungs, the usual full chest clamshell necessitates the use of a surgical chestdome described in this section and below in section 6 or artificial ventilation. The pleural cavities separate, surgical entry into the chest allows air to enter collapsing the lung on that side but leaving the contralateral lung unaffected and usable.

However, a clamshell thoracotomy spans the chest, causing both lungs to collapse, thus disallowing the use of one lung or selective lobar surgery with mask or intubated ventilation to oxygenate a patient under conscious sedation unable to breathe. Spontaneous breathing under regional anesthesia is preferable to the use of artificial, or mechanical, ventilation. Open-heart, or intracardiac, procedures include conventional but not switch method executed heart or heart and lung transplantation and the repair of less severe congenital malformities where the repair will result in normal circulation. Coronary artery plaque should be removed transcatheterically without surgical entry. Improved means for removing rather than crushing plaque are described in copending application Ser. No. 15/932,172 entitled Integrated System for the Infixion and Retrieval of Implants.

With a surgical chestdome, described in this section and below in section 6, essentially a cross between a laboratory or industrial glovebox and an irregularly shaped vacuum bell jar used to enclose harvested lungs during ex vivo lung perfusion, for example, but provided with large diameter airtight gloved openings for surgical use, intracardiac procedures require only cardio-, not pulmonary bypass. By maintaining equal intra- and extrapulmonary gas pressures during thoracic surgery, the chestdome prevents a unilateral or a bilateral pneumothorax, thus allowing the use of natural breathing, or spontaneous ventilation, under regional or local anesthesia. Concern for causing permanent neurological deficits justifies the use of regional if not local analgesia (see, for example, Chakravarthy, M. 2018. “Regional Analgesia in Cardiothoracic Surgery: A Changing Paradigm toward Opioid-free Anesthesia?,” Annals of Cardiac Anaesthesia (New Delhi, India) 21(3):225-227).

Currently the norm, when a graft organ becomes available after death, the concept of ex vivo lung perfusion can be applied to any organ to allow pre-transplantation therapy. However, even if this includes immune tolerance induction through the infusion of blood products from the prospective recipient, for example, that the organ has been severed from a normal physiological milieu severely reduces the value of such treatment. The optimal form of pre-transplantation therapy is that delineated herein, whereby the end-stage patient has been placed on life support, if possible, with normal breathing, or spontaneous ventilation, and circulation administered by an automatic implanted system as described herein, in advance of the terminal event so that essentially normal physiology has continued without avoidable disruption.

When the prospective donor is found shortly after death, the best option is to preserve the anatomy intact and reinitiate circulation and respiration administered by an implanted automatic system as described. That only a finite amount of time will allow this is a protective factor. The worst approach is to sever organs from the body and treat these apart from the context in which these developed and functioned. The surgical chestdome is intended for use during any procedure, endoscopic, video-assisted thorascopic, hemiclamshell, or clamshell, to equalize the pressure outside the chest where a lung collapse would best be avoided. Broadly, any procedure that might cause a pneumothorax, such as an endoscopic lobectomy, can avoid this eventuality with the aid of a chestdome.

Absent a pressure equalizing chestdome, a clamshell thoracotomy, for example causes both lungs to collapse, precluding spontaneous, and thus requiring mechanical ventilation. A chestdome offers the advantages of allowing any extracardiac procedure to be performed without the need for general anesthesia, cardioplegia, or cross-clamping, each of which is associated with serious sequelae. Unlike a laboratory or industrial glove box, the chestdome is provided with more than two large diameter airtight gloved entry portals where each allows unrestrained freedom of movement to allow operators clear and ready access to any enclosed point from the front, back, or sides.

To allow operating team members to pass tools and tubing in and out of the chestdome while minimizing if not preventing air from entering, the chestdome has high density bristle antistatic brush ‘air curtains’ or windows so that many bristles, each stiff enough not to bend due to the pressure difference, remain in contact with, running along the surface of the item passed, blocking air from entry. Briefly, molded as a clear transparent tough plastic shell made of bisphenol A and plasticizer-free abrasion, chemical, solvent, and microbial-resistant non-yellowing thermally cured polysiloxane-coated polycarbonate sheet, for example, the surgical chestdome encloses the space above and to the sides of the chest to allow equalization of extra- and intrapulmonary pressures.

The bottom of the shell is positioned below the diaphragm, that is, sufficiently inferiad along the body as not to restrict the diaphragm in breathing. The chestdome rests upon, fully encloses, and seals off the chest from the surrounding air, and bends downward at the sides to rest upon and place much of its weight on the table. Unadjustable, the chestdome must be made available in different sizes. To reduce the height and weight of the chestdome, the padded arms of the supine patient, are placed with the hands behind the head rather than upwards to expose the axillae (see FIG. 1 in Hayanga, J. W. and D'Cunha, J. 2014. “The Surgical Technique of Bilateral Sequential Lung Transplantation,” Journal of Thoracic Disease 6(8):1063-1069).

Precautionary measures specified by Hayanga and D'Cunha apply. An airtight thick viscoelastic polyurethane foam base surrounding a cast iron core is bonded to the bottom of the dome. The light vacuum should seldom require a coating about the base of vacuum grease. Construction thus is intended to assure self-seating and self-sealing thus eliminating the need for straps that lock beneath the table, impeding both positioning and removal. A more detailed description of a dome is provided in section 6.

The large gloved openings afford sufficient maneuverability to allow operators to move their hands up or down, forward, or sideways. Surgical tools, tubing, retractors, and so on are prepositioned inside the dome which also incorporates one or more brush-surround ‘air curtains’ to conserve the mild vacuum while other items are passed through as necessary. In a heart-lung transplant using the sudden or metered switch method, due to the size of the graft heart-lung bloc, a midprocedural pneumothorax can be avoided by placing recipient and donor in close adjacency under a common to both, or double-wide, surgical chestdome which extends over to include the space between the two operating tables.

For ease in transferring the harvested graft heart-lung bloc to, and removing the native bloc from the recipient, the surgical chestdome provides large diameter airtight front, rear, and side, glove openings and brush-surround air windows in the section connecting the two as well as at either end. In single heart, lung, or thyroid transplant which might result in a problematic pneumothorax, the relatively brief interval needed to move the graft from under the donor chestdome and into that of the neighboring recipient and to remove the native organ requires separate chestdomes with large glove holes to allow removal and entry of the hearts.

In a double, or bilateral simultaneous, lung transplant with a live or deceased single lung or lobe donor to either side, a triple configuration to span over the recipient at the center can be provided. Relatively lengthy collapse routinely occurs during single lung unassisted breathing, or spontaneous ventilation, as surgical, that is, intentional, or iatrogenic, pneumothorax, and the time to transfer a harvested lung from the donor to the recipient chestdome alongside the donor is a matter of moments. Therefore, except in large centers where the volume of such operations may eventually increase to justify double and triple patient domes, single patient domes with self-adjusting suction pump to maintain a light vacuum to keep intra- and extrapulmonary pressures equal inside the dome should prove satisfactory.

Neither does a momentary collapse justify complicating the use of individual chestdomes through the addition of a dome-to-dome connecting ‘skybridge,’ or side-tunnel with glove openings, for example. It is also possible to counteract a pneumothorax by increasing and sustain the volume of air inspired (see, for example, Kenny, B. J. and Ponichtera, L. 2019. “Physiology, Boyle's Law,” Treasure Island, Fla.: StatPearls Publishing, online at https://www.ncbi.nlm.nih.gov/books/NBK538183/; Sharma, S., Hashmi, M. F., and Rawat, D. 2019. “Partial Pressure of Oxygen (PO2),” Treasure Island, Fla.: StatPearls Publishing, online at https://www.ncbi.nlm.nih.gov/books/NBK493219;). Ventilation is preferably spontaneous with standby mechanical assistance if needed. For this reason, double and triple models to extend over more than a single subject are made available for pneumothorax-intolerant patients.

Hence, using the methods to be described herein for:

1. Extracardiac surgery, cardiopulmonary bypass is not required, and for
2. Intracardiac surgery, only the cardiobypass roller or centrifugal pump with tubing, cannulae, and the cardioplegia circuit are required, not the oxygenator, and
3. The need for general anesthesia with either extracardiac or intracardiac in terms of analgesia if not anxiolysis, or conscious sedation, is eliminated for both, and
4. Unless an intracardiac repair will restore normal circulation, a transplant is preferable, and performed as described herein, will be safer, less traumatic, and with increased availability of transplantable hearts, the number of intracardiac procedures and therefore the need for cardiopulmonary bypass should diminish.

Furthermore, a severe Type A aortic dissection, aneurysm, or especially difficult valve repair in an elderly patient with significant comorbidity, for example, is far more safely managed by not attempting to repair the defect but rather by replacing the heart along with an intact aorta and/or valves. The same pertains to a congenitally malformed heart where any attempt at repair would still not achieve normal circulation. Given the increase in available hearts made possible by an implanted automatic response system, many marginal hearts previously rejected can be transplanted, and double heart transplantation further increases the number.

Any transcatheteric means for the repair of an intracardiac defect without the need to open the chest, such as vulnerable plaque in a coronary artery near to its origin or a heart valve where the result should prove curative and durable likewise avoids the need for general anesthesia, cardioplegia, and so on, and does so with the least trauma—such means are always to be preferred. Invasive extracardiac and pulmonary surgery can be performed under regional anesthesia or analgesia without either lung collapsed and with the patient breathing normally as preferred or ventilator-assisted with a chest surgery glovebox, or surgical chestdome, described in this section and below in section 6.

In rendering heart transplantation an extracardiac procedure and eliminating the need for general anesthesia, cardioplegia, and intubated oxygen delivery, single or double switch heart transplantation eliminates the risk of serious complications, especially during gestation and delivery. In an infant, slightly less than one hour under general anesthesia and awake-regional anesthesia result in no meaningful distinction in neurocognitive development by five years old. Once implemented, switch transplantation will allow a curtailment in intracardiac procedures to include only those known to repair congenital heart defects so that circulation will be normal.

Because a congenitally abnormal heart initiates degradation in all the organs and tissues of the body, a malformed heart should be replaced in utero. And because extracardiac, switch transplantation allows the removal and replacement of the defective heart intact, more safely and less traumatically, without intricate intracardiac microsurgery, the operation will be far more likely to succeed—without cardiopulmonary bypass, cardioplegia, or general anesthesia. However, procedures of the complexity and intricacy contemplated herein will usually take more than one hour, often considerably more, anesthetization may be required more than once, and general anesthesia carries numerous risks unrelated to neurodevelopment. For present purposes, general anesthesia in infancy is best avoided.

The literature overall veers toward a preference for the avoidance of general anesthesia (McCann, M. E., de Graaff, J. C., Dorris, L., Disma, N., Withington, D., and 23 others with 76 collaborators 2019. “Neurodevelopmental Outcome at 5 Years of Age after General Anaesthesia or Awake-regional Anaesthesia in Infancy (GAS): An International, Multicentre, Randomised, Controlled Equivalence Trial,” Lancet 393(10172):664-677).

Complications in otherwise healthy, sick, immature, and pregnant patients caused by general anesthesia are infrequent in incidence but numerous in kind (Hepner, A., Negrini, D., Hase, E. A., Exman, P., Testa, L., and 6 others 2019. “Cancer during Pregnancy: The Oncologist Overview,” World Journal of Oncology 10(1):28-34; Smith, G. and Goldman, J. 2019. “General Anesthesia for Surgeons,” Treasure Island, Fla.: StatPearls Publishing, available online at https://www.ncbi.nlm.nih.gov/books/NBK493199/). Inseparable from the potential complications associated with general anesthesia are those attributable to intubation.

Used in conjunction with anesthesia and cross-clamping, the attribution to cardioplegia of adverse complications in isolation from these other factors is seldom distinct. An exception is when the method used to introduce the solution is the cause. On rare occasion, retrograde cardioplegia—when a calcium-free hyperkalemic solution containing magnesium and lidocaine, for example, is introduced into the coronary sinus—results in sinus rupture or perforation of the inner wall of the right atrium, considerably extending the duration of the procedure and increasing the odds for an adverse outcome.

Complications with selective antegrade cardioplegia can be quite serious; for example, a dissection of the left coronary main stem can complicate antegrade blood cardioplegia resulting in a myocardial infarction and death (see, for example, van Putte, B. P., Vink, A., De Bruin, P. C., and Defauw, J. J. 2007. “Selective Antegrade Cardioplegic Perfusion Complicated by Left Main Stem Dissection,” Journal of Cardiovascular Surgery (Turin, Italy) 48(2):247-248). Currently, the restriction of collapse to a single lung, or one lung ventilation are the two methods used to avoid deflation of lung tissue other than that operated on by minimally invasive techniques to include endovascular, endoscopic, robotic, and video-assisted.

Such methods are inadequate for sustaining lung inflation with or without the patient on a ventilator when undergoing a bilateral clamshell thoracotomy for radical cardiac surgery where general anesthesia, bypass, cross clamping, cardioplegia, and so on would best be avoided and regional anesthesia used instead. Provided the problem of iatrogenic pneumothorax could be avoided, extracardiac, a switch heart transplant could be performed under regional rather than general anesthesia, thus avoiding the need for cardiopulmonary bypass, cross clamping, cardioplegia, the risk of postperfusion syndrome, and so on. To perform an operation under general anesthesia and incur the risks associated therewith when the only reason for doing so is to avoid lung collapse is avoided through the use, described in section 6, of a surgical chestdome designed for thoracic surgery.

Contrary to the situation with conventional heterotopic heart transplantation, double heart transplantation using the metered switch technique comprehends in vivo support measures that allow the deliberate selection of a smaller donor heart. When the native heart is not in end stage failure, the added ejection force of a smaller heart with lower right ventricular ejection fraction, or absolute stroke volume, is less likely to result in pulmonary hypertension. To compensate for the temporary reduction in alveoli, the reduced lung will develop additional alveoli over the ensuing year to eradicate any dyspnea the patient would otherwise experience, and any cough and/or gastroesophageal reflux should subside. The volume of lung removed is not so large that alveolar regeneration should require the use of an incentive spirometer or a special exercise routine for the purpose. Within a year of placement of a second heart in the chest, breathing even during rigorous exercise should be little if at all affected.

The cardiotoxicity of chemotherapy and radiation used to treat a malignancy addressed below in this section is typical of the benefit to be gained from the capability to automatically target medication directly to an organ with ancillary means for preventing the exposure of nontargeted tissue to the medication is backward compatible with to retroactively increase the efficacy of all previous pharmacy and surgery. Methods of treatment since discounted, such as the Vineberg procedure, may warrant reevaluation in light of the later advent of such means. In solid organ transplantation, the implanted control system administers the transition from the circulatory system of the donor into that of the recipient as well as continues to target medication or electrostimulation to the graft organ.

Such medical surgery, or surgery to position catheteric piping and connectors to allow the direct targeting of organs or tissues through the blood supply or directly into the parenchyma is undertaken endoscopically through ‘keyhole’ incisions that readily heal. The vessels, such as the renal or hepatic arteries, and structures, such as the urinary bladder, are usually large and easily located without the need for contrast dye. The implanted microcontroller or microprocessor and small flat drug reservoirs are usually positioned in the pectoral region and hardly noticeable from a cosmetic standpoint

Apart from applications of an implanted automatic disorder response system in conventional surgical practice to directly pipe-target drugs to specific organs or tissues, to achieve a normal ventricular absolute stroke volume from, for example, a solitary or double sudden or metered switch transplanted heart as addressed below, intravascular diversion servovalves can be accompanied by and controlled to cooperate with choke servovalves, or servochokes, or sphincteric servovalvalves. Compound servovalves incorporate both diversion and choke capabilities.

Any basic side-entry, diversion, and/or chokevalve can be shielded to convey moderate dose rate radionuclides, for example, and can incorporate a circumferential layer magnetized radially in relation to the long axis of the substrate ductus and encircling jacket to draw nanoparticulate superparamagnetic carrier-bound drugs, for example. Radiation shielding is described and illustrated in copending application Ser. No. 14/998,495, entitled Nonjacketing Side-entry Connectors and Prosthetic Disorder Response Systems, and applies to nonjacketing side-entry connectors-in an implanted prosthetic disorder response system.

And if the pulmonary vascular resistance is more pronounced, the metered switch transplantation technique, administered by an implanted control system, applicable to any organ or combination of organs, can not only optimize the transplantation but provide immediate medicinal, and/or electrostimulatory support to improve the prospects for a heterotopic heart or a heart-lung transplant (see, for example, Reitz, B. A., Wallwork, J. L., Hunt, S. A., Pennock, J. L., Billingham, M. E., and 3 others 1982. “Heart-Lung Transplantation: Successful Therapy for Patients with Pulmonary Vascular Disease,” New England Journal of Medicine 306(10):557-564).

While the term ‘heterotopic’ does apply to experimental positioning of a second heart in the upper abdomen, infrarenally in the lower abdomen, or in the pelvis of laboratory test animals, it is not properly applied to the ‘piggyback’ procedure, which infrequently performed, represents conventional surgery in humans to position the merged hearts orthotopically. Conventionally denoting the nonexperimental conventional merged, or ‘piggyback’ configuration, even though positioning of the donor heart in double heart transplantation as described herein may indeed be heterotopic, use of the term ‘heterotopic’ in conjunction with a double heart transplantation is avoided. Metered switch transplantation in particular directly addresses the causes for rejection and physiological inadequacy, meaning the immune response to allogeneic tissue and the ischemia-reperfusion injury to which the tissue had been subjected.

The degree of reperfusion injury, injury to the electrical conduction system of the heart (cardionector, cardionecteur, natural cardiac pacemaker), and synchronization problems following transplantation is attributable and directly proportional to the ischemia time preceding insertion into the circulatory system of the recipient. Switch transplantation seeks to minimize both sets of interrelated sources of degradation in a graft organ which collaborate to reduce its survivability and secure its failure, both functions of the immune system, the first, present even were it an autograft after having undergone ischemia, and the second, the fundamentally more intense immune response provoked by an allograft.

In switch transplantation, ischemia time, hence, reperfusion injury, is zero, and especially when switch transplantation is metered, graft organ injury in the form of reperfusion injury, vasculopathy, and nervous impairment, all interrelated, should be minimized if not eliminated due to the immune tolerance induction integral in the process of graft organ transfer from the circulatory system of the donor into that of the recipient.

It thus deals with both sets of insults which collaborate to cause graft failure. “After heart transplantation, the allograft undergoes characteristic alterations in myocardial structure, including hypertrophy, increased ventricular stiffness, ischemia, and inflammation which, together with the natural process of aging, may lead to vasculopathy and fibrosis of the donor heart” (Wdowczyk, J., Makowiec, D., Gruchala, M., Wejer, D., and Struzik, Z. R. 2018. “Dynamical Landscape of Heart Rhythm in Long-term Heart Transplant Recipients: A Way to Discern Erratic Rhythms,” Frontiers in Physiology 9:274).

A frequent problem encountered in heart transplantation is the need to safely remove transvenous pacemaker, cardioverter defibrillator or coronary sinus leads which had been needed with the native heart of the recipient. This topic is pertinent to orthotopic heart transplantation regardless of the technique employed, and to heterotopic switch double heart metered switch transplantation, where due to the unavailability of a sound replacement for the native heart, clinical judgement should support replacement with a second heart.

Aside from the advantages over both conventional orthotopic and heterotopic methods, that some 25 percent of patients on a waiting list for a replacement heart die and that the waiting time in many parts of the world has been growing over the past two decades warrants the increased availability of hearts as well as other organs which the switch method of orthotopic and metered double heart transplants would allow.

Currently, end-stage or terminal patients who agree to donate their organs are allowed to undergo cardiac death without having been maintained on life support. Unless the primary cause of death is cardiogenic, such as in a sudden arrest or sudden cardiac death, then severed from the higher centers that govern the action of the heart, the intrinsic timing circuit, or conduction system of the heart, continues the beat but without stability, even before the graft organ, whether the heart, liver, kidneys, lungs, pancreas, gut, or a gland is harvested. This, even though the continuation of normal perfusion by normothermic autologous blood past cardiac death would spare degradation in the organs, glands, and tissues.

Thus, hypoxia commences prior to transplantation only to be joined by reperfusion injury and the immune reaction when the organ is transplanted. Of the two principal causes for transplant organ failure, ischemia-reperfusion injury can be avoided entirely, and rejection more effectively suppressed by the targeted release of immunosuppressants at the supply of the graft organ or organs. An implanted disorder response system can directly switch the circulation of the recipient to and through the donor organ by means of vascular valves, and moreover, directly pipe-target drugs such as immunosuppressants to the treatment site.

By continuously monitoring and directly pipe-targeting drugs to treat any of a number of comorbid conditions, the use of an implanted automatic disorder response system as described in copending application Ser. No. 15/998,002, entitled Ductus Side-entry Jackets and Prosthetic Disorder Response Systems should expand the zone of patients able to undergo a heart transplant, left ventricular aneurysmal reconstructive surgery, or enlarged volume reduction surgery, whether using the Batista reduction left ventriculoplasty procedure for partial left ventriculectomy, or Dor endoventricular circular patch plasty suturing procedure which has been found to provide more dependable outcomes.

That drugs such as immunosuppressants, angiotensin converting enzyme inhibitors, anti-inflammatories, and antimicrobials can be squarely targeted to the graft or surgically treated organ avoids exposure of other tissue and therewith side effects that might affect unintended organs and comorbid tissue that reciprocally stress the heart. The confinement of immunosuppressive uptake to the target organ reduces its graft versus host potential, increases its susceptibility to microchimerization by the host, and leaves the rest of the host body immunocompetent.

Provided the immunosuppressive used has a reversal agent, any release from the graft organ of the immunosuppressive can be eliminated by the release of the reversal agent from a side-entry jacket positioned along the venous drainage of the graft organ. If necessary, still greater restriction to the target is obtained by placement about the fibrosal outer layer of the organ of one or more patch-magnets as described in copending application Ser. No. 15/932,172, entitled Integrated System for the Infixion and Retrieval of Implants. Using this mechanical approach, the drug, if bound to a water soluble superparamagnetic carrier, need not have a reversal agent.

Further to eliminate any continued travel through one or more veins of the graft organ is to prepare the drug, even one for which no reversal agent is available, for magnetic extraction from the veins of the organ whether native or a graft organ. The drug, bound to a water soluble superparamagnetic carrier, is drawn out of the vein by a permanent magnet equipped side-entry jacket positioned about the venous drainage ductus of the graft organ. The magnet is incorporated into the jacket as a concentric layer thereof, radially magnetized to exert its extractive field toward the long axis of the substrate ductus.

An immunosuppressive, for example, is then delivered in the form of a water soluble superparamagnetic carrier-bound nanoparticulate. Methods for definitively targeting drugs to an anatomical target whether directly pipe-targeted or systemically dispersed are described in copending application Ser. No. 15/932,172, entitled Integrated System for the Infixion and Retrieval of Implants.

Although probable, whether this makes it possible for the patient to be given live attenuated, or weakened, vaccine, so that immunized, the rest of the body can fight off the infection, or immunocompromised so that the graft organ is selectively susceptible to infection, or is, but the infection would be obliterated by continuous circulation through it from the rest of the body warrants study. If necessary, the system can gradually or intermittently reduce the level of the immunosuppressive targeted to the graft organ in coordination with the release of the antimicrobial.

The variables include 1. The starting immune competence of the patient and therefore the strength of the response to the vaccine, or counter thereto, 2. The patient has a primary immunodeficiency disorder, or 3. Is elderly, 4. Frail, debilitated, or 5. Sick; 6. The dose and effectiveness of the vaccine; 7. Whether vaccination is accomplished before or after transplantation; in an infant, 8. Whether maternal antibody protection has disappeared; 9. Whether the infection would be systemic if secondarily affecting certain organs such as yellow fever (Flavivirus), which secondarily and differentially involves the liver and kidneys, chickenpox (varicella-zoster herpesvirus/Human alphaherpesvirus 3), or measles (Measles morbillivirus), mumps (Mumps rubulavirus), which differentially affects the parotid glands, and rubella (Rubella virus), commonly immunized against together with combined vaccine; or would selectively and primarily affect a particular organ, such as the liver by hepatitis, and 10. Whether the organ sought out is that transplanted.

Another benefit of the transplant as an isolated target is the ability to selectively and/or differentially target hematopoietic or mesenchymal stem cells, regulatory T cells, or gene-modified regulatory T cells, for example, to the graft organ, or to the rest of the body with or without an immunosuppressive where these are prepared as magnetized and/or electrified, with uptake, as described in copending application Ser. No. 15/932,172, increased with the aid of patch-magnets, for example.

By placing the patient on cardiopulmonary support, membrane oxygenation, or an artificial heart and thus sustaining perfusion past death, the damage to the organs associated with death and a delay in revitalization can be thwarted. In most instances, death occurs away from a medical facility, making the continuation of circulation past death unavoidable; however, a large proportion of patients do expire in a hospital at a time when the knowledge and apparatus required for preserving the body for organ donation are known and available throughout the developed world.

If not already on life support, prospective organ donors who regardless of cause are near death should be placed on life support and transplantation initiated with life support continued until the organ is removed from the donor and positioned within the recipient. To take the organs of a patient in a chronic vegetative state on life support with the potential to recover is universally regarded as a homicide and not suggested. Patients with intact brainstem function in a chronic vegetative state retaining the potential for regaining consciousness while remaining in this condition are not suitable donors. If to end what appears an interminable period of unconsciousness without hope of recovery, life support is intentionally stopped, then upon confirmation of brainstem death, life support should be reinitiated. Long term life support may itself adversely affect organs, which eventuality should be determined before harvesting.

Most literature on the duration of respiration and circulation by extracorporeal membrane oxygenation concerns survival and the postprocedural condition of the patient once taken off, or ‘weaned,’ from the apparatus, not the maximum time a patient having been placed on life support while still alive can be preserved after death. A given patient having died due to any number of causes, here the object is to sustain perfusion past death as long as possible to allow switch transplantation of the solid organs with zero ischemic time and zero reperfusion injury. Damage to blood components over this term as a factor militating against longer duration is ameliorable through transfusion.

Maintaining the patient always the primary consideration, here the question is less how long one can survive on life support than how best to prepare the patient for organ donation, and taken up below in this section, how long past death the body can be successfully perfused. The patient will generally be best prepared for donation if intensive care is maintained past death. If the next of kin wish a severely brain injured relative to be removed from life support, then intensive care should not be suspended pending a formal pronouncement of death. The deceased should be sustained as close to living as possible. Body systems interdependent, neither should disproportionate emphasis be placed upon the sustainment of selected organs slated for transplantation; even though no gland is to be transplanted endocrine support, for example, should be continued.

If the lungs are to be used, ‘normal’ breathing,” (normal respiration, spontaneous ventilation) should be continued through direct brainstem stimulation with or without the aid of a surgical chestdome, with mechanical ventilation kept to a minimum, preferably through the use of high-flow nasal cannula oxygen therapy. In a large center, for the purpose of preserving a deceased patient having been supported by extracorporeal membrane oxygenation past brainstem death in order to transplant the organs by switch transplantation and thus avoid reperfusion injury and organ degradation, this equipment should maintain the donor in condition to serve as a donor. However, the longer circulation and respiration can be sustained, the greater will be the opportunity to transplant the organs, and the longer can be the immune tolerance induction during metered switch transplantation.

Where survival is concerned, beyond the first week or two of support, current venoatrial extracorporeal membrane oxygenation for patients with cardiogenic shock, even with the aid of a left ventricular assist device, is of limited dependability. Unless the condition of ventricular dysfunction is the result of a repairable defect, such as a faulty valve or ventriculoseptal defect, such a heart is unsuitable for transplantation. Indeed, where repair is dependable, such hearts, not having become impaired due to a myocardial infarction, cardiomyopathy, coronary atherosclerosis, or sudden decompression, for example, may be considered for transplantation using a switch method. Otherwise, the other organs sustained on life support past death should be usable.

Mechanical circulatory support subject to lung injury, pneumonia, and time-limited, to allow prospective donors to remain usable for a longer period and prompt the establishment of centralized medical center storage depots, a more spontaneous means for obtaining sustained cardiorespiratory function without these complications would prove beneficial. In large centers with neurological specialists, anesthesiologists, and intensivists, an attempt can be made to eliminate complete and extended dependency upon extracorporeal membrane oxygenation for the sustainment of cardiorespiratory function.

A decompressive craniectomy to preempt the increase in intracranial pressure that will ensue upon death is performed while the prospective donor remains on life support. Nutritional, endocrine, body temperature, blood electrolyte balance, and so on for maintaining a deceased is addressed below in this section. The control of respiration and circulation are intimately interlocked centrally and peripherally. A conventional ventilator allows continued inhalation (inspiration) and exhalation (expiration) past death while the deceased remains on life support.

Infection and deterioration are warded off throughout intact preservation, transfer to the recipient, and postoperatively by incorporating antimicrobials, hormones, and other essential substances likely to have become depleted by infusion into the donor, which is then communicated to the recipient during switch transplantation. If the donor has been implanted with an automatic disorder response system, the detection of shortages in essential substances and the release thereof directly into the vascular tree through ductus side-entry jackets can proceed autonomously with intermittent checking by a specialist.

Unlike respiration, the control of heartrate is internal to the heart, the control center in the medulla applying adjustments thereto as necessitated by exertion, emotional or physical stress, or changes in pressure or acidity of the blood. Direct stimulation of the circulatory center in the medulla is, however, a means for adjusting rate or stroke volume, best accomplished through the direct targeting of epinephrine or norepinephrine directly to the brain through a basic ductus side-entry jacket or servovalve accessory channel placed along the internal carotid such as shown in FIGS. 25A and 25B. For the most part, control over its functioning can be accomplished locally to the heart.

The continuation of brainstem and parasympathetic innervation of the sinoatrial node past death of the higher brain, primarily by the medullary center through the vagus nerve, and its peripheral innervation by sympathetic nerves is thus avoidable; that is, in contrast to respiratory control, access to the central brainstem and autonomic control over circulation should be unnecessary. Neural rather than chemical in discharging command signals to the muscles of the chest, control of respiration is best accomplished by direct electrical, chemical, or optogenetic stimulation, or electrical neuromodulation of the respiratory centers in the brainstem.

Direct stimulation of the cardiovascular control centers in the brainstem, or secondarily the kidneys, are options, but accomplished more simply through the direct targeting of cardioactive substances, such as epinephrine, norepinephrine, antidiuretic hormone, atrial natriuretic peptide, nitrates, or nicotine, directly to the heart, as described and illustrated in FIG. 16, in copending application Ser. Nos. 14/121,365 and 15/998,002, entitled Ductus Side-entry Jackets and Prosthetic Disorder Response Systems. Much information concerning the pneumotaxic and apneustic centers of the pontine respiratory group, and the dorsal and ventral respiratory groups in the medulla, has already been elucidated.

Cardiopulmonary bypass support and even extracorporeal membrane is damaging to blood cells, so that even on extracorporeal membrane oxygenation and blood transfusion, artificial sustainment thus is time restricting. Mechanical ventilation does not damage blood cells but if not correctly adjusted can prove counteractive in damaging the lungs, and sores about the mouth from the mask or injury to the trachea from the endotracheal tube are additional and frequent complications or artificial ventilation. For lung transplantation in particular, it is preferable that circulation and to the extent possible, spontaneous respiration be preserved.

Spontaneous control over respiration by the brainstem sensory center responsive to exertion, altitude, emotional extremes, and temperature is bypassed by direct stimulation of the pontine and medullary dorsal and ventral respiratory groups by means of stereotactically positioned needle electrodes and/or to the extent that the dissipation of the higher temperature it emits will allow, the future application of optogenetics. Still early in development, optogenetics probes are positioned atop the skull to selectively illuminate light-engineered responsive cell types within the brain.

The optic fiber is encircled by an optic canula, which for angular stabilization, is affixed to the skull with ‘cranioplastic cement.’ Such an arrangement is satisfactory for experimentation with rats over a limited time, but not for permanent implantation in human patients to provide long term if not lifelong therapy. Permanaent fixation with adhesives inside the body is unsatisfactory for several reasons, to include hydrolytic and enzymatic breakdown, secretion into the interface, inadequate strength, and adverse tissue reactions. Optogenetics has the potential to suppress infection, a frequent cause for graft organ failure. Following kidney transplantation, for example, optogenetics can also be used to stimulate the pelvis to initiate peristalsis and protect the kidney and bladder against infection.

Adhesives used internally are meant to maintain incisional or fracture interfaces in juxtaposed relation long enough to allow healing by first intention. However, in the future, numerous type probes, diagnostic and therapeutic, of generally styloid confirmation will require to be angle-stabilized to maintain constancy of aim. For this reason, copending application Ser. No. 14/998,495, entitled Nonjacketing Side-entry Connectors and Prosthetic Disorder Response Systems, describes means for the permanent stable angular fixation of generally styloid conformed diagnostic and therapeutic implants to maintain their aim at a target within an organ parenchyma, for example. For optogenetic therapy, the styloid implant is the light source. These use no adhesives, and incorporate means for dispelling adverse tissue reactions.

Initially directed to the ability to selectively control neurons and neuronal circuits in the brain, additional areas of medical practice for which the fixation of optogenetic probes will be essential include noninjurious resynchronization of the heart, sustaining a deceased solid organ donor beyond the term afforded by extracorporeal membrane oxygenation, the intentional use of heat for therapeutic purposes, and stimulation along the urinary tract, primarily of the bladder. Copending application Ser. No. 14/998,495, entitled Nonjacketing Side-entry Connectors and Prosthetic Disorder Response Systems, was filed on 12 Jan. 2016 pursuant to Provisional Patent Application 62/282,183 filed on 27 Jul. 2015, in anticipation of the emergence of a significant medical literature pertaining to optogenetics.

Applications of optogenetics are adaptable with respect to different graft organs transplanted using the switch method. The wider purpose in providing these reliably anchoring connectors is to enable the implementation of implanted automatic disorder response systems capable of providing targeted diagnostic and therapeutic function inside the body indefinitely without the need for surgical reentry. Service thus requires the ability to stabilize diagnostic probes, such as a miniature fiberscope, angioscope, or intravascular ultrasound probe, and/or a therapeutic device, such as a catheter with hollow needle for the positionally targeted release of medication, or the aimed application of electrical or light stimulation as stipulated by the implanted disorder response system controller.

The anchoring needles of nonjacketing side-entry connectors can be electrified to deliver electrical current among the needles in any pattern, such as between the needles of a single connector or between needles of separate connectors, can be hollow to allow injection, and radiation shielded to allow the direct pipe-targeting of moderate dose rate drugs, and these modalities can be used in combination. Conventional injection is through the beveled tips of the needle ejection lumina, but can also be though apertures along the sides of the needles.

Using concurrent unilateral or bilateral injection and passing tissue surface-coplanar side-to-side electrical current between the needles of one or separate connectors can be used for electrophoresis, electromigration, and electro-osmosis with increased tissue permeability, hence deeper drug penetration—effectively, the intracorporeal administration of a drug through noncutaneous, or deep tissue, iontophoresis. Intracorporeal tissue iontophoresis is currently unseen due to the lack of essential apparatus and because the action must take place inside the body. A fully implanted disorder control system requires only to have the microcontroller, or in a comorbidity capable system, the master control microprocessor, loaded with the required subroutine. Copending application Ser. No. 14/998,495 describes and illustrates jackets shielded to allow the delivery of low to moderate dose rate radioactive substances.

The drug reservoirs and lines must be shielded thus as well. The medication is delivered to the anchoring needles through the connector accessory channel or channels just as specified for drug delivery using side-entry jackets on the blood supplies. An important difference is that whereas delivery through the blood supply through side-entry jackets completely perfuses the organ, delivery at the organ surface is targeted not just to the organ but a lesion in a subportion thereof—that is, the targeting is more highly defined. That the placement of nonjacketing side-entry connectors is through insertion through small incisions is invasive is another instance where it is argued that proper treatment at the outset which allows ‘remote control’ ever after is ultimately far superior to any alternative.

Restricted to cutaneous application, a ‘step up’ from a transdermal patch for access to the bloodstream with noninvasiveness the benefit, the potential for directly targeting tissue inside the body, such as to release into restrict an immunosuppressant into the lesions of an inflammatory bowel disease, thus avoiding side effects elsewhere is precluded. Means that deliver drugs into the circulation depend upon an intrinsic affiliation or functional relation of the drug to the intended target, such as the relation of iodine to the thyroid gland or of sex hormones to the gonads. In contrast, direct mechanical delivery of substances into the substrate tissue achieves markedly superior targeting to spare untargeted tissue exposure to the substance; the side effects of chemotherapeutics and steroids, for example, clearly warrant restricted targeting. Iontophoresis also has diagnostic potential. Applied intracorporeally, suitable sensors and removal means are also required. Conventional iontophoresis remains limited to cutaneous delivery.

Direct injection thus can be of any drug in fluid form into any tissue—the detrusor, bowel, rectum, or myocardium, for example, injection in accordance with the prescription program automatically or in response to sensor input data or a strict schedule. Automatic direct intracorporeal drug or other fluid agent pipe-targeting thus has wide potential application in the treatment of both native and transplant organs. Following a metered switch double heart transplantation to add a second heart to that native or the replacement of the native by two hearts, the benefit of this capability is considerable. The chemical alteration, rather than increased penetrability of drugs through the passage of electrical current upon ejection as in conventional iontophoresis, warrants further study. The design of nonjacketing side-entry connectors allows current to be passed between the needles on one side to those on the other in either direction according to any pattern of discharge.

In a deceased prospective organ donor maintained on intensive cardiorespiratory, endocrine, thermal, and other essential support as delineated by Shetty et al., for example (Shetty, V. L., Mali, S. S., Shetty, S. V., and Shinde, P. D. 2017, Op cit.) the effect is unknown as to the duration of preservation. Galvanism may facilitate the anastomosis of donor and recipient nerve endings, such as of the vagus, accelerans, or phrenic nerves (Ball, C. M. and Featherstone, P. J. 2019. “Medical Galvanism—A Prelude to Defibrillation,” Anaesthesia and Intensive Care 47(1):4-6; Cardozo Pinto, D. F. and Lammel, S. 2019. “Hot Topic in Optogenetics: New Implications of in Vivo Tissue Heating,” Nature Neuroscience 22(7):1039-1041).

Substantially normal circulation and respiration of the deceased donor encourage sustaining and harvesting of the heart and respiratory tract last. While subject to adverse side effects, the more so the longer such treatments are applied, processes such as hemodialysis, peritoneal dialysis, liver dialysis, affinity chromatography, and plasmapheresis will usually sustain the kidneys, liver, and most other organs and tissues of a deceased donor long enough so that these as well as the heart or portions of the respiratory tract will remain acceptable for transplantation without all the tissues of the body having become degraded due to the blood having been damaged through the use of cardiopulmonary bypass or extracorporeal membrane oxygenation, for example.

The potential applications for optogenetic as well as electrostimulatory technology pertinent to organ transplantation, control over urinary function, cessation of ventricular fibrillation, and the prevention of sudden arrest as addressed herein have additional potential in establishing the ability to sustain spontaneous ventilation, or ‘normal’ breathing,’ in a deceased patient for organ donation by direct stimulation of the motor or efferent respiratory centers in the brainstem. Such assumes that the donor has a competent diaphragm and lungs.

The preferability of ventilation through direct stimulation is based upon the numerous and serious complications associated with mechanical ventilation, some of no concern in a terminal patient supported for organ harvesting, but others of considerable concern, such as pneumothorax and pneumonia If the diaphragm and/or lungs are not competent, and/or the process of direct stimulation remains in development, mechanical ventilation, which poses a significantly lessened risk of complications in a corpse, is used.

The application of direct stimulation to the brainstem respiratory centers should be initiated well before, with the object of interdicting, and detaining if not preventing, the final stage of ‘coma dépassé,’ or the complete cessation of vegetative activity, whereupon autophagy would ruin the donor. Paradoxically, the application of these measures can be expected precisely to interfere with the ability to identify at exactly what point the donor would otherwise certainly die.

The object in imaging then, is to identify the earliest appearance of a degree in deterioration beyond which no matter how aggressively treated, the patient would certainly die. This, rather than a more advanced state of deterioration, is the time to harvest organs—when to do so would no longer impose any greater threat of death to the patient but endothelial function still remained intact, before ischemia fundamentally degraded graft organ viability. Accordingly, the taking of organs should commence pursuant not to a certification of death but rather to a certification that the patient had ceased to be recoverable.

Some background pertinent to the changes attendant upon death in the brain, hence, to preservation and the potential for the sustainment of spontaneous ventilation past death through direct stimulation of the respiratory and circulatory centers after death, for example, already exists. Death of the higher brain is premonitory for death of the brainstem but the transition is through stages of deepening coma until even the vegetative functions are abolished. Irreversible cessation of brainstem functions follows a loss of perfusion, of which the measures cited herein (see, for example, Shetty, V. L., Mali, S. S., Shetty, S. V., and Shinde, P. D. 2017, Op cit.) should allow the avoidance.

The events preceding complete death of the brain have been studied using several means (Walter, U. and Brandt, S. A. 2019. “Diagnosis of Irreversible Loss of Brain Function (“Brain Death”)—What is New?,” (in German with English abstract at Pubmed), Nervenarzt (Neurologist, Berlin, Germany) 90(10):1021-1030; Rastogi, P. 2016. “The Mystique of Brain Death,” Journal of Clinical and Experimental Hepatology 6(2):79-80; Dhanwate, A. D. 2014. “Brainstem Death: A Comprehensive Review in Indian Perspective,” Indian Journal of Critical Care Medicine (New Delhi, India) 18(9):596-605). Imaging methods allow monitoring the status of patients along this path of descent from partial to complete failure.

Until direct stimulation thus becomes a practical reality, mechanical ventilation, which likewise affords constancy in rate and tidal volume, should be used. Since other brain functions will have been extinguished, or effectively denervated, perceptual and emotional factors which normally adjust breathing in response to exigencies of the moment will have been eliminated. With no exertion, emotion, or seizures, for example, spontaneous respiration should proceed at a constant rate and depth, eliminating the variability that would normally arise in response to contextual factors to reduce constancy as a controlled variable. Given that collateral cerebral pathology, such as epilepsy, and breathing problems of pulmonary rather than cerebral origin can provoke cardiac dysrhythmias, the prevention of irregularities in breathing reduces the potential complications to be encountered in single and double switch heart transplantation, for example.

Pertinent to the sustainment of a fetal donor past death, literature concerning the anatomy and physiology of the brainstem breathing and circulatory control centers in the fetus is included among the references cited. Apart from avoiding the potential injury that can result from the applications of electrical shocks to the heart, light targeting is far more tightly focused. Neuromodulation through electrostimulation and prospectively, optogenetics, is addressed here with respect to sustaining a deceased donor beyond the term afforded by extracorporeal membrane oxygenation, hence, primarily with respect first, to respiration and below, circulation.

In this section, optogenetics is addressed not only as a means for direct stimulation of the respiratory and circulatory centers in the brainstem, but as an adjunct to reduce the intensity if not an alternative to avoid entirely the potential injury of electrical shocks released by cardioverter defibrillators, for example, as pertinent to heart transplantation. Innate control over cardiovascular function is largely elucidated. Along with electrical and chemical stimulation, optogenetics also has potential application to the sustainment of spontaneous ventilation in a brainstem dead organ donor by direct stimulation of the pneumotaxic and apneustic centers of the pontine respiratory group, and the dorsal and ventral respiratory groups in the medulla. Sustainment thus without mechanical manipulation should sustain ‘normal’ breathing, or spontaneous ventilation, longer and without the complications of mechanically assisted ventilation. Optogenetics also has potential applications to the urinary tract, both related and unrelated to kidney transplantation, for example.

Currently, ambulatory outpatients with end-stage disease at risk of death can have an artificial organ and sensor-actuated switch implanted as a bridge to sustain function pending transfer to a medical center which may already have, or if not, can call for a donor organ. However, rather than to have continued intensive somatic support of the body past death, life support of the donor will have already been shut down and the body moved to the morgue, where circulation will likely continue for days but unstably and eventually stop. The same pertains to patients in a persistent vegetative state with brainstem function but determined unlikely ever to recover, when taken off life support.

The small vessels of the graft organ then become more permeable, releasing fluid into the tissue, and the oxidative stress of oxygen deprivation results in chemical damage to the cell contents, so that following even a brief episode of ischemia, the restoration of blood flow draws leukocytes that release inflammatory factors causing further injury to the graft organ which can result in rejection. Still missing will have been a means for accomplishing transfer of the graft organ from the donor on life support or if unavailable, then from a perfusion machine to the recipient with no interruption in perfusion, no hypoxia in the deceased donor, and no ischemia of the graft organ before or during harvesting.

Whether due to anoxia or trauma, a patient maintained on life support is so brain damaged that to recover consciousness is no longer possible. With intact cardiorespiratory function, such a patient is a better source of graft organs; however, technically alive, it is unlawful to harvest organs until all signs of life are gone. In this circumstance, immediately reinstating cardiorespiratory function upon death is essential to preserve the organs in a living condition. By making possible sudden or metered switching of the recipient's circulation from his own end-stage solid organ to that of a donor, intravascular switching valves and servovalves allow transplantation from the donor without interruption in circulation.

There is, therefore, zero ischemia time, and with a heart, zero impairment of the cardionector (cardionecteur, conduction system of the heart), eliminating the risk of heart block and arrhythmia, making possible off-pump, or beating heart, and cross clamp, chilling, and cardioplegic solution-free heart transplantation. In metered switch transplantation, blood flow through the graft organ is continuously and gradually replaced with the blood of the recipient, thus transferring the organ from the circulatory system of the donor to that of the recipient.

Along with the direct pipe-targeting to the transplant organ of supportive agents to include immunosuppressants, anti-inflammatories, and antimicrobials, for example, the endothelia throughout the graft organ are washed over with the blood of the recipient. The release of these agents is executed by an implanted automatic disorder response system in response to data sent to its control processor by implanted sensors. The white blood cells of the donor substantially removed along the endothelia, the risk of graft versus host and host versus graft rejection is reduced and some measure of microchimerization accomplished, reducing the immune response of an abrupt confrontation.

Moreover, the fact that using the metered switch method the endothelium is exposed to the blood of the donor likely accomplishes a measure of immune tolerance induction that also pre-attenuates the sequelae due to hypercholesterolemia, then vasculopathy, which ensues following a heart transplant, more likely in a replacement heart with atherosclerosed coronaries, for example, and a previously implanted automatic disorder response system as described in copending application Ser. No. 15/998,002, entitled Ductus Side-entry Jackets and Prosthetic Disorder Response Systems used to accomplish the transplantation can also target medication such as evolocumab (Repatha®, Amgen, Newbury Park/Thousand Oaks, Calif.) and alirocumab (Praulent®, Regeneron Pharmaceuticals Incorporated, Eastview, N.Y.) and/or a statin directly to the heart following transplantation to further reduce the risk of hypercholesterolemia and vasculopathy (see, for example, Kühl, M., Binner, C., Jozwiak, J., Fischer, J., Hahn, J., and 5 others 2019. “Treatment of Hypercholesterolaemia with PCSK9 [proprotein convertase subtilisin-kexin type 9] Inhibitors in Patients after Cardiac Transplantation,” Public Library of Science One 14(1):e0210373; Alkhalil, M. 2019. “Proprotein Convertase Subtilisin/Kexin Type 9 (PCSK9) Inhibitors, Reality or Dream in Managing Patients with Cardiovascular Disease,” Current Drug Metabolism 20(1):72-82).

Sudden and metered switch transplantation eliminate:

1. Ischemia and reperfusion injury.
2. The need to place the recipient on cardiopulmonary bypass or extracorporeal membrane oxygenation with aortic cross clamping, carrying risk of complications.
3. The need for general rather than regional anesthesia with the risk of postperfusion cognitive deficits and decline in ventricular perfusion and function following heart-lung machine-supported heart transplantation.
4. The risk of problem bleeding, especially in liver transplantation.
5. Incision into the graft organ, and therewith, the risk of surgical trauma which can result in adhesions, scar tissue, and postoperative dysrhythmia that would interfere with or jeopardize a later retransplantation or surgical repair of the transplant organ or its blood supply, such as a later need for coronary artery bypass grafting or valve replacement, and
6. The side-entry jackets with accessory channels (sidelines; drug lines) if not the mainlines (bloodlines) left in place once the recipient is closed, a means for directly piping medication to the blood supply of the graft organ not only allows the immediately responsive, automatic direct targeting of immunosuppressants to the site but by allowing similar delivery of pathophysiologically counteracting substances, expands the zone of extended criteria donor (less than ideal, marginal) organs for transplantation, to some extent alleviating the severe shortage of organs available for transplantation.
7. In switch transplantation, the flow of blood through the donor organ and its supply and drainage vessels in the recipient is uninterrupted, and an anticoagulant or thrombolytic can be directly targeted to these, so that thrombosis is eliminated, making liver transplantation in particular less susceptible to complications.
8. By allowing excessive pressure to be diverted to nearby large vessels, diversion valves can be used to avoid ‘big-heart,’ or hyperperfusion syndrome where the output of a new heart larger than would spontaneously self-adjust to the smaller load or one not so large but posing a danger during the days preceding self-adjustment could then be used as a bridge or for the life of the implant (Shin, H. J., Jhang, W. K., Park, J. J., Yun, T. J., Kim, Y. H., and 3 others 2011, Op cit.), making an oversized graft organ usable, the shortage in infant hearts otherwise severe.
9. Metered switch technique allows immune tolerance induction concurrent with transfer of the graft organ from the circulatory system of the donor into that of the recipient.

By making possible continuity of perfusion during switch transplantation, minimizing surgical trauma in organ transplantation, ductus side-entry diversion valve jackets and servovalve jackets offer critical advantages that improve the odds for transplant success. In eliminating incision into both donor and recipient hearts, the extracardiac heart transplantation made possible by vascular valves or servovalves reduces the surgical trauma of heart transplantation to the extent that less robust hearts can be used, expanding the zone of available hearts. Also made possible by vascular valves, where the ischemia and hypoperfusion-free cross-shunt method described in section 6 for the extracardiac correction of a total or dextro-transposition of the great arteries can be applied, the complete exchange of blood flow between the aorta and pulmonary artery is accomplished without incision into the heart or great vessels, much increasing the odds for a favorable outcome.

Drugs for automatic release by an implanted disorder response system as described in copending application Ser. No. 15/998,002, entitled Ductus Side-entry Jackets and Prosthetic Disorder Response Systems must be formulated as fluids. Freely flowing fluids can be fed forward by gravity. Higher viscosity fluids are driven out of the drug reservoir and forward by the reservoir outlet pump. Access to the central line can be through a subdermally implanted port, which can lead directly to the substrate vessel or to a drug storage reservoir for automatically controlled release by a fully implanted response system. For excurrent as opposed to incurrent lines, an above-skin, or cutaneous, opening in the port allows a bodily fluid to be drained off into a collection bag. A single port can incorporate both above-skin and subdermal openings. The port requires at least two openings, one for the main drugline, the other for supportive substances such as an anticoagulant.

Connection to the substrate vessel with a basic side-entry jacket or intravascular servovalve rather than a hollow needle or hypotube allows delivery into the line, jacket, and vessel of antimicrobials, anticoagulants, and crystal deposition counteractants through the accessory channel or channels of each such connector. Central lines connected by side-entry jackets or vascular valves are minimally double or double lumen—one lumen or line to serve as the mainline to pass drugs, blood, or parenteral nutrition, the other or others—the sidelines that feed into the accessory channel or channels—to supply water, antimicrobials, anti-inflammatories, anticoagulants, and so on through the jacket and the vessel to which it is connected.

Prevention of parenteral nutrition line occlusion is by occasional flushing of the line with water. In an application administered by an automatic fully implanted disorder response system, the preservation of line patency thus can be made automatic. Side-entry jackets, servovalves, and connectors are specifically designed to eliminate leaks, perforations, adverse, or foreign body, tissue reaction, and other problems encountered with central lines which to treat chronic disease, must remain dependable completely and permanently. Properly put in place, allowed to fully heal, and used, ports of the type shown in section 6 allow worry-free swimming and bathing. Catheter fracture and migration, a rare complication with implanted central lines and pleural catheters, is avoided through the use of catheters made of highly pliant materials. Hygienic measures to prevent infection during insertion are no different for any other type central line.

Pending the development of permanently and dependably anticoagulant, or antithrombogenic, and antibiofouling, antibiofilm-coated or impregnated tubing, a slow drip of heparin or another anticoagulant transmitted through the accessory channels will serve to dispel the clogging of the blood conducting lines, or mainlines, by clot. If heparin-induced thrombocytopenia is a risk, then non-heparin anticoagulants such as argatroban, bivalirudin with or without aspirin and/or clopidogrel, danaparoid, or fondaparinux are used.

Because of its immediate response and hesitation to depend upon patient compliance, the postoperatively continued direct pipe-targeting into the coronary arteries of a transplant heart, and/or release into the general circulation of supportive agents, such as an angiotensin II receptor blocker, angiotensin-converting enzyme inhibitor, beta blocker, inotrope, digoxin, hydralazine nitrate, aldosterone antagonist, spironolactone or a diuretic, much less the immunosuppressive, by the implanted sensor-triggered automatic disorder response system can serve to expand the zone of usable replacement hearts and reduce the discarding of those considered less than ideal.

For example, this immediate level of support should make it possible to place two transplant hearts, each undersized or otherwise somewhat impaired and thus reduced in ejection fraction, to satisfactorily approximate the work of a normal native heart. In removing any objection to allowing a plainly essential transplant for a patient on the basis that the patient lacks the ability to adhere to the drug regimen essential to sustain the transplant, the response is that the patient will be brought to the clinic for periodic follow-up and implanted drug reservoir replenishment. In this circumstance, the implanted automatic response system can facilitate survival. The hierarchical control system coordinating the treatment of comorbidities and administering the therapy automatically, developments in gene therapy will allow the amelioration if not remediation of symptoms attributable to the underlying genetic defect or defects.

Time allowing, replacement of an impaired heart is preferably performed using the metered switch technique. If insufficient, the graft heart is reinforced by the addition of a second intact transplant heart. Heterotopic placement is ordinarily used to anatomically merge the better part of a second heart with an impaired native heart, but here the underlying concept, usually applied to overcome pulmonary vascular resistance, is extended to the transplantation of two intact hearts. Merger of the atria in a heterotopic or ‘piggyback’ heart transplant calls for much intracardiac surgery which the switch technique was devised to eliminate, however, the circulatory sufficiency of which two intact hearts, where each can be separately targeted for the release of drugs or electrostimulation, for example, can be made to function in cooperation to exceed the circulatory sufficiency of a conventional heterotopic transplantation.

The automatic system both administers the metered switch transplantation of the first heart, and thereafter supports both hearts, functioning individually and together as a unit, by instantly releasing routine maintenance or emergency medication directly targeted to either heart through drug delivering catheters according to sensor data received by an implant microcontroller. While the indicia sought exceed those which current cardioverter defibrillators, today often combined pacemaker/cardioverter defibrillators, can provide, these can participate as sensors.

Various implanted sensors allow the measurement of a number of constituents in a passing flow, and if necessary, the shunt can be given an interior coating that aids quantification, such as by attracting a constituent in passing blood up to a ceiling limit that prevents obstruction and allows a sensor to yield real time telemeterable diagnostic data, such as the blood glucose level (see, for example, Huyett, L. M., Mittal, R., Zisser, H. C., Luxon, E. S., Yee, A., Dassau, E., Doyle, F. J. 3rd, and Burnett, D. R. 2016. “Preliminary Evaluation of a Long-Term Intraperitoneal Glucose Sensor with Flushing Mechanism,” Journal of Diabetes Science and Technology 10(5):1192-1194). Before fibroblasts form a capsule about an implant, the foreign body reaction itself causes the adhesion of many blood constituents, allowing these to be quantified. Blood flow measurement does not require such a coating.

In some cases, the measures incorporated into the implanted automatic response system, to include adaptive servovalve adjustment and the direct targeting of antiarrhythmic drugs, will be sufficient to reverse if not avert a spontaneous desynchronization, so that the need for a cardioverter defibrillator, will be ruled out. Currently the single most effective means for preventing ventricular fibrillation and sudden arrest, a cardioverter defibrillator still carries the risks of infection, premature battery discharge, and inappropriate shock delivery, and the need for replacement once depleted of battery power. Furthermore, with respect to the heart or hearts already transplanted, the implant response system is provided with the resources to deal with any comorbidity or comorbidities that can result in mortality with which a cardioverter defibrillator cannot.

In other cases, the inadvisability of cardioverter defibrillator implantation pertains to hearts which had disease which would have disqualified these for transplantation regardless of the methods to be used. Although the administration of carvedilol can ameliorate the condition, and angiotensin-converting enzyme inhibitors and receptor blockers reduce the incidence of shocks, placed in a donor heart of more severe heart failure and reduced absolute stroke volume, an implantable cardioverter defibrillator can actually prove harmful.

Pacemakers and cardioverter defibrillators would best be superseded by means less likely to damage the heart than through the delivery of powerful (>25 joule) shocks which often warrant ventricular tachycardia or catheter ablation to reduce in frequency (see, for example, Krokhaleva, Y. and Vaseghi, M. 2019. “Update on Prevention and Treatment of Sudden Cardiac Arrest,” Trends in Cardiovascular Medicine 29(7):394-400; Yousuf, O., Chrispin, J., Tomaselli, G. F., and Berger, R. D. 2015. “Clinical Management and Prevention of Sudden Cardiac Death,” Circulation Research 116(12):2020-2040). Alternatively, implanting means for averting ventricular fibrillation other than through the discharge of electrical shocks should allow the reduction in frequency and/or intensity of the shocks.

Pacemakers and cardioverter defibrillators can also damage to the myocardium, cause tricuspid valve regurgitation, perforations of the tricuspid valve leaflet, and consequent to lead induced local injury to the myocardium and mistaken sensing, can induce abnormal pacing which can result in ventrical arrhythmias (Puri, M., Chapalamadugu, K. C., Miranda, A. C., Gelot, S., Moreno, W., and 3 others 2013. “Integrated Approach for Smart Implantable Cardioverter Defibrillator (ICD) Device with Real Time ECG Monitoring: Use of Flexible Sensors for Localized Arrhythmia Sensing and Stimulation,” Frontiers in Physiology 4:300). The inducement of a ventricular arrhthmia can also result from pacing of the right ventricle.

Fidgeting with the device when implanted in a subcutaneous pocket by twisting it, known as ‘Twiddler’s syndrome; already reported almost a half century ago can dislodge the leads, disabling the device. Overgrowth by tissue can displace the leads and prevent proper sensing. Still other hazards are battery depletion that goes unnotced or unattended and radio frequency, or electromagnetic interference (Puri, M., Chapalamadugu, K. C., Miranda, A. C., Gelot, S., Moreno, W., and 3 others 2013, Op cit.).

Inappropriate shocks sometimes occur, the heart must have sufficient robustness to withstand these, and beyond a certain level of impairment, the failing heart may lack the wherewithal to respond appropriately to shocks whether appropriate or not. Shortcomings associated with implantable or subcutaneous cardioverter defibrillators in hearts at Class II failure, for example, might argue for the less aggressive and less potentially harmful treatment provided by an implanted response system able to automatically target drugs to the venae cavae according to the onset of action of each and control the diversion and choke valves to adjust the pressures within the heart.

This is because an inherent problem with the use of electrostimulation is that, with preventable causes such as drugs, acidosis, and blood electrolyte and oxygenation imbalances eliminated, the threshold energy of the shocks required to resynchronize a heart is proportional to the degree of impairment; a high defibrillation threshold is associated with more severe failure, the long term use of antiarrhythmic drugs, and increased risk of sudden cardiac death (see, for example, Biton, Y., Daimee, U. A., Baman, J. R., Kutyifa, V., McNitt, S., and 3 others 2019. “Prognostic Importance of Defibrillator-appropriate Shocks and Antitachycardia Pacing in Patients with Mild Heart Failure,” Journal of the American Heart Association 8(6):e010346; Puri, M., Chapalamadugu, K. C., Miranda, A. C., Gelot, S., Moreno, W., and 3 others 2013, Op cit.).

The more severely impaired the heart, the greater is the microstructural and electrical anisotropy or discontinuity along the normal paths of conductance through the myocardium, hence, the greater is the chance that electrical propagation will incur interference which only an increase in shock energy can overcome. The significance and applications of defibrillation threshold test results across a cohort of patients presumes use of the same equipment.

Threshold testing does not reliably predict sudden arrest, and albeit rarely, carries the risk of errors and adverse effects associated with anesthesia. Injury-free, the results, as supplementary to an electrocardiogram, are obtained with a patient in need of an implanted defibrillator passively, rather than by exercise stress testing, to provide an additional indication as to whether the cardioverter defibrillator should be considered a short term bridging device and the patient placed on the waiting list for a heart. While implantable cardioverter defibrillators undoubtedly save many lives, defibrillation threshold testing before implantation of a cardioverter-defibrillator appears to have no effect on mortality.

In the treatment of a failing native heart, rather than apportioning venous return volume between either of two hearts as in a double heart transplant, diversion servovalves can tap off to or draw in blood from neighboring larger vessels and/or choke valves contract or open to increase to decrease more central or intracardiac pressures. Provided with suitable sensors and actuators, an additional approach with the potential to terminate ventricular arrhythmias without the potential to damage the heart as can the shocks from an implantable cardioverter defibrillator and which can be automatically administered by the implanted automatic disorder response system is based upon optogenetics, currently experimental and under study. Neural circuit selective optogenetics and electronic targeting is under intensive study.

One object in developing this technology is to reduce in number and/or intensity if not eliminate the shocks needed to achieve resynchronization. The diagnostic and therapeutic application of optogenetics to medical practice will require the development of less immunologically provocative opsins, reduced heat generation, and techniques for introducing the microbial (viral) opsin, for example, into the structure to be target-infected precisely and more quickly than has been accomplished this early in its development. The tiny light-emitting diodes are implanted to the sides of the infected cells in the brainstem for control by the microcontroller assigned to that level in the hierarchy or directly by the master control microprocessor.

The assistance if not replacement of current means for life support by optogenetic stimulation of the brainstem control centers governing cardiorespiratory function should allow organ sustainment over a longer term. Optogenetic neuromodulation is also pertinent to means for assisting control of the urinary bladder. Once sufficiently refined for application in the clinic, this technology can supplement if not supplant the need for an implanted cardioverter defibrillator which is limited to electrostimulation, or electrode shock based cardiac resynchronization therapy and the use of antiarrhythmic drugs.

Optogenetics has been combined with stem cell therapy in the repair of nervous tissue, which an implanted automatic disorder response system could administer (Yu, S. P., Tung, J. K., Wei, Z. Z., Chen, D., Berglund, K., and 7 others 2019. “Optochemogenetic Stimulation of Transplanted iPS-NPCs [induced pluripotent stem cell-derived neural progenitor cells] Enhances Neuronal Repair and Functional Recovery after Ischemic Stroke,” Journal of Neuroscience 39(33):6571-6594). The energy requirements to power an artificial heart are sufficiently onerous that work over decades on an implantable nuclear powered steam engine to power an artificial heart was eventually abandoned due to a predictable buildup of heat and the certainty of radiation poisoning.

Seeking the maximum benefit with the least expenditure of energy, hence, the longest service life and interval for recharging or replacement of a pacemaker/cardioverter defibrillator, yet another approach to cardiac electrical neuromodulation targets the cardiac autonomic nervous system at multiple levels, to include vagal, stellate ganglion, renal nerves, and spinal cord. Alternative means of electrical neuromodulation may eliminate the need for a potentially injurious shock-emitting cardioverter defibrillator, or supplementary means allow a reduction in the defibrillation threshold energy of the shocks. An automatic implant system can implement and coordinate the technology with the release of drugs each based upon its onset of action.

Optogenetic and autonomic means may not supplant the function of a cardioverter defibrillator but allow its less intensive and less potentially damaging use by reducing the threshold energy required to reinstate a normal sinus rhythm with lower intensity shocks. Such a heart would never be chosen for implantation; indeed, it would be targeted for replacement. Seen thus, in more severely impaired hearts, the combination of immediately responsive diversion servovalve adjustments and automatic delivery of supportive medication in a heart not yet scheduled for replacement would likely prove superior to the use of an electrostimulator or the use thereof not combined with medicinal support.

This pertains exclusively to treatment by the automatic implant response system of a native heart at stage IV failure, not one that would be considered for transplantation. For hearts with a defibrillation too high for the defibrillator to dispel, strategic positioning of the device may remedy the shortfall (Mitra, R. L. 2018. “Left Axillary Active Can Positioning Markedly Reduces Defibrillation Threshold of a Transvenous Defibrillator Failing to Defibrillate at Maximum Output,” HeartRhythm Case Reports 5(1):36-39). Unable to address an eventuality that laid in the future, the literature on cardioverter defibrillators still furnishes sufficient electrophysiological and pharmaceutical information to serve as a basis for planning double heart transplantations.

The use of the implanted cardioverter defibrillator as a primary sensor to signal the master controller of arrhythmogenic events, which in the context of two rather than just one heart, warrant adjuvant treatment consisting of valve adjustments and remediation through the directly targeted release of drugs to the affected heart or hearts, is by connection to the defibrillator cardiac sensors, which in improved defibrillators will include a remote radio transmission, typically, WiFi flexible cardiac sensor array to transmit not just the mere fact of defibrillation but the exact location thereof.

Using this approach, either heart alone might present signs of failure with reduced ventricular absolute stroke volume. If vascular resistance had been a problem for the native heart and a heart-lung transplant was not possible or contraindicated, then a second heart in tandem may allow the pressure of ejection needed to be achieved. When indicated, the likelihood of ventricular fibrillation is reduced by the periodic directly pipe-targeted release of one or more direct contact, or topical, antiarrhythmic drugs not dependent upon processing by the liver, each according to its time to onset, directly into the venae cavae of either or both hearts through accessory channels in the diversion servovalves.

If release into the circulation of the antiarrhythmic would pose the risk of adverse side effects and a reversal agent is available, the reversal agent is released into the aorta through accessory channels in the diversion servovalves for immediate delivery into the end-arterial coronaries. Moving through the Singh-Vaughan Williams classification of antiarrhythmic agents, and omitting those primarily directed to atrial dysrhythmia, those pertinent include Class Ia, quinidine, Class Ib, tocainide, Class Ic, flecainide, Class II, carvedilol, Class III, amiodarone, Class IV, diltiazem, and Class V, magnesium sulfate.

If no reversal agent is available, the drug is formulated as a nanoparticulate bonded to a nanoparticulate superparamagnetic carrier for extraction from the blood ejected either by diversion valve jackets with internal magnets or basic side-entry jackets incorporating a concentric magnet magnetized normal or perpendicularly to the luminal axis of the substrate vessel. H When the cardioverter defibrillator as primary level sensor feeding the implanted master control microprocessor signals a menacing ventricular fibrillation, the microprocessor initiates an algorithm to variously adjust the apportionment of the servovalves at prescribed frequencies to favor recovery of the affected heart or hearts by altering the apportionment between the two of the venous return volume respective of each likely to alleviate the fibrillation.

Using this approach, two marginal hearts that would otherwise have been rejected for transplantation do not go to waste, can be synergistically coordinated to approximate the function of an unimpaired native heart, and so can sustain circulatory function with greater durability than could an assist device were one available. The ultimate objects in this is not only to afford an additional option for ameliorating the condition of the individual patient, but alleviate the shortage in available hearts. The assist heart can, and unless and until a good heart is found, be left in place indefinitely. Double heart transplantation metered or otherwise absent from the literature, it cannot be stated with authority that an assist heart would allow the primary or native heart to at least partially recover function; however the segregable targetability of the assist heart makes this substantially more probable than the assist heart in a conventional heterotopic transplant.

Such a presupposition is, however, compelling, and because the function of each heart can be separately affected, the approach overcomes the deterrent to combine hearts considerably mismatched in size or ventricular absolute stroke volume. It is known from conventional orthotopic transplantation that over- and undersized hearts both gradually adapt to their new situation, an oversized heart gradually relaxing it force of output, and an undersized heart gradually increasing in left ventricular myocardial strength. With a significantly oversized heart, the serious sequelae due to hypertension that ensue following the operation, to include coma and convulsions, have proven controllable.

From this, it must be assumed that two intact hearts would gradually adapt to one another. Because the discrepancy in size of the hearts in a conventional heterotopic transplantation is limited, the potential for either heart to adapt to the other had it been separate is likewise limited. Of the causes cited for post-transplant ventricular malfunction—anesthetic, drug-induced, and/or surgical trauma related none pertain to switch transplantation. Counterintuitively, with an implanted automatic response system as delineated in copending application Ser. No. 15/998,002 to control the excessive ventricular absolute stroke volume or off-timing of an oversized heart, temporary post-transplantation impairment in left ventricle function might even prove beneficial.

That in a conventional heterotopic heart transplant, the difference in size between donor and native hearts is limited, is in itself an additional deterrent, which along with more stringent selection criteria, eliminate hearts in a marginal condition, adding considerably to the shortage of hearts available for transplant, or if having been intended ab initio as a bridge to recovery of the native heart, the size-mismatched heart is removed when it is no longer needed.

It can also be removed having allowed the native heart time to recover but then itself having become infected or otherwise impaired (Tsang, V., Yacoub, M., Sridharan, S., Burch, M., Radley-Smith, R., and 3 others 2009. “Late Donor Cardiectomy after Paediatric Heterotopic Cardiac Transplantation,” Lancet 374(9687):387-392). Other means described herein facilitate correction of a noncomplex extracardiac transposition of the great arteries using synthetic materials served by accessory channels able to dispense antimicrobials, anti-inflammatories, anticoagulant, thrombolytic, or immunosuppressive agents, and rely upon transcatheteric techniques to repair associated intracardiac defects such as septal and valvular. Where transcatheteric methods do not exist to repair associated intracardiac defects, conventional repair is used. Also addressed are aortic coarctation and aneurysm.

Similar means allow a total or partial exchange of blood flow between or among vessels to adjust the blood pressure in each, thereby ameliorating pulmonary or portal hypertension, for example, or the adverse effects of placing a heart in a neonate or infant, too large to adapt to the reduced load in a reasonable time. Applicable to any type ductus, valve and servovalve flow diversion jackets can also serve as a means for the permanent or night-time-only diversion of urine. The advantages in the urinary tract to be gained through the use of synthetic materials introduced with minimal trauma are considerable. Urinary diversion by means other than reconstructive eliminates the need to harvest native tissue, typically gut, which is ill-adapted and susceptible to develop cancer when used to pass urine. Harvesting ill-adapted tissue involves a preliminary procedure with its own potential risks and complications to include significant injury to the gut, and calls for much additional dissection, increased procedural duration, and when necessary, general anesthesia time.

In applying diversion jackets to transplantation of the heart, for example, the abrupt switching of circulation through the recipient from the native to the donor heart without the need for ventilator or cardiopulmonary bypass machine support eliminates damage to the erythrocytes, generates no microemboli or debris, avoids the risk of post perfusion syndrome, or ‘pump headedness,’ with cognitive impairment, and there is no breakdown or spalling of tubing with the release of debris into the blood as has been experienced. No less important, the pressure drop associated with cross clamping using the conventional technique is avoided.

Such valves incorporate an intravascular, or endoluminal, diversion chute which allows diverting all or a continuously variable fraction of the blood supply to and the drainage from the native to the graft organ before the graft organ is harvested, thus eliminating the ischemia-reperfusion injury that fundamentally degrades the graft, materially reducing the odds for success in any solid organ transplantation. This capability instated, medical centers should reserve an area—a donor, not just an organ, maintenance repository—for deceased organ donors on life support.

To sustain a corpse, with no autonomic function, impelled toward autolysis and decomposition, in an organ donor condition is fundamentally more complex than is sustaining a patient in a persistent vegetative state, without higher brain function but retaining a functioning brainstem and autonomic function (Beshish, A. G., Bradley, J. D., McDonough, K. L., Halligan, N. L. N., McHugh, W. M, and 4 others 2019. “The Functional Immune Response of Patients on Extracorporeal Life Support,” American Society for Artificial Internal Organs Journal 65(1):77-83). Whereas a functioning brainstem may sustain body temperature, endocrine, circulatory, pulmonary, digestive, excretive, metabolic, and other bodily functions, in a corpse, these must all be sustained in a coordinated, homeostatic manner artificially.

Following preoperative low dose radiation, microchimerization in preparation for transplantation is accomplished to the extent possible through the exchange of various cell types, which may include thymocytes, hepatocytes, splenocytes, blood and/or hematopoietic stem cells, bone marrow cells, umbilical cord mesenchymal stromal, and/or liver-derived stem cells. Since metered switch transplantation involves no interruption in blood flow, the process of concurrent organ transplantation and immune tolerance induction, if necessary, can proceed a period of days.

Cells obtained from the donor and recipient are exchanged for cross-infusion or alternative conventional administration. Where possible, the different type cells are co-packed. To allow such an exchange of viable cells, whenever possible, the donor should not be allowed to become unfit as the source of preliminary tolerance-inducing cells. If necessary, the cells are exchanged between donor and recipient by air, with the recipient transported to the donor for the switch transplantation. In this way, one donor can be reciprocally cross-microchimerized with multiple recipients to receive different organs.

Maintaining the deceased donor on intensive somatic support past death to include a normal sustained body temperature and endocrine function without interruption in perfusion not only lessens the immunological and hematological impact of brainstem death followed by the additional trauma of excision with no blood flow, best preserving the graft organ itself, but makes it possible for the dead donor as well as the recipient to participate in cross-microchimerization whereby the body of both donor and recipient can be reciprocally cross-microchimerized by cells taken from the recipient as well as the recipient microchimerized with living cells taken from the donor.

The graft organ thus less recoils upon transfer into the circulatory system of the recipient, and the immune system of the recipient less recoils to the presence of the graft organ in his body. When an interval elapses from this preparatory tolerance induction, the blood exchanged during this reciprocal cross-circulation, or cross-transfusion, is not only better matched but more able to prevent rejection. Preliminary reciprocal cross-transfusion in itself can impart a measure of graft rejection or acceptance but will not in itself accomplish mutual cross-microchimerization.

Rather, this is accomplished by immunological preconditioning by preprocedurally cross-transfusing products specified above obtained from either. Preliminary matching of donor and recipient must rule out a significant load or preformed antibodies in the recipient as would immediately precipitate a hyperacute rejection. If not excessive, a preformed load of antibodies may be reduced through plasmapheresis (plasma exchange, immunoadsorption) concomitant with the impairment thereof with the aid of immunoglobulins and rituximab combination therapy, for example.

It warrants emphasis that these findings have not only been disputed, but the claim made that treatment thus induces a significant increase in severe infectious complications requiring hospitalization (Piñeiro, G. J. De Sousa-Amorim, E., Solé, M., Rios, J., Lozano, M., and 10 others 2018. “Rituximab, Plasma Exchange and Immunoglobulins: An Ineffective Treatment for Chronic Active Antibody-mediated Rejection,” BioMed Central Nephrology (London, England) 19: 261). If confirmed, a significant load of preformed antibodies presages hyperacute rejection regardless of the preprocedural measures taken to promote acceptance of the graft organ.

Closely related to maintenance in general is the prevention or curing of disease of a prospective donor. A corpse on life support can contract an infectious disease, and with skilled immunological maintenance, be cured of most. Communicable or not, prospective donors must have been cured of disease before admission into a deceased donor repository. While incurable communicable disease must disqualify a potential donor from admittance, curable disease need not. The establishment of repositories for the maintenance of prospective organ donors alone increases organ availability; the sustainment of normal circulation, hence, organ perfusion, in itself facilitates antimicrobial uptake with improvement in the ability to cure disease.

Once cured, transplantation without interruption in normal perfusion means that organs will be more resilient than would organs preserved over an ischemic period. As a result, the larger pool of available organs established comprises grafts of critically improved survivability. The potential donor will likely require the administration of drugs, and in some cases, a deteriorating organ that would affect the others must be transplanted into the deceased. To accomplish this level of support requires the constant vigilance of highly specialized medical attendants with an array of diagnostic and therapeutic apparatus. For this reason, prospective organ recipients are brought to the site rather than the reverse.

It is central to the establishment of such an initiative that the effort and investment in preservation not be immediately undone due to the lack of a means for transplanting organs so that these are not damaged when harvested. The initiative is justified by vascular valves and servovalves, which eliminate ischemia in organ transplantation. Vascular choke servovalves (chokevalves, servochokes, vascular servochokes, vascular choke valves), are not electronic sphincters or servo chokevlves that compress or constrict the substrate ductus as described and illustrated in copending application Ser. No. 15/998,002 but rather valves of the same kind as those containing a diversion chute where the latter is instead an adjustable obturator or occluder that can be adjusted in the extent to which it extends into the native lumen.

It is the same as a diversion servovalve but the obstructive plate or tang extended into or retracted from the lumen is planar rather than curved. The ductus side-entry jackets, side-entry intravascular diversion servovalve jackets, extravascular and intravascular choke servovalve jackets, described all incorporate features to protect the substrate ductus and include at least one accessory channel, or sideline, to allow the direct targeting of drugs through the device and into the substrate ductus, both critical in protecting the substrate ductus from atheromatous degradation.

The side-entry diversion jackets and valve jackets to be described allow the creation of a dependable on-off switchable diversion path or takeoff from a native lumen and through a tube without anastomosis. Using side-entry devices, conventional anastomosis can be avoided, so that adverse tissue reactions due to direct contact between suture and the native tissue are eliminated. The same applies to catheters made of any other polymeric material when sutured in direct end-to-end or end to side anastomotic contact with a native ductus of lesser caliber rather than connected to the ductus by means of a side-entry jacket.

Irritation and/or the complete enclosure at the adventitial-foam interface, well established to promote atherosclerotic degeneration in the intima, is averted by perforations at numerous points about the outer shell of all jackets and valves which serve to expose enough of the adventitia to the surrounding environment to the body cavity. As evident in FIG. 1, in a basic side-entry jacket, water jacket/accessory channel 7 gives direct access into the jacket foam lining of counterirritant agents where the use of an open cell foam allows permeation to wet the interface.

Similarly, in diversion jackets and valves, potential complications that might be instigated at the adventitia-foam interface, primarily the inducement of atheromatous degeneration due to constriction of the adventitial vessels and nervelets, or vasa vasora and nervora, and continuous enclosure are averted by perforations, or ‘breathing holes,’ part number 40 in FIGS. 1, 2, 5, 7, and 8. In patients atypically sensitive to enclosure of the adventitia, a separate accessory channel positioned to wet the interface with an anti-inflammatory, for example, such as shown in FIGS. 5, 7, 8, 10A, 14, 22A, and 22B is added. With any jacket, diversion jacket, or valve, the adventitial surface can be made directly accessible to drug delivery by positioning a basic side-entry jacket with accessory channel connected to an implanted drug reservoir upstream. The upstream jacket releases the drug onto the surface of the ductus to run along the adventitia to the downstream jacket or valve.

Other approaches include passing a tiny capillary tube-gauged accessory channel-connected upstream ‘soaker hose’ from an upstream jacket or through the target jacket or valve itself through the interface and the use of biomaterials which incorporate anti-inflammatory nanoparticulates, for example, and that simplest and most direct—coating the adventitia with an anti-inflammatory such as a readily absorbed preparation of triamcinolone, for example, as will not block the perforations more than momentarily, before placing the jacket or valve. While a temporary measure, this protects the substrate ductus over the period following placement when the provocation of an adverse reaction is most likely.

The foreign body reaction to the anchoring needles described and illustrated in copending application Ser. No. 14/998,495, entitled Nonjacketing Side-entry Connectors and Prosthetic Disorder Response Systems, seeks to isolate the needles through envelopment within a fibrous capsule. From the standpoint of alleviating any discomfort, this is beneficial; however, just as it interferes with the access of implanted sensors to the tissue to be monitored, anchoring needles which are hollow to allow injection will be obstructed from doing so.

Substances to suppress adverse tissue reactions can be 1. Incorporated within the implant material, 2. Applied thereto as an outer coating, or 3. Released as a fluid from an accessory channel. Various substances for suppressing a foreign body reaction and fibrous encapsulation already exist and can be released either at the adventitial-foam interface or through the injectable anchoring needles of the nonjacketing connector themselves. Another, potentially concomitant approach, is the use of materials with intrinsic resistance to adverse tissue reactions.

Direct synthetic-tissue contact uses materials inherently or given a surface treatment least likely to provoke an adverse tissue reaction. Where critical, such as the interface between the adventitial surface and the foam lining of the jacket or connector, recent innovations in the embedding of antimicrobials into the surfaces of catheters cited below are applied to the incorporation into the thin compliant lining of the foam of agents to suppress infection and adverse reactions. More specifically, the foam lining can itself be lined with a micrometrically thin film of vapor deposited compliant Parylene® (Diamond-MT [owner initials], Johnstown, Pa.) or a similar coating embedded with antibiotic nanoparticles. To prevent atherosclerotic degeneration, jackets and valve jackets for use along arteries must include micropores.

An adverse tissue reaction can be ameliorated through similarly embedding a steroid such as triamcinolone, dexamethasone, acetylcholine, or a nonsteroidal anti-inflammatory such as curcumin, suitable antimicrobials many, and where an especially refractory adverse reaction might arise, one or more capillary-gauged, soft polymeric walled tubes with perforations along the sides much as a ‘soaker hose,’ or side dripping line, connected to an accessory channel can be used to deliver remedial agents directly into the adventitial-foam interface.

The direct pipe-targeting of drugs to nidi or lesions means that in contradistinction to systemic delivery, doses are at once fully utilized, do not affect nontargeted tissue, and can therefore be increased, possibly with improved efficacy or indications as to new applications (see, for example, Kern, D. M., Cepeda, M. S., Lovestone, S., and Seabrook, G. R. 2019. “Aiding the Discovery of New Treatments for Dementia by Uncovering Unknown Benefits of Existing Medications,” Alzheimer's and Dementia (New York, N.Y.) 5:862-870).

Specific anti-inflammatories suitable for treating an adverse tissue reaction topically, or by direct application, here, by direct piping to the treatment site or its blood supply, include dexamethasone, prednisone, prednisolone, phosphorylcholine, cortisone, and possibly, curcumin (diferuloylmethane), reported to possess antimicrobial, antioxidant, anti-neoplastic, and other useful, as well as anti-inflammatory properties. Intact turmeric appears to have wider application than its 2-5 percent constituent curcumin. Whether intact turmeric reverses some potentially adverse effects of curcumin alone is not addressed in the literature.

Vascular valves and servovalves are ductus side-entry jackets which incorporate a diversion chute that when intromitted within the substrate lumen of a ureter allows urine to be diverted directly into a collection bag or blood to be diverted from one vessel into another. Actuated by plunger, or push-pull, solenoids, bistable valves, switchable from fully closed to fully open for use in sudden switch solid organ transplantation, for example, are not continuously variable in response to commands issued by an implanted microprocessor executing a prescription-program in response to data fed back to the microprocessor by process reaction-detecting sensors, so that the valves used must be servovalves. This factor limits solenoid valves to one-for one, that is, initial solid organ replacement or later retransplantation. Double organ transplantation necessitates control over diversion valve extension after the transplantation has been completed and therefore also requires servovalves.

The major applications of vascular valves and servovalves include compound bypass of native to replacement solid organs to allow transplantation without interruption in perfusion, make possible the extracardiac correction of complete or dextro-transposition of the great arteries, bypass a coarctation of the aorta, and adjust the blood pressure between or among vessels, such as to dispel pulmonary hypertension. When the volume of blood to pass calls for adjustment, for example, the valve-jackets are servovalves responsive to an implanted blood pressure sensor signal-fed controller. For use along the urinary tract, endoluminal valves as ureteral takeoffs impart new control over the diversion of urine by ureteral takeoff into a collection bag.

The valve-jackets communicate with a tiny port at the body surface with openings for the introduction of drugs directly pipe-targeted to the treatment site or to outlet urine. The direct pipe-targeting of a drug that requires first pass metabolism in the liver can be released into the portal vein or a tributary proximal to it or if for any reason first pass metabolism would be counterproductive the drug can be released directly into the target tissue through a nonjacketing side entry jacket as described and illustrated in copending application Ser. No. 14/998,495. This capability significantly increases bioavailability and potency, and substantially reduced if not eliminates exposure of nontargeted tissue to the drug.

Most drugs, and certainly more frequently used steroids and chemotherapeutics, for example, harbor potentially and seriously harmful side effects for unintended organs and tissues, and while some negligible run-off into the systemic circulation from the targeted tissue is possible, direct targeting substantially reduces if not eliminates the effect. Another significant benefit is the relatively small dose required when a newer drug would otherwise be unaffordable. Most applications simple, in patients with complex comorbid disease, the ductus side-entry jackets, nonjacketing side-entry connectors, valve-jackets and servovalves are supported for the directly pi[e-targeted release of medication or electrostimulation, for example, by an implanted automatic adaptive hierarchical ambulatory prosthetic disorder response control system.

With such a system, different channels or control arms can be assigned to monitor disease processes whether interrelated, as in metabolic syndrome, or where each is more independent and respond therapeutically with medication or electrostimulation, for example, as appropriate. A master controller microprocessor is fed physiological sensory data which passes up through a series of intermediate hierarchical level microcontrollers. each coordinating the incoming data at its respective levels. Based upon this information, the microprocessor prescription-program is able to optimize the treatment of each morbidity individually and as a component in the combination of disorders to optimize the overall homeostasis and health of the patient.

All the organs and tissues of the body interdependent, the presence of multiple interdependent morbidities is usually characteristic in the elderly with heart failure, for example, where cardiorenal, cardiohepatic, and more recently having come to light, cardiooncologic impairment, are interdependent. That heart disease had been contributed to if not entirely produced iatrogenically as the result of chemotherapy- or radiation-induced cardiotoxicity, however had already become evident. The cardiotoxicity of cardiooncologic therapy joins the myriad side effects of medication systemically dispersed to reach all susceptible tissues and organs when direct pipe-targeting would not only avoid such potentially catastrophic consequences but allow the targeted dose to be increased.

Given that the automatic direct pipe-targeting of drugs to the nidus of a malignancy by an implanted disorder control system, not all cardiopathy or cardiomyopathy coincident with malignancy can be definitively attributed to chemotoxicity or radiation. This is certainly the case where the heart disease preceded the cancer and no radiation had been used. It is becoming clear that heart disease and cancer are not unidirectional—given that both have much the same risk factors, heart disease and cancer can arise together, and it appears that the former can even induce cancer.

Magnetic means for limiting the drug to the target are addressed in copending application Ser. No. 15/932,172 entitled Integrated System for the Infixion and Retrieval of Implants. Essentially, the drug is bound to a superparamagnetic nano- or microparticle carrier to be magnetically drawn radially outward. If the substrate is an artery and the drug is to be delivered into the intima or further abaxially, the surrounding side-entry jacket incorporates a neodymium layer magnetized perpendicularly to the long axis of the underlying vessel, or in the direction of blood flow.

To draw the drug into the parenchyma, it is delivered through the blood supply, and connectors fastened about the outer fibrosal layer of the substrate organ draw the drug with carrier into the parenchyma. The soluble carrier is then broken down. To remove the drug from the bloodstream chemically, a reversal agent is released through a basic side-entry jacket, for example, is described in copending application Ser. No. 15/998,002 entitled Ductus Side-entry Jackets and Prosthetic Disorder Response Systems.

There are then, two methods for preventing a targeted drug that would be harmful to nontargeted tissue from further movement past the target, mechanical and chemical. Where the agent to be interdicted is a radionuclide or other radioactive substance, the delivery pathway is radiation shielded, and to achieve its complete removal, both methods can be applied. If the drug is a solid tumor directed chemotherapeutic and the risk of metastasis is judged significant, a background systemic dose minimized to avoid such a consequence is provided. An unwanted ionizing residue is trapped inside a shielded nonjacketing sise-entry connector.

The importance of confining potentially injurious drugs to the tissue intended cannot be overemphasized; solid tumor chemotherapeutics in particular demand stringent targeting with intentional systemic dosing according to the ability of the malignancy to spread. It was primarily for this purpose that side-entry jackets, connectors, and valves were devised—a permanently implanted automatic response system that targets drugs to diseased or dysfunctional tissue is dependent upon leakless and nonfouling connecting and flow control devices.

Antileukemic myelosuppressives such as fludarabine disrupt hematopoiesis to adversely affect the production of all, not just leukemic blood cells, thus inherently defying positionally focused targeting as applies to spatially defined or delineated tumors. To reach all hematopoietic tissue, the drug must be administered systemically. Such targeting must be chemically, molecularly targeted, rather than positionally and mechanically based. For fludarabine and other myelosuppressives to be rendered harmless to other cell lineages, new drugs selective for leukemic cells or leukemic cell receptors must be developed, or if the use of fludarabine is to continue, then collateral medication to protect nontargeted cell lines or lineages by bonding to their receptors, for example, must be formulated.

The advantage in means that allow isolation of the heart from cardiotoxic agents and radionuclide chemotherapeutics in averting adverse side effects—such as the inducement of heart disease by a chemotherapeutic as addressed below—is incontrovertible, and attests to the advantage in direct pipe-targeting of anticancer agents to the nidus with restriction of continued passage into the circulation, as made possible by nonjacketing side-entry connectors in combination with reversal agents if available and/or magnetic extraction.

Addressed in copending application Ser. No. 14/998,495, entitled Nonjacketing Side-entry Connectors and Prosthetic Disorder Response Systems, radiation shielded delivery makes possible the use of radioactive chemotherapeutics and other substances harmful to untargeted tissue without the constraints that apply when such substances are dispersed throughout the body in the circulation. At the same time, unless the malignancy is too close to the heart or other organ that might be affected, direct beam radiation is usually focused enough to avoid a malignancy-inducing spillover of radiation (Seraphim, A., Westwood, M., Bhuva, A. N., Crake, T., Moon, J. C., and 6 others 2019. “Advanced Imaging Modalities to Monitor for Cardiotoxicity,” Current Treatment Options in Oncology 20(9):73).

The prevention of exposure in the first place is more effective than is the secondary administration of resveratrol and/or hesperidin, for example. Nevertheless, in conjunction with the immune tolerance induction incorporated into metered switch solid organ transplantation, the post transplantation targeting of resveratrol and/or hesperidin, for example, directly to a transplant organ such as the heart should ward off the vasculopathy, which as addressed above, is a frequent cause of late term graft failure. Antineoplastic drugs, to include those antiangiogenic, chemotherapeutic, as well as steroids, for example, are invariably harmful to other tissue. Absent means for contained targeting, radioactive chemotherapeutics will continue to be precluded and unstudied.

While the character of the relationship between heart disease and cancer, both promoted by obesity, smoking, a lack of exercise, poor sleep, metabolic dysfunction, hyperglycemia, hypertriglyceridemia, hypertension, and poor nutrition which includes much sugar and harmful fats to complicate the attribution of such factors to either or both of these disease processes as independent or related is subject to variability in a given patient, the association of chemotherapy and radiation used to eradicate cancer and cardiomypathy is well established.

Turning now to vascular valves, between bistable solenoid valves for one-time use in sudden switch transplantation and urinary prostheses, and continuously adjustable servovalves for use in metered switch transplantation, both addressed below, stand manually controlled valves. Like bistable valves, these are adjusted to be fully open or closed, but can also be adjusted to any position between the two. These are used to divert urine directly from the ureters and into an external collection bag during the night, then be switched to reinstate normal drainage into the bladder, thus preventing the interruption of sleep by the need to urinate.

To replace a resected or malformed lower urinary tract, the need to urinate is automatically signaled and/or controlled as a prosthesis. Devised to avoid injury to the substrate ductus, vascular valves and servovalves are lined with viscoelastic foam to protect vasa vasora and nervora, while in use, only the diversion chute is inserted into the substrate lumen. The structure and use of basic, that is, conventional, generic, ductus side-entry jackets is described and illustrated in copending application Ser. No. 14/121,365 and nonjacketing side-entry connectors in copending application Ser. No. 14/998,495.

As indicated, a luminal flow diversion side-entry jacket is a specially adapted side-entry jacket as described in copending application Ser. No. 14/121,365 or 15/998,002. As such, it can incorporate any of the features of side-entry jackets generally. These include the incorporation between the inside of the jacket shell and foam lining of tiny bioprotectively encapsulated neodymium iron boron alloy permanent magnets, or to allow on-off and graduated intensity, implanted timing module or microcontroller controlled electromagnets, which have been magnetized or oriented to project an attractive field perpendicular to the axis of the native lumen to which the jacket is fastened in encircling relation.

The jacket can therefore function at the same time as an impasse jacket using neodymium permanent magnets magnetized, or electromagnets wound to project an attractive field perpendicular to the axis of the substrate ductus as described in copending application Ser. No. 14/121,365 or 15/998,002. More specifically, a diversion jacket incorporating permanent or electromagnets can be used as an impasse-jacket to stop a superparamagnetic iron oxide nanoparticle-carried drug or drugs against the luminal wall. Because electromagnets allow on-off and adjustable field strength, these can be used with greater flexibility as detention jackets to draw such drugs against the lumen lining or by increasing the field strength, as incorporation jackets to draw such drugs into the lumen wall.

The use of a conventional side-entry or diversion jacket determined by the concurrent need to divert some portion of the flow, when access to the target site is blocked by bone, as with Ewing's sarcoma, the major blood supply artery is jacketed or the bone drilled with the side-entry magnetized on the contralateral side to give direct access to the red marrow of allogeneic hemopoietic stem cells. Other jackets can be strategically positioned proximal to the site for direct targeting of immunosuppressive drugs, the systemic dose then considerably reduced if not eliminated. A side-entry jacket on the blood supply allows the direct targeting of immunosuppressive drugs to a solid organ transplant.

A side-entry diversion jacket allows flow through the native ductus the jacket encircles to be reduced no more than is necessary to draw off a diagnostic sample through a line to the surface port. If outflow is lacking, a fine catheter can be passed through the body surface port, mainline or accessory channel, and into the native lumen to obtain the sample. Magnetized ductus side-entry jackets and diversion jackets positioned along the aorta, ureters, and larger arteries to deliver susceptible drug-carried rituximab and/or steroids locally in higher concentration than if dispersed systemically to treat local lesions should materially reduce the effects of IgG4-related disease without the serious side effects such as moon facies and infusion reactions. Such reactions include pain, infection, and embolism.

A side-entry jacket incorporating small electromagnets positioned just proximal or distal to a bifurcation in the arterial tree where high shear stress promotes the development of atherosclerotic lesions can thus be used to draw remedial drugs in magnetically susceptible form flowing past it into the lesions, for example. A side-entry diversion jacket, which is invulnerable to shear stress, similarly equipped can be positioned just proximal to the bifurcation to allow resection of a segment of an unsalvageable branch or arm of the bifurcation, and deliver medication into the remaining salvageable segment. The value of synthetic prostheses in the vascular tree is greatest in patients with systemic vascular disease who are unable to provide a suitable autologous graft and those debilitated in whom graft harvesting autologous or homologous would be ill advised.

A conventional drug, drugs, or ferrofluid containing a drug or drugs may be injected or infused through a conventional syringe, needle free disposable cartridge jet injector (pneumatic, jet gun, or air gun injector), insulin pump, central venous catheter into the body surface port opening or openings of the accessory channel or channels leading to a conventional or a flow diversion side-entry jacket, where the jacket is positioned superior (upward, cephalad, craniad) to the diversion jacket, or through an accessory channel or channels of the diversion jacket itself. Ductus side-entry jackets and side-entry diversion jackets can also position sensing probes with self-contained diagnostic electronics or electronics implanted in a different location to which the probe is connected in a fibrous encapsulation-resistant location which allows diagnostic data to be obtained in vivo without the awareness of the patient.

To allow medication or facilitated viewing of the junction created, both reducing if not eliminated exposure to unintended tissue, the jacket is provided with at least one accessory channel, which is a line subsidiary to the mainline, a sideline, with side entry to the mainline connected along the jacket side connector. An accessory channel allows the directly piped delivery into the jacket and the substrate ductus to which it is connected of any fluid agent. The utility of an accessory channel is critical, allowing the directly targeted delivery of drugs, medicinal solutions, or contrast into side-entry jackets, side-entry jacket-based extravascular and intravascular choke valves and choke servovalves, diversion valves and servovalves, as well as nonjacketing side-entry connectors and the substrate vessel to which the device is connected.

This not only facilitates observation of the treatment site, but the direct delivery of drugs to suppress any adverse reaction to jacketing the ductus might present and/or allow the directly targeted release of drugs to treat any downstream disease at any later date. A small port at the body surface, much like a larger single port mediport or portacath, and similarly positioned subdermally (subcutaneously, subcorially) in the pectoral region, incorporates the number of entry points for different drugs, or for a frail patient, a number of additional entry ports for future use when and if needed. Tiny tattooed marks on the skin indicate the transcutaneous injection or infusion points.

A urinary diversion line to a paracorporeal collection bag is positioned in an inferior location, preferably to a side of the mons pubis or mons veneris, with only the port opening at the body surface cutaneous, or above-skin. Unlike conventional subcutaneously positioned ports, the distal end of the much smaller multiple opening ports addressed here are securely connected to the target ductus by means of a side-entry jacket or valve jacket. In contrast, the distal end of conventional ports having been known to migrate causing serious complications, a side-entry jacket at the distal end of a conventional portacath would overcome this problem.

Because the foam lining complies with dynamic changes in diameter of the substrate ductus, so that the ductus side-entry jackets to which each drug entry opening, or subport, of the small multiport at the body surface is piped remains stationary in encircling relation to the peristaltic ureter or pulsatant blood vessel. The stable connection of the catheter to the vessel by the jacket ends the risks of dislodgement, fracture, and fragmentation experienced with conventional methods of insertion, to include the sticking of long term hemodialysis catheters following fibrous entrapment. The structure and function of intravascular diversion valves and servovalves is clearly described and illustrated in copending applications parent Ser. No. 14/121,365, continued as Ser. No. 15/998,002.

In such valves, a circular water jacket, just inside and concentric with the core-cutting trepan tube part number 5 in the drawing figures through which a vacuum if applied to the adventitia, is first used to discharge a circular water jet to prevent the extravasation of blood through the entry hole when cutting into a blood vessel. The water jet also repels and thus protects the endothelium or urothelium opposite the entry hole from the trepan cutting edge. Then, once the jacket has been positioned, the same inner water jacket serves as a pathway, the sideline or accessory channel, to deliver drugs in fluid form and medicinal solutions in the antegrade direction into the lumen of the substrate ductus to which the jacket is connected.

The lumen of the trepan tube, or mainline, which conveys blood or urine, can be used in the antegrade direction to deliver fluids into the ductus lumen or in the retrograde direction as an outlet to pass urine, for example. The trepan tube serves two purposes. It 1. Provides a razor sharp surgical circle cutter, or trepan, at its front end which when connected to a suction pump, allows the extraction of a small plug of tissue from the side of the ductus to allow the insertion of the insertion of the diversion chute, and 2. Constitutes the internal front end of the bloodline, or mainline, through which blood or urine will be diverted for shunting of bypass.

Citations to the literature provided both above and below, synthetic-organic interfaces can be prepared to passively resist adverse tissue reactions through the use of 1. Materials embedded with reaction-counteractive nanoparticulates, or 2. An outer coating of a material such as a thin vapor-deposited Parylene® (Diamond-MT [initials of the owner], Johnstown, Pa.), 3. The same additionally impregnated with a similar coating film including embedded nanoparticulate agents to combat infection and inflammation, and 4. At the surface of a solid organ where an adverse tissue reaction were it to occur would be of short duration, microneedle patches, especially those absorbable, can substitute for more costly methods for targeting an anti-inflammatory and/or antimicrobial to the interface on a replenishable basis.

Active resistance is by the direct targeting to the site requiring treatment of remedial agents such as anti-inflammatories and antimicrobials, through accessory channels, or if required at minute locations such as the adventitial-foam lining of side-entry jackets and connectors, through a capillary gauge soft polymer walled tube with numerous perforations in the sides to ‘bleed’ and wet the interface. In arteries, where a thin micromillimetric film lining the foam must incorporate micropores, the line or lines can pass through the foam to ‘bleed’ out of the pores into the interface. Biomaterials with integral nanoparticulates to counteract adverse tissue, or foreign body reactions are addressed elsewhere in this section.

Special cases where, for example, an immunosuppressive is targeted to the jacket to increase susceptibility to infection, may justify the directing of an accessory channel, of which more than one can be connected to the jacket side-stem, through capillary gauged tubing into the adventitia-foam interface to release a steroid or antimicrobial, for example. Albeit very small, except for use with arteries where complete enclosure induces atherosclerotic degeneration, perforation of the Parylene® (Diamond-MT [owner initials], Johnstown, Pa.) or a similar coating to allow the antimicrobial or anti-inflammatory to ‘bleed’ into the interface is not preferred. Any adverse tissue reaction or degenerative alteration at the jacket lining foam-adventitial interface is directly targetable into the interface with inflammation and antimicrobial counteractants.

Damped nonsparking solenoid-driven valves are bistable in completely withdrawing the diversion from the substrate native lumen, thus passing flow through the native lumen without diversion or completely extending the diversion chute into the native lumen, thus diverting flow, whereas manually and linear motor-driven servovalves controlled by an implanted microcontroller can maintain as undiverted or divert any fraction of flow. For temporary use, microneedle patches offer the simplest and least costly means for suppressing an initial short term adverse tissue reaction at the tissue surface-implant interface. Where the substrate structure is an organ with a substantial protective outer fibrosal layer or a ductus other than an artery, microneedle patches can be used to deliver medication to the jacket-adventitia interface.

The medication of a conventional microneedle patch nonreplenishable, these are suited to the suppression of adverse reactions brief in duration. The patch is best perforated, and to not leave behind material that could only be the cause of complications, absorbable. Accordingly, where an adverse reaction is time limited, absorbable polymeric microneedles suffice. The limitation in duration of drug delivery may be overcome by stacking the dissolvable microneedle patches in layers on absorbable backings in a compound patch. Nanometric in proportions, polymer microneedles facilitate the transport of medication across the skin, and could do so across the fibrosa of an organ when used in transfibrosal patches which load medication suitable for topical application in internal use.

As shown in FIGS. 3 and 6, ductus side-entry flow diversion valve or servovalve incorporates a diversion chute 18 mounted on a slideway track 21 which allows the chute with integral ostium or valve outlet obturator 30 at its front end to be advanced from within the trepan tube 5 within the side stem 19 forward into shape-compliant contact with the opposite side of the lumen wall 14 to switch flow away from the lumen and divert it through the trepan tube 5 within the side stem 19 into the shunt or bypass connected to the side stem 19. The details of ductus side-entry diversion jacket and servovalve construction are provided in section 6.

Control of the chute can be binary, or bistable, that is, from the entirely retracted to the entirely advanced position; or the displacement can be continuously variable in extent and rate along the track. Bistable control valves are driven by solenoids, whereas servovalves are driven by linear or other type servomotors. When used in a prosthesis, such as to divert urine past diseased or missing parts of the lower urinary tract, switching from fully retracted to fully advanced is the final adjustment when the prosthesis is placed.

Voluntary control of adjustable ureteral takeoff urinary diversion jackets is by the wearer who rotates a knob on a small surface port in the lower pelvic region, to move the Bowden, or push-pull cables that slide the diversion chutes. The surface port is usually fastened to a side of the mons pubis or mons veneris and also contains the outflow opening for connection to the collection bag of a drainage tube. Ureteral takeoff eliminates filling of the bladder, hence, urge sensation. Where this poses no problem, takeoff can be from the bladder, allowing reduction in the valves and cables to one.

In many cases, a unilateral ureteral takeoff with the contralateral ureter draining into the bladder will afford protection over the interval required. Positioned to one side of the mons pubis or mons veneris the surface port incorporates the opening through which urine passes through a hose into the collection bag. When facilities are nearby, the valves can be switched from diverted to normal, or urethral, outflow. Prior to a public performance, for example, the user incontinent and/or affected by frequent urination switches the valve or valves to divert urine from the ureters to the collection bag. The implantation of a voluntary urine evacuation system is properly responsive only to chronic, not short term conditions such as frequent urination consequent to a lower urinary tract infection readily dispelled by taking an oral antibiotic.

Any urinary voidance prosthesis such as that shown in FIG. 28 or assist system such as that shown in FIG. 30 can be unilateral or bilateral. With a combination outflow/inflow port such as those shown in FIGS. 26C and 28, drugs can be injected into the surrounding drug entry openings; however, inferior, or caudal to the renal pelves, a small pump such as peristaltic positioned close to the floor of the peritoneal cavity, for example, is necessary to move the drug against the force of gravity through the druglines leading into the drainage diversion jacket or valve accessory channels.

If preferred as presaging discomfort or consuming additional power, this can be avoided by introducing drugs at the pectoral level through a second supportive port of the kind shown in FIGS. 26A and 26B, so that delivery is by gravity. Chronic disease of one or both kidneys is treated by positioning basic side-entry jackets such as described and illustrated in copending application Ser. No. 15/998,002 entitled Ductus Side-entry Jackets and Prosthetic Disorder Response Systems, along the renal artery or arteries and delivering the medication through the accessory channels thereof.

Neoplasms within the renal parenchyma are treated with the aid of nonjacketing side-entry connectors as described and illustrated in copending application Ser. No. 14/998,495, entitled Nonjacketing Side-entry Connectors and Prosthetic Disorder Response Systems. Such connectors maintain a therapeutic probe, for example, correctly aimed as the lesion despite movement of the organ treated. Disease affecting the walls of the renal arteries or veins are treated by positioning radially magnetized perivascular side-entry jackets about these to draw superparamagnetic particle-carried medication radially outward from the passing blood into the vascular wall as described and illustrated in copending application Ser. No. 15/932,172, entitled Integrated System for the Infixion and Retrieval of Implants.

Adaptive control over vascular (nonurinary) servovalves is electrical, in a fully implanted system under the control of a microcontroller, or in comorbid disease, a microprocessor, and can be overridden only by a clinician having access to the microcontroller or microprocessor password through a secure online database or patient medical record. In sudden switch transplantation, the diversion of blood requires bistable control—full flow with the chute fully extended and none with the chute fully retracted. In metered switch transplantation, control of the diversion valves is continuously variable, but the implanted master control microprocessor in response to data streams fed to it from implanted physiological sensors.

Where the diversion jackets are to remain and controllably apportion flow between a bypass or shunt and the native vessel, control is generally responsive to feedback from implanted physiological sensors placed at separate locations in the body. Servocontrol by the prescription-programmed microcontroller is typically by continuous adjustment of small linear motor driven diversion chute valve jackets, or intravascular servovalves. Automatic monitoring and response with valve adjustment and the directly targeted release of drugs is essential for ischemia-free metered switch transplantation of solid organs with concurrent immune tolerance induction, the subsequent maintenance thereof, and is especially important in double heart transplantation.

The side-stem or trepan end-piece through which flow is diverted through the jacket shell and into the mainline is the side connector, and distinct from nonjacketing side-entry connectors as described in copending application Ser. No. 14/998,495. If blood, the mainline conducts the effluent through the side connector and into the lumen of the destination vessel—mainline to mainline, through the side connector of the receiving jacket and into the native lumen. If urine, the mainline conducts the effluent to and through the side connector and into the lumen of the ipsilateral or contralateral ureter, or to and through a nonjacketing side-entry connector on the bladder, or to an paracorporeal collection bag, usually cinched about a thigh.

The term ‘downstream’ means in the antegrade direction. In the head (caput), downstream arterial blood flow is craniad (cephalad, upward) and upstream venous flow caudad (downward). A side-entry diversion jacket is unrelated to a conventional vascular or urinary diversion stent, or ‘diversistent, or a J-stent,’ these used, for example, to preserve the integrity of the native ductus such as by preventing the diversion of flow through a nonsurgical fistula. Such stents provide no means for the dissolution of crystal, and usually require replacement at intervals of from one to three months. Contrast this with side-entry jacket or vascular diversion valve-secured urinary diversion lines which are not stents for insertion within native ductus but intended to replace these, and because crystal dissolution agents can be delivered directly into the prosthetic lumen, can be left in place indefinitely.

Where a side-entry jacket is essentially an implantable bisphenol A and residual plasticizer-free polymeric conduit fitting, molded or fabricated in the form of a tee or a wye-joint with unitized stem fixed to any angle relative to the jacket barrel, an adjustable or nonprosthetic side-entry diversion jacket additionally incorporates a continuously deployable curved chute, or tongue, which according to the extent of chute deployment into the native lumen, diverts flow away from the lumen of the jacketed or encircled substrate native ductus.

A choke valve jacket or side-entry diversion valve jacket is not positioned along the length of a catheter such as a mainline (bloodline, urine line) or a sideline (accessory channel, drugline, drug feedline) to adjust or continuously modulate the delivery of a drug through the line. Rather, the jackets connect the lines at the origin and terminus. Drug delivery and valve adjustment are controlled by an implant microcontroller in less complex disease or microprocessor in comorbid disease. This closed loop negative feedback control responds to data supplied to the controller from implanted physiological parameter sensors.

When the indicia indicate the need, in accordance with its preferably theranostic prescription-program, the control device, adjusts the diversion valves and targets medication to the site. Any side-entry component can incorporate an accessory channel to drip an anti-inflammatory, such as dexamethasone, prednisolone, triamcinalone, or cortisone, and/or an ameliorant, such as aloe vera onto to permeate and carry to the interface between the adventitia and cushioning foam, usually consisting of an open cell viscoelastic polyurethane.

Drug delivery is by control of the outlet pump or pumps of small flat reservoirs subcutaneously, or subdermally, positioned in the pectoral area, usually injected into and gravity-fed from a small body surface port for controlled released by the system microcontroller or microprocessor through one or more jacket accessory channels. Basic ductus side-entry jackets and side-entry diversion valves or servovalve jackets are positioned in surrounding or collaring relation to the substrate native ductus, not at junctions, such as at the incurrent and excurrent ends of surgical bypass anastomoses.

Entry into the native lumen is gained through an opening or ostium cut in the side of the native ductus by the razor-sharp leading edge trepan at the adductal end of the extendable and retractable side-stem side connector mainline tube which cuts an opening or ostium in the side of the native ductus, so that the lumen of the side connector, or side-stem, is made continuous with that of the native ductus. A benefit of the jacket is a reduction in the intimacy of interconnection of direct synthetic material-to-tissue contact such as the suture line along an anastomosis, where the synthetic is repeatedly run entirely through the wall of the ductus.

To ameliorate an adverse tissue reaction over the foam-adventitia contact interface, the foam lining is impregnated with an anti-inflammatory such as dexamethasone, prednisone, prednisolone, triamcinolone, or cortisone, and to ward off infection, an antimicrobial. The opening, or surgical ostium, is created by the extraction from the side of the ductus of a plug of tissue the same in diameter as the native lumen. To do this, a vacuum pump is connected to the side-stem side connector mainline tube. The application of suction to the side wall of the ductus through the mainline side-stem with trepan against the wall draws the tissue inside the trepan into the side-stem. Gouging injury to the internal wall of the ductus opposite the opening is prevented by the vacuum pump limit switch, which stops in response to the sudden loss of resistance to the vacuum at the instant the opening is completed.

The vacuum can then be used to assist in extracting the plug. Should the plug catch on the front end of the chute so that the momentary vacuum surge does not pulled out, it is removed with the aid of a small caliber, or capillary gauge, suction tube. Copending application Ser. No. 15/998,002, entitled Ductus Side-entry Jackets and Prosthetic Disorder Response Systems, also shows a hook-ended guidewire that can inserted into the side-stem to extract the plug. To aid visibility, the side-stem side connector mainline tube trepan leading edge is marked with contrast, and the polymeric parts, typically made of bisphenol A and residual plasticizer-free polyetheretherketone (PEEK, Invibio Biomaterial Solutions Division, Victrex, Public Limited Company, Thornton Cleveleys, Lancashire, England).

Alternative polymers are numerous and include optically transparent silicone and poly(acrylonitrile-butadiene-styrene) nanocomposites modified with silver nanoparticles (Zia̧bka, M., Dziadek, M., and Pielichowska, K. 2020. “Surface and Structural Properties of Medical Acrylonitrile Butadiene Styrene Modified with Silver Nanoparticles,” Polymers (Basel, Switzerland) 12(1). pii: E197; Zia̧bka, M. and Dziadek, M. 2019. “Long-term Stability of Two Thermoplastic Polymers Modified with Silver Nanoparticles,” Nanomaterials (Basel, Switzerland) 9(1). pii: E61; Zia̧bka, M. and Dziadek, M. 2019. “Long-lasting Examinations of Surface and Structural Properties of Medical Polypropylene Modified with Silver Nanoparticles,” Polymers (Basel, Switzerland) 11(12). pii: E2018; Zia̧bka, M., Dziadek, M., and Menaszek, E. 2018. “Biocompatibility of Poly(acrylonitrile-butadiene-styrene) Nanocomposites Modified with Silver Nanoparticles,” Polymers (Basel, Switzerland) 10(11). pii: E1257).

A side-entry diversion jacket can be used to divert flow from the artery it encircles through its side connector and the catheter, or mainline, connected thereto, to bypass an aneurysmal, traumatized, diseased, atherosclerotic, or healing downstream segment of the artery before returning flow to the artery through an upstream return or reentry side-entry diversion jacket placed to encircle the artery beyond or downstream to the defect. As to terminology, the bypass line is connected to the side connector in both the diversion and reentry jackets, and a sideline enters the side connector to deliver drugs through the side connector and into the lumen.

Using a conventional side-entry jacket with angled side connector as described and illustrated in copending application Ser. No. 14/121,365 or 15/998,002, FIGS. 19 thru 22, sized to tap off blood at the proper flow rate, or an adjustable side-entry diversion jacket with the chute extended to the proper distance to like effect, it is also possible to initiate the bypass at a point along any larger artery such as the thoracic or abdominal aorta; the bypass can thus be of any length and follow any course that will not strangulate an intervening structure.

If collateral surgery is needed, this allows the origin of the bypass to be positioned where it is least likely to interfere. Increase in length of the bypass does, however, increase the rate of anticoagulant drip required. If the bypass must remain, the synthetic bypass is used as a prosthesis with a small port at the body surface through which to inject an anticoagulant into the jacket of the bypass initiating jacket. Except for the relatively brief interval during which the bypass is applied, there is little if any change in blood pressure through the internal and external carotids.

If the port at the body surface is to house a battery, it must be cutaneous; if transdermal energy transfer is used, the port is subdermal. The battery powers a small pump at the outlet of a flat reservoir, usually in the pectoral region, refilled by injection through the port, the pump controlled by an implanted microcontroller, the prosthesis then fully implanted. In general, whereas a nonadjustable side-entry diversion jacket is fully extended to close off the bloodstream, partially extending the chute of an adjustable side-entry diversion jacket into the native lumen allows the diversion of blood that is partial.

As well as allowing the origin of a bypass along any large artery, this capability can be used to adjust the blood pressure through the bypass and the segment of the artery affected. However, barring a medical contraindication, it is less complicated to reduce the pressure by directly targeting a suitable drug into the native lumen through an accessory channel of the diversion jacket. To avert shear stress, bypass around the defective segment is to a downstream side-entry jacket with side connector angled toward the direction of blood return. If the bypass takeoff diversion jacket is adjustable, the chute can be slightly withdrawn to lower the blood pressure through the bypass, or its accessory channel used to deliver a topical vasoconstrictor to increase the pressure.

Use of the same or a different accessory channel is used to drip an anticoagulant into the line from a subdermally placed flat reservoir through control of its pump by a microcontroller likewise fully implanted. Targeting the anticoagulant to the treatment site in a dose too small to affect the systemic circulation protects the patient from problem bleeding in the event of an accident and/or the need for emergency surgery. Drugs delivered in like manner where uptake within the target segment is incomplete and might pose some risk if circulated are neutralized or counteracted through the release of a reversal agent if available by the upstream side-entry jacket or jackets. For this reason, the anticoagulant of choice at this time is warfarin. In an open procedure, control of the chute to include adjustment if appropriate is manual by the operator, anesthetist, or perfusionist.

Whether the bypass jackets and line are removed depends upon the circumstances. If not to be left in place indefinitely, these are removed before closing. Adjustability by a competent patient once closed is by turning a control knob on a small cutaneously (on the skin) positioned body surface port—usually to a side of the mons pubis for viewability—the size of a smaller button connected to the chute by means of a miniature push-pull control cable. If the patient is not competent, then control of the chute if appropriate is by radio remote control, eliminating the need for a surface port with control knob and push-pull control cable, requiring instead a highly miniaturized, fully implanted, transdermal receiver and antenna, the chute then moved by a tiny a fully implanted microcontroller following a prescription program through a tiny linear servomotor.

Thus, by slightly retracting the chute or infusing an anticoagulant, the blood bypassed can be lowered, restored to, or elevated in full mean arterial pressure. A remotely controlled adjustable side-entry diversion jacket positioned along an artery can be used as an electrovalve to control the volume of flow diverted through the catheter, thus differentially apportioning with continuous variability, the blood, hence, the blood pressure, flowing through the native and catheteric lines. This pressure can be dynamically adjusted by an implanted microcontroller responsive to sensors used to detect physiological parameters implanted in the jackets and/or elsewhere in the body.

An aneurysm or obstructive plaque, for example, can be bypassed during repair, essentially restricting any potentially harmful effects on the body as a whole as might result from requirements of the procedure to the treatment site. An aneurysm at less than operable size or one recently repaired, for example, can be bypassed with the blood returned to the artery past the vulnerable segment through a return jacket. In open repair, unless some circumstance becomes evident to recommend that the jackets and line or lines be left indefinitely as a prosthesis, these are removed prior to closing.

That the foam lining the side-entry jackets allow for growth, and the accessory channels allow for the direct targeting of drugs, closure with a small child is possible. The applications of side-entry and valve or diversion jackets should not be interpreted in a limiting sense. For example, vascular bypass using the means to be described is not limited to the carotids or to atherosclerotic plaque, fistulae, aneurysms, false aneurysms, and dissections in the vasculature can arise for any of several reasons, some more durably corrected with the means described herein.

Diversion or takeoff from the artery is through the jacket, bypass catheter, and downstream return side-entry or side-entry diversion jacket, thus avoiding the need for direct synthetic to tissue anastomoses. Thus, neither type jacket is end-to-end anastomosed to the artery at either end but rather placed in collaring or mantling relation to the substrate artery, eliminating suture lines and the complications these bode. The internal moisture vapor protected viscoelastic polyurethane lining the jacket is less likely to provoke an adverse tissue reaction, and should any arise, anti-inflammatory medication can be delivered into the synthetic-tissue interface along with antimicrobials and any other medication in fluid form.

Weaknesses in the vasculature that are highly susceptible to failure or repeated failure are more dependably repaired with side-entry diversion and side-entry jackets used to bypass the weak points. A circumscribed weakness in the vasculature that is highly susceptible to failure if not repeated failure is more dependably negotiated by replacing the segment with a synthetic bypass not susceptible to the weakening conditions rather than through surgical repair. Because the accessory channels allow establishing an anticoagulant drip and the targeted delivery of drugs into it, such a bypass can remain a permanent implant wherewith the drugs delivered are targeted and thus confined to the treatment site, meaning the bypass and the tissue at its origin and destination.

While the diversion of arterial blood is usually from a larger artery to a smaller artery supplying a different territory, another situation where continuous control over the differential division of flow between an artery and a bypass or shunt justifies placement of a valve-jacket is where there is the need to divert blood through a catheter not into its supply artery but rather in emulation of the Vineberg technique, directly into hypoxic tissue, such as a venous stasis ulcer or more likely (and retracing the history of the Vineberg approach, except using synthetic catheters rather than the middle thoracic as the conduits), into an artery supplying the supply territory or zone of the smaller artery. The use of nonjacketing side-entry connectors for this purpose is described and illustrated in copending application Ser. No. 14/998,495, FIGS. 17 thru 19.

If a slight increase in pressure is desired through the native vessel, a local, or topical vasocnstrictor such as phenoxybenzamine, oxymetazoline hydrochloride, or desglymidodrine is released through the jacket to pass over the treatment site without dependency upon the kidneys, liver, or heart and thus without significant effect on the systemic or pulmonary pressures. Where drugs would best be avoided, the blood pressure is precisely elevated through this circumscribed segment through the placement of an endovascular servovalve rather than a bistable solenoid-driven chute valve, outflow prevented by clamping the sideline. The jacket also makes possible the direct targeting of both the pressure drop and medication to facilitate healing rather than to adjust the blood pressure, advantageous where the drugs when combined pose problems.

Following the procedure, the pressure can be controllably reduced at the treatment site by drawing off (diverting, tapping off) a portion of the flow and systemically by continuing the release of dexmedetomidine. Placement of separate adjustable jackets just inferior to the segment to be treated or a single jacket with antipodal chutes each able to divert half of the flow through the common carotid to either of two return or reentry jackets, one each on the internal and the other on the external carotid, each providing half diversion away from the operated segment through their catheters respectively connected to the inferior to the superior jackets, together diverting all flow. allows diverting a controllable fraction of the normal flow through the artery with return through a downstream jacket past the repair.

A jacket with continuously variable chute extension and retraction constitutes an in-line vascular valve, which placed in the vascular tree with chute fully deployed or extended to fully obturate the native lumen, can be used to temporarily or permanently divert all flow through the native lumen into a shunt, leading to an extracorporeal hemodialysis or apheresis machine, for example, or into a bypass, and when attached to a ureter, can shunt the flow of urine into a paracorporeal collection pouch or a bypass about a missing or healing segment of the ureter, the urine then diverted into the bladder if unimpaired. When used as an externally controllable central line for a purpose other than temporary to disperse for example, a drug or parenteral nutrition systemically, such an in-line valve can be placed at any level along the superior or inferior vena cava, jugular, subclavian veins, or the thoracic or abdominal aorta, for example.

Otherwise, the side-entry jacket or side-entry diversion jacket can be positioned at any level along the ductus to which a drug is to be targeted and/or a sensor is to be positioned, for example, while at the same time functioning as a remotely controlled in-line valve which can be deployed to any extent to differentially apportion the flow of blood, drugs, or urine through the ductus with continuous variability between that fraction diverted and that fraction allowed to continue through the native lumen. Such an in-line valve is adjusted manually by the competent patient or medical professional by means of a rotary control knob placed on a small, secure, and infection resistant port at the body surface, ordinarily positioned in the pectoral area. When uptake of a drug is to be concentrated along the segment just upstream to the jacket, the drug is delivered as magnetically susceptible and the jacket magnetized.

In more complex applications where sensors in the jacket or implanted elsewhere in the body detect the need to release various drugs into one or more ductus at different times, control over the in-line valve or valves is entrusted to an implanted microcontroller governed by a prescription program or a clinical remote-control center in radio communication with the jacket, ordinarily, through a cellular telephone. Such Automated and adaptive control is described in copending application Ser. No. 14/121,365 or 15/998,002, entitled Ductus Side-entry Jackets and Prosthetic Disorder Response Systems. Regulated by an automatic control system, an in-line valve along the vascular tree is a medically adapted electrical servovalve and one along a ureter a medically adapted kind of electrohydraulic servovalve.

A side-entry jacket or side-entry diversion jacket can be used to establish a long term or permanent central line to deliver an infusant for dispersion throughout the body or to directly target a particular level along a vessel of the gut, for example, and a side-entry diversion jacket includes a means for adjusting the rate of flow through the line. The line is entered for injection through a small subdermal port at the body surface, or if a higher flow rate is needed, through an above-skin opening in a port of which only the infusion opening is above skin.

Systemic use is for any purpose served by conventional central lines, to include hemodialysis, apheresis, parenteral nutrition, and chemotherapy, for example. Connection to the target vessel is through the jacket or diversion jacket. Fractional directly targeted use with a background systemic dose as appropriate includes, for example, positioning the jacket upstream to a malignant solid tumor, for example, thus adding mechanical to chemical or metabolic targeting of chemotherapeutics, which might be nanoparticulate, supraparamagnetic carried nanoparticulate, and/or insulin potentiated.

Uptake of the drug or drugs over a delimited segment of the target ductus is obtained by injecting or infusing the drug or drugs through the surface port in the form of a ferrofluid wherein these are rendered magnetically susceptible whether by means of carrier bonding. Uptake is then mechanically accomplished with a perivascular jacket incorporating permanent magnets or electromagnets as described in copending application Ser. No. 14/998,495, entitled Nonjacketing Side-entry Connectors and Prosthetic Disorder Response Systems, and Ser. No. 15/932,172, entitled Integrated System for the Infixion and Retrieval of Implants.

If the injectant or infusant is radioactive, the port, accessory channel, and nonjacketing side-entry connector are radiation shielded. Radiation shielding treatment of drug reservoirs, outlet pumps, druglines, and nonjacketing side-entry connectors is shown in FIGS. 9, 10, and 10A of copending application Ser. No. 14/998,495, entitled Nonjacketing Side-entry Connectors and Prosthetic Disorder Response Systems, delineating radiation shielding, absorbable and permanent, at length. In making possible the direct pipe-targeting of radioactive chemotherapeutics to nidi, such components open the way for a new oncology.

Successive neodymium magnets in the downstream, or antegrade, direction are generally increased in magnetic strength as a gradient array, and electromagnets adjusted in field strength in like manner, the exact field strength in either case depending upon the flow rate and pressure of the bodily fluid passing the jacket through the substrate ductus. A strain gauge or pressure sensor can be incorporated into the jacket, as can any sufficiently miniaturized probe or sensor. For the purpose stated, if the pressure is normal, the nominal pressure range can be used; if the pressure is abnormal, accurate measurement is necessary. The use of side-entry and side-entry diversion jackets allows more safe and secure ambulatory monitoring (Garcia-Ortiz L1, Ramos-Delgado E, Recio-Rodriguez J I, Agudo-Conde C, Martinez-Salgado C, and 3 others 2011. “Peripheral and Central Arterial Pressure and Its Relationship to Vascular Target Organ Damage in Carotid Artery, Retina and Arterial Stiffness. Development and Validation of a Tool. The Vaso Risk Study,” BioMed Central Public Health 11:266).

For example, methotrexate therapy of a malignant tumor consists of positioning the jacket upstream along the blood supply to the location of the tumor as illustrated in copending application Ser. No. 14/121,365, publication number 2016/0051806 or Ser. No. 15/998,002, FIGS. 16 and 21. If necessary, uptake along the ensuing segment is expedited by magnet attraction of the drug delivered in a ferrofluid. If the drug is not to be allowed into or added to that already in the circulation, a second jacket at the distal end of the segment, likewise assigned a specific opening in the small port at the body surface, is used to release a reversal agent. Common reversal agents for methotrexate are glucarpidase, and especially for overdoses, the reduced folate leucovorin (5-formyltetrahydrofolate, folinic acid. When not directly targeted at the tumor thus, methotrexate in the circulation is eliminated with a reversal agent.

In the treatment of a malignant tumor with a tendency to metastasize, a background systemic dose is used to supplement, and along with radiation if necessary, assist in eradicating the shedding of malignant ‘daughter’ cells. A nonjacketing side-entry connector is attached directly above or to penetrate the tumor in the parenchyma with drug delivery or therapeutic tool within or close to the tumor. Such an application with proximate drug delivery is illustrated in copending application Ser. No. 14/998,495, published number 20170197028, FIG. 6. Treatment with drug and/or other therapy into the tumor itself through a nonjacketing side-entry connector fastened at the tumor is illustrated in FIGS. 13A and 13B, which can also include or take the form direct treatment with a miniature laser, electrode, injection needle, or any other styloid therapeutic tool at the distal end of a miniature cabled device. Moreover, with radiation shielding, contrast, tracers, and drugs directly targeted thus can be radioactive.

The targeted fraction seldom requires a downstream jacket to deliver a reversal agent, and less still when systemic dispersal is necessary. The permanent implantation of catheters is made possible primarily by the ductus side-entry jacket accessory or service channel or channels, which allow the direct targeting to the jacket and line of crystallization, coagulation, and biofilm formation counteractants to eliminate the occlusion that had prevented the use of smaller gauge synthetic tubing for such purposes. Significantly, the placement of any side-entry jacket except one that requires the cooperation of magnetically susceptible matter in the wall of the ductus used to stent the ductus, as described in copending application Ser. No. 13/694,835, involves no endoluminal entry.

Since no part of the distal end of the catheter is introduced into the vascular lumen, thrombophilia-inducing turbulence, augmented and compounded with further complications due to the presence of a foreign body in the lumen, to include stenosis and the fibrous encapsulation of a long term chemotherapy or hemodialysis catheter, for example, which the use of side-entry and side-entry diversion jackets, expressly intended for long term use, should eliminate. Long recognized, the use of catheters to convey blood has been prevented by eventual occlusion by thrombus and microbial infiltration resulting in the formation of an infectious biofilm, not warranting the high dose administration of an anticoagulant if such must be circulated systemically, creating additional complications. The deposition of crystal along the internal walls of urinary stents means that these must be replaced every three months.

A central feature of side-entry connectors and jackets, of which a flow diversion jacket is an adaptation that incorporates this feature and can incorporate any of the other features of conventional connectors and jacket, is a subsidiary side-entry line referred to as an accessory channel or sideline that allows drugs and other agents to be delivered directly to the substrate tissue underlying a connector and wet down the native or catheteric lumen and tissue to which a jacket is attached. Migration a significant problem, all side-entry jackets have suture loops or eyelets on the outer shell to through which to pass suture and thus secure the jacket at the level where it is placed. This allows fixing an inferior vena cava filter or any other side-entry jacket in position. Side-entry jackets, diversion jackets, and nonjacketing side-entry connectors can incorporate diagnostic, therapeutic, and viewing sensors, probes incorporating sensors, or connected to sensors positioned outside the jacket or connector.

Invulnerable to disease, shear stress, initiating a cytokine cascade upon being injured, endothelial breakdown when compressed, stenosis, or turbulent flow, the preferability of using smaller gauge catheters rather than harvested vessels has long been obvious but obstructed by two major obstacles. The first is that in the bloodstream, thrombus (clot) accumulates along the internal walls of the tubing, while in the urinary tract crystal deposits to reduce the luminal diameter. Another complication is that the interface between the native and synthetic components is subject to degradation as a foreign body.

Ductus, to include diversion, side-entry jackets and nonjacketing side-entry connectors alleviate the deposition of thrombus along the internal walls of vessels and crystal along the internal walls of the ureters, for example, by providing one or more collateral passageways, or accessory channels, into the side-entry jacket or connector and native ductus to which the side-entry device is connected for the delivery of remediating agents—anticoagulants in vessels and crystal solvents in the ureters, with antimicrobial, anti-inflammatories, and an anesthetic, for example.

At the same time, adverse tissue reactions that would induce tissue breakdown and irritation are minimized if not eliminated by avoiding direct-contact native ductus end-to-end or end-to-side anastomosis, by embedding the foam Parylene® (Diamond-MT [owner initials], Johnstown, Pa.) or a similar coating film lining with a nanoparticulate antibiotic, and as addressed above, exceptionally, by using an accessory channel to target adverse reaction-remediating medication into the jacket foam lining-native adventitia interface. Apposition of native tissue and synthetic materials are thus made substantially safe and secure.

Generally, a conventional side-entry jacket with surface port is used to target drugs or parenteral nutrition, for example, directly into the vascular tree, whereas a diversion side-entry jacket is used to partially or completely shunt away the stream for processing through a dialysis apheresis, or plasmapheresis machine, for example. Where anastomoses are desired, as in a solid organ transplant in a fetus or neonate to allow growth, the jackets with mainline removed and side connector capped off and accessory channels are left in place to target medication from the surface port to the anastomoses.

As will be addressed, unlike a conventional central line or central venous catheter, peripherally inserted central catheters, non-tunneled, tunneled, and totally implanted venous access catheters, the internal end of the catheter connected to the vessel by a side-entry or side-entry diversion jacket is not suspended as a foreign body within the native lumen where it presents a cross-sectional profile that continuously changes longitudinally, causing turbulent flow, promoting thrombosis, but rather holds the distal tip of the catheter in flush alignment with the endothelium, thus little if at all disrupting streamline or laminar flow passing the tiny hole in the side of the vessel. Using conventional means, thrombosis is due to turbulence, immune response to the injury of puncturing and entering into, and the presence of a foreign body within, the lumen.

This applies regardless of the reason for placing the central or peripheral such as femoral line, whether to infuse chemotherapy, provide parenteral nutrition, or to connect an extracorporeal therapy machine, such as a dialyzer. The advantages in using a central or peripheral line that connects a special port at the body surface with entry into the native lumen through a ductus or diversion side-entry jacket generally relate to shortcomings historically associated with the use of conventional lines rather than the process supported. For example, the use of a central line as delineated herein for total parenteral nutrition avoids much if not all thrombosis and the greater risk of infection with a conventional line but not the numerous problems intrinsic in long term total parenteral nutrition, such as constant hunger, fatty liver, intrahepatic cholestasis, cholecystitis, cholelithiasis, and intestinal atrophy.

Thrombosis, especially in patients with a malignancy, any other condition that induces thrombophilia, sickle cell, and other diseases, along with infection, has long been recognized as a major complication associated with the use of vascular access lines, and especially so in patients already prone to clotting such as those with cancer (references below). Whether side-entry diversion jackets should replace central lines with body surface port access depends upon the length of time the line will remain in place and the potential for complications in the specific patient. pengSuspension of the distal end of the line within the vessel is responsible for numerous serious complications to which side-entry jackets and side-entry diversion jackets are not, to include not only thrombosis, but stenosis, tissue ingrowth, fracture, fragmentation, and migration.

The ports for use with vascular access devices to be described fit flush and stably to the upper and/or lower surface of the skin and incorporate an antiseptic chamber below a cap as not to require frequent changes of a dressing and securement adhesive or adhesive tape. These features eliminate the need for a properly applied sterile dressing and securement essential to avoid damaging the skin, increasing the chance of infection and eliminating the need for critical maintenance for the very old or young.

The material of the surface port can be impregnated coated, or bonded with an antimicrobial (antiseptic or antibiotic) (Lai, N. M., Chaiyakunapruk, N., Lai, N. A., O'Riordan, E., Pau, W. S., and Saint, S. 2016. “Catheter Impregnation, Coating or Bonding for Reducing Central Venous Catheter-related Infections in Adults,” Cochrane Database of Systatic Reviews 3:CD007878). The references for protective materials and coatings cited below for use in catheters also apply to the surface ports described herein. When a side-entry diversion jacket is partially opened in the vessel, such as during hemodialysis, when, due primarily to the prospective time of treatment, it is desired that the segment shunted about be kept wetted by blood, the chute protrudes into the lumen; however, the accessory channel running beneath it can deliver an anticoagulant such as low molecular weight heparin into the lumen.

Because delivery is targeted, the dose, whether of apixaban, rivaroxaban, dabigatran, low molecular weight heparin, fondaparinux, or a vitamin K antagonist such as warfarin or acenocoumarol, for example, is isolated and too small in terms of the patient overall body mass to induce adverse side effects, such as problem bleeding, inadequate coagulation in the event of injury or the need for emergency surgery, ecchymosis, epistaxis, palor, malaise, dyspepsia, hepatotoxicity, increased risk of stroke upon cessation, or thrombotic microangiopathy as thrombocytopenia if not thrombotic thrombocytopenic purpura and hemolytic anemia throughout the vascular tree. In placing central lines, the wire used to guide the catheter or line into the vessel is as much a source of complications as is the line itself.

When the central line is or is also used to draw blood samples, the delivery of an anticoagulant through the accessory channel prevents clot, which the placement of the line itself promotes, from clogging the line. Double jacketing thus also means that the segment of the ductus beginning at the level of the upper jacket can be infused at a drug concentration intended to treat the affected segment of the ductus, which if harmful to other tissue so that it should not be allowed to continue through the circulatory system, can be neutralized by a reversal agent delivered through the distal jacket. By the same token, if the drug or drugs are needed systemically but at a lower dose, the degree to which these are neutralized can be reduced as necessary. The same applies not just to a segment along the blood supply or drainage of a supplied organ or region of tissue but to the organ or region of tissue itself, where the follow-up jacket or jackets are positioned along one or more major outflow veins.

In contrast, connection of the line to the native ductus is completely outside the native lumen, only a small hole in the side of the lumen wall present which inconsequentially if at all disrupts laminal or streamline flow past it. If an adjustable ductus side-entry diversion jacket as described herein, then the jacket diversion chute must be fully retracted into the trepan line ending piece. The incision or puncture wound to pass through the line into the native lumen and presence therein of a foreign object also initiates a cytokine cascade that induces inflammation and aggravates the tendency to clot, which, for any substance having a reversal agent, a supplementary side-entry jacket distal to that used to deliver the drug or drugs can neutralize by direct targeting into the native lumen of that agent.

Atherosclerotic degeneration of the substrate intima of arteries and adverse reactions are also eliminated by providing numerous perforations through the outer shell and foam lining of the jacket or connector to prevent endothelial or urothelial degradation due to compression of the fine vessels and nerves entering and leaving the adventitia to serve the entire thickness of the luminal wall and close off the segment enclosed from the interior environment. Copending application Ser. No. 15/998,002, entitled Ductus Side-entry Jackets and Prosthetic Disorder Response Systems, provides citations to the literature documenting such degradation. For this reason, a Parylene® (Diamond-MT Corporation, Johnstown, Pa.), or a similar coating film must be provided with micropores.

Bypass to avoid an injured, diseased, malformed, or operated upon segment in the vascular tree is by positioning an inverted adjustable diversion jacket above the lesion or defect and an adjustable upright jacket below the lesion or defect and connecting these together with a catheter in an additional accessory channel in either jacket. If no blood is to pass through the bypassed segment to wet the interior surface surrounding the lumen, then nonadjustable jackets are used. Medication to treat the bypassed segment is by delivery through the chute through-coursing accessory channel of the upper jacket. Medication to treat the segment below the return jacket is by delivery through the chute-through coursing accessory channel of the lower jacket, and medication to treat the vessel at a level superior to the bypass is through a separate side-entry jacket. The presence of certain substances, and/or antimicrobial resistant pathogens, and or transplant incompatible cell surface adhesion molecules in the blood not readily and/or sufficiently remedied by conventional means that would prove especially or selectively injurious to an already diseased or recently operated upon segment of a vessel or the organ the vessel or vessels serve, such as the bronchial arteries to treat the lungs or the hepatic portal vein and hepatic arteries to treat the liver, may justify the placement of a bypass created with ductus or diversion side-entry jackets with or without diversion to an extracorporeal apheresis machine or dialyzer to keep the substance away from, while targeting remedial medication such as antimicrobial into the vulnerable segment.

Unlike use in the urinary tract, where diversion is seldom partial, use in a vessel, usually and artery, diversion will almost always be partial by adjusting the chute to an intermediate extent. Partial diversion in the bloodstream can be obtained with a conventional side-entry jacket with inclined accessory channel used as the excurrent line as shown in copending application Ser. No. 14/14/121,365 or 15/998,002 or an adjustable diversion jacket. For this reason, the control knobs of the adjustable embodiment include positive bidirectional detents consisting of complementary elevations and depressions which divide the rotation into discrete increments. In the bloodstream and/or along the digestive, respiratory, or urinary tracts, real time hemodiagnostics or diagnostic apropos of the substrate ductus using implanted means can log values for numerous diagnostic indicia received from pathological condition detectors incorporated into the jackets which the implanted prescription programed microcontroller can translate into actuation of the surface port fed subdermally positioned small flat drug reservoir outlet pumps into the direct targeting of a drug or drugs.

Lower dose rate radioactive drugs are magnetically carried through radiation shielded lines, as described and illustrated in copending application Ser. No. 14/121,365 or 15/998,002 but to a nonjacketing side-entry jacket as shown in copending application Ser. No. 14/998,495. Also described in copending application Ser. No. 14/121,365 or 15/998,002, at the side-entry conventional or diversion jackets, a permanently magnetized jacket or jackets is used to hold a paramagnetically carried drug or drugs fast against the intima, while at any level intervening between the jackets used to create the bypass, above, or below these, an impasse jacket is used. Holding he magnetized drug or drugs is with a permanently magnetized detention type impasse jacket, while draining of the magnetized drug into the luminal wall is with an electromagnetized or pull-in type impasse jacket. If the medication used to treat the vessel superior to the bypass should be kept from the bypassed segment, the upper or entry jacket is closed off or temporarily closed off by complete retraction of its diversion chute. Keeping noxious substances from the injured segment is by periodic apheresis or dialysis (hemodialysis) as addressed below. A body surface port is then used not to complete a circuit through an extracorporeal machine but rather to provide openings into the accessory channel or channels through which to directly target medication to the bypassed segment to the upper (takeoff, proximal) of the two jackets. The jackets for such use are either conventional ductus side-entry jackets with angled trepan mainline end pieces for partial flow or adjustable diversion side-entry jackets to allow adjustment in the cross-sectional area flow between complete and partial diversion.

The accumulation of clot within the catheter used to connect the upper and lower jackets is avoided by injection or infusion of a heparin into an accessory channel of the upper jacket. The accessory channel or channels of both jackets in the same surface port, infusion of a heparin into an accessory channel of the bypass upper or takeoff jacket and its antidote (reversal agent, counteractant, neutralizer) protamine sulfate through the lower or return jacket targets and restricts the heparin to the synthetic bypass catheter so that the minimal dose is kept away from tissue. The potential for any type heparin, except, perhaps, a synthetic heparin, to induce adverse side effects are thus avoided.

With an anticoagulant delivered through a central line, or central venous catheter, from the body surface port and into a vessel through a side-entry or diversion jacket, provided a reversal agent is available, a second jacket positioned at the distal margin of the central line to be kept free of thrombus can deliver a reversal agent, substantially constraining the anticoagulant to the central line-vessel junction, thus minimizing continued flow of the anticoagulant through the circulatory system—vitamin K1 (phylloquinone) or vitamin K2 (menaquinone) to reverse warfarin (Coumadin), or protamine sulfate to reverse heparin, for example. The direct thrombin inhibitor dabigatran now has a reversal agent—idarucizumab.

Otherwise, a systemically circulated higher dose of an anticoagulant such as heparin has the potential to induce serious side effects, among which are heparin-induced thrombocytopenia, hemorrhage, and an elevated blood potassium level, or hyperkalemia, resulting in cardiac arrhythmia, weakness, and malaise. Bypass about an impaired segment of a major vessel such as the abdominal aorta or inferior vena cava allows that segment to be spared further exposure to injurious or infectious substances or drugs in the blood given to treat other tissue where apheresis or chelation therapy is neither appropriate nor warranted. Blood-borne pathogens, bacteremia, viremia, fungemia, and parasitemia can all further injure an impaired vessel (see, for example, Kozarov, E. 2012. “Bacterial Invasion of Vascular Cell Types: Vascular Infectology and Atherogenesis” Future Cardiology 8(1):123-138).

The term ‘transfusion’ denotes the administration of blood or blood products taken from another and delivered by means of a hypodermic needle. ‘Reciprocal or cross transfusion’ then, denotes this interrupted manual means for exchanging blood or blood products between donor and recipient. The term ‘infusion’ pertains to drugs rather than blood or blood products administered in the same way, ruling out use of the terms ‘reciprocal or cross infusion.’ The term ‘cross-circulation’ denotes the direct and exchange of blood through a continuous circuit connecting the donor and recipient. The term ‘reciprocal cross-circulation’ then denotes that donor-recipient cross-circulation is bidirectional. In cross-circulation as practiced by C. Walton Lillehei, the heart of the parent had to support two bodies—that of a child as a load added to that of the parent's own body. This severely limited its application.

In the metered switch technique, continuously variable control over the rate of diversion chute advancement and retraction, hence, the flow rate of reciprocal cross-circulation, is essential to optimally adjust the diversion chutes for immune tolerance induction concurrent with transplantation. Such control continues as essential in double heart transplantation to apportion blood between two hearts where cardiac hypovolemia would otherwise risk a dysrhythmia if not a sudden arrest.

One approach to dispel this eventuality is by switching between one vena cava of each heart rather than both at the same time briefly and no more frequently than is necessary to keep the right atrium expelling a volume of blood, albeit less than normal. For example, a minimal volume of the terminal portion of venous return during the diastole of that heart otherwise sent 90 percent of its usual volume is switched to either the superior or inferior vena cava of the other heart. One might, for example, switch between either the superior or inferior vena cava leaving the other to flows without valving, or alternate between the superior and inferior venae cavae of either heart, laboratory testing necessary to turn such speculaton into clinically applicable fact.

Since both venae cavae of both hearts are separately valved, this requires only to be entered into the control microprocessor prescription-program. It is also possible to switch between one vena cava of either heart to both of the other heart. Compared to switching or toggling almost the entirety of venous return, these schemes reduce intracardiac hypovolemia. Nevertheless, to toggle ninety percent of the venous return between the hearts at the optimal frequency determined would likely quell this problem.

To prevent its breakdown, the viscoelastic polyurethane foam lining is conformal surface-coated with a chemically isolating nondegradable foam-compliant barrier which consists of a biostable and biocompatible film. Such as a vapor-deposited or sputtered poly(p-xylylene) polymer material is Parylene® (Diamond-MT Corporation, Johnstown, Pa.), or a similar coating film, for example. The literature concentrates on coatings for implantable neuroelectrodes but provides relevant information as to durability and biocompatibility.

Transplantation using the metered switch technique requires the use of vascular servovalves, which can be continuously adjusted to accelerate or decelerate reciprocal cross-circulation between recipient and donor or differently apportion blood between either of two hearts where only the second or both are transplants. In a sudden switch transplantation or carotid endarterectomy whereby the bypass device will become a permanent prosthesis, the native structure then removed, the operator or an assistant depresses a small plunger switch to simultaneously actuate a number of push-pull or plunger solenoid or servovalve-operated valves, the latter affording considerable flexibility for follow-up diagnosis and therapy.

More often greater control is wanted and an example of such a versatile vascular servovalve, suitable for performing a carotid endarterectomy, for example, is shown in FIGS. 10A thru 10E and 25. When the carotids are salvageable, to monitor, diagnose, and promote healing post-endarterectomy by automatically switching between the prosthesis and the native carotids, three levels of use are available. Where the native carotids are unsalvageable, use as a carotid prosthesis is available. Accordingly, four uses can be delineated:

1. As a bypass during carotid endarterectomy. 2. As a prosthesis left in place during post endarterectomy healing to allow switching between the two for diagnostic and therapeutic purposes, relieve the native structure of shear stress to expedite healing, and to automatically activate in the event of an emergency. 3. As a permanent prosthesis left in place to allow switching between the bypass and native structure or between either the internal or external arteries for diagnostic and therapeutic purposes which can be programmed to automatically activate in the event of a detectable emergency, and 4. Where the native carotids had to be extirpated during tumor removal, aneurysm repair, irreversible disease, or accidental trauma, as a permanent prosthesis.

The benefit of leaving the prosthesis in place includes more than automatic emergency responsive switching to it were the native structure to become impaired. If the carotids have remained intact, spontaneous and quickly responsive baro- and chemoreceptive function will remain functional when passage is through the native structure. By the same token, when passage is through the prosthesis, which incorporates blood pressure sensors and oximeters furnishing feedback to the implanted automatic disorder control system microcontroller or microprocessor, normal function is simulated.

Periodic switching from the native carotids to the prosthesis allows immediate diagnosis as to the state of healing and recovery of the native structure. Such intermittent switching between native and prosthetic carotids can be automatic in response to sensor inputs, perhaps executing an automatic periodic diagnostic routine, or manual. Testing typically includes checking the response time to changes in blood pressure and glucose uptake by the brain responsive to shifts in posture, exertion, or intense emotion, for example. These conditions can automatically activate such diagnostics.

As to therapy, upon being alerted by these sensors, the controller commands the release of medication to restore function to within the normal range. The controller does this by signaling the outlet pump of a small flat drug reservoir or a number of these subcutaneously implanted in the pectoral region to release the medication in the dose specified in its prescription-program. Every ductus side-entry jackets, diversion jacket, and vascular servovalve incorporates at least one accessory channel to target medication directly to the treatment site. Supplementary drug targeting can also make use of the accessory channels in jackets and valves positioned upstream.

The drug or drugs released then pass through catheteric lines to be directly pipe-targeted through the servovalve accessory channel or channels to the common carotid, and if necessary, the valves on either or both its branches. Remedial measures might include reapportioning the volume of flow between the external and internal carotids, which too can be written into the prescription-program, which capability is lost if the mainlines, or bloodlines are removed to leave the accessory channels in place. Continued diversion or periodic diversion through the prosthesis post-endarterectomy makes it possible to prevent shear stress while the intima and anastomoses heal. Reciprocally, periodic wetting of the intimas with blood can be beneficial.

Where the carotids are irreparable or the native carotids would benefit from retention of the bypass as a temporary or permanent prosthesis, the device is designed to allow its continued use indefinitely. If following injury or resection as inseparable from malignant tissue, for example, so that the condition of one or both carotids does not permit removal of the device using vascular servovalves of the type shown in FIG. 25, the device is left in place as a permanent prosthesis. Following an endarterectomy or repair of an aneurysm, whether used for the procedure or to stay as a prosthesis, the three valves remain in place so that the accessory lines of each can be used to directly target medication to their respective supply territories.

Carotid endarterectomy is the most frequently performed surgical procedure, and the loss or need for removal of the carotid baro- and chemoreceptors among the rarest. Up to 1.6 percent of elderly patients with comorbidities undergoing a carotid endarterectomy experience a myocardial infarction, a preoperative evaluation of significant coronary artery occlusion most predictive thereof (Clouse, W. D. and Brewster, D. C. 2004. “Cardiopulmonary Complications Related to Vascular Surgery,” in Towne, J. B. and Hollier, L. H. (eds.), Complication in Vascular Surgery, Second Edition, New York, N.Y.: Marcel Dekker, pages 15-48).

A carotid bypass device such as that shown in FIGS. 10A thru 10C and 25 makes possible an ischemia-free carotid endarterectomy under local anesthesia with spontaneous ventilation, or natural breathing, and freedom from the complications often encountered in a fraction of the time conventionally required. Where the native vessels are heavily diseased or have been irreparably damaged as the result of trauma or tumor resection, the device can be applied as a permanent prosthesis, the native tissue extirpated.

Usually, the device can be used midprocedurally with the postendarterectomized carotids anastomosed, in which case the vascular servovalves can be left in place with bloodlines, or mainlines, removed to allow continued use of the accessory channels by the automatic disorder response system. Alternatively, valves preserved for their accessory channels are replaced with upstream basic ductus side-entry jacket such as those shown in copending application Ser. No. 15/998,002, entitled Ductus Side-entry Jackets and Prosthetic Disorder Response Systems. Measures to allow the valves to be minimized in size and weight and the considerations that allow this reduction addressed below.

Accordingly, should duplex ultrasound and/or magnetic resonance imaging or computer tomography angiography reveal a carotid having undergone an accumulation of plaque as to have become remodeled and severely dilated, tenuously stretched, malacotic, or otherwise impaired due to a vasculitis or some other vascular or connective tissue disease as would pose a risk of aneurysm, for example, a decision is made as to whether to retain and repair the native structure or to leave the bypass device with direct drug delivery accessory channels in place as a permanent prosthesis and excise the diseased tissue.

In a case where the results of imaging indicate that the best conventional technique would not achieve a cure, a carotid endarterectomy is never performed—instead, the prosthesis is applied as a permanent prosthesis ab initio. Due to the likelihood for troublesome swings in blood pressure upon the loss of carotid baro- and chemoreceptor function, provided one of the sides is more severely affected, an attempt is made to limit removal to the one side.

That the device shown in FIG. 25 eliminates the deprivation of oxygen or the passage of microemboli to the brain should reduce if not eliminate the incidence of stroke, myocardial infarction, and postprocedural deficits in cognitive function, especially since the procedure is performed endoscopically under local or regional rather than general anesthesia, usually with the patient breathing spontaneously inside the oxygenated chestdome, with or without mechanical assistance. Using the side-entry vascular servovalve-based device shown in FIG. 25, any debris released midprocedurally is trapped in the native carotid bulb and therefore bypassed.

The precise control over the servovalves essential to avert a temporary ischemic attack if not a stoke or myocardial infarction makes necessary the addition to a conventional side-entry vascular servovalve such as used to perform a metered switch transplantation of a mechanical fitting which allows the operator to directly observe and finely adjust the position of the diversion chute manually during the initial period of no current flow when the valves are applied. Such a side-entry vascular servovalve with the addition of a direct manual control feature is shown in FIGS. 10A thru 10C. When the microcontroller or microprocessor allows manual override as an incorporated set of commands, the direct manual control is eliminated, and while negligible, allows a reduction in the size and weight of each valve.

The fine control gained with a servovalve incorporating this feature may make such valves preferable for use in a metered switch transplant where a high degree of precision is desired. The direct manual control device is housed in a clear transparent plastic enclosure, has internal parts contrast, such as tantalum, coated, can incorporate measuring scales, and include a tiny diode for illumination. Experience with the consequences on blood pressure, blood gas homeostasis, and glucose regulation of a unilateral or bilateral loss of the carotid body is known from patients who had undergone a carotid body paraganglioma, glomus tumor, or malignant metastasis, which infiltrative, compelled resection.

Extended radiation often results in deficits of baroreception. While the need for removal, especially bilateral, is rare, the consequences of a loss in carotid baroreception, chemoreception, coordinated function of the solitary nucleus (nucleus tractus solitarii) in the medulla oblongata, a segment of the carotid sinus nerve, and the impairment in cerebral autoregulatory function of the blood pressure consequent to the disabling or extirpation of the carotid bulb or bulbs, much less the further related physiology cannot be more than incompletely addressed.

The consequences of even a bilateral resection subside over time and are medically manageable even without the immediate support of an implanted automatic response system that instantly detects and is able to respond to excessive deviations in blood pressure, heartrate (tachycardia), imbalances in blood gases, and/or glucose levels with medication and electrostimulation, or neuromodulation, as appropriate. This instancy of diagnosis and therapeutic response is critically different than the current situation, and could not have been anticipated in the existing literature. When a bilateral resection is unavoidable, removal is staged so that only one side is operated on for as long a time as possible.

Malignant tumors force resection of the contralateral tumor more promptly than do tumors that infiltrate the adjacent neurology as to prove impracticable to allow separation. Persistent fluctuations in blood pressure, typically hypertensive, and glucose metabolism appear not to result (see, for example, Timmers, H. J. L. M., Wieling, W., Karemaker, J. M., and Lenders, J. W. M. 2003. “Denervation of Carotid Baro- and Chemoreceptors in Humans,” Journal of Physiology 553(Part 1):3-11).

The need for a follow-up patch angioplasty procedure and potential complications as could significantly extend the duration of the procedure or necessitate reentry or a transluminal intervention, primarily stroke, and the possibility of injury to one or more cranial nerves—the hypoglossal, vagus, recurrent laryngeal, marginal mandibular, superior laryngeal, glossopharyngeal, spinal accessory, transverse cervical, and greater auricular (Rockman, C. and Riles, T. S. 2004. “Nonstroke Complications of Carotid Endarterectomy,” in Towne, J. B. and Hollier, L. H. (eds), Complication in Vascular Surgery, Second Edition, New York, N.Y.: Marcel Dekker, pages 475-482)—should be evaluated, as should the risk for the rupture of a vein patch if created.

Unlike a strictly organic surgical treatment, the prosthesis includes means for detecting and instantly responding to a need for medication to be directly targeted to the treatment site. Although a need for bilateral removal of the carotid bulbs is rare and replacement with prostheses best avoided, if bilateral resection, hence, denervation, and therewith, the disabling of carotid baroreceptive function cannot be avoided, the prostheses are equipped with pressure and chemical sensors to signal the implanted controller to instantly release quick onset of action blood pressure adjusting medication through the servovalve accessory channels as appropriate. Drugs for directly pipe-targeted application bypass the liver and must be converted into the post-liver metabolized form.

For directly pipe-targeted application, antihypertensives such as calcium channel blockers, angiotensin converting enzyme inhibitors, angiotensin receptor blockers, thiazide-diuretics, and thiazide-diuretics should not require post hepatic first pass conversion. Calcium channel blockers, for example, directly affect the nephrons, and exposure of the liver to these drugs can produce adverse hepatic and secondary side effects. Direct pipe-targeting eschews the toxic side effects of which such drugs are capable and by bypassing nontargeted tissue, allows targeted drugs to be administered at dosage levels that could not be dispersed throughout the systemic circulation.

Responding within seconds regardless of patient location or mental state is critically superior to current therapy. Essentially, a bilateral removal results primarily in fluctuations in blood pressure unresponded to within the normal few seconds for a period following the operation. Comparable to the loss of baro- and chemoreceptive function originating within the aortic arch, where resection necessitated by aneurysmal repair, for example, is followed by gradual recovery, the recovery of response time or performance level of adaptation can be approximated using electronic means, but left to itself, will never fully recover to that level afforded by intact receptors.

With the implanted automatic response system, the level of therapy subsides as does the postoperative havoc, thus materially improving the quality of life. Preferably, postoperative retention of the native carotid structures can be accomplished bilaterally with the accessory channels coursing through the servovalves used to directly target a statin, known to have topical efficacy, and an anti-inflammatory into the common carotid to pass through both the internal and external carotids. Alternatively, the internal carotid is directly targeted. With either, a background dose of a statin and any other drug judged essential can be dispersed throughout the circulatory system by any conventional route as well as directly pipe-targeted to other loci or nidi.

Most of the literature pertaining to carotid baro- and chemoreception concern pathological conditions affecting these when the anatomy remains intact rather than following resection. Normal function and the consequences of removal pertinent here, references concerned with the effect of various pathological conditions have been omitted. The control of blood pressure is through a network of baroreceptive, hormonal, and renal components, each having wider control in that order.

Deviations from the normal range in blood pressure following baroreceptive dysfunction or resection are still returned to within the normal range within minutes by the hormonal component, joined by the renal fluid pressure control system within hours, showing that baroreception originating within the carotid bulb exerts its control over the blood pressure more quickly but hardly exclusively (Guyton, A. C. 1991. “Blood Pressure Control—Special Role of the Kidneys and Body Fluids,” Science 252(5014):1813-1816).

FIG. 25 shows a ductus side-entry vascular servovalve-based bypass device, or appliance, for performing a carotid endarterectomy without ischemia, hyperperfusion syndrome, extravasation, increased obstruction to blood flow, the risk of releasing debris or a thromboembolus that mid- or postprocedurally could cause what is effectively a temporary ischemic attack if not a stroke or even a myocardial infarction. To eliminate needless detail, the anatomy has been schematized in omitting several ‘twigs’ that supply neighboring structures such as muscles and the thyroid gland, which if bypassed for a brief period should not result in persistent sequelae attributable to the temporary ischemia.

In fact, each common carotid and its branches is protected through contralateral collateral communication—“ . . . by the free communication between the carotid arteries of opposite sides . . . ” (Gray, H. Anatomy of the Human Body, Goss, C. M. (ed.), 1966. Philadelphia, Pa.: Lea and Febriger, page 582). However, where the placement of a bypass cuts off a contralateral branch completely, collateral circulation to branches of the contralateral external carotid may prove inadequate, and collateral circulation from the contralateral common and internal carotid to the ipsilateral carotid cannot provide sufficient oxygen to the brain to prevent a number of serious consequences.

Over the time needed to perform a carotid endarterectomy, cutting off the small branches of the external carotid should leave the patient without any lasting adverse effects. A temporary loss of sensation in the tongue, for example, should quickly dissipate. Where, however, carotid disease, a vasculitis, connective tissue disorder, tumor resection, or trauma makes retention of the bypass device as a permanent prosthesis imperative, alternative measures must be instituted to make certain that unless spontaneously accommodated by collateral circulation, bypassed branches of the external carotid are not completely cut off.

The best solution is to create anastomoses between these and neighboring arteries not of carotid origin. In FIG. 25, of the branches of the external carotid artery included in the figure, ST is the superior thyroid artery, L is the lingual artery, F the facial artery, M the maxillary artery, and SupT the superior temporal artery. Part number 82 is a ductus side-entry servovalve with direct manual control assist device attaching the device to the common carotid artery, 83 the servovalve attached to the internal carotid, and 84, that attached to the external carotid.

Jacketing the carotids poses a problem of encroachment of the diversion valves upon neighboring tissues. The common carotid reaches downward to just short of the root of the neck, where the anatomy is compact toward the central axis of the body but the integument is loose enough that a valve with side stem directed away from the exterior can be placed to neither irritate adjacent tissue nor pose a cosmetic problem. The same cannot be said for the external and internal carotids which arise from the common carotid at about the jawline, “opposite the superior border of the thyroid cartilage” (Goss, C. M. (ed.) 1966, Op cit.).

As in many other loci where the anatomy is compact, it is important to emphasize that a local constriction as allows the diversion jacket or valve to be significantly reduced in size does not in itself provoke medical problems. Mere constriction is readily adapted to; it is the release of microemboli from atherosclerotic plaques in conjunction with arterial narrowing that result in cerebral and coronary infarction. Once the narrowing and diseased tissue have been eliminated, here, through a carotid endarterectomy, the patient should not experience an ischemic event, or accident, any more than before the stroke or heart attack when the artery had adapted while no release of debris had occurred.

Where practicable, the lumen of mainlines, or bloodlines, consisting of the trepan tube in the diversion jacket or valve sidestem and the tubing to which the trepan tube is connected, is made as close in caliber to that of the substrate vessel as possible. However, where the perivascular tissue (less functional perivascular fat which if thick can be trimmed) can be jacketed along with the vessel itself) is tightly fitted about the vessel, once the diseased condition is eliminated, can be jacketed with a much smaller jacket or valve, the body equipped with means for adjusting to a reasonable reduction in the flow-through cross sectional area for the blood to pass.

Following plaque removal, narrowing the carotids to allow a significant reduction in the size and weight of the diversion servovalves eliminates the risk of thromboembolism release from within the common or internal carotid. However, the patient having presented the condition will have atherosclerotic disease, leaving the constricted segment more vulnerable to the passage through the segment or its occlusion by a thromboembolism of remote origin. Hence, if the bypass device is used only during the endarterectomy so that the native tissue remains, the carotids may become restenosed, making the regular release of a statin imperative and ezetimibe appropriate.

In a patient with familial hypercholesterolemia, the development of significant carotid blockages indicates that the proprotein convertase subtilisin kexin-like 9 (PCSK9) RNA interference inhibitor such as evolucumab with ezetimibe had not been used and should now be considered imperative. If the carotids had been so diseased due to pathology other than dyslipidemic or have been left irreparable due to tumor removal or trauma that the bypass must be left in place as a permanent prosthesis, then the narrowing of the carotids by the prosthesis should not require treatment.

As exemplary for similar situations elsewhere in the body, these considerations mean that the diversion servovalves on the internal and external carotids used to create a bypass in order to allow an ischemia-free carotid endarterectomy can be made small enough as not to create postoperative problems that cannot be corrected by endoscopically rotating these and/or placing a cushioning filler.

Moving on now to the switch transplantation of solid organs, in a metered orthotopic heart transplant, the switch transmits a wireless signal to the external master controller which oversees the microcontrollers or microprocessors implanted in the donor and recipient that provide the data to coordinate the reciprocal cross-circulation and concomitant release of supportive drugs in either as necessary to transfer the graft organ from the donor to the recipient.

The controllers then open the diversion servovalves to initiate reciprocal cross-circulation, which the donor and recipient controllers subordinate to an extracorporeal master controller closely monitor for signs of an adverse immune response, dysrhythmia, or other disruptive sign. In a double heart transplant, adjustment of the servovalve setpoints allows hearts different in size and ejection fraction to be paired, while toggling the valves allows the variable entrainment of a number of successive systoles of either heart to be combined thereby allowing adjustment in the net ejection fraction.

Absent toggling, that is, with the valves fixed in position at about the half-way point, the total volume of blood provided to either heart is about half that normal, so that two hearts in one person should eventually experience a hypovolemia-induced ventricular fibrillation. Notwithstanding that the hearts are impaired in absolute stroke volume (or ‘ejection fraction’), this represents a considerable chronic underload relative to that normally driven. Preserving normal rhythm necessitates a method for alleviating the hypovolemia. One method for averting dysrhythmia, alone or in combination with other means such as pharmaceutical and mild, nonshock, electrostimulation, is diversion chute toggling to alternately deliver close to the normal volume of blood to either heart often enough to elude this consequence.

Should a dysrhythmia occur, the control microprocessor, using implantable for each heart of an, first executes an algorithm to find that action of the valves which dispels the condition most quickly. If not quickly dispelled, electrical stimulation followed by shock discharge is used. Thus, in dispelling ejection deficiency, toggling dispels an antecedent or underlying problem of chronic intracardiac hypovolemia that would induce a dysrhythmia. Toggling is programmed to preclude a dysrhythmia with the least movement of the diversion chutes, hence, the least expenditure of energy. Dispensed with is a heavy battery pack requiring frequent recharging.

When the native and donor hearts are different in size and/or ejection fraction, or absolute stroke volume, the diversion chute setpoints are adjusted accordingly, and if necessary, reciprocating motion, or toggling, of the diversion chutes to either side of their set points between partial and full extension allows differential apportionment of blood to either heart, thus causing blood to pass through both hearts frequently enough to avert the adverse consequences of intracardiac hypovolemia with eventual disuse atrophy. Toggling seeks to alternately entrain systoles from either heart into a unified bloodstream to optimize the net ejection fraction.

In a double heart transplant, as long as the hearts beat asynchronously so that successive systoles contributed by each achieve a functional ejection fraction, which the implant sensors will report to the controller, toggling may not be needed. The probability is that left to be ‘free-running,’ the conduction system-independent hearts will drift into and out of this condition. The less often toggling support is necessary, the longer will be the intervals between the need for recharging. Otherwise, toggling may be required until the hearts and overall cardiovascular system to include its neuroendocrine control adapt to the new milieu, and if toggling is needed continuously, the power consumed is still much less than mechanical assist devices.

Toggling involves immediate control over diversion chute degree of extension or retraction, and rates of advancement as determined on the basis of the physiological data continuously fed to the external and implanted controllers. Valves respective of specific veins and arteries in the donor and recipient, and their hearts once the donor heart is placed in the recipient, must pass the same volume of blood; however, if the hearts differ in size, the absolute degree of chute extension to send and receive the same volume may be different. Should either heart begin to malfunction, the bulk of flow can be passed through the other heart.

Using radiation shielded lines and nonjacketing side-entry connectors as shown in copending application Ser. No. 14/998,495, for example, malignant nidi can be directly targeted with chemotherapeutics and radioactive agents, again, in higher concentration with minimal if any exposure to untargeted tissue, the means therefore involving neutralization or removal by magnetic extraction or if available, the use of a reversal agent. The direct piping of drugs to the nidus not only eliminates the exposure of unintended tissue but also the limitations placed upon the oral drugs, to include limited dosages and the wasteful greater cost of systemic dispersal. Eliminating exposure of the entire body to antibiotics, for example, should significantly reduce the increased resistance of bacteria to these drugs, as well as the use of higher dosages.

The tissue just below the level of the jacket might present an initial adverse tissue reaction that would be suppressed were an anti-inflammatory drug directly applied to the inflamed tissue. Although some adverse reactions to glucocorticoids, such as an adverse effect on hypothalamic-pituitary-adrenal-axis function which can induce adrenal suppression and possibly Cushing's syndrome, are intrinsic and would ensue regardless of the administration route, the direct application of triamcinolone, for example, to the affected lower tract, encouraged by a direct path to the affected site, averts an injection site reaction. Injection poses the risk of numerous adverse tissue reactions, to include the destruction of muscle and fat down to the bone at the injection site, as well as numerous other major and minor adverse events local and less local.

The topical extension afforded by the apparatus eliminates the site concentration inherent in injection. To protect small supportive vessels and nerves at the adventitia of the ductus to be jacketed and close off any path for the luminal contents to leak out through the ostium or aperture created, the internal surface of the jacket is lined with moisture barrier-protected viscoelastic polyurethane foam. Leakage and exposure of an open wound to blood or urine can hinder healing, considerably so if colonized by a pathogen. If the ductus to be jacketed is resistant to incision, the operator can rotate, then lock, the connector in position. To protect the urothelium, the jacket shell and foam lining are perforated to provide the adventitia with open access to the surrounding milieu.

Regardless of what type ductus is treated, if needed to prevent jacket migration, the jacket shell includes suture loops to allow tacking the jacket to neighboring tissue. Notwithstanding that the foam lining is wetted with anti-inflammatory medication, usually a corticosteroid, such as cortisone, prednisone, prednisolone, dexamethasone, or triamcinolone, for example, when placed and that nonallergenic materials are used, should an adverse tissue reaction appear, counteracting medication is targeted to the site of the irritation through the accessory channel by injection at the surface port (see, for example, Kastellorizios, M., Tipnis, N., and Burgess, D. J. 2015. “Foreign Body Reaction to Subcutaneous Implants,” Advances in Experimental Medicine and Biology 865:93-108).

If the reaction is refractory to more conventional treatment, a ductus side-entry jacket with catheter leading to the surface port is laparoscopically positioned higher (upward, superior to, cephalad, craniad,) on the ureter through a small incision to target palliative medication to the site of irritation. Amino-amide drugs, such as lidocaine, prilocaine, bupivacaine, iontocaine-epinephrine and other nonsteroidal anti-inflammatory drugs, such as aspirin, naproxen, 2-arylpropionic acid, and proprionic derivative drugs, such as ibuprofen, ketoprofen, fenoprofen, and flurbiprofen directly routed to the nidus or affected segment of the ductus, or if an artery, the territory supplied afford both anesthetic and anti-inflammatory function with little risk of unwanted side effects.

If necessary, an implant timing module or microcontroller can be programmed to release the medication automatically from a small flat reservoir positioned subdermally in the pectoral region. In most cases, it should not be necessary to place other jackets upstream or downstream of the ductus such as a ureter to release drugs which can be targeted into the jacket and the ductus it surrounds without additional jackets. Accessory channel fed microneedles to release medication at the fibrosal-jacket lining interface are best avoided through the use of a ‘soaker hose’ branch from an accessory channel.

If for a certain application the use of microneedles is preferred, this can take either of two forms, the first, nanometer-sized projections mounted to a patch backing which press into the surface of the ductus and gradually release medication by diffusion rather than injection, the other replenishable, tiny hollow needles connected to an accessory channel fed drug supply line and network to each needle through which the drug or drugs are released or if of higher viscosity, injected under pressure. The need for either should be rare.

A diversion jacket to be positioned along a blood vessel to create a shunt, for example, incorporates a water jacket at the trepan line end as described in copending application Ser. No. 14/998,495. Once the jacket is connected, the water jacket continues in use as an accessory channel which ordinarily passes medication from an implanted drug reservoir but can be internally configured to pass through miniature cabled devices, such as atherectomizers, thrombectomizers, intravascular ultrasound probes, angioscopes, laser, and so on. Upon placement, the water jacket is used as a miniature pressure washer to restrain blood from extravasating and entering the diversion line, or mainline.

Water delivery through the water jacket is through an accessory or service channel. Once the jacket is in position, the accessory channel remains available to deliver medication into the jacket and substrate native ductus as an accessory channel. Another factor likewise due to the difference of continuous flow through a blood vessel and intermittent flow through a ureter is that the former is almost never adjustable. If adjustability is desired, it is to establish diverted flow temporarily, only so long as the native vessel needs to heal, or is meant to allow evaluation of the shunt created, or to be able to switch flow from one vessel to another is of value in research. The potential need for diversion should be taken into consideration when the system is implanted.

Nonjacketing side-entry connectors are securely anchored within the substrate tissue as to recommend additional stabilization only when the tissue is malacotic or might become so. Then anti-migration means for nonjacketing connectors as well as ductus side-entry jackets consists of tying either to the underlying structure. Suture connected to the suture loops molded into jackets and connectors about the surface is spirally wound about the ductus above and below the jacket or to a neighboring ductus with an adequate number of turns to assure stability.

When the distance between the ends of the suture is substantially constant, fixation can consist of or include suturing to neighboring tissue. In an emergency when time is lacking and connection to thicker planar rather than ductal tissue must be accomplished instantly, the use of one or more tiny barbs emulative of a honeybee stinger can be used. The use of barbs has been used to fixate implanted medical devices such as neuromodulators for decades. The ‘stinger’ is fastened at the end of suture much as a lure or fly is to fishing line (Ling, J., Song, Z., Wang, J., Chen, K., Li, J., and 5 others 2017. “Effect of Honeybee Stinger and Its Microstructured Barbs on Insertion and Pull Force,” Journal of the Mechanical Behavior of Biomedical Materials 68:173-179). The analogy can be extended to the use of a hollow barb that would allow the injection of drugs into the substrate tissue under the control of an implanted microcontroller no differently than medication is released into a side-entry jacket, valve, or connector.

When placed in a patient with frequent urination, enuresis, or nocturia, for whom access to a bathroom eliminates the need for urinary diversion, the bistable (on/off, binary) embodiment is not limited to one-time advancement unless forcibly retracted by the operator but can be advanced and retracted as necessary. One-time binary diversion valves are also suitable for use in vascular prostheses and bypasses, which unlike the urinary applications, where the patient can manually switch the valve on either side with a manually operated push/pull control cable, a number of valves must be switched once from fully retracted to fully advanced at the same instant.

Valves which must move from completely open to completely closed at the same instant, used, for example, in sudden switch transplantation and extracardiac correction of complete, or dextro-transposition, of the great arteries, incorporate highly damped nonsparking plunger solenoids controlled from a single switch. Valves controlled thus implement recipient-to-donor solid organ inflow and outflow compound bypass essential to suddenly and completely switch recipient circulation from his native to the donor organ. Except in transplanting a single kidney or lung, the donor has been kept on life support past death.

Transplantation thus is intended for use when the donor on life support is deteriorating and/or the recipient is nearing death due to end-stage organ failure. Where exigency is not a factor, metered switch transplantation uses servovalves to control the gradual movement of the diversion chutes between completely open to completely closed so that the relative proportion of blood diverted from the recipient to the graft organ is progressively increased as a means for accomplishing immune tolerance induction concurrent with transplantation.

In this way, metered switch differs from sudden switch transplantation in transferring circulation to accomplish reciprocal cross-microchimerzation to reduce immunological incompatibilities from the recipient to the donor organ gradually rather than abruptly as in sudden switch transplantation. With sudden switch transplantation, blood draws and tissue samples can be manually exchanged perioperatively and intraoperatively between the donor and recipient, donors and recipient, or donor and recipients, or donors and recipients. In drawing figures, this appears as a stage intervening between complete separation of recipient and donor circulatory systems and the completed transfer of perfusion from the recipient to the donor organ.

During this interval, the blood of the participants becomes more and more mixed, completely so if the process, continuously variable in speed and release of materials into the mixing circulations is continued long enough. Bistable and controlled from the same switch, damped nonsparking plunger solenoid controlled diversion valves slide the diversion chute from fully retracted to fully extended into the substrate native lumina to fully obturate and divert flow from these into the blood-conveying mainlines each connecting counterpart organ inflow and outflow vessels of the recipient and donor.

Such valves are also used in urinary diversion as addressed below. In contrast, metered switch transplantation is accomplished with intravascular servovalves, which are precisely controlled by an implanted microcontroller driven by data fed to it by implanted immune factor and other physiological sensors. Servovalves are not limited in motional pattern or timing. The elimination of ischemia-reperfusion injury a benefit of sudden and metered switch transplantation, both plunger solenoid and servovalve operated vascular valve-jackets allow transplantation without interruption or reduction in perfusion through a single graft organ and virtually none in a metered switch double heart transplant with implanted automatic support as described in copending application Ser. No. 15/998,002.

In so doing, this should avert much of the damage done to the organ when the donor on life support expires and life support is shut own, at which time cytokines react in a storm or cascade to materially reduce the quality of the organ. Following transplantation, the implanted prosthetic disorder response system that had been placed to treat heart failure, for example, remains in the recipient to automatically detect the need for and dispense transplant maintenance medication. Another value in the elimination of an interruption in perfusion past the terminal event is that the entire problem of ischemia-reperfusion injury upon placement in the donor is eliminated, and therewith, a prominent cause for both injury to other organs and tissues and graft rejection or reduced term of viability.

An intravascular servovalve is no less able to suddenly move the valve diversion chute from fully retracted to fully open or the reverse and therefore able to perform a sudden switch transplantation. The complexity of reperfusion injury is so multifaceted, that to reverse its consequences in detail after these consequences have already set in will likely take many more years and may never be fully achieved, making the eradication ab initio of ischemia during transplantation the far more propitious strategy. However, an intravascular servovalve, driven by a small leadscrew or linear motor under the control of a microprocessor acting as the master node in a hierarchical control system, is also able to divert flow through the organ from the donor to the recipient by guarded degrees, during which the condition of both donor and recipient are tightly monitored for adverse allogeneic reactions for response by the control system.

This gradual blending of donor and recipient blood should induce or if preprocedurally carried out manually, then continue the induction of immune tolerance which the system is able to increase until either responds with an adverse reaction. In addition to tolerance induction by means of blending the bloodstreams of each, drugs and other agents can be delivered into the blood of either to mix into the blend (monoclonal antibodies, such as interleukin-2 receptor cluster of differentiation 25 and cluster of differentiation 3-targeting antibodies; cyclosporine and tacrolimus calcineurine inhibitors combined with mycophenolate mofetil; the mammalian target of rapamycin sirolimus, interferons, opioids, and so on). The system then adjusts the valve jacket diversion chutes as directed by the prescription-program of the microcontroller or microprocessor implant, either partially or fully retracting the diversion chutes, and releases reversal and ameliorative medication such as an anti-inflammatory and/or an immunosuppressive.

Introduction thus is by injection through a small port at the body surface leading into an a valve-jacket accessory channel and therefore directly targeted to the vessel the jacket encircles. In such a control system, sensors provide physiological and/or pathophysiological data to their local nodes which process and pass the data to higher level data coordination nodes until this reaches the master node microprocessor which coordinates the data from the different reporting channels. Before transplantation is initiated, the donor is provided with such an implanted prosthetic disorder response system. The system is intended to allow the optimization of his condition, preferably avert the need for a transplant, and if so, allow the continued automatic detection and the release of maintenance drugs. Should the patient die, the system will have been prepositioned to prepare the patient for immune tolerogenic treatment to better match the prospective recipient or recipients.

Generally, in metered switch transplantation, the prosthetic disorder response systems of the deceased donor kept on life and medical support initiated before the terminal event and the recipient:

1. Monitor the baseline immune and comorbid status of both donor and recipient.
2. Regulate the delivery by dose or volume of drugs and other exogenous agents and adjust these to the optimal level for immune tolerance induction by
3. Controlling the release of drugs and
4. The rate and direction of advancement of the diversion chutes in gradually switching the graft organ from the circulatory system of the recipient to that of the donor.

To the extent possible, an organ transplant prescription-program is normalized or generalized. That is, the program is written to maintain optimal homeostasis during and following transplantation of any solid organ or combination thereof, and while adjusted for body mass and the specific organ or organs to be transplanted, is written to cover all infant or adult individuals undergoing the same procedure. The program thus acts not as a gross adjustment preparatory routine, entrusted to medical specialists using extracorporeal apparatus and higher volumes of drugs and other agents, but rather as the fine adjustment mid- and postprocedural administrator of the transplantation process implemented once the normal range of any index falling outside this range has been instated.

Because adults have a mature immune system and multiple ways in which the system had been challenged, the process is more complex than in infants. Then, later in life, immunity is compromised by immunosenescence which results in greater tolerance not just for pathogens but an allogeneic solid organ. In the references cited just below, the 2016 paper by Chih, and Patel is of special importance for adult transplantation. Provided maternal or paternal chimerization are medically insignificant, the immune system in a neonate is premature so that immune tolerance induction through metered rather than sudden switch transplantation should be avoidable, and associated defects notwithstanding, comorbidity as such is seldom a complication, whereas in an adult, concurrent, or comorbid, disease with inflammation and often infection pose a preliminary problem of existing antibody sensitization.

While the implanted process control units of both the deceased donor and recipient could be made to command extracorporeal machines to apply processes such as liver dialysis, plasmapheresis, leukapheresis, and kidney or liver dialysis, or release volumes of agents too large for an implanted reservoir, this is not accomplished either mid- or post-procedurally using implanted means. Further, of necessity, since far too many sensors would be required to monitor and respond to cytokines in detail, for example, many if not most of which would be susceptible to the same treatment in any event, the program, in complete compliance with accepted practice, is written to the empirical or summary level to detect and respond to inflammation with anti-inflammatory medication targeted directly to the treatment site or sites.

More often, intercurrent or concomitant requirements of individual patients are entrusted to the more broad and detailed expertise of medical staff physicians, to include a hematologist, endocrinologist, internist, and immunologist. Infant and adult patients requiring a heart transplant differ not only in the absolute doses of medication required and the common causes therefor such as a heart severely malformed in an infant and end-stage heart failure in adults but also in the degree of preparation essential to proceed with the transplant. Distinctions in body mass and the specific procedure embarked upon, the control programs for both are essentially the same.

Nevertheless, where drug volumes to treat concomitant disease are small, programs for adults can differ from those written for infants in incorporating these drugs into the program as a callable unit (subroutine, subprogram). Empirically again, agents used to eradicate pathogens in adults presenting intercurrent disease might include an antimicrobial or antifungal, and those affected with concomitant disease might include antibody desensitizing agents, such as tacrolimus, mycophenolate mofetil, or rituximab, intravenous immune globulin and preoperative apheresis and/or dialysis relegated to extracorporeal equipment.

When recipient and since expired donor are connected for transplantation and possibly cross-tolerance induction, the master node microprocessor of both are placed as subordinate to the control of an extracorporeal monitor/processor. The prospective recipient having lived with a failing heart over a period such that organ impairment secondary thereto, such as cardiorenal syndrome and/or congestive hepatopathy are likely, once the transplantation procedure has been completed, the microprocessor of the recipient once again becomes the master node, executing a prescription-program devised to maintain the graft organ and treat any additional morbidities with the automatic release of drugs as necessary and assure optimal overall homeostasis for the host.

To simplify the induction of immune tolerance, where secondary organ impairment is severe, the impaired organs are considered for transplantation from the same donor. The implant system in the brain-dead donor is recovered after all organs slated for harvesting have been transplanted. To continue targeted diagnostics and therapeutics of the organ once transplanted, diversion jackets and sensors are transplanted with the substrate organ. Both in the donor and the recipient before and after transplantation, autonoumous microcontroller circuits can be placed under the higher control by a microprocessor in a hierarchical control system to optimize overall homeostasis by applying treatment across the different sources of morbidity in both donor and recipient.

By switching the blood supply and drainage of the recipient from his own diseased organ to that of the donor, sudden and metered switch transplantation eliminate any interruption in perfusion, cross-clamping, and the need for cardiopulmonary support for the recipient, which involves circumventing the heart of the recipient, much less cold storage of the graft organ in a static perfusate. Transplantation using these techniques therefore avoids reperfusion injury, which along with the risk of rejection, are the main sources for most of the complications encountered in solid organ transplantation.

Valves that must be precisely controllable in a graduated manner to adjust blood pressure through a particular vessel such as the main pulmonary artery or pulmonary trunk, for example, are controlled by a miniature linear motor or leadscrew. Any ductus side-entry jacket or nonjacketing side-entry connector can be used to target chimerizing and antirejection medication to a transplanted no less than to target such medication or antimicrobial or anti-inflammatory medication directly to a native organ or release this or any other kind of medication into the systemic and pulmonary circulation.

Sustainment of the donor affords an interval to identify a potential organ recipient or recipients and initiate chimerization and supportive therapy of the prospective donor organ or organs as well as to optimize his condition. Because the chimerizing material can be pipe-targeted directly to each organ with relatively little spillover into the systemic circulation, the ability to differentially chimerize each organ for different recipients is increased. Direct targeting is of considerable benefit with any drug, but especially so with immunosuppressants, steroids, anticancer drugs, and antibiotics that would otherwise destroy beneficial microbiota in the gut. This is so where a bacterial infection within the urinary bladder can be directly targeted for the piped instillation of an antibiotic which is then voided without having been passed through the digestive tract.

In sudden and metered switch transplantation of the heart, provided the body mass of the donor is not significantly less than that of the recipient, the heart of neither is ever confronted with an increase in load: rather, each heart supports only one body at a time. For this reason, only wide differences in body mass between donor and recipient necessitate the use of pumps to achieve a suitable flow rate. Moreover, when switched into the circulatory system of the recipient, the much more highly competent new heart is all the more competent to its new body. Furthermore, Lillehei's first operation required that the additional load of the child be sustained for 19 minutes. His later operations were to repair significantly more serious malformities than a large septal defect, and the operations took longer than 19 minutes.

In comparison, the interval over which the heart of the recipient must send the full complement of blood to the heart of the donor is that following the switching of flow from his own defective heart to and through the donor heart plus the time to place and anastomose the donor heart in the chest of recipient. Since initial anastomosis can be interim accomplished with a surgical grade cyanoacrylate cement without waiting until the anastomoses have been sutured, the overall interval over which the additional length of tubing must be cleared runs from switching of the vascular valves until the new organ is secured in the recipient, an interval lasting substantially less than 19 minutes. While unlikely, a need to interpose a small pump along the connecting lines would be obvious to an experienced operator and quickly satisfied.

In open heart surgery, machine support is essential because the heart is disabled. In sudden switch transplantation, the organ is never disabled; rather it is just switched from the recipient to the donor. In any transplant operation where the mainlines connecting the valve jackets must be too long to implant with the graft organ and left in place indefinitely as prosthetic, direct end-to-end connect jackets are used. Such jackets have raised rims about the ends of the jackets facing one another which are locked in end-to-end edge apposition by means of a miniaturized version of a factory drum lid type rim/cover surround lock clamp. Before connecting the ends, circulation continues to bypass the junction through the diversion chutes.

The stumps of the vessels to be anastomosed are trimmed back to about two millimeters in extension past the facing jacket ends and the raised rims at the jacket facing ends clamped together with the slightly overextended edges of the vessel positioned flush together, coated with a substance such as sterile Tualang honey, found to facilitate the healing of anastomoses, and bonded with a surgical cyanoacrylate or fibrin cement. The balance of the anastomosing is devised to allow healing without the need for reentry. The edges of the vessels thus fused together to form a continuous conduit across the jacket junction, the mainlines are removed and the junction allowed to heal together within the time the cement will continue to bond the juxtaposed edges.

In most instances, it is preferable not to remove the valve jackets, because the accessory channels allow the direct pipe targeting of stem cells, an anesthetic, thus avoiding nonsteroidal anti-inflammatory drugs which weaken anastomoses, anticoagulant, and/or antimicrobial as necessary by injection into the small port at the body surface during healing and at any time thereafter. Such means are most pertinent to solid organ transplantation in prenates, neonates, and infants, where time for such measures as tissue expansion is lacking and space is a major consideration.

Thus, reciprocal cross-circulation should not be misconstrued as the unilateral support of a patient by a larger volunteer whose heart is used to support the patient as well as himself with the aid of a pump positioned along the line connecting the two. The term ‘reciprocal cross-circulation’ is used to distinguish the unilateral form of cross-circulation used by Lillehei to allow open heart surgery from the bilateral form meant here. Thus, where the term ‘cross-circulation’ denotes unilateral supply with substantially passive return, ‘reciprocal cross-circulation’ as used here denotes two-way active exchange.

With the advent of ventilators and improved cardiopulmonary bypass machines, historical cross-circulation has been abandoned, largely because it exposes the volunteer to too great a risk and is limited to a volunteer much larger than the patient. It survives in other forms where the risks for both volunteer, if applicable, and patient are less severe. Here, the donor is beyond injury and the heart of the recipient suddenly replaced with that of the donor.

A side-entry diversion jacket can be used not only to redirect flow, but can incorporate components that provide imaging capability, diagnostic data, and by delivering a drug or drugs at and distal to the site where it is positioned, therapy. Moreover, while not incorporated as an integral component of the jacket, cabled devices such as a fine gauge excimer laser, fine fiberscope, intravascular ultrasound probe and so on can be passed through an accessory channel or channels of the jacket to provide increased viewing and treatment capability. Ingestible magnetized drugs intended to enter the bloodstream and/or urine are in development.

Ferrofluidic drug application or the inducement into the luminal wall by a conventional or a flow-diverting side-entry jacket adapted to function additionally as an impasse jacket applies to any bodily conduit, whether urinary, reproductive, digestive or circulatory, or to any combination thereof. While not addressed herein, a needle free disposable cartridge jet injector for the self-administration in the home of plural drugs to treat comorbid conditions can be multiply nozzled to mate with complementary openings into corresponding surface port openings in a matching surface port, as will be described.

Moreover, under the control of a fully implanted timing module or microcontroller responsive to a prescription program, small pumps, in some instances very small, and in some instances, allowing drug replenishment from the surface port to a “micropump for drug delivery with integrated storage and electrically controlled timing” at the outlets of subdermally positioned small flat reservoirs at the back of the port into which injected drugs are delivered can be delivered along separate accessory channels to multiple side-entry jackets and nonjacketing side-entry connectors.

This allows the direct targeting of agents to image, diagnose, and/or treat comorbidities. While compatible with such pronounced miniaturization, most applications contemplated herein do not call for a micro or nano degree of miniaturization. The ductus side-entry jackets can be positioned at different levels along one ductus or to different ductus, while nonjacketing side-entry connectors are affixed to one organ or to several organs belonging to different organ systems, as described in copending application Ser. Nos. 14/121,365 or 15/998,002 and 14/998,495. When the drugs or other agents are radioactive, depending upon their dose rate and latency, the accessory channel and connector are radiation shielded as shown in copending application Ser. Nos. /121,365 and 14/998,495.

This data can then be applied in accordance with an implanted prescription-programed microcontroller to adjust drug delivery to any of a number of locations automatically in a fully implanted and ambulatory feedback system. The result is that the patient need only replenish the set of subcutaneously (subdermally) implanted flat drug reservoirs periodically, and—when all accessory channel openings of the body surface port are incurrent and subcutaneous—replenish the power of the implanted rechargeable battery by transcutaneous energy transfer. Otherwise, the patient is oblivious to the automatic diagnostics and direct piping of drugs responsive to his disorder or disorders as he goes about his daily activities.

A body surface port is essentially a specially adapted nonjacketing side-entry connector as addressed in copending application Ser. No. 14/998,495, usually placed in the pectoral region, as would a portacath chest port or totally implantable venous access device, or in the upper arm. The direct delivery to a diseased ureter is accomplished by positioning a ductus side-entry jacket above the lesion. When the ureter might be salvaged through remedial measures, so that temporary diversion to the contralateral ureter or to the bladder would allow time for healing, the adjustable ductus side-entry diversion jacket chute is adjusted to allow medication to allow the bulk of urine to be diverted while a lesser volume is allowed to flow down into the diseased segment.

The recipient-to-donor solid organ circulatory system sudden switch procedure, to include heart transplantation, might be characterized as a form of cross-circulation, but only in a loosely analogous sense. Cross-circulation began in Belgium in 1890 when the physiologist L. Frederic moved blood between the carotid arteries of two dogs to accomplish shared circulation and thereby blend the blood borne immunological constituents of the two. The controlled, or pump-assisted, cross-circulation demonstrated in 1954 to circumvent the heart of the patient used cannulae and transparent plastic tubes to move blood between a volunteer and patient.

More than one combination of vessels in use at the time, in the procedure led by Lillehei, one cannula was inserted remotely from the heart into the femoral artery of the volunteer, his blood moving through clear plastic tubing through a pump and from the pump through tubing through a cannula inserted into the aortic arch of the patient. The venae cavae of the patient were ligated, his desaturated blood sent through tubing to the great saphenous vein and lungs of the volunteer for oxygenation and return to the patient, whose azygous vein served in lieu of his venae cavae to return blood to his heart at 10 percent the normal rate. Except for the justapositioning and exchange of blood between two subjects, instant switching between the circulatory systems of a donor having been maintained on life support past death and a recipient shares nothing with such a procedure, which seeks to obtain intracardiac access, not accomplish a heart transplantation.

The procedure arose instead as but one application for ductus side-entry diversion jacket electrovalves, which are no less applicable anywhere else along the vascular tree or the urinary tract—not the reverse, not by development of a device for the limited purpose of solid organ, here, heart transplantation. Unless volunteer and patient differ markedly in size or weight and the recipient does not present significant pulmonary vascular resistance, the load on both hearts is substantially constant. Such an approach to solid organ transplantation is made possible by the availability of ductus side-entry diversion jacket electrovalves, which did not exist for Lillehei.

In contrast to historical cross-circulation, the technique represented herein, reciprocal cross-circulation:

1. Does not place a volunteer in jeopardy.
2. Does not bypass the heart as is necessary to perform a conventional heart transplant, involves zero cardioplegia and zero ischemia time.
3. Compared to a conventional transplant or intricate surgical procedure to temporarily palliate a complex congenital defect; is accomplished in relatively little time.
4. Demands far less skill than conventional transplantation or reconstruction, allowing application in other than tertiary medical centers, and
5. Does not require the heart of the volunteer to support the bodies of both, severely limiting the size and weight of the patient to about 86 pounds, but rather suddenly switches the heart of the donor from his own machine-supported circulatory system to that of the recipient.

The relatively quick exchanging of one heart for another can replace the reconstruction of a complex congenital defect; the long-term outcome with a good if immunosuppressed heart is better, and the jackets used allow the immunosuppressive, or antirejection, medication to be kept to the minimum. Not only is ischemic time zero but procedural time a fraction of that required for repair by a pediatric cardiac surgeon. Switch technique heart transplantation avoids the need for cardiopulmonary bypass, aortic cross-clamping, and by allowing the use of regional rather than general anesthesia, reduces postoperative cognitive impairment, decline in ventricular function, perfusion defects, and myocardial deformation.

It also and myocardial deformation to the extent that such consequences, which typically resolve within a year following transplantation, are attributable to the use of general anesthesia rather than the extensive incision involved in non-switch methods of heart transplantation. Orthotopic heart transplantation in the face of preexisting malformity is rendered more flexible as to precise positioning in the chest if, as side-entry diversion jackets make possible, shunting can be substituted for direct anastomosis. Stimulated by somatotropin, or human growth hormone, anastomosis of donor and recipient vascular stumps in a fetus or neonate is not essential for growth.

This more difficult and time-consuming step can therefore be deferred until the patient fully recovers from the original operation. Never achieving physiological sufficiency and therefore allowing a progressive continuing hypoxia and degeneration throughout the body, provided a replacement heart is available, reconstruction or any other palliative measure should not be considered with transplantation delayed. The proper place for such methods is as a bridging strategy pending the availability of a heart, just as are assist devices and bilateral banding of the pulmonary arteries.

Side-entry diversion jackets and the lines connected to these are left in place following transplantation to directly target medication and line maintenance fluids to the treatment site and postpartum when the body surface port is placed prior to discharge. Thereafter, allowing a longer period for the patient to fully accommodate, the vessels can be anastomosed. In the unusual circumstance that another heart will become necessary, these will already have been placed to expedite reoperation. This applies to numerous surgical techniques, as well as hybrid surgical/interventional methods.

More generally, as immunosuppressive measures continue to improve, a technique of heart transplantation simpler, more flexible as to orthotopic positioning, and far more likely to succeed than any conventional technique is of value where the condition of the patient is urgent, especially where the patient is too distant to the closest quaternary medical center and/or where the inordinate expertise and skill required to reconstruct the malformed heart is lacking—such as an infant in a rural area with a single ventricle heart, a hypoplastic left heart that includes an atresic aorta demanding exigent repair, or failed Norwood procedure, for example. In contrast with heart transplantation in adults, most often done in response to end-stage heart failure, in pediatric cardiac surgical practice, transplantation, especially if expedited by new means, can serve in lieu of attempting the repair a heart so malformed that even an optimal correction would not establish normal flow and probably prove lacking in durability, making such a fallback bridging strategy, not an adequate repair.

When a complex reconstruction fails so that transplantation becomes advisable, a technique for transplantation which is simpler, avoids hypoxia, and involves less dissection and procedural time, is highly advantageous. Such applies to the two-stage Fontan repair of a univentricular heart in a child, which, primarily by altering blood flow, induces progressive heart failure and therewith, increasing damage to the digestive and other organ systems, and until recently, adversely affected remediation by heart transplantation. It is now well established that the postnatal association of congenital anomalies of the heart with anomalies in other organs had been preceded during the prenatal period, maldevelopment of the brain included.

If not corrected, a failing Fontan leads to irreversible multiple organ damage which may necessitate that other organs be replaced as well. Hence, where an original repair is survivable but would result in abnormal circulation that would lead to such a consequence, whenever possible, transplantation as the first response is clearly preferable to allowing such degeneration to progress. Much the same may be said for the three-stage Norwood procedure to repair a hypoplastic left heart, where a univentriclular heart which experience shows remains functional over a growth-limited interval is created. Even the most recent hybrid procedures do not yield acceptable development, health, or longevity.

Notwithstanding recent improvements (see, for example, Kenny, L. A., DeRita, F., Nassar, M., Dark, J., Coats, L., and Hasan, A. 2018. “Transplantation in the Single Ventricle Population,” Annals of Cardiothoracic Surgery 7(1):152-159; Davies, R. R. and Pizarro, C. 2015. “Decision-making for Surgery in the Management of Patients with Univentricular Heart,” Frontiers in Pediatrics 3:61), during the ensuing year following surgery, over one-quarter of these patients die or require a heart transplant, the best outcomes forestalling death no more than five years, and exceptionally, two or even three decades. Despite the advancements made in preventing continued damage of congenital malformities which can be repaired transcatheterically in utero, for the foreseeable future, repair other than a septal defect or valve will have to wait until delivery.

While the relations between the in utero development of the heart and brain continue to be elucidated, where magnetic resonance imaging of the fetus indicates that abnormal cerebral flow concomitant with focal white matter injury may be due to anomalous development of the heart, either palliative surgery in utero with the intention of replacing the heart promptly following delivery or an in utero heart transplant as soon as a suitable heart can be found is essential to save the brain from further maldevelopment.

Procedures other than transplantation, or if pulmonary vascular resistance is present, a heart and lung transplant, result in defective circulation and therewith, progressive multiple organ damage which invariably leads to a sick life and premature death. The superiority over such staged radical procedures of a facilitated method for transplantation following the initial diagnosis is clear, and the preferability of transplantation as the initial response is all the clearer because however good the heart, the degradation in other organs that results when a single ventricle is left to drive both the systemic and the pulmonary circulation leaves transplantation with irreversibly impaired organs incapable of sustaining life much less good health for long.

With the object of regenerating infarcted myocardium, cardiosphere-derived cell therapy, mesenchymal stem cell-derived exosomes, bone marrow aspirate concentrate, exosome-mediated regenerative cell-to-cell communications, dental pulp stem cells, and other agents for myocardial regeneration remain in development with numerous obstacles to practical implementation, primarily the deterioration in protein and cell survival, which an automatic delivery system can overcome by immediately responsive sensors and release of cell and protein biomaterials devised to achieve effective delivery. Protected agents must be administered at the prescribed interval, making automatic release by an implant system decisively more dependable than voluntary compliance, and delivery directly piped to the coronary arteries supplying the infarcted portion of the myocardium is critically more efficient than nontargeted infusion into the venous system, and the coronaries end-arterial, the targeting is especially effective.

Provided clinical efficacy is established, these cells, autologous and therefore not arousing an immune reaction, administered by infusion, will be suitable for delivery by an implanted disorder control system placed to competently medicate a patient with comorbid disease who had experienced a myocardial infarction so that the loss of myocytes will not progress to heart failure before reaching end-stage, when only heart transplantation will allow survival. Since few if any hearts would still be needed for patients with end-stage heart failure, the ability to heal the infarcted heart and stop the ensuing degeneration into end-stage heart failure would effectively increase the supply of hearts available for transplant. Such an approach has the potential to regenerate myocardial tissue and thus cure, not just palliate, the infarcted myocardium.

As proposed, stem cell therapy may improve cardiac output pending the availability of a replacement heart and compensate for a temporary reduction in ventricular absolute stroke volume following transplantation. However, once mature, this technology can be expected to have applications beyond mending of the post-heart attack heart, post-transplantation support of the heart, for example, high on the list. Adding a ledge or lip at the jacket outlet opening or ostium leading out of the native lumen and into the side connector can be used to change the proportion of flow tapped, but for any one such jacket this proportion will be fixed.

By contrast, side-entry diversion valve jackets are either bistable, that is, adjusted as to be fully open or fully closed with no portion of the flow tapped, or continuously variable to allow adjustment in the relative proportion of flow tapped. The mainlines to be described can either be removed following anastomosis of the transected vessels, or if the bypassed segment remains irreparable or vulnerable, can be left in place indefinitely. The mainlines are removed and the side connector capped off, the jackets with sidelines, or accessory channels, connected to the port at the body surface are left in place to target immunosuppressants and other medication directly to the anastomoses, sparing exposure of these to the rest of the body.

To be described, the three side-entry diversion jackets used as endovascular valves to create the bypass needed to perform a zero-ischemic-time carotid endarterectomy, the four used to correct a transposition of the great arteries, or ventriculoatrial discordance, and the additional jackets needed for the conotruncal (conotruncus or bulbus cordis)-derived vessels and venae cavae in a heart transplant are of the bistable type. That is, the valves are switched from completely closed to completely open or the reverse simultaneously from a switch operated by the operator or an assistant, the valves moved by tiny plunger solenoids.

Where the operator, even without the aid of an assistant, should be able to adjust the jackets in carotid and other endarterectomies, aneurysm repairs or bypasses, and trasnpositions quickly by hand, in heart transplantation and other more complex operations, the larger number of vascular valves needed are adjusted simultaneously, all the valve-jacket chutes moved at the same instant by damped push-pull solenoids actuated by the operator with a plunger switch. Side-entry diversion jackets of any type can be used to bypass blood during open repair or remain indefinitely as prostheses.

A circumtrepan vacuum jacket, or mantle, comprising an inner tubular layer with perforations separated by a thin space from an outer layer positioned just beneath the jacket shell connected through the mainline to the same aspiration pump as is used to aid cutting by the trepan of the ductus side entry hole acts to distribute the trepan vacuum to the adventitia, preventing gouging injury to the intima opposite the trepan used to remove a tissue plug from the side of the ductus. Thereafter, the vacuum jacket serves to target anti-inflammatory and other drugs directly to the jacket foam lining-adventitial interface through an accessory channel.

The foam jacket lining inside, that is, adluminal to the inner layer of the vacuum mantle is clear of the vacuum perforations, the vacuum therefore drawing the outer wall of the ductus outward to protect the nontrepaned circumference of the ductus from gouging injury. For solid organ transplantation, basic side-entry and side-entry valve jackets address both major obstacles to success, rejection through the direct targeting of immunosuppressants to the treatment site, sparing the exposure of other tissue, and the avoidance of direct-contact native-to allogeneic tissue anastomoses, which are susceptible to failure. Graft organs susceptible to the development of malignant tumors, the jackets are also prepositioned to directly target anticancer drugs to the affected sites.

Side-entry and side-entry valve jackets can be used to create long term or permanent externally controllable central lines capable of any longer than temporary function served by conventional lines and can do so with greater versatility and safety. While a central line connected by a side-entry jacket is not inserted by direct puncture but rather through a ‘keyhole’ incision, the avoidance of leakage into neighboring tissue on placement and thereafter can represent a significant benefit. In transplantation, provided donor and recipient are positioned side by side, placement of side-entry diversion jackets on the incurrent and excurrent vessels of both donor and recipient organs allow the immediate switching of perfusion through the donor organ from the bloodstream of the donor to that of recipient.

Placement of a central line aside, to the extent that a patient, as when suffering from cancer, is more prone to clotting, an anticoagulant must be systemically circulated. However, to the extent that the central line aggravates the tendency to clot as a consequence of the trauma caused to the vessel in placing the line (Polderman, K. H. and Girbes, A. J. 2002. “Central Venous Catheter Use. Part 1: Mechanical Complications,” Intensive Care Medicine 28(1):1-17) and due to the turbulent flow of the blood caused when passing a foreign object suspended in the lumen, this tendency can be reduced if not eliminated by constraining the delivery drugs in more concentrated dose directly to the line insertion site and the affected segment of the ductus moving away from the incision, puncture, and/or lesion caused by disease, treatment of the injury of insertion with an anti-inflammatory and cytokine cascade proliferative-counteractive drugs and of clotting with an anticoagulant.

With medication delivered through a singular line, doses intended for local and systemic application cannot be suitably apportioned. Either a dose intended exclusively for local application that would best be constrained to the target segment continues, albeit progressively more and more diluted as it passes through the systemic circulation, to reach other tissue which it can adversely affect, or a strong initial dose intended for greater concentration within a restricted zone and lower concentration throughout the balance of the systemic circulation becomes too dilute. When the medication should be circulated, the systemic concentration should not be dictated by that of the local dose, and the local dose should not be constrained by the dose intended to pass into the systemic circulation.

Provided reversal agents are available, positioning a second line and side-entry jacket downstream from the first allows the intervening segment to be substantially isolated for proper dosing with the appropriate drug or drugs. In fact, three zones are created, that preceding the first line, that between the first and second lines, and that following the second line. This is because both lines can feed both drugs and reversal agents for drugs other than each delivers. Thus, a drug targeting the cutdown or puncture site in higher concentration and intended for uptake only there and over the segment between the two lines can be completely or partially neutralized at the second line.

Drugs for delivery at higher concentration at and over a short segment following the cutdown or puncture site include an anticoagulant and an anti-inflammatory. In a patient prone to clotting or one made susceptible to clotting as the direct result of placing the line, a lesser concentration of the anticoagulant should continue through the circulation. In a patient with normal clotting factors, placement of the lines with a ductus side-entry jacket or diversion side-entry jacket is less promoting of clotting, because there is no object suspended in the native lumen as would cause turbulent flow, and placement is less traumatizing. That reversal agents must not themselves introduce significant problems is superfluous.

The realization that an anticoagulant can be infused along with chemotherapeutics, for example, so as to preclude the formation of thrombus has been implemented, but not as to allow automatic delivery of both anticoagulant and reversal agent in an ambulatory patient with totally implanted central line, such as a central venous catheter, running from a body surface port, reservoir, catheter, and through a side-entry jacket (see, for example, Brandão, L. R., Shah, N., and Shah, P. S. 2014. “Low Molecular Weight Heparin for Prevention of Central Venous Catheterization-related Thrombosis in Children,” Cochrane Database of Systematic Reviews (3):CD005982).

While a conventional portacath is adequate for the infusion of one or more drugs directly into the superior vena cava or the jugular or subclavian vein, for example, an automatic disorder response system that requires the targeted injection or infusion of different drugs into different ductus in order to treat comorbid conditions in a coordinated manner would require multiple portacaths, creating the need for a compact subdermal port with multiple injection and/or infusion entry points. Deviations from normal renal and micturative function, to include diagnostic monitoring and response, can be incorporated into such an automatic system.

### Urological

Few conditions are limited to treatment using any one type valve. For example, an adjustable urinary voidance control system such as that shown in FIG. 30 could substitute a servovalve such as that depicted in FIG. 10A for the push/pull cable mechanism shown. While a urinary assistance device or prosthesis is never to be implanted where conventional treatment would allow the avoidance thereof, once diversion jackets such as shown in FIG. 28 or servovalves such as shown in FIG. 30 have been placed, the accessory channels can be used to eradicate a urinary tract infection by running an antimicrobial such as hydrogen peroxide, benzalkonium chloride, chlorhexidine, or povidone-iodine into the ureters to coat the ureteral, bladder, and upon voiding, the urethral urothelia. Antiseptics that cause a stinging sensation should be preceded by lidocaine.

Thus, where the lower urinary tract is bypassed by a prosthetic voidance control system consisting entirely of synthetic materials such as that shown in FIG. 28 so that infection generates no experiential correlate as the sensation of irritation or pain, but poses the threat of serious sequelae such by retrograde migration to induce a pyelonephritis, pathogen intrusion, with the deposition of a biofilm is eradicated by periodically directly pipe-targeting a nonantimicrobial, allowing the avoidance of an antibiotic such as ciprofloxacin or vancomycin if bacterial (usually Escherichia coli), for example, through an accessory channel, which all valves and servovalves incorporate. To avoid interfering with pyeloureteral peristalsis, ureteral diversion valves, just as are arterial valves to not interfere with pulsation, are lined with viscoelastic polyurethane foam which also protects the substrate ductus from degeneration or atrophy.

When urinary diversion is not required, the direct targeting of drugs or a stone solvent into a ureter can be accomplished through a basic ductus side-entry-jacket with accessory channel as shown in FIG. 16 in copending application Ser. No. 15/998,002 entitled Ductus Side-entry Jackets and Prosthetic Disorder Response Systems and its parent application Ser. No. 14/121,365 which shows which depicts such a jacket connected to the left descending coronary artery. When dealing with a coronary or carotid artery, placement should always be preceded by the administration of an anti-vasospasmic (anti-angiospasmic).

Basic ductus side-entry jackets very small and light in weight, in some rare circumstances, the diversion of blood into—not out of—such a crucial vessel can be from a vascular valve or servovalve on a large nearby artery into a basic side-entry jacket on the carotid or coronary. Due to increased size and weight and the forcefulness of systolic contraction, vascular servovalves are not used on a coronary artery; instead, a nonjacketing side-entry connector as described in copending application Ser. No. 14/998,495, entitled Nonjacketing Side-entry Connectors and Prosthetic Disorder Response Systems, can be positioned over the supply field of the coronary to directly deliver the drug into the myocardium.

Such an automatic system is also appropriate for use in a patient with urinary tract sensation but mentally impaired, disturbed, or presenting with intractable urinary incontinence. By contrast, a resistant nocturia is best alleviated through the use of a urinary assistance system such as shown in FIG. 30 rather than the prosthesis shown in FIG. 28 This direct route of administration renders superfluous the route of infection whether through ingestion, inhalation, breaches in the skin caused by ectoparasites such as arthropod, or vertebrate bites, or sexual activity. Drugs delivered thus are not injected intramuscularly to become dispersed throughout the circulation and dispersed throughout the circulation, but rather infused in such manner as to bypass nontargeted tissue.

For this reason, direct pipe-targeting allows the use of dose levels which due to adverse side effects, dispersion would preclude. Also rendered superfluous is the agent of disease whether bacterial, mycotic, protozoan, helminthic. The wearer of an adjustable urine voidance assist system such as that shown in FIG. 30 is of necessity sound of mind, retains normal urinary tract sensation and is therefore able to control voiding and self-administer drugs, so that a urinary tract infection can be treated through the oral route with ciprofloxacin or another oral antibiotic, for example. These considerations as to drug administration pertain no less to the use of crystal solvents in stone formers.

### Vascular Valves and Servovalves in Urinary Diversion

The temporary unilateral or bilateral diversion of urine from a ureter or both ureters to a paracorporeal collection bag usually cinched about a thigh can facilitate healing of the bladder and lower urinary tract with drugs following surgery. The diversion jacket incorporates at least one accessory channel for the delivery of drugs directly into the lumen of the segment to be treated. For patients with irreversible neurogenic bladder dysfunction, agenesis (aplasia) of or irreparable trauma to the urinary tract, radical cystectomy, bladder exstrophy (ectopia vesicae), or a pelvic exenteration (evisceration), use is continual and permanent as a prosthesis to the end of life, supplanting the need for an ileal conduit or a neobladder, which if lacking the necessary volume, compels frequent urination.

Gut is not adapted to conduct urine, and when called upon to do so, undergoes metaplastic transition that can result in malignancy and the need for revision. Should this occur, the placement of a prosthesis made of synthetic materials can prevent further disease and the need for revision. This is far less traumatizing or prone to complications than is the harvesting (retrieving, recovering, procurement) of gut, or the prospect of a radical cystectomy if not exenteration to eradicate cancer or to end unrelenting pain from interstitial cystitis. For these reasons, the urothelium is usually protested following the creation of a urinary conduit from gut through the placement of a urinary diversion stent, and the ureter or ureters protected with J-stents.

Conventional stents separate the urine from the urothelium but lack any means for maintaining the patency of the synthetic lumen such as through the direct targeting into the synthetic lumen or the interface between the outer surface of the stent and the urothelium of occlusion counteractants. Repeated exposure to urine without intermittent rinsing with clean water or an antiseptic solution fouls a catheter or stent in itself, to which must be added the gradual accretion of crystal if not a biofilm making these devices short-lived and requiring the unpleasant inconvenience of replacement at relatively frequent intervals, typically, every one to three months.

Such represents not a solution but the perpetuation of nuisance. In marked contrast, the means described herein for replacement of portions if not the entire lower urinary tract are cleaned and as necessary decrystallized automatically by an implanted microcontroller which signals the pump at the outlet of the antiseptic and/or decrystallizing solution drug reservoir to release the amount into the prosthesis as prescribed in its prescription-program. Provided the reservoir is replenished, the patient can remain as oblivious to the condition as had the urinary tract remained normal.

Where side-entry jackets can divert or return part of the flow through a ductus such as a blood vessel or ureter, a side-entry diversion jacket can divert all or any fraction of the flow. If the carotid diversion device to be described is to remain in place indefinitely as a prosthesis, a subdermally implanted port is placed to allow the periodic injection of an anticoagulant for direct targeting to the treatment site at the carotid bulb or bifurcation. The significance of using valved rather than unvalved or basic ductus side-entry jackets along the urinary tract is that unvalved jackets are static, while valved jackets are controllable, allowing the flow of urine to be switched from the bladder during the day to an external collection bag during the night, for example.

Flow through the valve mainline is controlled by adjustment of the diversion chute by the implanted microcontroller, which to control the delivery of drugs through the accessory channel or channels adjusts the drug reservoir outlet aperture or pump. In a ureteral takeoff side-entry jacket, the primary passageway or mainline is used to divert urine, and one or more sidelines, or accessory channels are used to allow the targeted delivery of drugs into the jacket and substrate ureter directly from a small port at the body surface.

For example, by placing a nonjacketing side-entry connector as described in copending application Ser. No. 14/998,495 to drain the renal pelvis and another on the bladder dome, the two can be connected with a catheter kept from fouling by the direct delivery through the accessory channel of the upper connector of suitable agents fed from a body surface port. The considerable advantages in the directly targeted delivery of drugs in an effective dose to primary and secondary sites of disease rather than its dispersal throughout the circulation where unintended tissue is exposed so that the dose must be minimized and still pose the risk of long known adverse side effects, drug-drug, and drug-food interactions (see, for example, Sanjay, S. T., Dou, M., Fu, G., Xu, F., and Li, X. 2016. “Controlled Drug Delivery Using Microdevices,” Current Pharmaceutical Biotechnology 17(9):772-787).

Given multiple capabilities, to include the diversion of flow, direct delivery to lesions of medication, and the ability to incorporate sensors, side-entry jackets, diversion valves, and nonjacketing side-entry connectors can join and diagnose native tissue, and maintain the patency of prosthetic replacement parts in the lower urinary tract, for example. A nonprosthetic urinary assist system allows the patient to sleep without interruption due to urge sensation and to appear in public even though affected by urinary incontinence or nocturia, for example, not caused by an infection such as a ureteritis, cystourethritis, or pyeloureteritis dispelled by a ten-day course of an oral antibiotic, for example.

Conditions demanding surgery such as a hemorrhage (see, for example, Thompson, J. S. and McAlister, W. H. 1975. “Subepithelial Hemorrhage in the Renal Pelvis and Ureter Simulating Pyeloureteritis Cystica,” Pediatric Radiology 3(3):156-157), to include those requiring excision such as ureteral endometriosis, can usually be repaired endoscopically, ductus side-entry and side-entry diversion jackets incorporating the required components thereafter used to expedite healing and protect against infection.

Temporary diversion from the distal urinary tract of irritating if not endogenously or exogenously contaminated or toxic urine, sometimes as a consequence of a disturbance in kidney function, expedites healing of diseased or operated upon tissue of the tract below that level, to which the diversion jacket can also target medication, and avert serious complications, such as interstitial cystitis. Where bladder pain syndrome or interstitial cystitis is already present, medication directly delivered into the lower urinary tract in addition to pentosan polysulfate, sodium can include such agents as dimethyl sulfoxide, heparin, lidocaine, botulinum neurotoxin A, oxychlorosene sodium, liposomal phospholipid vesicles, and stem cells, usually in various combinations.

To treat superficial lesions of the urothelial lining of the urinary tract or the endothelium lining a blood vessel, the electromagnetized jacket is adjusted to project a lower field strength. To treat a deeper lesion, the field strength is increased. If the former is a carcinoma and/or the latter a sarcoma, a jacket with a radiation shielding outer casing is used to allow the direct targeting of a radioactive agent such as a nanoparticulate radionuclide, or superparamagnetic iron oxide nanoparticles, to the treatment site. Because irradiating the epithelial lining of a ductus is no less capable of initiating a metaplastic cascade culminating in frank carcinoma, such use warrants close evaluation of the extent of malignancy and studied clinical judgement,

Magnetized side-entry and diversion jackets overcome a central problem in the lack of an ability to directly target magnetically susceptibilized, or carrier bound, stem cells to the diseased or lesioned tissue (“A fundamental impediment for the evolution of stem cell therapies has been the difficulty of effectively targeting administered stem cells to the disease foci.”—Cores, J., Caranasos, T. G., and Cheng, K. 2015. “Magnetically Targeted Stem Cell Delivery for Regenerative Medicine,” Journal of Functional Biomaterials 6(3):526-546).

In rare instances of metastasis from the prostate to the ureters, the direct targeting of radioisotopes to the lesions may detain if not eliminate the need for a ureterectomy.

| | NONADJUSTABLE | | ADJUSTABLE | |
|---|---|---|---|---|
| | TEM- | PROS- | DAY- | NIGHT |
| COMPONENTS | PORARY | THESIS | TIME | TIME |
| Cables | no | no | yes | yes |
| Chute control knobs | no | no | yes | yes |
| Ureteral end-plugs | no | yes | no | no |
| Jacket outlet obturator | no | no | yes | yes |
| Ejection assist device | yes | yes | no | yes |
| Ejection assist battery | yes | yes | no | yes |

### Adjustable and Nonadjustable Urinary Diversion Components.

Urine even more an irritant for a diseased rather than an initially healthy segment, irritant counter active agents are among those used. For example, acidity as an irritant is counteracted by including sodium bicarbonate. Body surface ports positioned on the outside of the skin are referred to herein as cutaneous, while those positioned subdermally are referred to as subdermal, subcutaneous, or hypodermic. Except where the condition treated is terminal, central line and portacath catheters are not intended for lifetime use. In contrast, a central line with distal end connected by a ductus side-entry or diversion jacket to the target ductus, typically a major vein in the upper chest, is fixed in position as not to limit the activities in which the patient will be free to participate.

Along the urinary tract, the small body surface or extracutaneous port—about 1.0 centimeter in outer diameter with on-skin openings for accessory channels, button cell battery, control knobs, and push/pull control knobs, 1.0 centimeter with only a battery added to the effluent mainline—much smaller than and not unsightly as is a stoma. In an adult, a stoma is well over an inch, or 25.4 millimeters, in every dimension, except for a period of weeks after its creation, when it remains quite swollen. Unlike a stoma, the small plastic port allows dispensing with repeated endoscopic treatment, and in some cases, stenting, both of which risk additional complications and the need for drugs risking additional complications. In a ureter, the avoidance of repeated endoscopy can avert strictures or the aggravation of strictures as a consequence of stimulating spongiofibrosis.

In a surface port that includes an effluent opening such the port shown in FIG. 26C with urine outlet, the protrusion through the skin of this opening assists in fixing the port in position. Including openings for the accessory channels in the same extracutaneous port allows reduction in the outer diameter of the port, the injection or infusion entry points clearly seen. To make it smaller in diameter, hence, less noticeable, a surface port with an effluent opening in lieu of a urinary stoma can be provided with subdermal, that is, subsurface, accessory channel entry holes.

When placed to a side of the mons pubis, the port is permanently out of sight, so that this consideration does not apply. Unless the esthetic factor is more important to the patient who will self-administer any necessary medication at home, this is not preferred. When the port does not include an effluent or other excurrent opening, it is placed subdermally or subcutaneously, where it is out of view. A port for placement thus is made larger in outer diameter—about 2.0 centimeters—with entry holes likewise larger.

Even though lodged within subcutaneous fat, this reduces any potential for the port to shift in position and the risk of misaligning the injection or infusion needle or fine cannula to the correct entry hole. The accuracy of injection or infusion is improved by identifying subdermal openings with a small dot, or number or letter identifier on the overlying skin. If the device is removed, these markings can be removed by laser. The strong shallow dome cover optimally disperses an impact from any angle to provide the maximum protection for a device which must have some protrusion from the skin. In contrast, a stoma, whether end or loop, is exposed, vulnerable, and will cause much disruption, and possibly, and if significantly traumatized, the need for revision.

Tiny (millimetric) in size, not weakening the abdominal muscles, the port is not dependent upon tissue adhesion and therefore not subject to skin irritation dehiscence, or structural failure in the form of prolapse, retraction, or herniation, and if stuck, is unlikely to result in injury comparable in consequence to exposed gut or gut enclosed within material sheeting used to aid cleanliness and prevent odor. Upon healing, the port is easily removed. Ureteral stents become encrusted, requiring replacement, which the ability to directly drip a stone solvent through the accessory channel of a ductus side-entry jacket positioned at a higher level would eliminate. The small size, negligible if any protrusion as seen, and invulnerability to injury compared to a stoma liberalizes its positioning along the body surface.

For example, with miniature pump assistance, a port placed in the mons pubis can directly target medication through their respective accessory channels to one or more side-entry connectors and/or jackets in different locations cephalad (superior, craniad), thereto. The area previously cleared by electrolysis, a mons pubis port is easily seen for self-injection, especially by the elderly and other infirm with comorbidities requiring several port accessory channel openings. If necessary pending the completion of electrolysis, the port can remain loose enough as not to interfere with completion of the electrolytic process. A urine outlet requiring a cutaneous or excurrent opening in any event, viewability is further enhanced by making the openings for the accessory channels likewise cutaneous rather than subcutaneous.

Areas where a stoma cannot be positioned because it would interfere with clothing become available. Relative freedom in port positioning also allows greater flexibility in avoiding less desirable routes for the lines connected to the port internally. In some instances, this allows finding a less tortuous path, allowing gravity to eliminate the need for assist pumping to drive the injectant to the target tissue. Relative freedom at line routing also reduces the risk of strangulation or entrapment. The risk of discomfort due to the encroachment upon neighboring tissue by electrical control lines even when strategically routed is provided by blue tooth remote control from the microcontroller.

The direct targeting of drugs through an accessory channel in the surface port to the site under treatment allows dosaging small and overall less frequent, which can be increased in concentration, the avoidance of untargeted tissue averting many if not all of the usual adverse side, drug-drug, and drug-food side effects. The addition of a basic side-entry jacket on the venous outflow allows the release of a reversal agent, or counteractant, to remove even trace amounts from the circulation. Except to allow direct viewing, relatively few procedures currently accomplished through endoscopy, and those mostly in the gastrointestinal rather than the urinary tract, such as bulking of the lower esophageal sphincter to prevent gastroesophageal reflux, cannot be supplanted by the means described.

Moreover, vesicoureteral reflux can be suppressed with an antacid drip readily established with these means, and a fine fiberscope can be passed through any opening in the surface port. An accessory channel can be used in lieu of endoscopy to coat the internal wall with contrast and to deliver a stone solvent, averting the need for and the complications associated with endoscopy, to include ureteral avulsion, perforation, and mucosal abrasion.

The accessory channel can be used to deliver medication such as a corticosteroid to avert inflammation and lidocaine, for example, to reduce pain that might otherwise necessitate stenting with its potential complications, which include migration, infection, fragmentation, residual fragmentation following removal, vesicoureteral reflux, encrustation, ureteroarterial, fistulization, necrosis, dysuria, hematuria, frequent urination, pain, and usually require removal of the stent (el Khader, K. 1996. “Complications of Double J Ureteral Stents,” (in French with English abstract at Pubmed) Journal d'urologie (Paris) 102(4):173-175).

When catheters were placed to convey urine on a long-term basis, it soon became clear that these would inevitably become fouled and eventually occluded by a buildup of infectious matter and/or crystalline stone, and no counteractant could be systemically circulated at the high dose that would be required to overcome the resistance of biofilm-forming bacteria or prevent the buildup of mineral encrustation. Early experience at fully implanting catheters to carry sterile blood became occluded with thrombus, and those placed to convey urine were soon found to develop a biofilm, sometimes with a crystalline encrustation.

The table above shows the different components required in each of the four embodiment types. Ureteral takeoff diversion jackets fall into two categories, one nonadjustable, comprising two embodiments, one for temporary use to expedite healing of the lower tract, the other for permanent use as a prosthesis when the lower tract is missing or dysfunctional, the other category adjustable. Adjustable embodiments are either for daytime use when the wearer is erect but unable to use a bathroom, or for use while the wearer is asleep.

With all four embodiments, diversion is to an external collection bag, requiring an abdominal or pelvic surface outflow port having features that make these less likely than a stoma to create skin problems. These include a removable cover that when removed exposes openings through which skin irritant counteractants can be instilled or injected. Takeoff at the ureter or ureters has the advantage of proceeding without the voluntary participation of the wearer. External urinary drainage devices such as single-use (Texas, urisheath/uriliner, “condom catheter,” diver's dry suit p-valve catheter) do not allow this. For daytime use, there is, therefore, no distraction from or need to interrupt a public performance.

Furthermore, the incorporation of suitable self-contained urinalysis biosensor microprobes, microprobes connected to a separately implanted microfluidic urinalysis laboratory on a chip, and/or pressure sensors in the diversion jacket and/or ductus side-entry jackets positioned at different levels along the urinary tract allow the automatic continuous collection of time-stamped diagnostic data which can be transmitted to a clinic without the need for the patient to go to the clinic in order to provide a urine sample of no more than instant, or point of care, significance.

Advances in Chronic Kidney Disease 20(6):516-535).

With a prosthesis, when the dysfunction or agenesis is restricted to an intermediate portion of the urinary tract so that the urethra remains intact, diversion can be directly to the bladder, allowing voiding in a normal manner without the need for an external collection bag, the means therefor described and illustrated in copending application Ser. No. 14/998,495. When diversion is to a neurogenic bladder lacking in urge sensation and/or voluntary contractibility, a pressure sensor is used to actuate a tiny, fully implanted, electric motor having a rotor driving an eccentric load, which signals the wearer of the need to void by vibrating. When a synthetic continent neobladder is used, takeoff is from the ureteral stump or stumps, which are connected to the neobladder by a nonjacketing side-entry connector.

If urination is frequent, a port with two button cell battery compartments are used to power the evacuation impeller and the synthetic neobladder stopper flap used to attain continence. The same device signals the need to void through a mons pubis port in a relatively normal manner. The wearer then initiates micturition by pressing a switch to obtain contraction through electrostimulation of the detrusor, either or both urinary sphincters, or all three. These alternatives to passive drainage into an external collection bag require supplementary electronics addressed in copending application Ser. Nos. 14/121,365 or 15/998,002 and 14/998,495. Copending application Ser. No. 14/121,365 or 15/998,002 described ductus side-entry jackets, to include such details as the use of spring-loaded hinges to snap the jacket about the substrate ductus when placed. by the operator.

The diversion chute in a nonadjustable or prosthetic embodiment is advanced into the ureteral lumen on placement and never retracted unless and until it becomes necessary to remove the device. Since a temporary embodiment will have been placed to divert urine away from the lower tract to expedite healing, eventual removal will be necessary. The diversion chute in an adjustable embodiment is advanced through the side-entry hole into the lumen with the shape-memory and bisphenol A and residual plasticizer-free polymer side-entry hole outlet obturator folded at the nose of the diversion chute much as a half open umbrella. Once in the ureteral lumen, body temperature causes the obturator to spontaneously unfold or deploy. If a temporary embodiment, it will become necessary to remove the device. In use, the obturator will move between covering the side-entry hole and the luminal lining opposite, but will never move through the side-entry hole.

This is accomplished by chilling the obturator with a cold air gun. Because it must remain in constant use, the nonadjustable embodiment for use as a prosthesis omits miniaturized push/pull control cables and control knobs at the surface port used to advance and retract the side-entry jacket diversion chutes needed for elective use. The diversion chute is integral with an outlet obturator at its front or distal tip to close off the ureteral lumen when the chute is retracted. A central design consideration is the avoidance of all but the least urothelial compression that in both adjustable and nonadjustable embodiments will suffice to prevent leaks past the diversion chute when deployed, and in an adjustable embodiment, will suffice to prevent leaks through the side-entry hole as diversion outlet when the chute is retracted.

The significance of avoiding all but essential compression of the urothelium and suburothelium is addressed below. Diversion directly to the urethra so that the sensation of urgency is lacking necessitates the interposition of a pressure sensor in the diversion line to actuate a vibration motor, signaling the patient of the need to void, something not required with a neurologically functional bladder. The prosthesis does, however, require an evacuation assist device should the patient, especially a patient with impaired ureteric peristaltic function, adopt a posture as leaves gravity alone unable to achieve expulsion. The back pressure, not sensed by the wearer whose bladder is bypassed, then reaches the level where vesicoureteral reflux (regurgitation, backwash) of urine back up into the ureters results, boding serious complications.

Vesicoureteral or ureteral reflux in a neonate or infant can lead to hydroureteronephrosis and/or hydronephrosis, which if not treated promptly, will eventually result in end-stage renal failure. In a neonate or infant, the moisture barrier-protected viscoelastic polyurethane foam lining of the diversion jacket is made thick enough to allow the device to remain in place for the first several years. However, while the diversion jacket might last for life, if placed in early childhood, the diameter of the ureteral lumen will likely exceed the range of diameters the chute can prevent from leaking past it. With a young patient, the chute is therefore made wider and thinner to allow increased width with increased caliber or internal diameter of the ureteric lumen.

In an adult, the same diametrical compliance conforms to the significant variability in diameter of the ureter along its length, necessitating valves made in different sizes. The primary fact that the dimensions of the diversion chute must conform to the cross sectional area of the substrate native lumen at the level where it is to be placed makes this necessary. Adjustable embodiments are of two kinds, one for daytime use by a wearer who has normal urinary function but is unable to use a bathroom, and one for night-time use, when voiding will be with the wearer other than erect. There are other possibilities, such as a daytime wearer with impaired ureteral peristalsis who may require using the night-time embodiment. The causes of impaired ureteral peristaltic function are numerous, and as addressed below, include numerous genetic, developmental, and acquired conditions.

Ordinarily, where one ureter is missing a segment due to trauma or deformity as to require that a small segment be removed, a unilateral prosthesis as described herein is unnecessary, end-to-end anastomosis of the cut ends by ureteroureterostomy practiced universally. Defects that extend over too great a length of the ureter for end-to-end anastomosis require anastomosis of the damaged or abnormal ureter at the most proximal part of the functional urinary tract. While diversion jackets allow a ureteral obstruction to be bypassed analogously to a coarctation of the aorta, and the accessory channels can be used to release an antimicrobial, for example, most causes should be treated by accepted surgical methods.

Ureteral flow can be shunted rather than bypassed about a diseased or otherwise obstructive segment, Shunting using conventional surgery may be to the other ureter by transureteroureterostomy, to the renal pelvis by ureteropyelostomy, to a renal calyx by ureterocalicostomy to the bladder by ureteroneocystostomy (neoureterocystostomy) with or without a vesicopsoas hitch or pull-up and psoas ureteropexy or a Boari bladder flap (http://emedicine.medscape.com/article/1893904-overview), or reconnection to the bladder through ureteral reimplantation, all long since optimized, and with the exception of the Boari flap, which as addressed below, harvests a portion of the bladder dome, to be preferred, certainly in a non-immunocompromised patient, as avoiding the need for an external collection bag (for illustrations, see, Santucci, R. A. 2017. “Ureteral Trauma Treatment & Management,” online at http://emedicine.medscape.com/article/440933-treatment).

A Boari flap can be supplanted by attaching a side-entry jacket toward the end of the ureteral stump and using the accessory channel to pass urine to the bladder through a nonjacketing side-entry connector fastened at the bladder dome. The use of basic side-entry jackets, nonjacketing side-entry connectors, and diversion valves and servovalves in lieu of conventional surgical repair to bypass a diseased segment or shunt an obstruction might be appropriate where the advantage of direct drug targeting though the accessory channels is desired. Such an approach should not necessitate reentry to recover the side-entry devices.

A jacket used thus would normally have a second accessory channel to allow the direct delivery of drugs into the catheter and bladder. Ideally therefore, barring complications, even though the means described in copending application Ser. Nos. 14/121,365, 15/998,002, and 14/998,495 as well as herein might be used to supplant the accepted procedures altogether, the conventional repair most familiar to the operator should be done with the addition of means described in the foregoing applications and herein to target drugs or a crystal solvent, for example, directly to the wounds, anastomoses, and susceptible segment of the tract.

| PORT OPENINGS | ADVANTAGES | DISADVANTAGES |
|---|---|---|
| CUTANEOUS | Clearly viewable injection | Skin perforated. |
| | points when placed | Noticeable outside |
| | in the mons pubis. | of mons pubis. |
| | Does not require backplate | |
| SUBCUTANEOUS | Skin unperforated. | Except in mons pubis, |
| | Little noticeable outside | palpation or |
| | mons pubis. Unseen when | tattoos needed to locate. |
| | placed in mons pubis | Not external/unseen. |
| | | Requires faceplate when a |
| | | urine outlet is required. |

### Epidermal, or Cutaneous, and Subdermal, or Subcutanous, Port Openings.

For this purpose, a conventional portacath, or mediport (Medi-Port®), positioned in the pectoral region is used to infuse the drug, drugs, or other agents into the accessory channel leading directly to the nonjacketing side-entry connector, side-entry jacket, or diversion valve. Where ductus side-entry jackets are for partial shunting or bypassing as pertains to channeling blood to ischemic tissue on a permanent basis, for example, diversion jackets shunt or bypass all flow through the ductus.

For dialysis or apheresis, such shunting is intermittent. The means described herein are intended for long term use, which may involve short or long interval intermittent or multiple daily use. The synthetic to native long term direct contact sutured interfaces of conventional polyethylene terephthalate (Dacron®, Terylene®, woven Dacron® or Terylene©), and polytetrafluoroethylene shunts and bypasses—of which the failure is a medical emergency—are susceptible to complications which the protected interfaces of ductus side-entry jackets, diversion jackets, and connectors are not.

Automatic sensor monitoring of urinary constituents, rate of output, and volume may signal the need for examination or if programed to automatically respond to such deviations, register the need for and initiate the system response. Especially when collateral functions are involved, there exists the need for a subdermally placed injection and infusion port with multiple openings to allow the direct piping of different drugs into different blood vessels or levels along the digestive tract. Ambulatory urinalysis and hemoanalysis can be used to signal er, here, as deviations in renal as well as other organ system functions as reflected in the composition of urine and/or blood, and the need to initiate delivery of remedial medication.

For example, ketoacidosis and hyperglycemia signal the need for insulin, which provided promptly, averts the need for sodium bicarbonate and the risk of hypernatremia. Significantly, the ability to directly target the delivery of drugs to specific levels of arteries and veins allows control over whether processing of drugs will precede or follow first pass processing by the liver or kidneys. As the present content pertains more narrowly to repairs of the urinary tract, the application of side-entry diversion jackets is here illustrated in terms of that organ system. Also detectable with prompt response are nitrituria, acute renal failure, hypokalemia, hyperkalemia, adrenal insufficiency as hypoaldosteronism, hypercalciuria, hypocalciuria, hypophosphaturia, hyperparathyroidism; hypertension, diabetes mellitus, jaundice, or hyperthyroidism seen as proteinuria or albuminuria. Many other tests might be cited.

In certain circumstances, certainly where tissue is harvested creating a separate preliminary site for the risk of complications, the means described in copending application Ser. No. 14/121,365 or 15/998,002, 14/998,495, and herein can be used to avoid these. Attachment to a calyx, the pelvis, or the bladder is with a nonjacketing side-entry connector, and attachment to a ureter is with a ureteral takeoff jacket. Where the condition of the tissue for anastomosis prevents such, these means not only avert the need therefor but afford a path to the affected tissue for the direct delivery of drugs. A native or allograft ureteropyelostomy performed with a nonjacketing connector at the pelvis, or a ureterocalicostomy calyx with the connector at the calyx and side-entry jacket on the ureter eliminates the need for a partial nephrectomy at the inferior pole of the kidney.

Using the means described and illustrated herein and in copending application Ser. Nos. 14/121,365, 15/998,002, and 14/998,495, a nephrostomy is accomplished with a nonjacketing side-entry connector with nephrostomy tube run through to the pelvis to be positioned as shown in Ser. No. 14/998,495, FIG. 11, with the nephrostomy tube connected to the most proximal intact segment along the tract. If this is to the ipsilateral ureter, a ductus side-entry jacket as described and illustrated in copending application Ser. No. 14/121,365 or 15/998,002 is used to connect to the ureter, with the accessory channel or channels of the ureteral jacket used to deliver any needed medication. At this level, the surface port is positioned in the pectoral region. The takeoff for a pyelostomy is a nonjacketing side-entry connector with connection the same as for a nephrostomy.

Takeoff from a ureter is by means of a side-entry diversion jacket as described herein, with connection either to an abdominal surface port, or if a distal portion of the tract remains intact and can be bypassed to allow normal voiding, then to that portion. If to the contralateral ureter or to a portion of the same ureter beyond the diseased or traumatized segment, then connection is by means of a ductus side-entry jacket. If to the bladder, then connection is by means of a nonjacketing side-entry connector, with placement of the surface port dependent upon the level of the diversion jacket along the ureter. Side-entry connectors, ductus side-entry jackets, and side-entry diversion jackets can all be provided with one or more accessory channels to allow the direct delivery of medication from the surface port to the site of injury or disease or the site to which the diseased or injured segment is bypassed.

Depending upon the circumstances, takeoff from a dysfunctional bladder can be by means of a synthetics-based suprapubic cystostomy connected to an abdominal surface port. Because the surface ports described herein are much thinner and less noticeable than either a stoma or a conventional portacath, the positioning of these along the body surface is much more flexible. If diversion is to a bladder that is functional or can be made to contract with the aid of electrical stimulation, then as shown in copending application Ser. No. 14/998,495, FIG. 12C, connection is to the urethra, the need for a collection bag eliminated. Thus, while diversion as illustrated herein is to an paracorporeal collection bag, depending upon the detailed condition of the urinary tract, the need for a bag can often be eliminated.

Whether a conventional ureteropyelostomy or ureteroneocystostomy is more susceptible to complications in kidney transplantation is a matter of dispute with most references antiquated and those recent opting for ureteroneocystostomy where the means described in the foregoing applications and herein are believed superior to both. Much the same applies to comparisons of ureteroureterostomy versus conventional ureteroneocystostomy.

Considered sound by conventional standards, the Boari flap technique tubularizes a portion of the bladder dome or roof, injuring and risking complications that a ureteral diversion jacket and drainage catheter emptying into the otherwise intact bladder through a nonjacketing side-entry connector would avoid, as well as allow the direct targeting of drugs to the site from a portacath. Reports of successful results initially may not specify long-term outcomes beyond three months.

Diversion with a nonjacketing side-entry connector or ductus side-entry jacket and catheteric drainage line described in copending application Ser. No. 14/121,365 or 15/998,002, and 14/998,495 or a diversion jacket as described herein still have a distinct advantage in allowing the direct targeting to the treatment site of drugs. Certain complications also recommend the use of synthetics over reconstructions. These include a transureteroureterostomy when the risk of injury to the recipient ureter is high (Sandoz, I. L., Paull, D. P., and MacFarlane, C. A. 1977. “Complications with Transureteroureterostomy,” Journal of Urology 117(1):39-42). While more successful than most other reconstructive procedures, distal obstruction, recurrent stricture, and leakage may necessitate more than one follow-up procedure (Iwaszko, M. R., Krambec, A. E., Chow, G. K., and Gettman, M. T. 2010. “Transureteroureterostomy Revisited: Long-term Surgical Outcomes,” Journal of Urology 183(3):1055-1059; Strup, S. E., Sindelar, W. F., and Walther, M. M. 1996. “The Use of Transureteroureterostomy in the Management of Complex Ureteral Problems,” Journal of Urology 155(5):1572-1574).

For refractory urinary incontinence, either the nonadjustable (prosthetic) embodiment when incurable, and the adjustable embodiment when correctable can be used in lieu of an indwelling Foley catheter, eliminating restriction on the duration of continuous use of a single device, as well as numerous complications, primarily of infection, but also discomfort, genitourinary trauma, clogging, leaking, hematuria, retention and fragmentation, cystolithiasis, pyelonephritis, urosepsis, bacterial endocarditis, and problems due to breakage during insertion and removal. Where incontinence affects an otherwise intact lower urinary tract, the addition of an intracystic urethra-noncompressive continence device with directly targeted therapeutic support, application to be filed shortly, allows the wearer to use an adjustable diversion device in the open position during the day without a collection bag.

External urinary drainage devices (single use or Texas, urisheath/uriliner, “condom catheter”, diver's drysuit p-valve) are less problematic than urethral catheters but still pose risks in addition to infection. Long term use, especially when not properly fitted and maintained, can lead to ulceration, necrosis, urethrocutaneous fistulae, urethral diverticulum, and fibroepithelial polyp of the prepuce. Improvised such devices can result in severe complications (see, for example, Paul, S., Dalela, D., Prakash, J., and Sankhwar, S. 2013. “Penile Elephantiasis: A Rare Consequence of Inappropriate Use of Condom as External Urinary Collection Receptacle,” BMJ [British Medical Journal] Case Reports 2013). Implantation of the devices to be described for permanent use supplants the use of conventional urinary catheters of all types for long-term use, which overcome the significant problems associated with noncompliant use, even by patients and healthcare personnel, who may be competent but neglectful.

Combining a lower abdominal or pelvic, that is, subcutaneously implanted body surface port to a side of the mons pubis or mons veneris, which excurrent to emit urine, must provide an opening to the exterior, with a subcutaneously placed, totally implanted port positioned in the pectoral region for targeting drugs through its accessory channel to the diversion valve positioned along the ureter can be argued for as reducing the risk of infection as can the design of the port which allows the direct application of antimicrobials or antiseptic.

Cutaneous, that is, above-skin injection incurrent openings just beneath the port outer dome cap, are covered over by a self-sealing silicone membrane, or septum, (Yoo, J. J., Magliochetti, M., and Atala, A. 1997. “Detachable Self-sealing Membrane System for the Endoscopic Treatment of Incontinence,” Journal of Urology 158(3 Part 2):1045-1048) during use, then covered over by closing the hinged port dome cap. Open and closed, the port is wetted with an antimicrobial or antiseptic, and subdermal ports are closed off from the environment, so that pathogens can enter only on the needles or cabled devices that are passed through the skin.

Since as with any injection, the skin is swabbed with an antiseptic before insertion, the likelihood of microbial invasion is slight. However, combining these in a single port with excurrent urine outlet and subdermally positioned injection openings for drugs eliminates the need for a second port. The surface port not only replaces a stoma, itself the site of numerous complications demanding constant maintenance, but serves as an entry portal for the direct targeting of medication, maintenance agents, and the passage of cabled devices such as scopes and lasers through the apparatus. Where no excurrent flow is required as it is with urinary diversion, the port is completely subdermal. Any difficulty in distinguishing different entry holes in the port by palpating the slight bump the port makes at the surface is clarified by tattooing tiny dot indicators.

While seldom exposed, hence, unlikely to pose a cosmetic problem, if the urinary tract no longer requires a prosthetic outflow path, or exogenous or therapeutic support, the dots are readily removed by laser or minipunch skin excision. General information pertaining to ductus side-entry jackets and diversion valves is provided above in this section. The urine diversion chute situated behind the trepan shown in the drawing figures is retracted during, and little if at all interferes with plug extraction. In prosthetic, that is, unadjustable, or fixed embodiments, once the tissue plug is removed, the diversion chute is advanced into luminal position by pushing on the accessory channel until male and female detents on the trepan floor and beneath the diversion chute engage with an audible click.

With an intermittent embodiment, placement is likewise with the diversion chute in the retracted position, except that either push-pull solenoids or a Bowden or push/pull control cable adjusted at the surface port is used. The adjustable embodiment can be used to target a drug or drugs to the lower tract at the same time that urine is diverted away from the lower tract, which bypassed, is kept free of contract with urine, which is caustic, and thus afforded an improved opportunity to heal following the initiation of treatment for a bladder dysfunction or surgical procedure.

Exposure of urothelium to urine, which contains “ . . . harmful and noxious substances that must be stored for hours . . . ” can leave it defective for having undergone “ . . . urothelial barrier breakdown and developed ” . . . inappropriate signaling from urothelial cell to underlying sensory afferents and potentially interstitial cells . . . ” (Hill, W. G. 2015. “Control of Urinary Drainage and Voiding,” Clinical Journal of the American Society of Nephrology 10(3):480-492), resulting in lower urinary tract dysfunction, to include frequency, urgency, incontinence, overactivity, and pain.

During placement of the diversion valve, the opposing wall of the ureter is kept from being drawn against the trepan by insufflation at the least pressure needed to keep the opposite urothelial surface at a safe distance. That with the system described, doxorubicin would be directly pipe-targeted to the nidus, avoiding its life-threatening side effects, to include dilated cardiomyopathy tending toward congestive heart failure, neutropenic enterocolitis, or typhlitis, and less severe palmar plantar erythrodysesthesia, or chemotherapy-induced acral erythema even at elevated dosages, should be clear.

Insertion of a ureteral stent or Foley catheter to cover over the urothelial lining protects it from urine but also makes it inaccessible to the direct targeting of medication through a side-entry diversion valve. In this application, urine is diverted from the ureter or ureters and is passed directly into an paracorporeal collection bag having been detoured about the affected lower tract. At the same time, medication flows in through an accessory (service channel, sideline, drugline) to wet the ureteral urothelium to expedite recovery while spared exposure to urine.

The foam lining of the diversion valve takeoff jacket allows the diversion valve to be positioned at any level along the ureter. Moreover, where the ductus deferens or broad ligament of the uterus cross the ureter, the dimensions of the trepan and diversion chute allow avoiding any constriction; instead, the jacket is placed slightly above (cephalad, craniad, superior) or below (caudad, inferior) to this small segment. Other points of constriction along the ureter can if difficult for placement of a diversion jacket can be connected through a miniature nonjacketing side-entry connector and catheter, as delineated in copending application Ser. No. 14/998,495.

At 2.0 millimeters in diameter, the trepan, hence, the hole it cuts, is substantially the same in diameter as the mean internal diameter of the adult ureteral (ureteric) lumen when passing a bolus. The same jacket is used for the average adult human blood vessel. In distinguishing fixed prosthetic from intermittent use embodiments, it warrants emphasis that a spectrum of conditions lies between these extremes. For this reason, either type embodiment incorporates some features that would support the purpose of the other. For example, the fixed embodiment incorporates an accessory channel which is connected to a groove running along the center underside of the diversion chute and end at the distal tip thereof inside the native lumen, and a set-once manually operated flow, or prosthetic diversion, valve can actually be retracted, but requires more force.

Accessory channels afford paths for the direct targeting of drugs to any portion of the ureter or other part of the lower urinary tract that may require medication. For example, the tissue of the dysfunctional lower tract might become infected and heal more quickly with the direct (surface-wetting, topical) application of an antimicrobial, anti-inflammatory, and/or anesthetic where appropriate, allowing a background systemic dose much reduced if still needed at all. For use along the urinary tract, where flow through the ureter is less forceful than that pulsatile under pressure encountered with an artery, the problems of apparatus leakage or inflow can be averted. This is by administering an antidiuretic, or oliguric, drug such as a shorter acting antidiuretic hormone and octapeptide vasopressins and placement of a cross clamp over the brief interval it takes to place the jacket.

As antidiuretics, carbamazepine and chlorpropamide must be systemically circulated to reach the neurohypophysis. Exposing the brain as well, these drugs are unsuitable for direct targeting of the urinary tract, of which the object would be precisely to bypass the central nervous system. Furthermore, systemically administered, chlorpropamide, now seldom prescribed to treat diabetes, is contraindicated for use in the obese and elderly, and carbamazepine, used primarily for neuropsychiatric disorders, is no less likely to cause more harm than good.

If necessary, once the jacket has been placed, an antidiuretic drug reversal agent is administered. The administration or withholding of an antidiuretic is a clinical judgement based upon the general health and drug regimen of the patient, especially with respect to diabetes. Blood is prevented from extravasation only while the jacket is being placed; once placed, the shunt or bypass created allows the free outflow of blood to the target tissue. Accordingly, a diversion type side-entry jacket for placement along a blood vessel but not one for placement along a ureter must incorporate a water jacket as shown in copending application Ser. No. 14/121,365 or 15/998,002.

Conventionally, a nephrostomy is used as a temporary bridging or holdover measure pending surgical correction where trauma or deformity of the distal urinary tract prevents urinary diversion at or to a lower and considerably less physiologically complex and heavily vascularized, hence, risk-prone level. Copending application Ser. No. 14/998,495, entitled Nonjacketing Side-entry Connectors and Prosthetic Disorder Response Systems, describes a method and apparatus for creating a permanent nephrostomy using synthetic materials. Unless an intact bladder is present or can be created, diversion is to a conventional paracorporeal, or externally worn collection bag. However, urinary diversion, or takeoff, at the kidney or kidneys assumes that no less desperate alternative is offered at a lower level.

Whenever possible, takeoff at the ureter avoids the greater risk incurred with interception at the kidney, which includes hemorrhage, infection, septic shock, iatrogenic injury to include renal puncture damage, increased time to establish renal drainage, coagulopathy, and “ . . . hematoma, pseudoaneurysm, arteriovenous fistula, injury and disruption of the renal pelvicaliceal system, arteriocaliceal fistula, and renal foreign bodies such as retained wires, sponges, or other surgical or endourological instruments” (Esparaz, A. M., Pearl, J. A., Herts, B. R., LeBlanc, J., and Kapoor, B. 2015. “Iatrogenic Urinary Tract Injuries: Etiology, Diagnosis, and Management,” Seminars in Interventional Radiology 32(2):195-208). 24(4):224-228).

The risks of percutaneous nephrostomy are greater when the kidney with ureter is a transplant and a functional bladder missing, for example, the diversion jacket is positioned above would otherwise be an end-to-end ureterocystic anastomosis.

Using ureteral takeoff by means of a diversion side-entry diversion jacket rather than percutaneous nephrostomy requires that the ureteral obstruction be so positioned as to afford sufficient length in the post pelvic ureter to position the jacket; however, post-transplantation stricture most often ensues at the ureterovesical anastomosis, and the jacket, millimetric in dimensions, this requirement will be satisfied. Copending application Ser. Nos. 14/121,365 or 15/998,002 and 14/998,495 address the concept of directly targeting immunosuppressive drugs to the arterial supply of the transplant organ rather than exposing the entire body to such drugs, increasing susceptibility to disease.

For temporary use, such as to allow diagnostics preparatory to prospectively curative therapy not requiring continued monitoring, the tract is emptied of urine, the ureteral diversion jacket removed, the aperture created cleared of any fibrosed tissue that may have formed about the periphery of the 2.0-millimeter aperture in an adult, and the aperture then allowed to heal, preferably by first, or if necessary, second intention. First intention is by closure with an adhesive such as surgical cyanoacrylate cement and wrapping about the wound with absorbable tape; second intention by wrapping about the ureter without first closing the wound but wrapping about the ureter with a strip of absorbable tape or artificial skin, and allowing the aperture to fill in by second intention.

Requiring entry through a ‘keyhole’ incision, takeoff at the ureter is no more, and probably less, risk prone than are the existing alternatives of a percutaneous nephrostomy, which is not long-term, much less ambulatory, or either antegrade or retrograde insertion of an indwelling ureteric J- or double J-stent. Neither ureteric stents nor percutaneous nephrostomy can remain in place for more than a short time, and cannot be exchanged interminably so as to be considered permanent treatments. Approach from outside the ureter with a jacket is less susceptible to perforation than is transluminal insertion. However, stone induced reflux resistant to lithotripsy is best served with the mechanical clearing of the lumen afforded by a stent. Additional reports in the literature of ureteral stent-related complications are numerous.

In a hospital setting, percutaneous nephrostomy does, however, allow independent urinary diversion from either kidney into a separate external collection bag, and therewith, the ability to monitor the function of each kidney for renal uropathy, that is, abnormal differential function whether one kidney is infected, physiologically impaired, injured, obstructed due to a stone or structural defect, or has been operated on or transplanted (see, for example, Sharma, U., Yadav, S. S., and Tomar, V. 2015. “Factors Influencing Recoverability of Renal Function after Urinary Diversion through Percutaneous Nephrostomy,” Urology Annals 7(4):499-503) through separate urinalysis as an adjunct to:

a. Renal biopsy.
b. Dynamic renal scintigraphy.
c. Other modes of nuclear renal scanning, such as the use of the isotope mercaptoacetyltriglycine.

That takeoff at the ureters allows the kidneys to be separately monitored on a long-term basis without confinement to the clinic improves on this capability. Differential urinalysis is possible either by diversion from one of the kidneys, the other allowed to continue outflow through the lower tract so that only urine from the side to be monitored enters the collection bag, or by providing independent drainage lines leading to separate collection bags, isolating the urine emitted by either kidney. With an adjustable or intermittent embodiment, the admixture of urine from the contralateral ureter is avoided by retracting the chute on the contralateral ureter to allow urine to flow into the lower tract.

If a need for side to side follow-up kidney differential urinalysis is desired in a nonadjustable or prosthetic embodiment, the neoureters are each led to separate neoureter collection chambers and separate collection bags worn on the front or a side of either thigh. The output from each ureter is then implemented as a separate channel, if necessary, with independent rather than convergent chamber as shown in FIGS. 1, 4, and 5, with separate pressure actuated microswitches and neoureter convergence chamber impellers, which is a subminiature fluid driver similar to an axial box fan with blade guard, adapted to forcibly expel urine from the chamber if for any reason ureteric peristalsis fails to propel urine fully out of the line and into the collection bag so that the pressure in the chamber rises to a level portending ureteric reflux.

Ordinarily, no neoureter convergence chamber impeller, pressure actuated switch, button cell battery in the surface port, or wiring to connect the two are needed in the neoureter convergence chamber. This is because calyceal or pelvic and ureteral peristalsis forcibly drive the urine boli down into the convergence chamber where the course of least resistance to continued outflow, with or without the added force of gravity, are the chamber outlet and line leading to the collection bag. The administration of a diuretic or obtaining diuresis by instructing the wearer to drink water can be used to stimulate intensified calyceal peristalsis and the jet effect (citations below) to overcome the resistance to outflow, or neurostimulation at the renal pelvis might be used to accomplish this.

However, diuretics and loop diuretics risk many potentially serious side effects, mostly by creating acid-base and electrolyte imbalances. These include hearing loss, lethargy, seizures, nausea, emesis, constipation, tachycardia, neuropsychological impairment, such as confusion, depression, anorexia, dizziness, and coma, pancreatitis, interstitial nephritis, skin rashes, thrombocytopenia, and myopathy, such as cramps, other myalgia, weakness, and paralysis. To intensify peristalsis through electrical neuromodulation at the calyces responsive to a pressure actuated switch housed in the convergence chamber used or not used to actuate a chamber impeller is also possible but considered to needlessly place inordinate stress on normal peristaltic function at the pelvis.

Intrinsic motility alone may not succeed when the wearer adopts certain postures, however, such as to lie on the side opposite the surface port, which can be discouraged by wearing the collection bag on the front rather than the outer side of the thigh. The surface e port can be positioned midway along a short Phannenstiel incision; however, the outlet hose must still veer toward the side of the collection bag. Most potential users will meet the requirement for peristaltic sufficiency, so that components to assist in the evacuation of the convergence chamber will not be not required. However, pelvic and/or ureteral peristalsis may be congenitally impaired, and can become temporarily impaired to remain impaired without treatment.

Adjacent to the ureters, appendicitis and diverticulitis as well as “ . . . inflammatory, infectious, or malignant processes of the colon, appendix, oviducts, or ovaries” (http://emedicine.medscape.com/article/1949127-overview#showall) can impair ureteral peristalsis, tiny uroliths present or since passed can, as can ureteritis such as ureteritis cystica and urothelial neoplasms benign or malignant.

The causes of permanent ureteric impairment, often developmental or genetic as to metabolism and/or structure, which may prove resistant to treatment, as well as structural deficits following trauma, are equally if not more numerous, and/or the wearer falls asleep in a position that minimizes or eliminates gravity as a contributing force to voiding, an automatic assist device averts reflux. When the need for an assist is temporary, the wearer simply inserts the button cell battery in the surface port as needed.

Synthetic tubing not susceptible to “ . . . inflammation, uremia, hypoxia, sheer stress, and increased thrombogenicity’ leading to intimal hyperplasia, another advantage of takeoff at the ureters using synthetic materials is avoidance of the procedural risks and the 40 percent obstruction due to intimal hyperplasia experienced at one year following arteriovenous fistulation. Moreover, the apparatus described herein is able to eradicate the thrombus that ordinarily occludes catheters used to move blood by using the side-entry diversion jacket accessory or service channel to run an anticoagulant such as a low molecular weight or unfractionated heparin and/or vitamin K antagonists through the line.

To avert the risk heparin can pose for an impaired kidney, protamine sulfate is kept available as a reversal agent. Ureteric obstruction due to distal ureteral ischemia can be addressed by diverting arterial blood to the ureter. A larger jacket is placed about the source artery and the oxygenated blood is passed through a catheter and smaller jacket placed about the ureter. Accessory channels at both the takeoff or source artery and the input or destination jackets allow drugs to be targeted through the catheteric line for maintenance and the native lumina for therapy.

With either a diversion or simple junction side-entry jacket, anastomotic stricture, which can arise in several different contexts along the digestive tract so long as such procedures are continued, a gut-ureter junction as in an ileal conduit will continue to pose problems of stricture and metaplastic transition to malignancy, necessitating revision.

This is the more likely under various condition of disease, notably, tuberculosis of digestive and excretory tissue where anastomosis is not a factor (see, for example, Goel, A. and Dalela, D. 2008. “Options in the Management of Tuberculous Ureteric Stricture,” Indian Journal of Urology 2008 24(3):376-381). With or without initial or subsequent endoscopic or balloon intervention, strictures anastomotic or due to disease can be treated through the direct targeting to the strictured segment of ameliorative if not curative drugs through a simple junction, or if a ureter where diversion is needed, a urinary diversion jacket.

Automatic delivery directly to the site of the stricture from a port at the body surface, flat pectoral storage reservoir, and neoureter convergence chamber impeller, if necessary, overcomes patient noncompliance with drugs to be “ . . . given in combinations of four to six drugs for at least 6 months for drug-sensitive TB and up to 24 months for drug-resistant TB . . . ” (Sacksteder, K. A., Protopopova, M., Barry, C. E. 3rd, Andries, K., and Nacy, C. A. 2012. “Discovery and Development of SQ109: A New Antitubercular Drug with a Novel Mechanism of Action,” Future Microbiology 7(7):823-837). Tuberculosis is cited in an exemplary sense, any infection for which an antimicrobial is available no less treatable thus. The term peristomal denotes the skin surrounding a stoma, while the term parastomal pertains to stomal herniation.

While less susceptible than a colorectal stoma, which must pass septic matter containing injurious enzymes and acids, an ileal conduit is susceptible to early and late postprocedural complications, to include renal deterioration and the need for dialysis, ischemia, retraction, parastomal hernia, prolapse, stenosis, or stricture, necrosis, ulceration, fistulation, or fistulation, and herniation, predisposing to incarceration and strangulation of the gut, as well as posing a greater risk of infection, these complications more likely in patients with Crohn's disease. A body surface port made of synthetic material is critically less susceptible to such complications. The stoma is the cause of most complications following a relatively simple ureterocutaneostomy. Additional references to early and late complications of stomata are cited below.

Rather than to replace a segment of the ureter with tubularized gut, another part of the digestive tract, oral mucosa, has been recruited to replace strictured (or otherwise no longer functional) segments along a ureter. Given that degenerative metaplasia leading to malignancy is usually a long process so that cancer may not appear for 15 years or longer, the technique has not been in use long enough for sufficiently long-term follow-up; however, there is no reason to assume that exposure of this absorptive digestive tissue to urine would not exert the same degenerative effect over time as it exerts on the lining of the ileum. Such degeneration a slow process, the technique is best limited to the elderly and in then nonelderly, to the urethra, to which the means described herein do not apply.

Synthetic materials are not subject to complications such as ischemia, stricture, prolapse, retraction, swollenness for two months following stoma placement, necrosis, et cetera, where the means to deal with such conditions introduce additional complications. In addition to misappropriating a segment of gut, extending risk to another organ system, a stoma requires a significant breach of the abdominal musculature, critically weakening it, then puts the ileal segment through the breach, and once through, may roll the ileal segment back, where it often fails to heal with adequate adhesion to the skin and leaves a relatively large protrusion at the outside of the body, making it vulnerable to injury. Furthermore, long term exposure of nonurinary tissue to urine invites metaplasia and malignancy.

The consequences are then, needless risk of injury and pronounced susceptibility to failure, whether present as retraction or herniation. By contrast, the external ports to be described do not require a large breach through the abdominal musculature and inconspicuous, do not present a relatively large stoma which fails to adhere to the skin, making the integument mounting such a small port invulnerable to rupture due to the port, and a synthetic port can provide any, indeed more openings for incurrent and excurrent passage than any type of stoma.

While the comparative failure rate of gastrointestinal and urinary stomata is undocumented, heavier loading would indicate that the former fail more often; nevertheless, urinary stomas fail often. For a synthetic port to fail structurally as herniation, prolapse, or retraction is very much against the odds, and significantly, failure is a “ . . . prevalent problem and treatment can pose difficulties due to significant rates of recurrence and morbidities of the repair.” (Gillern, S. and Bleier, J. I. 2014. “Parastomal Hernia Repair and Reinforcement: The Role of Biologic and Synthetic Materials,” Clinics in Colon and Rectal Surgery 27(4):162-171).

By contrast, a surface port made of a suitable polymeric material, such as gear hardness grade nylon, especially with easy access to instill a germicide, is less susceptible to infection and in an adult, requires a small breach of 3.0 millimeters for the excurrent or effluent opening to connect the outlet hose leading to the urine collection bag, with 2.0 millimeter holes for as many accessory channels as needed, 2.0 millimeter spaces to mount push/pull control cable knobs if needed, and a small compartment for a neoureter convergence chamber impeller battery if present. Overall, the port has an outer diameter of about 8.0 millimeters when the accessory channel or channels are above the skin and 5.0 millimeters when the accessory channel or channels are subdermal.

Neither prevention through the placement of prophylactic mesh or fascial fixation during the procedure to construct the ileal conduit dependably eliminates parastomal herniation. Moreover, herniation may be repetitive, resulting in multiple procedures to correct the problem. Unlike simple junction side-entry jackets as described in copending application Ser. No. 14/121,365 or 15/998,002, which are cylindrical, to allow sufficient radius for the diversion chute and mechanism used to deploy—and in an intermittent embodiment, to retract—the chute, a blood or urine diversion side-entry jacket is elongated on the side of the chute. Seen from above, it can therefore be described as oblong or ‘coccobacillar,’ that is, substantially rectangular with rounded corners, the hole for the ductus on the unprojected side off-center in focus.

Diversion jackets are therefore placed to encircle the vessel or ureter at the rotational angle that least if at all encroaches upon neighboring tissue. However, due to the small size of the jackets used for ureteral takeoff, at about 1.0 centimeter in length and 0.5 centimeter in height, encroachment should seldom if ever pose a problem. These dimensions can be reduced if necessary, such as for application in a neonate or infant, but are preferable to the minimum to expedite manipulation during placement.

Diversion jackets differ from side-entry jackets generally in that flow through the mainline is excurrent for drainage rather than incurrent, usually to target a drug into the encircled ductus. If used to divert arterial flow, the excurrent flow is continuous with peaks in pulsatile pressure, necessitating a water jacket to prevent extravasation during placement. However, if used to divert urine, lengthy interruptions in outward flow allow the unconflicted incurrent delivery of medication between outflows, without the need for a water jacket. With either the continuous outflow of a vessel or the intermittent outflow of a ureter, to prevent a drug or other agent intended for the portion of the ductus beneath (distal, inferior, or caudal to) the jacket from being flushed out through the mainline, the accessory or service channel or channels must continue entirely into the native lumen.

This is accomplished by continuing the accessory channel as a groove with open bottom running along the underside of the diversion chute. Both fixed and intermittent use embodiments do, however, incorporate at least the one accessory channel running along the underside of the diversion chute to allow medication to be introduced into any portion of the lower tract remaining that may require treatment or be potentially recoverable. Drugs or agents to target a portion of the ductus superior (cephalad, craniad) to the diversion jacket which are not to be administered systemically are injected into the surface port for entry into the ductus through another jacket positioned in the higher location. In an intermittent use embodiment, retraction of the diversion chute allows additional accessory channels to release drugs or other agents into the native lumen.

Thus, except in an embodiment with diversion chute fixed in position for use as a prosthesis when the lower urinary tract or a significant portion thereof is missing, an exceptional accessory channel needed in addition to that connected to and feeding through the diversion chute is a simple tube or pipe bonded to the internal surface of the mainline and extended into the native lumen to empty in the direction of flow. An exception to the generality that a prosthesis is always nonadjustable pertains when there will be an ongoing need for differential urinalysis of the urine produced by either kidney, and the urinalysis must apply to samples taken over more time than the wearer must remain in the clinic. In that case, an adjustable or intermittent embodiment must be used; however, taking samples by passing a catheter through an accessory channel and into the trepan end-piece allows differential spot checks to be made with a nonadjustable embodiment.

The distal ends of such an accessory channel are tucked inside the trepan section during incision and pushed out into the lumen before attaching the mainline to which the trepan section is connected and closing. The need for one or more additional accessory channels pertains only to an intermittent use embodiment, often for treatment of the bladder, and only in the unusual circumstance that drugs for treating the tract below the jacket to be administered locally, or topically, must be kept separate. An intermittent use embodiment placed to bypass an intractably overactive bladder, for example, poses such a potential (see, for example, Srikrishna, S., Robinson, D., and Cardozo, L. 2014. “Important Drug-drug Interactions for Treatments that Target Overactive Bladder Syndrome,” International Urogynecology Journal 25(6):715-720).

For this reason, with arterial diversion, a drug or drugs can be delivered without regard to the phase in pressure variation of the excurrent flow. A urinary diversion system is distinct in requiring that to treat the proximal ureter, it be possible to deliver drugs through the mainline, while to treat the distal ureter, it be possible to deliver drugs through an accessory channel, or sideline. Because the accessory channel continues into the native lumen in the direction of flow, drugs can be delivered regardless of whether micturition is in progress. However, drugs not to be mixed and diluted with urine, even sterile, are meted out during nonflow intervals.

Where the urine is contaminated and the drug should be administered as sterile, both main and sidelines allow lavage, an initial wash usable to furnish a diagnostic test sample. The timed administration of drugs or system maintenance agents can be accomplished manually by a competent patient or automatically by a system that includes the required sensor and microprocessor or timing control module. When urinary diversion is the only object, no timing module or microprocessor is needed, forced ejection of urine from the neoureter convergence, or confluence, chamber—which is required in all but an embodiment meant for use exclusively during the daytime, is executed by a pressure actuated switch in the chamber which is attached to and activates the neoureter convergence chamber impeller.

In unilateral application where only one kidney is affected and it is desired to independently analyze the effluent on that side so that a transureterouretostomy, for example, is not wanted, or in bilaterally separated application where each ureter is to empty through its own surface port into its respective collection bag, a confluence chamber is omitted, each side provided with its own independent channel whereby an evacuation assist impeller is bilateral without confluence, with a button cell battery in each surface port for power. In adjustable embodiments, the incorporation of a third push/pull cable control knob in the mons pubis port with cutaneous openings for the accessory channels allows control of a synthetic neobladder with stopper-flap for continence, thus allowing voiding in a reasonably normal manner.

A strain gauge and sensor and miniature rotor with eccentric load as mentioned above signal the need to void. Unless the need to empty the neobladder is frequent, the increase in storage capacity of the port battery needed to power both the neoureter convergence chamber elimination assist impeller and the motor used to signal that the neobladder is full is negligible. A second port to allow separate power for each function has diagnostic advantage in confirming that the source of inoperability of either is not due to low power. If the frequency is high, a larger battery is used. If causes discomfort, a second battery can be positioned in a second surface port positioned elsewhere, to include the contralateral side. When a future need for such a neobladder is anticipated, a surface port with button cell battery compartment is placed at the outset.

For use in an arterial shunt, however, the diversion chute shown here in FIGS. 5 and 10A, for example, would be positioned within the water jacket rather than directly on the internal surface of the mainline. If the plug catches, a fine guidewire with hooked tip is used to remove it from the line. With a ureter, the plug will usually be flushed out through the line spontaneously by the progressive buildup in pressure. For the plug to resist removal to the extent that it induces hydroureteronephrosis (ureterohydronephrosis) or hydronephrosis is unlikely but remediable by retrieval with a fine gauge hook tipped guidewire passed through the urine diversion effluent or outlet line, or mainline.

When no opening in the port need be open to the outside, the openings are all subdermal, that is, covered over by dermis. Such a port is typically placed in the pectoral region for injection of drugs or line maintenance agents by fine hypodermic needle or jet injection. A port which provides a urine diversion outlet will typically be positioned as is the stoma of an ileal conduit, at the surface of the skin in the lower abdomen. When only one of the openings into the port must remain open to the outside, as is the case with a urine diversion outlet or an opening to pass a cabled device on a frequent basis, the opening is mounted on the outside of the skin, other openings not requiring to be surface mounted then kept beneath the skin.

Openings into the port which must serve for fine hypodermic needle or jet injection so frequently that the skin would be injured and not allowed to heal are kept above the skin; however, if multiple openings are led into the same drug delivery line thus minimizing the trauma to the skin, then these openings are positioned subdermally. While open access through the dermis requires a permanent opening through copending application Ser. Nos. 14/121,365 and 15/998,002 describes a multiple opening port for mounting on the skin. The port, shown in FIGS. 27 and 28, is described in the specification. Tissue adhesion using synthetic tubing is approximated when the outer surface of the tubing is configured to encourage tissue ingrowth.

By contrast, the presupposition that a stoma provides serosa to fascia tissue adhesion or fusion between the ileum and the surrounding hypodermal fascia to form a permanent bond to seal out the environment is mistaken: given enough time, the faulty junction will eventually give rise to a parastomal hernia, which can cause discomfort and disfigurement necessitating use of a custom girdle or abdominal support to further degrade the quality of life. More serious parastomal herniation can lead to incarceration, strangulation, and considerable pain, necessitating surgical revision, or repair. Other complications associated with ileal conduit stomas made of gut as opposed to synthetics are prolapse, retraction, and stenosis. By contrast, a properly designed surface port made of synthetic material to replace a stoma constructed of native tissue is substantially if not entirely ‘immune’ to such complications.

Moreover, even when attention is paid to maintaining the stoma so that peristomal complications such as skin irritation and lesioning, or skin breakdown, to include (Marshall, C., Woodmansey, S., and Lyon, C. C. 2017. “Peristomal Psoriasis,” Clinical and Experimental Dermatology February 13 [in press]; Szymanski, K. M., St-Cyr, D., Alam, T., anf Kassouf, W. 2010, Op cit.: “ . . . irritant contact dermatitis, pseudoverrucous lesions, alkaline encrustations), mechanical injury (pressure ulcers, skin stripping injuries, mucocutaneous separation), infection (candidiasis, folliculitis), immunologic disorders (allergic contact dermatitis), and disease-related lesions (varices, pyoderma gangrenosum, malignancy . . . ”) do not occur (see, for example, Boyles, A. and Hunt, S. 2016. “Care and Management of a Stoma: Maintaining Peristomal Skin Health,” British Journal of Nursing 25(17):S14-S21), A stoma, whether the terminus of an ileal conduit, an ileostomy, or a colostomy, is subject to several serious complications, both early (before 30 days) and late (after 30 days).

Proportional in size to the ductus encircled, a ductus side-entry jacket for placement along a ureter, typically 1.0 centimeter in its longer outer diameter, is likewise millimetric in dimensions. The need for surgical reentry and replacement depends upon the relative diameter or caliber of the ductus upon placement and the later date; a jacket placed in an infant eventually will require replacement. However, because the accessory or service channel allows almost any other cause of fouling to be eradicated, replacement should not be necessary for years. An accessory channel leading into the lumen of the jacket afford access for maintenance of the jacket and lower urinary tract, while one leading into the foam lining the jacket allows the direct delivery to the foam ductus interface should an adverse tissue reaction beyond the normal foreign body response materialize.

The incorporation of a luminal contents tap or takeoff jacket diversion chute (takeoff chute, diversion chute) into a ureteral side-entry jacket to allow urinary diversion represents but a minor adaptation of such a jacket, which generally is used to establish a continuum between a synthetic and a native lumen. More widely applied without a takeoff chute, a ductus side-entry jacket can be used to encircle any type ductus to allow direct communication with a port at the body surface. This allows, for example, the directly targeted infusion of a drug into a blood vessel. While for urinary diversion, the subdermally or subcutaneously positioned port incorporates a hole opened to the outside to allow the diversion of urine into an external collection bag, the need for an opening to the outside is exceptional and such a port for injecting or infusing medication through accessory channels will be fully subdermal, which is to say, fully internal, fully implanted.

When systemic drugs prove unsatisfactory, once the port and line have been sterilized by lavage with wash water containing an antimicrobial, drugs can be targeted into the tract lying above (cephalad, craniad, superior to) the level of the jacket by injection through the urine outlet opening in the surface port. When the tract lying above the diversion jacket requires intensive drug therapy, a second jacket is positioned higher up on the ureter. Retraction of the diversion chute in the lower diversion jacket allows drugs delivered through the upper jacket to wash down through the lower tract. When the diversion chute must remain advanced to close off the ureter and divert urine into the outlet line, a channel running along the center of the underside of the diversion chute serves as a passageway to allow the delivery of drugs directly into the tract beneath (caudad, inferior to) to the level of the diversion jacket.

The chute is made of soft, shape-compliant material such as a Tygon® (Saint-Gobain, La Défense, Courbevoie, France), a more stiffly formulated tissue filler, or similar material in a composite or laminated to achieve the mechanical properties essential for the chute to adopt the contour without injuring the ureter. In an intermittent or adjustable embodiment, this is essential first, because an overhanging rim (awning, upper eyelid, palpebrum, palpebrarum) along the upper edge of the accessory channel opening into the ureteral lumen is needed to prevent retrograde or back-flow of medication or other agents up the ureter when the wearer is decubitus (recumbent) with the chute retracted. By contrast, in a nonadjustable or fixed embodiment for use as a prosthesis, such an overhang is unnecessary, because the chute is fixed in the extended position. With the chute in a fixed embodiment and in an intermittent (adjustable) embodiment when deployed (extended, advanced), a good seal requires that the surrounding edge of the chute slightly protrude into the wall of the lumen.

The edge is therefore made to fold upwards without gouging the urothelium, the overhanging rim in the adjustable embodiment folding flat when placed in abutment against the wall and therefore not protruding more than the rest of the edge. Were a stiff material used to make the chute, with either a fixed or an adjustable embodiment while the chute is advanced, the surrounding edge of the chute and this overhang would protrude into, and inevitably incise, the ureteral wall. Were the chute not made of soft shape-compliant materials with thin upturned, feathered and rolled edge bias molded to fold upward rather than protrude into the urothelium, erosion and device failure would soon result. In a fixed embodiment for use as a prosthesis, the chute once deployed is fixed in extension beyond the leading edge of the trepan.

Avulsion of a ureter at both pelvic and cystic ends sometimes occurs as a rare complication of ureteroscopy or accidental trauma; however, even in an adult with a wall thickness of only 1.0 millimeter, —a structure which includes an outer longitudinal and an inner circular layer of smooth muscle to support peristalsis should be great enough in tensile strength that this would follow the use of excessive or misdirected force and/or the ureter includes a segment rendered malacotic by disease or injury following the passage of a stone. Once reattached with microsutures, the ureter can be fitted with a diversion jacket during the same procedure. At 2.0 millimeters in width and length, the chutes in both adjustable and nonadjustable embodiments sized for the average adult exceed the internal diameter of the native lumen even at its peak caliber under diuresis during peristaltic bolus expulsion.

In an adjustable embodiment, the complete obturation of the side-entry outlet hole is essential to prevent the entry of urine when the chute is closed, but complete obturation of the portion of the tract below the jacket is not essential, leakage simply passing into the lower tract which the wearer will expel during voiding. In a prosthesis, however, where the lower tract is missing or dysfunctional, diversion is constant, eliminating the need for a side-entry obturator but necessitating the prevention of any leakage past the chute over time that would collect in the pelvis, or if the free end of the ureter is closed off, then in the irregular space between the underside of the chute and the ureteral terminus to pose a potential threat of infection.

In both adjustable and nonadjustable embodiments, the thinned out or feathered edges of the chute, molded to ride up the lumen wall, and the compliance under compression of the urothelium, the ureteral lumen should be completely cut off from the lumen above and thus unable to leak urine past the chute. In an adjustable embodiment, the compliance of the side-entry hole obturator needed to prevent the entry of urine into the diversion outlet line when the diversion chute is retracted to cause all flow through the distal tract is similarly compliant. To prevent the entry of urine into the diversion outlet lines when the diversion chutes are retracted to allow flow through the distal tract, adjustable embodiments require side-entry hole obturators. The obturators, unnecessary in a nonadjustable embodiment which diverts all flow of urine out through the side-entry holes and never to the distal tract, are likewise highly compliant.

So that this compliance does not allow any portion of the obturators, which must remain flush against and cover over the side-entry holes, to be pushed into the entry-holes creating a breach and partial outflow, the obturators must exceed the side-entry holes in area. The obturators must pass through the side-entry holes only when placed, and if applicable, when removed. To allow the obturators to pass through the side-entry holes and not catch on the trepan or injure the urothelium bounding the trepan if removed, the obturators must be dimensionally changeable. This is accomplished by making the obturators of a shape-memory polymer. The obturators are circular and fastened at their centers to the distal tips of the diversion chutes.

On placement, the prechilled obturators have the overlapping pleated form of half open umbrellas. At body temperature, the obturators are circular and pliant as to lay flush against without injuring the urothelium both when advanced and retracted. So that the obturators will not be damaged when flush against the urothelium with the chutes retracted, the trepans are slightly recessed. The diversion chute is retracted to allow passage of the side-entry plug when vacuum-extracted during placement. Once within the lumen, the operator deploys the side-entry hole obturator and advances the chute until it presses against the opposite wall of the ureter in rounded and flush relation thereto. Upon retraction, the obturator is brought flush against the near wall of the ureter with the trepan receded by 0.25 millimeters relative to the internal edge of the side-entry hole.

This leaves the near wall of the ureteral lumen surrounding the side-entry hole under negligible compression when the obturator is retracted to allow voiding without diversion. Since the urothelium is 1. Adapted to compression during and to the degree posed by peristalsis (which is less than sphincteric) when passing a bolus of urine, 2. The degree of compression is less than it is during peristalsis, 3. The duration of obturation is limited to time performing or sleeping, and 4. The lining of the side-entry jacket allows the unimpeded flow of oxygenated blood through the fine vessels of the adventitia without compressing the fine nerves thereof, the urothelium should remain unaffected. In a prosthesis, to preserve what peristaltic function remains, the jacket is placed as distally or caudally as possible along the ureter. If the distal tract remains, the diversion chute continues the accessory channel to its tip, allowing the direct delivery to the lower tract of medication. Rather than to make an additional embodiment merely to omit continuation of the accessory channel through the chute, the channel is simply not used.

In a prosthesis, that is a nonadjustable permanent embodiment, with the jackets situated near to the distal termini of the ureters, no passage and collection of urine past the diversion chute to leak into the pelvic cavity, while unlikely, can be tolerated. This is prevented by transecting the ureter about an eighth of an inch below the chute, injecting the irregular spaces beneath the diversion chute with a permanent injectable tissue filler, pinching the open end of the ureter closed with surgical cyanoacrylate cement, and suturing the ureter closed. The distinct improbability of leakage and little if any consequence were it to occur mean that a temporary nonadjustable embodiment placed to divert urine away from and deliver medication to expedite healing of the distal tract following surgery, for example, requires no ureteral stump end-plugs.

A tissue filler used to make the chute is not jellylike as are those used in plastic surgery but just firm enough to deform as required to maintain obturation of the ureteral lumen. In addition to the shape conformity needed, the material requirements are stringent, in that the material must be invulnerable to hydrolysis and enzymatic or microbial breakdown while provoking no more than a mild and transient adverse tissue reaction In conventional use, hydrogel-based tissue fillers can cause several serious complications. However, here the chute is made of less fluid material and is so positioned that these complications cannot arise.

Hyaluronic acid and fillers containing nonsynthetic components, for example, are more likely to arouse an immune or foreign body reaction and are not recommended. Fillers are used for cosmetic facial surgery where the filler can spread away, or migrate, from the injection site. Since for the present purpose the hardness-adjusted filler is constrained in position within the ureteral lumen, this detraction pertaining to fillers does not apply. Depending upon the medical context, medical-grade liquid injectable silicone and polymethyl methacrylate would appear to pose few if any problems for the use stated (Joseph, J. H. 2015. “The Case for Synthetic Injectables,” Facial Plastic Surgery Clinics of North America 23(4):433-445). Additionally, in an adjustable embodiment, complete obturation would prevent the direct delivery of drugs to treat the tract inferior to the jacket.

The material of the chute must not be susceptible to biodegradation, or breakdown due to enzymatic action, acidity, or hydrolysis by almost constant exposure to urine, or by bacteria in the urine or in the blood supply to the ureter in disease. Depolymerization of certain polymers yields hazardous monomers. Subject to breakdown by certain pathogenic bacteria such as Pseudomonas putida, once formulated to obtain the required hardness, cross-linked polyacrylamide hydrogel (the soft tissue filler is sold as Aquamid®/and Bulkamid®, Contura International, Montreux, Switzerland and Soborg, Denmark), for example, is unacceptable where infection from this and similar pathogens exists.

Wetting the urothelium distal to the jacket would then necessitate continuation of the tubular portion of the accessory channel into the distal lumen. To avoid this, the accessory channel is continued into an open-bottom groove that runs centrally along the bottom of the diversion chute to its distal tip. Second, in any embodiment, were the sides of the chute less than fully shape compliant with the changing diameter of the ureter as the chute is advanced, retracted, or left in the closed position, and the diameter changing due to the longitudinal peristalsis characteristic of the ureters. the press of the chute edges against the lumen wall would likely result in abrasion and incisions.

The chute, with slightly turned up edges to completely obturate the ureteral lumen and prevent leaks, is slightly larger than the largest internal diameter of the ureter during peristalsis, thus closing off completely the leakage of urine to either of its sides. Authoritative information on the range of normal dimensions of the adult human ureter is sparse. The largest mean resting external diameter of the living normal adult human ureter on unenhanced computed tomography is 2.7 to 3.0 millimeters. Documentation in the literature on the width of the normal adult ureteral wall is likewise scarce, but may be taken as having a mean thickness of 0.9 to 1.0 millimeters.

For the present purpose, deviation from this value due to gender or body size is not significant, the soft rubbery or spongy highly elastic character of the diversion chute and compliance of the ureteral wall making a chute width of 2.0 millimeters applicable in 2 sigma, or 95 percent, of normal adult ureteral lumina when passing a urine bolus. For the present purpose, the diameter of the normal adult ureteral wall when quiescent may be taken as 0.9 to 1.0 millimeters. If significantly deviant from these values, meaning that the elasticity of the chute will not adjust during peristalsis to completely seal off the ureteral lumen, the trepan end-piece and diversion chute are custom made. For a small child, these values for stocked ureteral takeoff jackets are generally halved.

The compliant elasticity and bending bias molded into the diversion chute assure that incisions, gouges, and breaches for urine to continue past the chute will not occur. To eliminate the need for precise adjustment midprocedurally and dynamically adapt to individual variance in the internal diameter of the ureter during the passage of urine boli, the chute, as indicated, is made of a soft nonallergenic rubbery or spongy polymer such as hardness-adjusted expanded polytetrafluoroethylene, or prepared from a four percent polyalkylimide suspension in water trademarked Bioalchamid 2.0 millimeters wide with upwardly molded soft rounded edges. Injected while fluid in larger volume just beneath the integument as in facial surgery, rather than in the preformed semisolid state pertinent to the present context, fill material is more susceptible to contamination and instances of migration following injection have no more relevance to use within a ureter than does the leukocytopenia reported following the injection of polyacrylamide.

When pressed against the transitional cell epithelium lining, or urothelium, the edges of the chute continue to bend upwards rather than incise or gouge the urothelial lining. The flexibility of the chute allows the leading edge to extend 2.0 millimeters forward of the trepan to fully obturate almost any normal human adult ureter. In both adjustable and nonadjustable embodiments, compression, incisions, and gouges of the urothelium by the outer edges of the chute are avoided through the use of a soft and elastic suitably molded chute that dynamically changes shape to comply with the instantaneous internal diameter of the ureter.

In an adjustable embodiment, it is necessary to prevent the complete covering over (overlayment, masking, blanketing) and compression of the urothelium by the outlet obturator. Compression is avoided due to the elasticity of the chute to which the outlet obturator is fastened. The complete covering over of the urothelium by the outlet obturator is accomplished by liberally perforating the obturator with 0.13-millimeter holes, which are too small to allow urine boli to pass through and enter the outlet when the obturator is retracted to allow urine to continue to the lower tract. Any seepage in this regard would simply pass to the collection bag. Minimizing compression not only protects the urothelium and suburothelium from injury but allows relatively normal neurophysiological function of the urinary tract.

Extended constriction of the urothelium and suburothelium, or the mucosa lining the urinary tract except during voiding can lead to physical injury that would facilitate infection, but no less significantly, can interfere with the function of these tissue layers, which more recent evidence has established do not comprise a ‘dumb’ conduit lining to fend off infection but are communicative, or ‘smart.’ While patients with a prosthesis will be missing part if not all of the lower tract, those wearing an adjustable embodiment will retain partially, temporarily impaired, or completely normal structure and function, which should be disturbed as little as possible.

Much as the vascular endothelium by its neuromuscular and secretory function exerts far-reaching as well as local effects upon circulation, the tissue lining the urinary tract releases adenosine, adenosine triphosphate, nitric oxide, and acetylcholine to modulate its underlying smooth muscle as an essential factor in the initiation of voiding, for example. This dynamic adaptive function is essential for normal adjustment to the ordinary internal and environmental as well as pathophysiological stresses that affect urinary function.

Furthermore, interference with urothelial neural and/or chemical signaling function can be the cause of existing or can induce new urinary dysfunction. Normal urinary function demands intact bidirectional communication between central and peripheral systems, to include the urothelium lining the urethra and the tissue of the urinary sphincters. Outlet (internal urethral orifice, uvula vesicae, and neck) obstruction having been addressed in copending application Ser. No. 14/998,495, this application pertains primarily to urinary incontinence.

Requiring at least one hole to be open to the outside for urine to drain, the surface port used for urinary diversion differs from surface ports used only to inject drugs in that the drain hole opens to the outside and is not covered over by the skin. Barring the presence of an interposed drainage reservoir or neobladder, the urine outlet hole leads directly into the jacket mainline. In contrast, when only drug openings are present, the surface port is fully implanted subdermally or subcutaneously, that is, just inside the integument, with drug delivery into the sidelines or accessory channels by jet injection. When drug administration is automated to effect release at programed intervals, the drug openings lead into reservoirs interposed between the entry openings and the lines leading to the target site respective of each hole-reservoir-line channel.

When not gravity fed upon the opening of a valve, withdrawal of each dose is by a pump at the outlet to each reservoir, the pump actuated by a timing controller or microcontroller, these drug delivery components all small and fully implanted. When the need to be able to pass a cabled device or devices through one or more drug delivery openings in the surface port on a frequent basis is established before the port is placed, the junction of the sideline (accessory channel, service channel) respective a frequent passage through a hole in the surface port other than the drain is angled to facilitate passing of the device into the mainline and native ductus. An exceptional need to pass a cabled device through a sideline is accomplished by slitting the skin overlying the opening and thereafter sealing the slit with surgical cyanoacrylate cement. The direct targeting of drugs in this manner allows antimicrobials, for example, to be delivered in greater concentration to eradicate a bladder, or lower urinary tract infection, and an anesthetic to alleviate the discomfort.

Another difference is that urinary diversion is an excurrent or outflow function, without an incurrent drug delivery metering or dose dispensing size and timing regimen. Instead, operation is continuous, urine passively diverted as it is produced. In a drug infusion application, the implant set will usually include one or more small flat drug storage reservoirs typically positioned subdermally in the pectoral region. An excurrent function lacking, the port will not include an opening to the outside, instead being overlain with skin, hence, fully implanted, through which reservoir replenishment is by jet injection. The timed delivery of one or more drugs is controlled by an implant timing module or microcontroller which actuates small pumps connected to the reservoirs. If appropriate, the timing of drug or maintenance agent delivery can be triggered by the pressure actuated switch in the neoureter convergence chamber.

Much leeway in internal diameter is afforded by the foam lining the jacket, which can be made thicker to allow for growth or swelling. Only in more extreme cases, as an exstrophic neonate, should it eventually become necessary to explant and replace the jacket. Takeoff at the ureters precedes the bladder, and therewith, bladder dysfunction or disease regardless of type, and preempts all sense of urgency, instead diverting urine passively and unconsciously to a paracorporeal collection bag. The takeoff jacket to either side can be positioned at any level along its respective ureter, and each jacket communicates by means of an accessory channel (service channel, sideline) with an entry portal at the body surface through which the urine diversion line exits and the accessory channels enter.

Thus, implant-maintaining substances can be targeted directly to the jackets and synthetic lines and drugs to any preserved length of the ureter. Takeoff at any level along either ureter keeps urine from contact with, while allowing the direct delivery of medication to, the lower tract. This isolation of the lower tract facilitates healing following surgery of the lower tract and/or the application of medication to treat a disease condition where systemic medication would otherwise have to be given at higher doses with nontargeted tissue exposed and washing over by urine of topically applied medication would flush away and dilute medication to impede recovery and necessitate more frequent dosing at increased expense. This is true when the urine is nonpathogenic in health, much less when it is infectious in disease.

Accessory or service channels of nonjacketing side-entry connectors inserted into the parenchyma of a solid organ tumor can be radiation shielded to allow treatment with lower dose rate radionuclides (radioisotopes). In contrast to a blood vessel, which is continuous in flow-through so that the lumen remains patent, the lumen of a ureter remains substantially unchanged in outer diameter while the tissues lining the lumen compress during urine outflow to increase the luminal cross-sectional area some 27-fold (Woodburne, R. T and Lapides, J. 1972. “The Ureteral Lumen during Peristalsis,” American Journal of Anatomy 133(3):255-258). When the surgical procedure is unilateral, only the one side need be addressed thus.

While in this case installation is temporary, the apparatus is best removed following healing; however, removal, especially if accomplished robotically, is by entry through a small ‘keyhole,’ incision. Unlike the clearly patent lumen of a blood vessel, to prevent the trepan from cutting into the opposing wall, the tiny lumen of a ureter requires to be protected by the temporary insertion of a stent or lightly insufflated. Direct connection of the ureters to the skin without involving the gut, or ureterocutaneostomy, was soon realized to result in complications, primarily ureteral stenosis.

Now it is practiced only as an exigent measure when the gut is unusable or the patient has little lifetime remaining (see, for example, Stein, R., Hohenfellner, M., Pahernik, S., Roth, S., Thüroff, J. W., and Rübben, H. 2012. “Urinary Diversion—Approaches and Consequences,” Deutsches Ärzteblatt [German Medical Journal] International 109(38):617-622). The construction of an ileal conduit, the first means of urinary diversion realized using various techniques developed in the 1940s by Eugene M. Bricker and eponymously designated a Bricker conduit, begins with the harvesting of a 15 centimeter segment of ileum at a distance of 15 centimeters from the ileocecal valve.

An Indiana pouch, developed by a team at the Indiana University Medical Center, provides a continent internal reservoir or neobladder, as do several alternative procedures. Construction begins with the harvesting of a length of ileum plus 2 feet of the ascending colon. Usually successful in experienced hands, these initial procedures in creating a ureteroenteric anastomosis nevertheless impair the gut and risk complications even before the ileal segment taken is tubularized, or tabulated, and resituated.

In what is essentially a second procedure, the ileal and colonic tissue is diverted to a different function for which its lining is not adapted, where previously irradiated tissue can undergo postprocedural degeneration and the development of abscesses, to which synthetics are oblivious, and postprocedural stenting and its complications are averted the resorptive surface of the bowel segment used can no longer play its original physiological role in the gastrointestinal tract, even though its absorptive and secretory functions are still intact . . . . This has metabolic consequences, because the diverted urine here comes into contact with a large area of bowel epithelium. Early preventive treatment must be provided against potentially serious complications such as metabolic acidosis and loss of bone density.

The resection of ileal segments can also lead to malabsorption. The risk of secondary malignancy is elevated after either continence-preserving anal urinary diversion (>2%) or bladder augmentation (>1%)”; Lazica, D. A., Ubrig, B., Brandt, A. S., von Rundstedt, F. C., and Roth, S. 2012. “Ureteral Substitution with Reconfigured Colon: Long-term Follow-up,” Journal of Urology 187(2):542-548. The same applies to any procedure that channels urine through gut, whether an ileal conduit, continent ureteroileocystoplasty or a neobladder constructed from gut, the former posing the risks associated with a stoma, the latter two more susceptible to midprocedural complications, and all subject to the degradation of digestive tissue with constant exposure to urine.

Another problem with stomata constructed of autologous gut is that digestive enzymes and acids which come into contact with the skin cause much irritation. Following the insertion into the urinary tract and transition of gut to malignancy apparently takes about 15 years, making such use unsound in patients less than age 60 and the correct application of synthetics the only alternative that will avoid major complications for the foreseeable future.

Pending the successful use of engineered tissue urothelium lined conduits, the use of synthetic materials, especially in the young, will remain the best option for averting the inception of malignancy; however, to generate tissue from stem cells taken from the patient may require more time than the condition of the patient may allow, and the technology remains far from practicable, making the use of synthetics advantageous for now. Conventional procedures which pass urine through urothelium, such as a ureterocutaneostomy are to be preferred, although the stoma will still pose complications.

The intestinal mucosa is damaged by constant exposure to urine, making the transplant prone to metaplastic transition, which—with greater likelihood the younger is the patient—can ultimately progress, apparently de novo, first to primary premalignancy in the form of benign adenoma and thence to frank malignancy in the form of adenocarcinoma. In otherwise substantially healthy young children, this metaplastic transition appears to take longer than 15 years, making an initial reassignment of gut a viable means for bridging the gap until the incongruous tissue can be removed, preferably, to be replaced with synthetic materials.

Furthermore, it has been known for almost four decades that apart from de novo, or primary, development of carcinoma in the conduit due to histological and physiological incompatibility with urine, a radical cystectomy with the creation of an ileal conduit responsive to preexisting bladder malignancy can eventuate in secondary malignancy of the conduit as well as the upper urinary tract. Such consequences are possible whether the diversion fabricated from gut is to a pouch according to Bricker, Wallace, Wallace II, Mainz II, Koch, Florida, Miami, T-pouch, Bricker end-to-side, Le Duc, of Indiana type, or any of several variants thereof.

While urinary rather than fecal diversion is addressed, numerous complications such as leaks, infection, peristomal irritation and dermatitis, retraction, prolapse, stenosis, hernia, necrosis, and iliocutaneous fistula, pertain to both urinary and fecal stoma. In a cancer patient, stoma resolution or the need for corrective measures can delay chemotherapy (Sherman, K. L. and Wexner, S. D. 2017. “Considerations in Stoma Reversal,” Clinics in Colon and Rectal Surgery 30(3):172-177).

Even though the risks of malignancy and stoma complications rise with the length of time the tissue taken from the gut remains in use, many such procedures are performed on young patients. Moreover, unlike the use of prosthetic means, surgical diversion is far more likely to necessitate a second, reconstruction, procedure to correct the original operation, and even when temporary, reversal demands a second surgical procedure which risks more complications to reinstate normal structure and function than does the use of synthetic parts.

Irreversible without risking numerous complications, an ileal conduit should not be considered for the distinct majority of patients, who present with a number of different bladder disorders which may be temporary, better treated were the bladder bypassed, or respond, if incompletely or slowly, to drug or electrostimulatory therapy, as to leave a consequential surgical procedure without justification. The advent of nonproteinaceous, nonallergenic tubing was immediately recognized as offering the benefit of not having to harvest autologous tissue with its attendant risks.

It soon emerged, however, that placed in the urinary tract, the accretion of crystal on the inner wall posed a risk, that placed in the arterial tree, there would quickly become an accumulation of thrombus along the internal surface, and that situated anywhere in the body, infectious biofilm can accumulate as a potential threat to patency as well as health. Occlusion and contamination ended the practice of using small caliber tubing as a substitute for the harvesting of a native ductus, such as the great saphenous vein and later, the internal thoracic artery, which also resulted in complications at the harvest sites. Today synthetic tubing is used only to bypass the largest vessels as most elusive of reconstruction from autologous tissue and least susceptible to occlusion.

Synthetic materials are not susceptible to complications such as stricture, infection, metaplastic transition, shear stress, or stomal deterioration, and the incorporation of accessory or service channels into the ureteral takeoff jackets allows biofilm, coagulation, and accretion-preventive agents to be drip released into the lines as necessary. Moreover, adverse tissue reactions or other complications associated with the implantation of synthetics are readily overcome. Unlike a conduit and stoma constructed from native tissue, the parts in a synthetic assembly fit together precisely, precluding leakage and are easily fitted with sensors as part of an automatic prosthetic disorder response system, which depending upon the type and severity of the disorder, can be ambulatory.

Moreover, in surmounting the central problems of urine leakage and incorporating an accessory or service channel to allow the direct targeting to the jacket and line of occlusion counteractants, a ductus side-entry jacket eliminates the incapacities of small caliber catheters that have prevented the use of these for permanent implantation. Dependably fixed in position by suture, anchoring round needles, and tissue ingrowth, the surface port for the excurrent passage of urine and the incurrent flow of drugs is unlikely to become dislodged or allow any of the complications that result from inadequate adhesion such as parastomal herniation or prolapse.

While less susceptible to clogging, the placement of a ductus side-entry jacket proximal to the superior end-to-end anastomosis of such a synthetic graft allows the direct targeting to the anastomotic junction and prosthesis of antimicrobial and/or anticoagulative medication as necessary. This is no less the case with synthetic endoluminal grafts such as those used to contain an abdominal aortic aneurysm. Antimicrobial, anti-inflammatory, anticoagulative, anesthetic, stone formation preventive, and other supportive drugs to maintain patency and cleanliness are delivered directly to the jackets, lines and urinary tract distal to the level along its respective ureter of each jacket.

Another fundamental advantage in the use of synthetics is clear superiority for the fitting of sensing and actuation electronics, a central consideration where automation must be enlisted to treat comorbid conditions such as cardiorenal syndrome in a patient incapable of self-care. Diversion to isolate a kidney transplant from the possibility of reflux is addressed unilaterally with an ipsilateral jacket having a diversion chute, mainline, or urine diversion channel, and accessory channels or sidelines to give direct access for the delivery of drugs or the passage of a cabled device, such as an endoscope or laser. Itself a type of nonjacketing side-entry connector, the surface port is not susceptible to degradation as is a stoma, and proper hygiene makes irritation or infection of the surrounding tissue unlikely.

The caliber of the lines determined before placement, a fine fiberscope can be passed through any of the openings in the surface port to view, manipulate, swab, or focus a laser at any level along the line. To pass the scope or other cable like device past the diversion chute, an intermittent embodiment that allows the chute to be retracted must be used. When use is to be intermittent or elective, such as only during the night, or when diversion is to be suspended to allow to allow medication to pass through a post diversion segment of the ureter or lower urinary tract to be treated, retraction and advancement of the chute is with a push/pull control cable. Control thus averts the need for complex and costly electronics.

In FIG. 28, accessory channel or drugline catheters and the push/pull control cables can be run either as separate lines normally bonded together or are run in adjacent relation through double lumen catheters made of a polymer such as polytetrafluoroethylene. The lumen adjacent to the accessory channel contains the inner control wire of the push/pull cable. The cable should be made with sufficient precision that a barrel adjuster at the port end for accessibility should be unnecessary, especially because adjustment if needed would be too small to achieve by hand. Similarly, intentionally partial retraction or deployment of either or both chutes to reduce the rate of collection bag filling where facilities are available requires precise adjustment. To accomplish this, the knob ends, that is, the distal or port-ward end segments of the control cable inner wires are threaded over the length of 2.0 millimeters.

The inner wires are controlled by manually rotating knobs on the surface port which draw in the screws to retract, and move out the screws to advance, the diversion chutes. The thread is cut to a pitch so that one half rotation of either knob moves the inner wire the full 2.0 millimeters. To provide a tactile cue for the half way point of chute deployment, the knobs are provided with detents. The precision and durability required of the internal or female thread in the knobs and external or male threads on the control wires is achieved by making the knobs and control wire of titanium alloy Ti-6Al-7Nb. When use is to be continuous and permanent, the typically 1.5 millimeter wide by 2.0 millimeter long curved and slightly scooped diversion chutes are advanced once upon placement to fix these in position, no push/pull control cables needed for subsequent retraction and advancement.

In a nonadjustable or prosthetic embodiment, the chute is permanently deployed and voiding to an external collection bag is passive or automatic when the pressure actuated switch in the neoureter convergence chamber energizes the impeller. Where the wearer of an adjustable embodiment must be mentally competent, the wearer of the prosthesis need not. As indicated, in situations where ureteral disease recommends interim diversion proximal to the affected segment of the ureter or any lower portion of the urinary tract to allow treatment, medication can be directly targeted through the accessory channel or sideline and jacket to the affected segment.

When a drug or drugs would best be limited to only an affected segment, provided there is a reversal or neutralizing agent for the drug or drugs used, the extent of exposure to these agents can be restricted. If the distal terminus of the segment to be treated is tubular, this is accomplished by placing a second ductus side-entry jacket at the appropriate level to receive the reversal agent through a separate accessory channel. If the terminus is relatively planar or curved, the segment is ended for exposure to a drug or drugs with a nonjacketing side-entry connector. Certain additional capabilities, essentially relevant only for patients unable to adhere to the regimen or self-administer drugs, such as the very young or old, can be further assisted by means of automation.

For example, when the drug insertion apertures in the surface port are covered by a membrane and connected to flat reservoirs positioned subdermally for replenishment by jet injection so that only the urine expulsion line is patent, a fully implanted microcontroller can oversee the delivery of each drug and counteractant as appropriate. Moreover, if malfunctioning, the interruption in timely drug delivery can be telemetrically indicated and radiocasted to the clinic, with responsive adjustment or reprograming accomplished remotely. The ureters are densely innervated and perfused from different sources according to level, so that to transect or sever these at any level without providing a pedicle should not induce atrophy.

At the same time, the accessory channel allows an antimicrobial, anti-inflammatory, antiangiogenic, anesthetic, and/or other drugs as necessary to be directly targeted to the jacket, mainline, and target tissue, thus minimizing if not eliminating the need to reenter other than to remove a temporary jacket or jackets. As shown in FIGS. 6, 11, 12a, 12b, and 12c, of copending application Ser. No. 14/14/998,495, a nonjacketing side-entry connector can position a fiberscope along the bladder dome to allow viewing the ureterovesicle, or ureterovesicular, junction, allowing flow into the bladder from the ureters, ureterovesical junction obstructive nephropathy, the presence of diverticula, or the condition of the bladder lining to be directly viewed, which both anatomically and functionally has considerable diagnostic value.

Even when diuresis induces the jet effect (see, for example, Chung-Chieng, Wu 2010. “Ureteric Jet,” Journal of Medical Ultrasound 18,141e146) the more distal or farther down the ureter the takeoff jacket is positioned, the more is intrinsic expulsive pressure likely to be preserved, and the more intrinsic pressure is preserved, the greater will be the number of bodily positions the patient will be able to assume and still void without the need for an assist device in the form of a neoureter convergence chamber impeller.

Thus, to preserve the maximum peristaltic pressure, each jacket is best placed as distally along the ureter as the pathology will allow. Nevertheless, for the patient who restlessly tosses and turns, a pressure responsive assist device in the form of a neoureter convergence chamber impeller is provided. This allows the patient with frequent urination, regardless of cause, to sleep through the night whether supine, prone, on a side, or when changing positions. When automation is applied, the ejection pressure can also be intensified through electrostimulation to amplify the calyceal or pelvic peristalsis.

Free movement is also possible because the diversion line, while made in its intracorporeal length of a fluoropolymer for slipperiness, is connected to a length of highly flexible nonallergenic rubbery tubing such as Saint-Gobain Corporation Tygon® S-50-HL running from the surface port to the collection bag. Resistance to irreversible deformation kinking also allows the wearer to engage in sports without special concern for sustaining an impact at the outlet leading to the collection bag. Additional information pertaining to the application of vascular valves to the urinary tract is furnished in the section below entitled Description of the Preferred Embodiments of the Invention.

FIGS. 2, 7, and 8 show vascular valves for the diversion of flow, usually of urine from a ureter to a neobladder or collection bag. That in FIG. 2 is switched in position by the operator, and in a urinary prosthesis for which the valve is suitable, can be switched back by a clinician but not the patient. In the urinary tract, synchronization among the one or two valves required is not a factor, By contrast, synchronization is essential in a solid organ transplant such as that of the heart. The solenoid push-configured valve shown in FIG. 7 and solenoid pull-configured valve shorn in FIG. 8 are suitable for a sudden bypass, or switch organ transplant under exigent circumstances but not a metered bypass, or switch organ transplant as is shown in FIG. 10A, which progresses gradually under a program that immediately responds to signs of rejection.

Actuated simultaneously from the same electrical switch, the solenoid-driven vascular side-entry valves shown in FIGS. 7 and 8 are also suitable for exchanging flow between great vessels when amenable to an extracardiac correction of a total transposition. With a manually controlled mechanical valve, the operator grips the accessory channel stabilization collar 12 to slide the diversion chute and accessory channel forward into the native lumen until the detent 16/17 beneath the chute engage to lock the chute in the forward, or fully deployed position with an audible click. Inaccessible noninvasively, mechanical valves are best limited to applications very unlikely ever to require reversal. By contrast, solenoid-driven valves are reversible when energized with sufficient reverse surge current to overcome the detent, less current needed where the solenoid incorporates a return spring.

Even through mechanically driven valves and solenoid-driven valves without extracorporeal controls are less costly than servovalves, in any application where post-implantation adjustability, much less immediate response to commands from an implanted master controller are beneficial if not essential, the controllability of servovalves makes these preferable for the simplest transplant. This is true if only a few valves are needed, and precisely synchronous deployment of the diversion chutes is not critical. The side-entry opening stopper, or ostium obturator, 30 is permanently is permanently affixed to the diversion chute and no less present in the retracted, or undeployed, position where it is collapsed by constraint inside trepan tube 5 as to obscure the diversion chute.

The outlet of the lower accessory channel 8 is shown as part number 31 below the distal tip of the diversion chute. Whether channeled through the chute as accessory channel 8, relegated to upper accessory channel 8′, or to a separate basic side-entry jacket upstream, both the upper and lower surfaces of the chute are accessible to medication. Before advancement from the trepan tube into the substrate native lumen, the ostium obturator must be collapsed to be compact as possible, affording sufficient clearance to allow removal of the side-entry tissue plug through use of the suction pump alone, then upon entering the native lumen from which it is not normally withdrawn, self-deploy by unfolding. To spontaneously-unfold when advanced into the native lumen, ostium obturator is made of a shape-memory polymer.

The polymer is soft enough in the elastic state that should it become necessary to remove the diversion device in the absence of a chilling means that would cause it to compact, or fold, the obturator can simply be pulled through the side-entry hole and out into the trepan end-piece with little if any injury to the margin of the side-entry hole. That the trepan is slightly recessed from the luminal border of the side-entry hole reduces any damage to the obturator if any. Upon placement, the diversion jacket, hence, the obturator is at room temperature, or more likely in terms of the shape state changing temperature differential attained to date, pre-chilled, keeping it in the compact rigid state, and when brought to body temperature, unfolds, or deploys, becoming soft and pliant.

Accordingly, highly damped miniature nonsparking plunger solenoid-operated vascular valves such as shown in FIGS. 7 and 8 are useful for sudden bypass transplantation where there is a low expectation of rejection following the preprocedural administration of immunosuppressives and immune tolerance inducing drugs and cells, and where synchronous operation of a larger number of valves than can be manually switched at the same instant is essential. The solenoids shown in FIGS. 7 and 8 are schematic in omitting dampers, springs, and other elements without novel significance. When placed, the application of current drives or retracts the plunger (thrust rod, slider, shaft.

As shown here, when readied to be placed, the solenoids are energized to maintain them in the retracted position with the return spring compressed until switched to cut off the current so that the restorative force of the springs ejects the chutes which will then sustain the solenoids in the ejected, vessel-obturated, state without a further need for energy input. In sudden bypass, or switch, solid organ transplantation, adjuvant therapy is by pre- and postprocedural infusion or injection without the interposition of a third midprocedural stage between the two. A triple stage procedure, metered bypass, or switch transplantation allows the controlled progressive release of immune tolerance-inducing substances over the course of time that the diversion chute traverses the distance to complete occlusion of the native lumina and incorporation of the new organ into the recipient.

This transit time is immediately responsive to the detection of an immune response by sensors feeding data to the implanted controlling microprocessor, for example. FIG. 10A shows a linear servomotor-operated vascular valve made to the dimensions of the prospective substrate vessel. When other than the vessels departing and returning to the heart, these will be referred to as miniature or subminiature. Such a servovalves affords continuously variable control of chute deployment and retraction in distance, direction, and speed, any or all of which may be commanded adjusted by the implanted controller at any time during transplantation or postoperatively.

Unique to metered bypass transplantation, midprocedural control consists of moment by moment adjustment of the diversion chutes by the controlling microprocessor responding to physiological sensor feedback in accordance with its prescription-program. Each chute is gradually advanced, and an adverse reaction detected, the advancement is paused and reaction reversal agents released through one or more servovalve accessory channels. Thus, the chutes are controlled in coordination with the release into the lumen of immune tolerance-inducing drugs and the detectable effect these register on the sensor feedback to the controlling microprocessor.

The controller then resumes adjustment of each chute, which may call for continued advancement, partial retraction, holding in place, or even truncating the procedure, largely avoidable by proper matching of the donor and recipient and the administration of tolerance inducing agents preoperatively. For this reason, the automatic release of agents midprocedurally should not involve doses larger in volume than can be held within small flat reservoirs positioned in the pectoral region. Nevertheless, where the process of tolerance induction must be truncated, the servovalve is capable of sudden and complete retraction, returning the substrate lumen to normal flow. Such action will usually apply equally to all of the valves.

A proportional flow diversion servovalve feedback controlled thus is superior to pulmonary or portal banding, for example, for alleviating hypertension. Any linear motor incorporating or coupled to a positional feedback device in support of proportional-integral-derivative control which can be reduced to subminiature dimensions can be used. In metered bypass, or switch, transplantation, the microprocessor coordinates the positional with the physiological feedback to control the diversion chutes, hence the relative proportion of donor and recipient blood passing through the valves.

FIG. 28 provides an overall view of a nonadjustable embodiment suitable for use as a permanent prosthesis in a patient with an irreversible congenital or traumatic condition involving the lack or loss of the lower, or distal, urinary tract. Where corrective surgery is contemplated, must be deferred to a later date, or has been accomplished, the embodiment is also suitable for use on a temporary basis as a means of diversion away from the affected or operated region, whether unilateral or bilateral. The urine outlet opening 110 through the center of the surface port also serves incurrent use for directly targeting agents to maintain the components and allow the insertion of cabled devices for cleaning and examining the prosthesis.

Urinary diversion jackets such as shown in FIGS. 2, 28, 30, or a diverter such as that shown in FIG. 31, all of which omit the ostium obturator 30 required in any valve suitable for the diversion of blood but which obstruct passage, one can insert diagnostic and therapeutic cabled devices through a pectoral port to look downward through the system or through a pubic port to look upward. Thus, passing the cabled device up through effluent line 110 and passing the device upwards through the diversion jackets allows the clinician to obtain a direct view of the upper or proximal tract by extraurethral pyeloscopy, ureteropyeloscopy, or ureteroscopy extraurethrally, without a topical anesthetic, patient discomfort, or risk of injury.

To directly treat an infection, remove biofilm or crystal, or to obtain a biopsy test sample, the same extraurethral route gives access to every level of the urinary tract outside the kidneys. FIG. 28 showing a prosthesis and FIG. 30 showing an adjustable embodiment, the accessory channels (service channels, sidelines) omitted appear in the drawings of valves and in FIG. 31, also suitable for use in a prosthesis. Because gravity is working with, rather than against, the flow of fluid maintenance agents, such as a crystal solvent or an antiseptic, into the diversion jackets then down through the rest of the system, and unlike the pelvis, the pectoral region affords space to position drug reservoirs subcutaneously, the accessory channels are best accessed from above rather than below.

In non-prosthetic embodiments, push/pull Bowden cable control knobs for the wearer to adjust mechanical valves, or electrical control knobs to control solenoid-driven or servovalves are best positioned around the urine outflow, or effluent, pipe 110 at the center of the port, for ease of wearer viewing and use placed to a side of the mons pubis. The surface port in a urinary diversion apparatus requiring an external opening to pass urine in any event, the addition of small control knobs does not compel the externalization or paracorporealization of the surface port that could otherwise be positioned subdermally. Unless the ureter distal to the jacket is missing, the surface port additionally incorporates a cutaneous, or on the skin, opening into an accessory or service channel which provides a direct passageway into the distal tract for implant maintenance, diagnosis, and therapy.

As shown in FIG. 30, advancement and retraction of either diversion chute is controlled by separate control knobs 153 and 153′. Conventionally, the wearer rotates both knobs the full half-turn between the stop to either side, thus fully advancing or retracting the diversion chutes. The diversion chutes typically in the range of 2.0 to 4.0 millimeters in length, rotation of either control knob by one half-turn ejects or draws in the threaded end segment of the inner push/pull control cable wire to advance or retract its chute by the equivalent amount.

Separate control of either chute rather than of both with a single knob allows renal differential urinalysis by the clinician, and by rotation less than a half-turn, the wearer with convenient facilities to apportion the rate of drainage from either side into the collection bag, reducing the rate of bag filling, or into the lower tract for normal voiding. FIG. 5 provides a longitudinal section and FIG. 6 a cross-sectional view through a wearer-adjustable diversion jacket parallel to the view of a nonadjustable embodiment shown in FIGS. 2 and 3.

Miniature push/pull control cables allow the controlling motion to pass from small control knobs at the body surface port through a tortuous path to the remote jacket to make possible the simple rectilinear motion needed to advance and retract the diversion chute. As it would create problem spots for encrustation, microbial colonization, and thrombus, control cables 28 and 28′ are not run through mainline 9. In the lower left-hand corner of FIG. 5, the distal terminus of cable 28 shows where the push/pull cable used to advance and retract chute 18 is connected to the chute through collar 12 encircling and the accessory channel 8 at the bottom of each diversion jacket. The mechanism which allows a half turn of either know to fully advance or retract the chute to its side is described below in section 6. Description of the Preferred Embodiments of the Invention.

To afford accessory channel 8 clearance to drive diversion chute 18 along its slideway or raceway, FIGS. 5 and 6 show how diversion chute 18, inside trepan tube 5, along the bottom center of the jacket is provided with a slot 27 through the jacket shell 3 and foam 4. To prevent the leakage of urine outside the jacket, these parts, made of a hard and slippery polymer such as gear grade nylon, must be flush fitting.

FIG. 2, provides a detailed view of a side-entry flow diversion jacket which once set by the operator, remains fixed or nonadjustable, and as such, suitable for a prosthesis. As seen in FIG. 2, the detents in a set-once solenoid-driven diversion jacket for use in a urinary prosthesis consists of a small eminence, or protuberance, 16 along the underside of diversion chute 18 and a depression 17 molded in the surrounding jacket such that engagement of the eminence in the depression with an audible click immobilizes the chute at that extension into the native lumen with sufficient resistance to displacement that moderate force is required to dislodge the eminence from the depression. If necessary, diversion jackets in prostheses can be detent and/or spring overridden by a clinician. FIGS. 31A and 31B show a diverter with accessory channels which can also be used in a prosthesis; however, an endoluminal, or non-side entry, anastomosis coupling or flow diverter is structurally limited to diversion and cannot be used to apportion its outflow between two lumina.

While flow diverters such as shown in FIGS. 31A and 31B and the straight versions of these both accomplish diversion by redirecting flow, diversion jackets and valves differ from a basic side-entry jacket as described in copending application Ser. Nos. 14/121,365 and 15/998,002 in incorporating a chute to divert the flow of blood or urine. The significance of this is that unlike the diverter or its straight counterparts used as anastomosis couplings, which are fixed, or ‘dumb, a flow diversion jacket driven by a solenoid with a return spring with or without a detent allows bistable switching between total and no flow diversion, and one driven by a servovalve allows continuously variable control over the relative proportion of flow, leaving an undiverted proportion of flow to continue along its normal path.

Variable controllability is essential for automatic adjustment by a servomotor-driven valve as commanded by an implanted microcontroller or microprocessor in response to sensor feedback. As shown in FIG. 30, in an embodiment which allows the wearer to select between normal voiding and the passing of urine into a collection bag, adjustability is ordinarily by means of push/pull cables controlled by turning knobs on a surface port positioned on the mons pubis. In a bilateral embodiment, both knobs can be on the same port or each can be on separate ports to either side of the mons pubis. Solenoid-driven diversion jackets are readily producible but require throwaway or rechargeable button cells surrounding the urine outflow pipe 110 as are the injection opening in FIG. 28 and the control knobs in FIG. 30.

Both a diverter such as shown in FIGS. 31A and 31B and a diversion jacket enable the creation of fluid shunts and bypasses using even small caliber synthetic tubing without the need to harvest a native ductus such as a vein and in so doing, create a preliminary site for the development of complications with the potential to prove serious. Both incorporate accessory channels which allow the direct pipe-targeting of anticoagulant and crystal dissolving agents into their synthetic lumen and drugs into the substrate native lumen. Not requiring adjustability, both diverters and solenoid-drive diversion jackets are suitable for use in a prosthesis such as that shown in FIG. 28 to replace the lower urinary tract.

By contrast, in an embodiment for use on an intermittent or discretionary basis, diversion must be selectable, making a deployable and retractable diversion chute and thus a diversion jacket rather than a fixed diverter necessary. An accessory channel allows the direct targeting of antimicrobials, anticoagulants when blood and crystal encrustation counteractants when urine is diverted without which smaller catheters invariably become fouled and/or clogged. Synthetic shunts and bypasses have been used for decades, but this factor has prevented the use of catheters to like purpose over the same period.

In a urinary jacket or valve, the integral drug delivery accessory channel, seen as part number 8 in the drawing figures, continues toward the ureteral lumen through a groove running centrally along the underside and extending to the distal tip of the chute to emit through a small opening 31 along the underside of the chute 18, allowing medication to be dripped into the substrate vessel or ureter. FIG. 3 provides a detailed cross-sectional view of the diversion chute shown through trepan tube 5 along line A-A.′ in FIG. 2. In a nonadjustable or fixed prosthetic embodiment, the chute is never retracted as would allow urine to continue through a functioning lower urinary tract.

The design of the side-entry diversion jackets and valves shown allows the diversion chute to be advanced into and retracted out of the trepan piece without abrasive contact of the chute against a surrounding edge of unprotected tissue. The moisture barrier-protected foam enveloping the adventitia (outer surface) of the ureter or artery is continuous entirely about the trepan, assuring leak free operation. While an adjustable embodiment must continue to be controlled remotely after placement, a prosthesis need be set only at the time of placement when the operator has direct access to the treatment site. In a vascular embodiment, a side-entry hole stopper, or ostium obturator, at the front end of the chute is necessary to prevent the flow of blood back into the trepan tube.

Before the diversion jacket or valve is placed, the ostium obturator is collapsed with the chute retracted inside the trepan tube, as depicted in FIGS. 7 8, and 10A. When the chute is advanced into the native lumen, full extension of the chute for full diversion of flow is with the ostium obturator flush against the opposing wall of the native lumen. Retraction of the chute so that flow is not diverted places the ostium obturator flush against to fully cover over the side-entry opening as a stopper. Thus, once deployed, at no time does the ostium obturator reassume its preplacement position inside the trepan tube so that outflow would be obstructed when the obturator is positioned flush against the opposite or far side of the ductus. This is shown in FIG. 5, where a dashed line represents the ostium obturator as covering over the opening into the trepan tube with the chute retracted, and solid lines show the chute as fully deployed. Used to drain urine continuously, a urinary prosthesis requires no ostium obturator.

Upon placement, to allow sufficient cross-sectional clearance to allow the tissue plug to be removed, the ostium obturator is best sufficiently pliant so that the suction hose will be able to withdraw the plug past it without requiring a level of vacuum pressure as would drive the trepan against the opposing wall of the native lumen prevented by a limit switch on the pump. Should the plug catch on the obturator, the clamp shown in FIG. 4 will allow the suction hose to be disconnected and a miniature dental pick used to pull out the plug. Clamping thus is harmless with most arteries but not the carotids where even a momentary interruption in flow can provoke a cerebral infarction, or in coronaries where a myocardial infarction might ensue. The coronaries have been known to spasm even when another nearby artery has been accessed. 7

This eventuality can be averted through the upstream administration of antispasmic agents such as prostacyclin, nitroglycerin, isosorbide dinitrate, nimodipine, verapamil, diltiazem, nifedipine, propranolol, an L-type calcium channel blocker, or β-blocker, each according to its onset to action and active duration. Extension 162 and retention barbs 158 necessarily covered over by a layer of sugar or frozen butter when a straight ductus coupling or stent version of the diverter without the bend is positioned transluminally, the risk of vasospasm is dispelled through the administration of an antispasmic such as one of those just specified. Adverse side effects are generally associated with continued use over a period much greater than that required, and an infarction should not ensue within the time needed to retrieve the side-entry plug and place the valve. An endoluminal diverter such as that shown in FIGS. 31A and 31B allows no portion of flow to continue along its normal path and is not intended for use in carotids or coronaries.

As may be noted in FIG. 28, because it always diverts urine away from either or both ureters, a nonadjustable embodiment such as used in a urinary voiding prosthesis requires no side-entry hole obturator. In an adjustable embodiment such as that shown in FIG. 30, once advanced into the native lumen, the obturator moves between two positions, either flush against the far wall of the ductus, here a ureter, for fully diverted flow or flush against the near wall, thus clearing the native lumen for fully normal, or undiverted, flow. In either position it must comply in concave or convex curvature to the luminal wall.

Rotating the control knob in the surface port retracts the obturator so that it covers over the side-entry hole, sealing off the diversion route limiting the flow of urine to the lower tract, while rotating the knob in the opposite direction advances the obturator against the urothelium opposite the side-entry hole. Should medical or surgical treatment allow the lower tract to recover, the obturator should be sufficiently elastic at body temperature to allow its removal by pulling it out through the side-entry hole with little if any injury to the ureter.

To allow healing with closure of the small side-entry hole, a piece of absorbable tape is placed over it to both seal the ductus and appose its sides, thus prompting primary healing, or healing by first intention. Should extended positioning of the obturator in flush relation to the urothelium at either side provoke an adverse tissue reaction, the accessory channel affording access is used to deliver a counteractant, usually steroidal to include an anesthetic. A basic side-entry jacket positioned upstream to automatically release medication on a scheduled basis can be placed at the same time the voiding assist device is implanted. The automatic release of drugs from subcutaneously, or subdermally, positioned small flat reservoirs is addressed in copending application Ser. Nos. 14/121,365, 15/998,002, and 14/998,495.

The diversion chute, 18 in the accompanying drawing figures, is sized according to the internal diameter of the substrate ductus. The lumen will normally vary between individuals and along its length between 2.0 to 5.0 millimeters in diameter and when pressed flat, the chute, made of a suitable polymer sufficiently plasticized to impart a soft rubbery or spongy character, will generally vary between 5.0 to 10 millimeters in length. When advanced into the ureteral lumen, the distal tip of the chute and its sides must flush fit conform to the internal contour of the lumen. Any plasticizer residue must be fully removed. Running along the bottom center of the chute is the continuation of the catheteric drugline which upon reaching the chute becomes an accessory channel.

Accessory channels coursing thus or entering the diversion jacket or valve separately are available to directly pipe-target drugs and diversion system maintenance agents to the jacket. System maintenance agents can be automatically delivered according to the microcontroller prescription-program schedule. Disease associated analyte sensors can be incorporated into the diversion jackets or the diverter shown in FIGS. 31A and 31B or positioned separately. While ordinarily used for urine outflow, the effluent pipe 110 in FIGS. 28 and 30 can be used for drug inflow or to insert a cabled device for passage up through the prosthesis or urinary assist device for examination. Use of this path to delivery drugs depends upon which portion or portions of the lower urinary tract remain intact.

In a user controlled embodiment such as the voiding assist system shown in FIG. 30, the entire lower tract is intact. While a bladder infection is much reduced where the urethra is missing, retrograde use of the effluent pipe to spray the bladder interior with a biofilm-breaking antiseptic can eliminate the need for an oral antibiotic. The accessory channel running along the bottom center of the chute allows drugs to be delivered into the urinary tract situated below the chute. Retrograde delivery of drugs through the mainline and trepan tube coursing through the sidestem 19 allows drugs to be forced up though the ureter and into the renal pelvis and calyxes, the use of contrast allowing the level reached to be viewed.

In a prosthesis such as that shown in FIG. 28, the free or stump end of the ureter with a diversion jacket as shown extends down a ways before it is cut and completely closed off with a fibrin sealant. In contrast, when the prosthesis diverts urine with a diverter as shown in FIGS. 31A and 31B, the endoluminal flow diverter is inserted into severed end of the ureter. Thus, with a diversion jacket, urine is prevented from entering the irregular space beneath chute 18, whereas with a diverter, no such space is present. The chute in a prosthesis is fixed in the fully extended, or deployed, position and therefore omits means of adjustment such as the push/pull control cables shown in FIG. 30.

When the urothelium is normal and the lower tract expected to heal so that no part of it is resected, a diversion jacket offers the advantage of operator-discretionary return to undiverted flow while allowing the prosthesis to remain in place during a follow-up period for ‘watchful waiting.’ Where the lower tract has been surgically removed such as to prevent the spread of cancer or so damaged as not to be practicably reparable in an elderly patient, for example, a diverter such as shown in FIGS. 31A and 31B is less expensive than a diversion jacket.

In the adjustable embodiment shown in FIG. 30, control over the extent of chute extension by push/pull control cables avoids the greater cost of servomotors. Snug apposition against the surrounding lumen wall is achieved without the need for precise adjustment during placement by making the chute of a highly pliant thin soft rubbery material having a feathered surrounding edge molded with a slight upturn about the chute periphery. The surrounding edge of the chute therefore continues to bend upward in compliance with minimal resistance posed by the surrounding lumen wall.

Shown in context in FIGS. 28 and 30, FIG. 29 provides a perspectival section view through a neoureter convergence or confluence chamber 145, into which each jacket outlet drainage catheter, or neoureter, empties. Here synthetic components are substituted for a neobladder and line terminating in a stoma surgically constructed from gut, which is unadapted to and degenerates in contact with urine, so that a primary operation with its complications and adverse sequelae are avoided. Depending upon the length and peristaltic sufficiency of the vestigial ureter above the level of the diversion jacket 143 and/or diversion jacket 143′ when the patient is upright, the pressure of urine upon entering the ureteral side-entry jacket supported by gravity should be sufficient to expel urine out through neoureter 144 and/or 144′ and the surface port into the collection bag.

With urge sensation lacking, this passive process may be satisfactory to the patient who does not mind wearing a collection bag at all times, allowing voiding directly from the ureters through the effluent pipe 110 into the collection bag 148. However, even the patient without a bladder and urge sensation may prefer to avoid the need for a collection bag. For such patients, a means for the temporary storage of urine and signaling that the storage area is full allows voiding directly into a bathroom receptacle without the need for a collection bag. Where the urethra is intact and can be accessed to place a basic side-entry jacket or diverter connecting it to a synthetic neobladder or neoureter confluence chamber, normal voiding should be attainable.

In most cases, urethral emission will prove impracticable so that voiding will be through the effluent pipe 110 out surface port 104 into a bathroom receptacle. Without urge sensation or voluntary control, retention by the internal and external sphincters will not prevent the uncontrolled release of urine. With a fully passive system leading to a collection bag, this is unobjectionable, but in a system intended to allow voluntary voiding, means for signaling to the wearer that the confluence chamber is full and means for controlling its emptying are imperative.

As shown in FIG. 29, the confluence chamber incorporates an impeller 150 and pressure actuated switch 152, in addition to a continent flap at the outlet of the confluence chamber (not shown) functioning as a synthetic neobladder. The impeller compensates for any loss in peristalsis. When the pressure in the chamber exceeds a threshold level nearing that associated with reflux, the assist device signals the need to expel the chamber contents. The preferred means for signaling is vibratory as is standard in smartphones. The vibration can be by an eccentric rotation mechanism internal to the confluence chamber or a signal radiocasted to a smartphone.

An effective urinary assist prosthesis or assist device must accommodate several variables that distinguish potential users. These variables cover intact or missing anatomy and urge sensation. For example, the ability to always void voluntarily while erect will be true of the user with intact urge sensation provided with a controllable or adjustable embodiment for use during the day. Without urge sensation, this will not be true while the user is asleep. To cover all of these eventualities except in some cases where drainage is passive directly into a collection bag, both controllable and most fully prosthetic systems include a synthetic neobladder, or neoureter confluence chamber.

Shown in FIG. 29, the confluence chamber incorporates a pressure actuated switch 152 and an impeller connected to a battery housed in the surface port to empty the chamber into a collection bag whenever the user, usually while sleeping, does not notice the vibratory signal indicating that the chamber is full so that a trip to the bathroom is needed. That is, in some cases, a collection bag is worn at night even when the patient would otherwise not require one. As shown, the jacket to either side receives a single accessory channel 8; however, where for example, drugs are best administered separately or one accessory channel should be reserved for drugs and another for maintenance of the apparatus, a second accessory channel 8′ is provided.

Additional druglines leading directly from a surface port where these can be injected or from an intervening implanted drug or maintenance agent reservoir can be provided for either or both jackets, different entry point therefor appearing in the drawing figures of valves. The wires connecting the pressure actuated switch 152 to the battery are passed from one or more single use or rechargeable button cells position about the central effluent pipe 110 if present on the surface port as would be subcutaneous drug entry opening or control knobs. Pressure switch 152 actuates impeller 150 when the chamber reaches the threshold fill level or there is an increase in pressure that would induce reflux. The fan-conformed impeller allows flow past it, and forcible ejection when actuated by switch 152.

When the power source is not located within the impeller housing 150 for transcutaneous recharging, electrical connection of the power source, here a battery, to pressure actuated switch 152 can be through a separate suitably insulated wire or coursed (run, ‘snaked’) through a dedicated catheter entered through a leak-proof biocompatible, grommet, such as one of nylon, to the switch with terminal connection also protected by a similar leak-proof grommet. The pressure actuated switch is a microswitch comprised of parallel conductive leaves, one of which is bonded to the side of the impeller. The conductive leaves are separated by a leaf spring which under the threshold pressure, flattens, bringing the leaf above and below the spring into contact.

In all cases, electrical and fluid lines must be routed to avoid the possibility of strangling intervening tissue regardless of whether the patient is standing, sitting, or recumbent. When this cannot be accomplished with confidence, fluid and electrical lines connected to a surface port which does not allow this must be reconnected to another surface port positioned to allow a safer route. Involving additional expense, the avoidance of hardwiring through the use of radio remote, such as Bluetooth, connections should be reserved for patients with multiple comorbidities in whom several hardwires are needed.

In an intractable stone former having been treated with no more than partial success, to avert the risk of reflux into the native ureter of a stone solvent such as potassium citrate, magnesium potassium citrate, allopurinol, or sodium bicarbonate at a concentration higher than would be allowed to contact unaffected native tissue, the solvent can be led directly from a subcutaneous drug or agent opening in a surface port to the diversion jacket to flow down into the convergence chamber, thus preventing encrustation and obstruction at the chamber outlet. Sites of stone accretion at higher levels in the kidney, calyces, or pelves can be directly pipe-targeted with the aid of one or more nonjacketing side-entry connectors as described and shown in copending application Ser. No. 14/498,495 of like title, FIGS. 6, 11, 13A, and 13B.

Means for increasing the force of expulsion include electrostimulation by a neuromodulator implant and the use of a diuretic to stimulate calyceal peristalsis. FIGS. 26A and 26B provide sectional views through a subcutaneous pectoral surface port suitable for use with the nonadjustable, or prosthetic, embodiment shown in FIG. 28. This surface port differs from a port for an adjustable, or user-controlled, embodiment in omitting small push/pull control cable knobs of electrical switches to advance or retract the diversion chute. A suitable port for use of a controllable embodiment is positioned to a side of the mons pubis for ease of viewability and use is shown in FIG. 26C. Such a port retains drug entry opening subcutaneously and combines these with a urine effluent pipe 110 which passes through the port to open outside the body at the port center. The end of pipe 110 is protected by a cap containing a material such as gauze which can be wetted before replacing the cap in position.

As indicated, when more than one opening is subcutaneous, or subdermal, and each calls for a different drug or agent, small tattoo marks shown in FIGS. 17 and 27C allow identification of the different openings and thus indicate the proper alignment of a multiple reservoir replenishing injection head such as those shown in FIGS. 27A and 27B. With one or more implanted drug reservoirs, the administration of each drug or apparatus maintenance agent such as a thrombolytic or crystal solvent can be controlled by a microcontroller in single morbidity or less complex comorbidity, or by a microprocessor in more complex comorbidity.

With patients who are mentally disturbed, impaired, or too young to be entrusted with an on-the-skin, or cutaneous, surface port, especially one that includes one or more controls, ports are positioned so that only an adult or clinician can gain access. A port with drug openings is best positioned subcutaneously, and one with one or more controls between the shoulder blades with as little protrusion or discomfort when supine or resting against the back of a chair as possible. The port is best backed with a thinner cushion of the type used to alleviate joint pain in arthritis. In order avoid the need to place the surface port on the skin rather than subdermally only to house a battery for quick replacement, a transdermal or transcutaneous antenna can be implanted to allow an implanted battery to be recharged by transdermal energy transfer.

While it is improbable that a sufficient volume of a drug cannot be stored within an implantable reservoir so that replenishment is required at inconveniently frequent intervals, each time with some injury to the skin, a volume of a drug or agent not accommodated can justify the placement of a second reservoir. Both can be filled through a single opening in the port for delivery through the same accessory channel. Where the need exists to insert a cabled device is frequent as would require a small incision to gain access to the nidus, a port such as that shown in FIG. 26C is used.

The insertion of a cabled device such as a fine fiber scope or laser to examine or service the urinary tract or the interior of the apparatus such as shown in FIGS. 28 and 30 are present and conventional access is not possible is through the urine outlet opening into the effluent pipe 110 at the center of the surface port shown in FIG. 26C. FIG. 30 shows the overall plan of an adjustable, or user-controlled, embodiment suitable for use on an intermittent basis to prevent the need to void when the wearer is indisposed or would be subject to frequent interruption during sleep. Intermittent use embodiments accommodate both daytime use for the wearer with access to bathroom facilities and able to void while standing, and before going to bed can put on a collection bag for passive drainage without being awakened.

### Anastomosis Coupling or Flow Diverter

FIG. 31A shows a tube-within-a-tube endoluminal anastomosis coupling when straight and flow diverter 155 when bent as shown for insertion into the cut end of a vessel other than one that supplies or drains the brain or the heart, or into a ureter where some part or all of the lower urinary tract is missing. When straight for use as a stent rather than curved, the device can be positioned transluminally. Diversion in the vascular tree allows bypassing an intervening defect and can be used to shunt a small proportion of blood to a larger vessel in order to reduce a localized hypertension, for example.

However, these devices cannot divide their outflow. In the lower urinary tract, diversion into a synthetic bypass ‘neoureter’ for drainage into a synthetic neobladder or directly into a paracorporeal collection bag makes possible a prosthetic lower tract where that native would otherwise have to be reconstructed out of gut. Gut is ill adapted to such use, and its harvesting creates an additional site for potential complications, and a urinary diversion stent to carry urine through a surgically constructed conduit requires replacement every one to three months.

The use of synthetic materials allows clear transparency and eliminates the complications associated with tissue, to include susceptibility to infection and malignancy. At the same time, clot in the vascular tree is easily dispelled by the direct release of an anticoagulant drip and crystal in the urinary tract by a crystal solvent. FIG. 31A depicts the endoluminal urinary diverter as a bent double tube comprising a tube within a tube 155 where that inner 156 is continuous and that outer 157 is interrupted by pointed retention tines (prongs, barbs) 158 molded or die cut and pressed outward to project down and outward into the tunica mucosa of a ureter or intima of an artery, at the same time providing an open space or aperture 159 beneath each prong through which drugs released into the space between inner and outer tubes gain access to the surrounding tissue. Tines 158 are convex at the upper surface and inclined in the direction of flow, or antegrade.

Whether molded in plastic or made of metal, outer tube 157 is coated with a polymer that incorporates an anti-inflammatory and antimicrobial. Drugs are delivered into the space between the inner 156 and outer 157 tubes through drugline 8, and drugs and device maintenance agents are delivered into the inner tube 156, hence, into the substrate native lumen, through drugline 8′. If die cut and punched metal (usually stainless steel or titanium), retention tines 158 are bent so as to be convex at the upper surface, and if plastic (usually polyethylene terephthalate or polyether ether ketone), molded thus, so that medication or device maintenance agents released into the inner tube will leave no more than a slight residue when flowing over these, so that little of the agent will fail to flow out through sub-tine, or sub-prong, apertures 159.

As a flow diverter, the diverter is limited in application. Much the same device, usually straight rather than bent and also double-ended, has other applications, such as maintaining certain transplantation anastomoses where immunosuppressives are best applied directly and kept from other tissue, and securing the end of a synthetic line in a ductus. Depending upon the anatomical context and the potential need to pass a transcatheteric cabled device through a small caliber ductus, a basic side-entry jacket, because it presents no part within the substrate native lumen to interfere with passage of the device, is preferable.

By the same token, whether the device is bent as a urinary flow diverter or straight as an anastomotic coupler or stent, where alternative methods for suppressing the runoff of a residue such as magnetic or the downstream release of a reversal agent involve additional complexity and expense, the coupling diverter allows the direct targeting to the endothelium or urothelium surrounding it of medication with negligible if any continuation of the residue through the bloodstream or urinary tract. This advantage is maximized in the targeted transport of chemotherapeutics.

This direct urothelium or endothelium contact drug targeting is also available when a straight embodiment is transluminally positioned as a stent. Means for preventing incisions of the endothelium or urothelium when placed thus are simple and specified below. As an anastomotic coupling, such as between donor-to-recipient stumps following transplantation, insertion is into the cut ends of the stumps where one such device is placed to bridge over the cut. Since the device itself interrupts peristalsis or a pulse, suture should not be necessary either to facilitate healing or to prevent migration. As a stent, the ductus coupling and flow diverter is superior to a conventional drug-eluting stent with a finite limited dose of a single medication. It can deliver different drugs as commanded by the implant microcontroller or microprocessor in response to sensor inputs upstream along the ductus, for example, where, as shown in FIGS. 26A-C and 27A-C, the drugs can be quickly replenished.

Hypothetically, a flap with edge conformant to the curvature to either side of inner tube 156 suspended in the center thereof with magnetically susceptible tip antegrade tip might be drawn by small electromagnets to bias flow to either side leading into separate lumina, and a fully circular damper-like flap would allow switching between full and partial flow. However, incapable of diverting all flow through either branch, this approach is limited in its ability to apportion flow between two downstream ductus. It therefore is unsuitable for use in bypass, or switch, transplantation, carotid endarterectomy, or the extracardiac correction of a transposition of the great arteries.

The passage of flow through a length of tubing with opposing diverters at either end for use as an insert patch to reconnect the ends of ductus which due to disease had to be excised or injury had been lost. Otherwise, pending sufficiency in the generation of the type ductus by means of tissue engineering, the need to harvest a length of native ductus from the patient, usually a vein, will continue to demand a preliminary invasive procedure and create the risk of serious complications at the site of harvesting.

To assure transluminal passage free of any artificial obstruction as could hinder a future need for angioplasty or examination with intravascular ultrasound, coronary and carotid arteries, for example, are best kept from the insertion of any device or part thereof within the lumen, making side-entry jackets superior to a straight double-ended embodiment of the urinary flow diverter shown in FIGS. 31A and 31B. Retention tines (prongs, barbs) 158 and apertures 159 are positioned at radially diverse locations about the circumference and length of outer tube 157. Delivery of a fluid drug into the space between inner tube 156 and outer tube 157 through drugline/accessory channel 8 is beneath the closed off upper ends of double tube 155 under the slight pressure imparted by the implanted drug reservoir outlet pump.

This causes the drug to flow about and down between tubes 156 and 157 to flow out through the spaces 159 beneath each retention tine 158 to wet, and depending upon the chemistry and viscosity of the drug, become absorbed into the tunica mucosa of the ureter or endothelium of the vessel. FIGS. 31A and 31B show the outflow pattern of a drug released from an implanted reservoir delivered into the space between inner tube 156 and outer tube 157 through the space 159 beneath each retention tine 158.

This pattern serves to deliver an anti-inflammatory, anesthetic, and/or antimicrobial directly into the small wound created by penetration of each retention tines (prongs, barbs) 158 that would otherwise cause discomfort or pain referred to one or both flanks, the back, upper sartorius, pectineus, and/or adductor longus, and/or the testes or labia majora until the body responded by enclosing each prong within a fibrous capsule. Retention tines 158 are depended upon to counter dislodgement due to downward migration. The palliation of pain should be limited to the direct topical application of an anesthetic, not a crushing of nervelets as could adversely affect peristalsis and/or induce atrophy. Fitted properly, peristalsis will eventually secure the full penetration into the tunica mucosa of the prongs.

For transluminal placement, it is imperative that the retention prongs, or barbs, 158 and drugline attachment extension 160 not scrape against the endothelium or urothelium. Various substances can be used to cover over these over before being dissipated once the device has reached its destination. Coating the outside of a ductus coupling to be placed in the manner of an endoluminal stent with a concentrated solution of a sugar solid during placement can if necessary, be followed by the release through the barb apertures 159 of warm distilled water to accelerate its dissolution. A refrigerated slice of butter in a stack thereof separated by wax paper can be wrapped around the device, then placed in a freezer. Upon reaching the destination, accelerated dissolution of this barrier layer is through the release through barb apertures 159 of suitable enzymes (lipase, lactase).

Accordingly, the tube-within-a-tube ductus coupling or flow diverter 155 is inserted into the cut end of the artery so that it fits snugly on the systoles but does not significantly press radially outward as would compress the small vessels whether caudally proceeding branches of the abdominal aorta, common iliac, renal, superior and/or inferior vesical, gonadal (testicular or ovarian), middle rectal and/or uterine arteries, and closer to the bladder, the superior vesical, middle rectal, in men the inferior vesical, and in women, the uterine and vaginal arteries which provide tiny branches that inter-anastomose just inside the adventitia (fibrosa, fibrous coat) and submucosa; accompanying nervelet branches of the renal, aortic, and/or hypogastric autonomic plexuses; and or lymphatics of the aortocaval and common iliac nodes in the abdominal ureter or the internal and external iliac nodes in the pelvic ureter as would promote atrophy.

Depending upon the state of the urinary tract, diversion can be to the native bladder, constructed (unpreferred) or synthetic neobladder as preferred through a nonjacketing side-entry connector as shown in FIGS. 12A and 12C of copending application Ser. No. 14/998,495. When possible, rejoinder from prosthetic neobladder 145 to the urethra rather than connection to an outlet hose 110 is preferable as allowing normal voiding. However, in the absence of urge sensation, voluntary control requires a tactile or audible signal, which is easily accomplished, and except in prostatectomized males with favorable anatomy, clear access to the urethra for this purpose is likely to prove elusive.

If practicable at all, much dissection is involved. Given the limited working space, connection to the urethra might be more easily accomplished with a double-ended version of the device shown in FIGS. 31A and 31B rather than a side-entry jacket. Referring to FIGS. 28 thru 30, with the nonvalve diverter as in valves systems, when the lower tract is actually or effectively missing, diversion is to a prosthetic neobladder 145 and out drainage line 110 opening to the exterior at the center of port type 147 and into a collection bag 148 ordinarily cinched about a thigh, or where no neobladder is present, then drainage is directly through line 110 and into the collection bag 148. The positioning of either a side-entry diversion jacket or a diverter along a ureter, for example, is best at the level with the largest internal diameter remaining intact.

The justification for the placement of any implant is that it is necessary or would provide greater benefit than harm. There is no good place to position a foreign object inside the body. Anywhere an implant is placed without adverse reaction protective countermeasures can and will lead to complications. A completely synthetic object free of plasticizer of any other irritant can provoke a tissue reaction—no provocative chemical or special allergy is required. One tactic is to allow the body to overreact to the foreign body by initiating a cascade of cell signaling peptides, or cytokines, often as self-destructive as remedial or protective, but immediately suppress if not terminate the reaction through the directly piped delivery to the nidus of suitable medication to include a glucocorticoid and synthetic immunosuppressive, for example.

Drug eluting stents exemplify this approach in a simple form but are limited to a single drug or mix of drugs which once depleted are spent and no longer targetable. The choice between an intraluminal and extraluminal device must be a clinical judgment based upon the specific conditions encountered. The devices and methods delineated herein are not necessarily meant to supplant accepted practice but rather provide alternative options to make possible a more appropriate treatment of specific conditions. Inflammation generates detectable analytes which sensors can report to an implant microcontroller for response with the direct pipe-targeting of a palliative, remedial, or reversal agent. Side-entry jackets are positioned in surrounding relation to the substrate ductus leaving the lumen clear of a foreign object but must present radial projection.

By the same token, lined with foam, peristalsis or the pulse is accommodated, and migration prevented, usually without even an occasional tethering to nearby tissue by running suture through a suture eyelet, part number 15 in the accompanying drawings. Inside a peristaltic ductus, to minimize stress on the smooth muscle restrained from movement by a solid insert device, the endoluminal diverter or end-to-end stump connector should be kept short. In the devices described here, the adverse sequelae associated with intra- or extraluminal contact between an endoluminal implant such as a diverter with the endothelium or urothelium or between a surrounding jacket and the adventitia or fibrosa have been taken into account and responded to by providing druglines that immediately target medication directly to the site of the irritation.

Except for the druglines which enter to become accessory channels 8 and 8′, which are easily rotated to avoid it, an endoluminal device such as that shown in FIGS. 31A and 31B is positioned within and does not project outside the substrate ductus, thus eliminating the risk of abrasive contact with neighboring tissue. However, absent the direct delivery of medication to the implant-endothelial or endothelial interface within the ductus, until endothelianized, or incorporated into the endothelium, the implant would likely irritate the ductus lining, and if long, unduly interrupt the passage of peristaltic or pulsatile waves and in small gauge lumina, may prohibit or make more difficult passage with an intravascular ultrasound probe or excimer laser, for example.

Endoluminal, the urinary diverter or a straight embodiment thereof such as a stent is accessible in the catheter laboratory but not endoscopically. With a coronary or carotid, where clamping poses sufficient risk of vasospasm as to be avoided, placement of a side-entry jacket or valve is accomplished without clamping through a ‘keyhole’ incision under local or regional anesthesia, the administration of a vasospasm suppressant available as a protective measure. Used in a urinary prosthesis, a urinary diverter is simpler and less expensive than a side-entry device. However, at the end of a severed ureter, it is fixed in diverting flow and cannot switch between diverted and nondiverted flow, much less apportion flow between the two making it unusable in a urinary assist device for the diversion of urine only while the wearer is asleep or appearing before an audience. By the same token, in a prosthesis, which must remain fixed, this nonadjustability is advantageous as a safeguard against improper use.

### 3. SUMMARY OF THE INVENTION

Described is a ductus side-entry jacket with an extendable and retractable lumen-inline flow-diverting chute having a wide range of applications, to include urinary diversion at the ureter and the creation of shunts or bypasses in the bloodstream. Use to achieve instant switching of the blood supply from the native through a synthetic bypass device allows repair or replacement of the native structure without the need to harvest, tubulate, transplant, and physiologically reassign native tissue as a diversion conduit, such as using gut to convey urine.

The lumen through the jacket side-stem is first connected to a vacuum pump to remove a plug of tissue of like diameter as the native lumen in the wall of the substrate ductus through which the diversion chute will pass. The vacuum draws the ductus wall past the trepan leading edge of the side-stem tube to cut the opening for the chute into the substrate ductus. The opposite internal wall of the ductus is protected from gouging injury by a limit switch that stops the suction the moment the plug has been cut. The plug can be extracted through the mainline by the vacuum pump, through a capillary gauge catheter connected to the pump, or a guidewire with a hook at the end.

The control of vascular valve diversion chutes can be manual or automated. Manual control pertains to the night-time diversion by the user of urine from either or both ureters to a paracorporeal collection bag by adjustment through Bowden cables controlled at a body surface port, or by the simultaneous switching of plural solenoid controlled valves during an open procedure. In transplantation, control is automatic by an implant microcontroller or microprocessor used to coordinate the vascular servovalves connecting like vessels of donor and recipient in response to physiological indicia of the recipient detected by sensors implanted at appropriate locations.

Use to switch the blood supply of a recipient from one or more of his own organs to those of a donor allows the gradual transfer of the organ or organs from the donor into his own circulatory system to constitute a new ischemia-reperfusion injury free method of solid organ transplantation. If an intact donor is unavailable, an ex vivo machine-perfused organ can be used. Heart transplantation thus eliminates the need for general anesthesia, cardioplegia, clamping, and allows avoiding a significant interruption or transient in mean arterial pressure and its potentially serious consequences such as graft organ perfusion-reperfusion injury and cognitive dysfunction.

Depending upon the application, the scope of control ranges from bistable, meaning fully open or fully closed with a damped push-pull solenoid that moves the diversion chute from fully retracted to fully extended to divert urine, to variable when manual, to precise continuous variability under the control of a microprocessor-governed linear servomotor, for example. Such a jacket allows the diversion of flow from the lower urinary tract if missing or injured indefinitely, precluding any sensation of urgency, while for intractable frequency that interferes with public performance or sleep, the wearer can control diversion as needed.

### 4. OBJECTS OF THE INVENTION

An object of the invention is to provide a means for connecting a catheter to a blood vessel from a subdermally or on-skin positioned port, which for all but short-term use in-a medical center, is superior to a central line or central venous catheter, such that controllable in outlet cross-sectional area from outside the body and securely fastened, it can continue in use indefinitely.

Another object of the invention is to provide a means for connecting a catheter to a vessel from a subdermally or on-skin positioned port, which avoids the many complications associated with conventional central lines and surface ports, and is versatile, in that it can be used unidirectionally to deliver drugs, fluids, or parenteral nutrition over a wide range of flow rates, can pass cabled devices such as a fiberscope, laser, or intravascular ultrasound probe, and/or using a double lumen line can be used bidirectionally to shunt blood to and from an extracorporeal hemodialysis or apheresis machine, for example, or to bypass urine around an intervening section of the urinary tract or shunt it to an paracorporeal collection bag, and can incorporate physiological, such as venous pressure and other hemodynamic monitoring and metabolic sensors.

An object of the invention is to provide a means for connecting a central line, used for a multiplicity of purposes, to a vessel through a small incision, hence, without the need for a guidewire and suspension of the line within the vessel, thus eliminating a multitude of adverse complications, recommending the replacement of central lines for extraclinical ambulatory use.

Another object of the invention is to provide tubular anatomical structure, or ductus, flow diversion side-entry jackets suitable for permanent implantation through 1. Avoiding compression of the fine vessels and nerves entering and exiting the substrate adventitia, 2. Achieving access without entry into the luminal stream with the least trauma through the smallest possible puncture wound, 3. Avoiding any presence of the line or catheter or any part of the side-entry jacket within the native lumen, and 4. Providing sufficient openings through the jacket to maximize exposure of the adventitia to the internal environment.

Yet another object of the invention is to provide ductus flow diversion side-entry jacket and body surface port sets which make it virtually impossible for the jacket or body surface port to migrate.

An object of the invention when used in the urinary tract is the diversion of urine at any level along one or both ureters to an external collection bag, thus bypassing an impaired or missing lower tract, urge sensation, and/or the need for mental competency as would allow use of the facilities provided therefor.

An object of the invention when configured as a prosthesis for continuous diversion is to create a permanent path for the release of urine where patient competence to void in a normal manner or pathology, such as neurogenic, trauma, resection of malignancy, or birth deformity had left part or all of the distal tract missing or dysfunctional.

Another object of the invention when configured to allow the temporary diversion of urine by takeoff at the ureters is to expedite healing of the lower urinary when diseased or operated upon, the invention making possible the direct delivery of drugs, to include checkpoint inhibitors or blockers, into the distal tract as well as crystallization preventive agents through the diversion line.

Yet another object of the invention when configured to allow temporary diversion is to enable the patient to divert urine away from the distal tract on a voluntary basis to allow uninterrupted sleep or public performance, and allow normal urge sensation to be suspended and voiding deferred when bathroom facilities are unavailable.

Another object of the invention is enable the diversion of urine through components made of synthetic materials devised to least provoke an adverse tissue reaction and not susceptible to the multiple causes of degradation and medical complications that affect living tissue, especially when harvested from and thus causing injury to a different organ system in a preliminary procedure itself susceptible to complications, the side-entry diversion jackets used readily fitted with sensors and other electronic components to allow diagnostic monitoring and as necessary, the automatic release of drugs to effect therapy and agents to maintain the patency of both the innate and synthetic lumina.

A central object of the invention is to provide a urinary or vascular diversion coupling jacket with one or more collateral or accessory channels to allows fine scope access for viewing and the direct targeting to the line, jacket, and native lumen of thrombus, crystallization, inflammation, and biofilm counteractants, thereby making possible the use smaller caliber synthetic tubing, or conventional catheters, whether solid, flexible, or spun (woven), to redirect the flow of content through a native conduit without becoming clogged on a permanent basis.

Another object of the invention is to eliminate the need for an ileal conduit and stoma constructed of harvested gut, which absorptive is unsuited to conduct urine and susceptible to infection, metaplastic degradation, and other disease.

Yet another object of the invention is to provide flow-diverting side-entry jackets, which made of synthetic materials, are readily fitted with and ideally serve as a platform for electronic sensors, imaging probes, a laser, ultrasound probes, as well as therapeutic means such as a warming element, alone or in combination.

Another object of the invention is to provide stream or flow-diverting side-entry jackets, which made of synthetic materials, are readily modified to incorporate permanent or electrical magnets magnetized normal (perpendicularly) to the axis of flow through the encircled ductus, thereby allowing magnetized contents in the passing flow, such as superparamagnetic iron oxide nanoparticulate drugs, to be detained against or drawn into the wall of the native lumen.

An object of the invention in a bilateral embodiment, wherein each ureter is provided with an independent adjustable takeoff and body surface port, is to allow the obtaining of urinalysis test samples from either kidney.

An additional object of the invention when used to divert flow through a vessel is to provide a conformation that eliminates any part of the jacket except the gradually curved upper surface of the chute or any bare metal surface in or alongside the stream, thus considerably reducing if not eliminating the inducement of clot due to turbulent flow and clot-inducing contact, and therewith, the initiation of a cytokine cascade capable of stimulating the formation of clot anywhere in the circulatory system, the more so in a patient already thrombophilic (hypercoagulable, prothrombotic), or prone to clot due to cancer and/or other disease.

An additional object of the invention used to divert flow through a blood vessel is to allow the direct targeting into the distal vessel inferior to the chute of drugs to treat the distal vessel.

Another object of the invention is to provide a much improved connection to a vessel from a port safely and securely fastened at the body surface for use as a central line, such as a central venous catheter.

Yet another object of the invention is to allow the shunting of blood to a hemodialysis or apheresis machine by plugging the machine lines into a small, safe, and secure subdermal port with no line ends exposed making these more susceptible to infection when disconnected, thus eliminating the need to repeatedly accomplish connection requiring much more time to a vessel using a more disturbing method or causing the patient to present a more disturbing appearance.

A governing object of the invention is to provide new methods for solid organ transplantation to significantly increase the pool of available organs.

An object of the invention is to make possible emergency sudden or metered immune tolerance inductive switching of the circulation of a recipient from his own defective solid organ to the healthy organ of an a brainstem dead donor on life support, and in so doing, eliminating the cytokine storms associate with death, excision, metabolic and endocrine isolation of the graft organ, cross clamping, cardioplegia, ischemia, cold storage, and reperfusion injury, for example, lessening the odds for rejection and the late development of vasculopathy, and in so doing, improve considerably the odds for success following a heart or other solid organ transplant.

An object of the invention in organ bypass or switch transplantation is to provide a means for avoiding the need for cardiopulmonary support with cardioplegia, aortic cross-clamping, and the need for general anesthesia during vascular surgery, to include open carotid endarterectomy and heart transplantation, where conventional techniques interrupt the flow of blood, often results in cognitive dysfunction, and sometimes, cerebral stroke, myocardial infarction, and reperfusion injury.

Another object of the invention is to make possible the replacement of a severely myopathic failing heart with two less than ideal replacement hearts, thus expanding the zone of hearts acceptable for transplantation while placing each heart in support of the other.

An object of the invention is to make possible double heart metered heart transplantation, which allows adjustment in the ejection fraction of both hearts working together, as an alternative to the use of a ventricular assist device when the complications, availability, and/or expense of such a device contraindicate its use.

Yet another object of the invention is to make possible vascular bypass connections for carotid endarterectomies and procedures that otherwise require entry into the heart, such as correction of transpositions of the great vessels and heart transplantation, which eliminates ischemic time, the need to clamp, transect and anastomose vessels, and therewith, the need for cardioplegia, artificial cardiopulmonary support, chilling, reperfusion injury, and the adverse sequelae these produce.

Another object of the invention is to eliminate the causes for transplant failure other than immune rejection, such as reperfusion injury.

Another object of the invention is to provide a means for accomplishing carotid endarterectomies, the correction of transposition of the great vessels, and solid organ, to include heart transplantation, where said bypasses need not be followed by anastomosing the vessels as repaired or redirected but can remain in place to the end of life.

An object of the invention is to provide a means for correcting an uncomplicated transposition of the great arteries without the need to enter the heart.

Yet another object of the invention is to provide a means for adjusting the blood pressure locally or regionally to protect lesions along the pertinent segment of a vessel or impaired tissue without effect on the systemic circulation, the means additionally capable of delivering medication and cabled devices to the treatment site, where the medication can consist of nonradioactive drugs or other agents-subintimally implanted magnetically susceptible grains or bands, and the cabled devices can be fiberscopes, lasers, intravascular ultrasound probes and physiological parameter sensors.

Another object of the invention is to provide a means for directly targeting drugs into ductus to include vessels, the urinary tract, and the ductus of glands in response to an adaptive prescription program executed by an implanted microcontroller using negative feedback based upon sensors of physiological parameters to maintain homeostasis as an adaptive ambulatory prosthetic disorder response system to instantly apply remedial measures responsive to morbidity and comorbidity.

### 5. DESCRIPTION OF THE DRAWING FIGURES

FIG. 1 shows a nonadjustable flow-diverting basic side-entry jacket as described in copending application Ser. No. 15/998,002, entitled Ductus Side-entry Jackets and Prosthetic Disorder Response Systems, positioned along the substrate ductus prior to insertion of the trepan tube through the wall of the lumen.

FIG. 2 shows a simple urinary flow diversion jacket adjustable by the operator during placement but not afterwards by the patient, such a diversion-irreversible, or nominally fixed embodiment serving as the ureteral takeoff in a prosthesis such as that shown in FIG. 28 for a patient without a functioning lower urinary tract.

FIG. 3 is a cross section through side-stem 19 taken along line A-A′ of the diversion jacket shown in FIG. 2A. shows

FIG. 4 shows a forked vessel segment-straddling spring-return double clamp, or straddle-clamp, for briefly stopping flow through a small segment along a blood vessel shown clamped to stop the flow of blood with the segment to be jacketed trapped with blood, thereby facilitating placement of a side-entry jacket, choke valve, or vascular valve thus averting the difficulty of extracting the tissue plug for insertion of the trepan tube when, for example, obstructed due to the presence of a diversion chute in the trepan tube

FIG. 5 shows a urinary diversion valve, manually adjusted by turning a control knob on a small body surface port, where the control knob is connected to a push\pull control cable positioned to drive the diversion chute forward or backward.

FIG. 6 provides a cross sectional view along line B-B′ through the diversion valve shown in FIG. 5.

FIG. 7 is a longitudinal midline sectional view through a flow diversion jacket only capable of fully retracted or extended deployment of the diversion chute, for use along the vascular tree, where a plunger solenoid makes it possible for the operator to deploy multiple diversion chutes simultaneously as is required, for example, in a sudden switch heart transplant, shown with the solenoid positioned to have pushed the flow diversion chute to full extension for full flow diversion.

FIG. 8 shows a flow diversion jacket of the kind shown in FIG. 7, except that the solenoid is reversed in positioned to pull, rather than push, the diversion valve, this configuration placing the plunger solenoid beneath rather than in line with the accessory channel line used to advance the diversion chute, allowing the jacket to be made shorter, shown with flow diversion chute fully extended for full flow diversion.

FIG. 9 shows a pulse tubular linear servomotor suitable for miniaturization for positioning in a vascular servovalve.

FIG. 10A provides a longitudinal midsection through a vascular servovalve driven by a pulse tubular linear servomotor of the kind shown in FIG. 9 with the addition of a direct manual fine adjustment feature which allows the operator to apply minor adjustments in retraction or extension of the diversion chute with ostium obturator into the substrate native lumen. FIGS. 10B and 10C illustrate how the one control knob allows separate control over engagement of the direct manual fine adjustment device as required in a carotid endarterectomy bypass device, FIGS. 10A and 25A and 25B showing such a vascular servovalve with divided outlet, FIG. 10D providing a more detailed cross sectional view of the mechanism shown in FIGS. 10B and 10C, and FIG. 10E providing a more detailed view of the miniature endoscopic-type grasper used to stabilize the piece used to connect the detachable manual fine control device to the servomotor housing before and after the servovalve is placed along the substrate vessel.

FIG. 11 shown the use of diversion valves to exchange (switch, ‘swap’) the continuation of flow between two vessels as in an extracardiac transposition of the great arteries within one patient or between corresponding vessels in two patients.

FIG. 12 shows the use of bidirectional side-entry diversion servovalves to take the place of separate valves where, as in neonates and infants, space is at a premium.

FIGS. 13A and 13B show paired bidirectional diversion jackets without duplication along the same ductus at a nearby level in FIG. 13A and with duplication in FIG. 13B to allow flow to periodically be switched between the upper and lower pairs when, for example, permanent placement along the forcefully pulsatant great arteries to correct a transposition where an extracardiac repair is practicable but a levering movement of the valve, even though stabilized through use of the suture loops, could cause irritation.

FIG. 14 provides a longitudinal section through a vascular choke servovalve, or servochoke.

FIG. 15 is a simplified schematic representation of a metered switch transplantation, where the positions of the diversion chutes are set at the midway point of extension in either a metered switch orthotopic or heterotopic heart transplant, which in a metered switch heterotopic double heart transplant represents the setpoint when the heart of the donor matches that of the recipient in absolute stroke volume, or ejection fraction.

FIG. 16 elaborates upon the simpler schematic provided in FIG. 15 in showing that in a heterotopic switch, or double heart, transplant as exemplary for a heterotopic switch transplant, flow through the graft organ is channeled normally through all the organ arteries and veins, eliminating disuse atrophy as occurs when the graft is connected to the abdominal aorta and inferior vena cava as is commonly done in immunological laboratory testing, for example.

FIG. 17 shows the positions of the hearts in a completed double heart heterotopic bypass, or switch transplant, the added heart placed to the right hand side of that native as preferred and showed in FIGS. 15 and 16, for visual clarity, flow between only the native and added aortae, lower venae cavae, and lower right pulmonary veins shown.

FIGS. 18A and 18B show the action of the arterial blood flow diversion chutes in a sudden switch organ transplantation wherein each solenoid driver moves its flow diversion chute from the fully retracted position shown in FIG. 18A to the fully extended position shown in FIG. 18B whereby flow is bypassed from the native heart on the right and through the donor organ on the left.

FIGS. 19A and 19B show the action of the venous blood flow diversion chutes in a sudden switch organ transplantation wherein each solenoid driver moves its flow diversion chute between the fully retracted position shown in FIG. 19A whereby flow is through the native organ to the fully extended position shown in FIG. 19B whereby flow is bypassed from the native and through the donor organ.

FIGS. 20A and 20B show the action of the arterial blood flow diversion chutes in a metered switch organ transplantation wherein each servomotor driver moves its respective flow diversion chute between the fully retracted position in FIG. 20A, through the intermediate position in FIG. 20B, to the fully extended position in FIG. 20C under sensor feedback indicative of an adverse immune response to the controller, programmed to halt or slow down the process until spontaneously or medicinally resolved.

FIGS. 21A, 21B, and 21C show the action of the venous blood flow diversion chutes in a metered switch organ transplantation wherein each servomotor driver moves its flow diversion chute between the fully retracted position shown in FIG. 21A, through the intermediate position shown in FIG. 21B, to the fully extended position shown in FIG. 21C under sensor feedback indicative of an adverse immune response to the controller, programmed to halt or slow down the process until spontaneously or medicinally resolved.

FIGS. 22A and 22B show the disposition of the jackets and bloodlines before the exemplary left lung as graft organ has been harvested from the donor in FIG. 22A and after it has been placed in the recipient in FIG. 22B.

FIGS. 23A, 23B, 23C, 23D, and 23E provide a perspectival overall view in FIG. 23A, an overall side view in FIG. 23B, a closer side-view in FIG. 23C to show the near side of a surgical chestdome with surrounding cast iron weight and foam base hid behind the arm of the patient in FIG. 23A, an upper, superior or topside, view in FIG. 23D, and a bottom or foot-ward (inferior, caudad) view in FIG. 23E, of a surgical chestdome to be placed over the chest of the patient prior to entry into the chest for eliminating a surgical pneumothorax without general anesthesia and little if any mechanical ventilatory support.

FIG. 24 shows an anterior, or frontal view of a heart cage to protect a metered switch heterotopically transplanted second heart with inflow and outflow vessels from compression and the heart from tamponade when positioned outside the ribcage or otherwise left not sufficiently shielded by bone.

FIG. 25A shows a double, and FIG. 25B a single bypass, intended primarily to implement a carotid endarterectomy, which—depending upon the state of degenerative disease or surgical or accidental trauma—can be used as a permanent prosthesis, or an alternative pathway switched to during healing, for example, or as a periprocedural bypass, to accomplish the endarterectomy without ischemia, fluctuations in blood pressure, and reduced incidence of cardiovascular accidents to include those due to a release of debris that could result in an intra- or postoperative stroke, or a cerebral or even a myocardial infarction, positioning of the valves drawn side-on to the plane of section for clarity, even though the valves are actually rotated to least encroach upon neighboring structures.

FIGS. 26A, 26B, and 26C show a vertical sectional side view of an entirely below-skin (subcutaneous, subdermal) surface port 146 not requiring an outflow opening as does a urological port, with multiple openings for the injection of drugs into their respective implanted drug reservoirs for automatic release, or when the reservoir and pump are omitted as in line K to a kidney, for blood to be withdrawn for diagnosis, and in FIG. 26B a rear view of the subcutaneous multiport shown in FIG. 26A, while FIG. 26C shows a port 147 which includes both a subcutaneous drug inlet and central above-skin scope inlet and urine outlet openings.

FIG. 27A shows a front view of a manually operated four head hypodermic syringe injector and FIG. 27B the equivalent multiple jet injector (needle-free injector, pneumatic injector) for allowing the simultaneous replenishment of drug reservoirs through a surface port of the type shown from outside the body in FIGS. 17 and 27C, wherein the openings are positioned complementary to the injection points of either type multiple injector with minimal discomfort for the patient and the risk of human error considerably reduced if not eliminated. FIG. 27C shows the external tattooed markings placed to assure precise alignment of the hypodermic needle or jet injector nozzles with the injection openings in the subcutaneous port. Multiple nozzle jet injectors of which the nozzles are designed or adjustable to release drugs of different viscosities are omitted from the drawings as familiar to medical personnel.

FIG. 28 provides a schematic anterior view of a bilateral automatic urine collection and voiding prosthesis using one-time manually set valves such as those shown in FIGS. 2 and 3 or solenoid-driven valves such as shown in FIGS. 7 and 8 for permanent placement in a patient with a missing or defective lower urinary tract and therefore missing urge sensation, to divert the effluent into a synthetic or surgically constructed neobladder, thence or directly into a paracorporeal collection bag.

FIG. 29 shows a more detailed view of a neoureter confluence chamber, or synthetic neobladder, such as that shown in FIG. 28 for catching urine for automatic expulsion into a paracorporeal collection bag when filled.

FIG. 30 provides a schematic anterior view of a bilateral automatic urine collection and voiding system for a patient having urge sensation with nocturia or frequent urination, for example, whose sleep is interrupted by frequent urination intractable to conventional treatment, or for a public performer who must defer voiding until a proper receptacle can be used or drainage diverted to a collection bag voluntarily, where voiding is controlled manually by the user who rotates knobs mounted to a side of the mons pubis or mons veneris which advance and retract push/pull, or Bowden cables used to adjust valves of the type shown in FIGS. 5 and 6 or servovalves such as shown in FIG. 10A but without the fine adjustment device.

FIGS. 31A and 31B show a nonadjustable ductus flow diversion coupling for insertion into a vessel other than a carotid, jugular, or coronary or into a ureter for diverting flow into a synthetic shunt or bypass, FIG. 31A providing a front view, and FIG. 31B a perspectival view looking down at an angle toward the diverter showing the outflow pattern of a drug delivered into the space between the inner and outer tubes through the opening beneath each upwardly convex prong.

### 6. DESCRIPTION OF THE PREFERRED EMBODIMENTS OF THE INVENTION

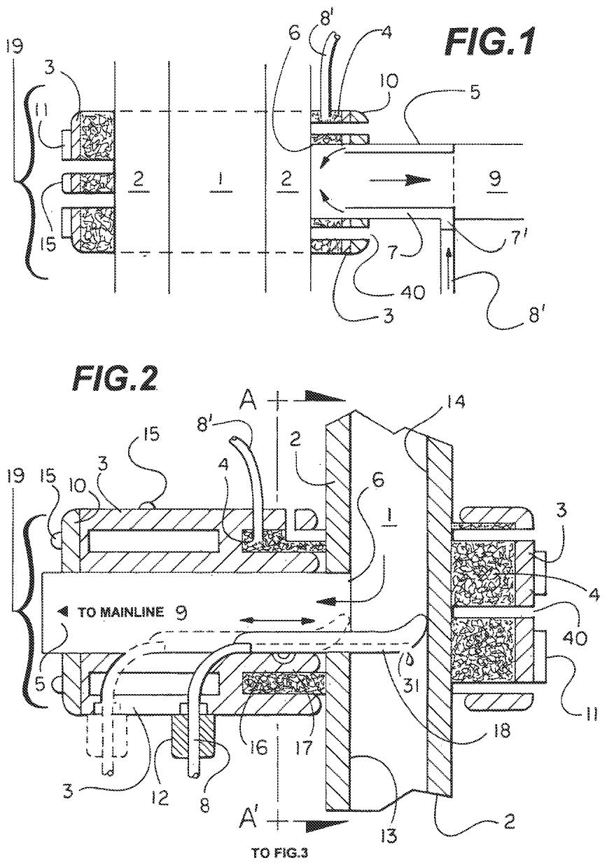

### Valves—FIGS. 1 Thru 3, 5 Thru 8, and 10 Thru 14

To allow the internal structures to be shown clearly, valves have been drawn disproportionately large, prompting an impression of excessive size and weight. Each type valve has also been shown with every element necessary to deal with any potential application. In reality, not all of these features are likely to be needed for a specific application, so that the cost for specific valves will be less than the drawings suggest. To prevent backfilling with blood and the possibility of obstruction by clot, accessory channels along blood flow passageways are filled with water and/or provided with an anticoagulant drip. The implanted controller adjusting the diversion chutes, water fill and the release of an anticoagulant are coordinated automatically.

In FIG. 1 showing a basic side-entry jacket, number 1 is the lumen of the substrate ductus to be jacketed, and 2 the wall surrounding the lumen, both products of nature and not part of the invention. Number 3 is the outer shell of the basic side-entry jacket, usually made of polyether ether ketone, 4 a viscoelastic polyurethane foam layer lining shell 3 containing perforations along its length to avert atheromatous degeneration of the encircled ductus. The adventitia-protective open cell viscoelastic polyurethane foam lining the valve jackets shown in FIGS. 1, 2, 3, 5, 7, 8, and 10A are thoroughly wetted with an anti-inflammatory, immunosuppressive, and/or antimicrobial upon placement which is periodically replenished through accessory channel 8′.

Represented entering perpendicularly into the foam for visual clarity, entry is equally effective when the proximal end of accessory channel 8′ is inserted along ‘breathing hole’ perforation 40 running about the circumference of sidestem 19, shell 3 necessarily interrupted by solid segments for structural integrity. Part number 5 is the trepan tube with razor sharp leading front edge 6, which in a basic side-entry jacket, unlike a diversion valve jacket, requires no stationary surrounding tube to house and align driving means such as a miniature solenoid or linear servomotor, so that it alone comprises jacket side-stem 19. Features common to all valves are that to eliminate abrasive and poking of neighboring tissue, all corners and edges are rounded.

Little heat is generated or conducted through the motor enclosure to surrounding tissue. If necessary, the entire valve is further enclosed within a sock made of a soft biocompatible thermally insulating material to provide both increased freedom from abrasive encroachment on neighboring tissue as well as reduce any heat which the servomotor might generate. The basic ductus side-entry jacket shown In FIG. 1 lacks a magnetic envelope to draw superparamagnetic micro- or nanoparticle carrier-bound drugs from the passing luminal contents and/or a radiation shielding envelope sufficient to protect surrounding tissue against moderate dose-rate radionuclides, for example.

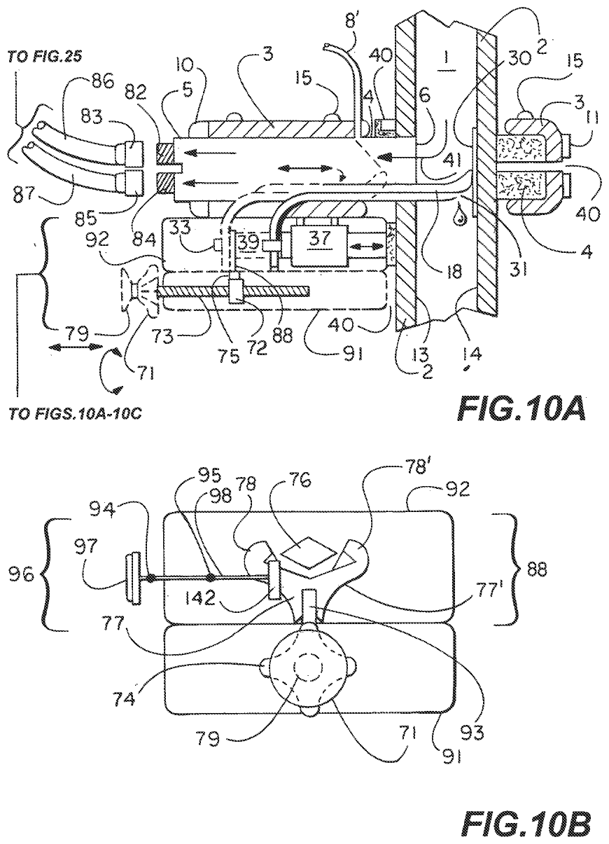

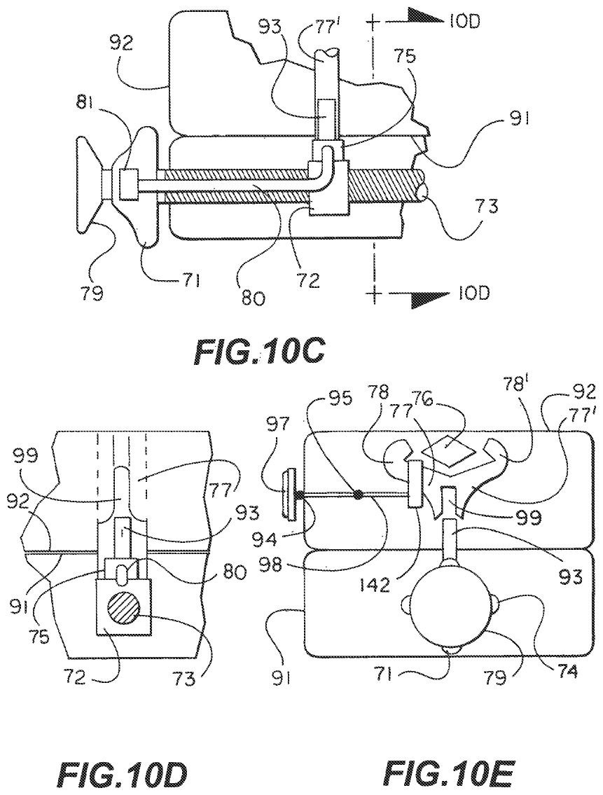

Valves of which the function would best allow direct observation by the operator of their internal function are made of a clear transparent plastic which provides thermal insulation as well. Suitable polymers include those biocompatible based upon poly(aryl-etherethereketone) or PEEK, polyethylene, and polyethylene terephthalate, for example. To facilitate endoscopic examination should the need arise, valves are best made with a polymer of high optical clarity. Vascular servovalves used to create a compound bypass to transfer a solid organ from the donor to the recipient are shown in FIGS. 10A, 25A, and 25B, FIGS. 10A thru 10E, 25A, and 25B also showing the addition of a direct manual fine control device that allows the operator to make small shifts in chute position to assure complete obturation of the ostium by the ostium obturator as necessary.

For example, when the diversion chute is passed through the ostium created in the side of the substrate ductus, the obturator (ostium obturator, stopper) is likely to fully open a tiny distance from the proximal wall of the ductus through which it was inserted. Enclosed within clear transparent plastic, the direct manual fine control adjustment device allows the operator to retract the ostium obturator that tiny distance so that the ostium obturator rests flush against the ostium, thus preventing blood from exiting through the ostium and into the protective foam or the trepan tube.

Unless an open surgical field is necessary for unrelated reasons, vascular valves are placed endoscopically. Part number 6 is the razor sharp leading edge of the surgical circle cutter, or trepan itself, of trepan tube 5. Lacking the ductus side-entry opening, or ostium, obturator, of a valve jacket, a basic side-entry jacket minimizes bleeding when the tissue plug is extracted by directing a circular jet of water over the ostium through water jacket 7 fed through water jacket inlet 7′ sideline, and accessory channel, 8.

In any instance where the valve or servovalve jacket accessory channel or channels incorporated into the jacket such as those shown in FIGS. 2, 5, 7, 8, and 10 numbered 8 or 8′ are insufficient to deliver the volume or number of drugs sought to be directly pipe-targeted to the treatment site, a separate basic ductus side-entry jacket is positioned upstream to make up the difference. Immediately after the jacket has been placed in position, accessory channel 8 becomes available for delivering drugs, for example.

The ostium is cut by a suction pump connected to trepan tube 5, the suction passing through the luminal continuum comprising an attached catheteric line, or mainline 9 connected to trepan tube 5 in the sidestem 19. Trepan tube 5 is encircled and travels within side-stem 19. absent an accessory channel, part number 8 is a small handle to advance diversion chute 18; when an accessory channel is present, 18 is its conduit.

The application of suction against ductus wall 2 through trepan tube 5 draws ureteral wall 2 past trepan 6, excising a tissue plug from the side of ureteral wall 2. The suction pump has a limit switch that instantly stops the pump when the resistance to suction suddenly drops off and the water jet minimizes bleeding while continued suction and if necessary, a guidewire with hooked distal end is used to extract the plug. If for any reason—such as an hydroxyapatite plaque cap is encountered—plug excision is resisted, the operator can loosen locking nut 10 to rotate and reciprocate trepan tube 5.

Once trepan 6 is aligned to the inner surface of ductus wall 2, locking nut 10 is tightened to lock trepan tube 5 in position. Vascular diversion jackets, valves, and servovalves are unique in shape factor and in perforative, flow diversionary, and accessory channel features but not in terms of driving means, which are miniaturized versions of solenoids and tiny servomotors, mechanisms well known to mechanical and electrical technicians and engineers. Nonadjustable and limited to use as static flow diversion means, manually applied diversion jackets have no driving means. In the vascular tree, these are positioned at the inlets into bypasses and shunts that require no adjustments in flow rate.

Manually operated urinary valves such as used in a ureter-to-collection bag diversion prosthesis usually employ simple mechanical controls for adjustment such as push\pull, or Bowden cables, but can just as well use a remotely controlled solenoid, for example, especially if to route the Bowden cables creates the potential for strangulating intervening tissue. In order from most to least simple, vascular jackets include fixed flow diversion jackets, manually controlled bistable flow nondiverted-flow diverted valves, such valves and chokevalves when solenoid driven, and continuously variable servovalves under automatic control by an implanted sensor-fed microcontroller or microprocessor.

TABLE 1
Servo diversion and choke valve-jackets.

| | Diversion valve | | Choke valve |
|---|---|---|---|
| | | controllable | controllable |
| | all or nothing | fraction | fraction |
| Chute | yes | yes | yes—must be manually |
| | | | or servo-driven |
| Dome | no | yes, but not | yes |
| | | zero flow | |
| Cone | no | Yes, but not zero | yes |
| | | flow | |
| Driver | manual if no | manual or linear | manual or linear |
| | more than three | servomotor | servomotor |
| | solenoid if more | | |

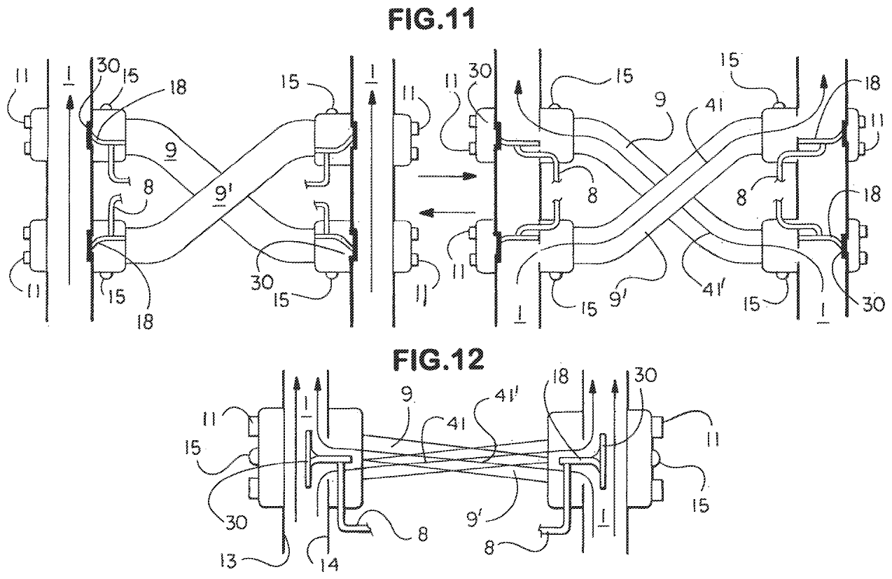

A sphincteric valve such as that shown in FIG. 11 of copending application Ser. No. 15/998,002 entitled Ductus Side-entry Jackets and Prosthetic Disorder Response Systems externally cinches about to constrict the substrate ductus from without and is structurally and functionally distinct from other vascular jackets, valves, and servovalves. Nonadjustable, the ureteral takeoff diversion jacket show in FIG. 2 is not a valve, which connotes adjustability, but rather a static prosthesis for placement in a patient with a missing or irreparable urinary tract. The diversion jacket is shown fully extended with vacuum hose having been disconnected from the rear end of trepan tube 5, after the side-entry tissue plug has been extracted and the diversion chute 18 extended into the ureteral lumen.

Nonadjustable, it requires a diversion chute, but since urine passing down the ureter should always flow freely out through the trepan tube and into the catheteric mainline, here referred to and further explained below, as a neoureter, and not accumulate, an ostium obturator, for preventing outflow into the diversion jacket is omitted. In contrast, in a urinary diversion valve which to allow the user to switch from normal urination during waking hours to diversion with no urge sensation as would awaken the wearer during the night, a ostium obturator is necessary.

In FIG. 2, part number 1 is the lumen, usually ureteral, 2 the luminal wall, 3 the outer shell of the diversion jacket, 4 viscoelastic polyurethane foam, 5 the trepan tube, and 6 the sharpened trepan leading edge of trepan tube 5. A water jacket is omitted as not required for placement, and an accessory channel not prescribed, diversion chute handle 8 connects to the rear end of flow diversion chute 18. When direct drug targeting is prescribed, accessory channel 8, a hollow capillary tube seen in drawing figures to follow, continues forward where diversion chute handle 8 is connected. Numerous apertures 40 entirely through outer shell 3 and foam lining 4, most outside the plane of the drawings, assure that the adventitia is not completely enclosed as would quickly induce atherosclerotic degeneration in the intima of the substrate ductus.

Accessory channel 8 would then move along the midline underside of diversion chute 18 and terminate in an aperture on the underside distal tip, or nose, of diversion chute 18, positioned to release a crystal solvent or drugs into ureteral lumen 1 to wet down urothelial lining 13 and 14 and then enter the bladder. Diversion chute 18 fully extended as shown, positive detent element 16 is engaged within receiving element 17. A mainline—in this case, an outflow catheter or neoureter, connected to the back end of trepan tube 5—is not attached. Diversion chute 18 is made of a material resistant to the adhesion of crystal, so that for many applications, an accessory channel will not be necessary.

Accordingly, when it is desired to target medication directly into the substrate ureter for passage down into a diseased urinary bladder, fixed as well as valvular ureteral takeoff jackets incorporate an accessory channel 8 rather than a diversion chute handle 8, that in the diversion jacket fixed in position once placed. However, where the bladder with the aid of medication is expected to recover, adjustable rather than nonadjustable diversion jackets should be used. Flow into the bladder rather than into a neobladder or collection bag is through nonjacketing side-entry connectors as described and illustrated in copending application Ser. No. 14/998,495, entitled Nonjacketing Side-entry Connectors and Prosthetic Disorder Response Systems,

Valves for placement along a ureter such as that shown in FIG. 2 are connected when the ureter is passing little if any urine and therefore pose no problem comparable to that encountered with diversion jackets and valves meant for placement along a vessel, where unless prevented, blood would run out. In basic ductus side-entry jackets such as that shown in FIG. 1, a water jacket 8 is used to restrain blood from outflow by directing a jet of water under sufficient pressure to prevent bleeding over the opening or ostium cut in the side of the ductus, typically, an artery where the blood pressure and pulse would pump blood out through the opening.

Once the jacket has been placed, accessory channel 8 makes possible the direct pipe-targeting of drugs into the jacket and the native lumen it encircles. In a urinary diversion jacket, where placement follows voiding so that no outflow occurs, or where some negligible extravasation from the substrate vessel is readily aspirated and unproblematic, a water jacket is omitted. However, when placed in conjunction with an implanted automatic disorder response system able to directly pipe-target drugs to the jacket automatically according to the prescription-program of the implanted microcontroller, one or more incorporated accessory channels is always present.

To cut the side-entry opening, or ostium, into the ureter, the trepan is moved forward with the chute retracted. As distal terminus of the mainline, the trepan is manipulated by loosening lock nut 10, freeing the trepan to be moved reciprocally or rotated. Once the opening has been cut, trepan 6 is retracted into alignment with the proximal urothelial surface, lock nut 10 used to fix it in position, and chute 18 deployed. To prevent jamming or sticking of diversion chute 18 when deployed after the jacket has been positioned, the segment at the distal end of the mainline with trepan distal edge is made of low friction fluoropolymeric tubing, such as polytetrafluoroethylene.

The accessory channel or sideline catheter is made of a polymer such as polytetrafluoroethylene in a thickness as will bend only so much as to allow the operator to advance the diversion chute into the ureteric lumen. If urging by edge of the entry hole to the side removed from the ureter at the bottom of the jacket is insufficient to guide the accessory channel tube forward, a small curved quarter round guide is bonded to that edge to assure smooth and properly aligned movement of the service channel tube through the entry hole. Sideways bendability of this material is, however, sufficient as not to interfere with any slight rotation or reciprocation of the trepan the operator may need to create the opening into the ureter.

If necessary despite the use of a suction pump as described above under Background to the Invention, mainline 9 with trepan 6 at its distal terminus can therefore be rotated from side to side to facilitate cutting the opening into the native lumen, then locked in position. Accessory channel 8 is otherwise made of a conventional catheter synthetic. Chute 18 is attached to the distal extremity of accessory channel 8 and moved forward into ureteral lumen 1 by pushing accessory channel 8 forward.

Visualization is unnecessary, full deployment signaled to the operator by an audible click when detent eminence 16 enters detent depression 17 posing resistance of the chute to further advancement. Sliding of accessory channel 8 with chute 18 is facilitated by collar 12 made of a hard and slippery gear grade nylon. The ostium cut and plug removed, diversion chute 18 is advanced to fully close off native lumen 1 and divert the flow of urine through mainline 9, here a neoureter as shown in FIG. 28. Passage continues through the components of the prosthesis required by the condition of the patient, and eventually into collection bag 149.

Fully extended (deployed, advanced), the typical 2.0 to 4.0 millimeters to span ureteral lumen 1, flow diversion chute 18 is slightly larger than the luminal diameter; so that made of a soft rubbery material and progressively thinned, or feathered, moving toward its periphery, and placed in apposition or abutment against a native surface itself compliant, chute 18 fully seals off the ureteral lumen beneath, or antegrade to it, from that retrograde above.

Such a fixed urinary and blood flow diversion jacket, shown in FIG. 2B, is suitable for use in a prosthesis where the need for adjustment after placement is not an issue. With the embodiment shown in FIG. 2, an upstream basic side entry jacket can be placed to periodically target medication to the downstream intima as an automatic drip controlled by the implanted microcontroller or microprocessor as might any accessory channel, while water jacket unrelated, or incorporated, accessory channel 8′ allows the diffusion through open cell viscoelastic polyurethane foam 4 of medication to target the downstream adventitia.

Mainline 9, temporarily connected to a vacuum pump to retract the plug of tissue cut from the side of the ductus thereafter serves as the permanent conduit conducting blood or urine out of, or in a shunt or bypass, into the ductus. Devised for application along a ureter rather than a vessel, incorporated accessory channel 8 with exit pore 31 beneath diversion chute 18 as well as an ostium obturator, or stopper to first prevent extravasation and once introduced into substrate native lumen 1, apportion blood between that tapped off and that to continue along the vessel is optional.

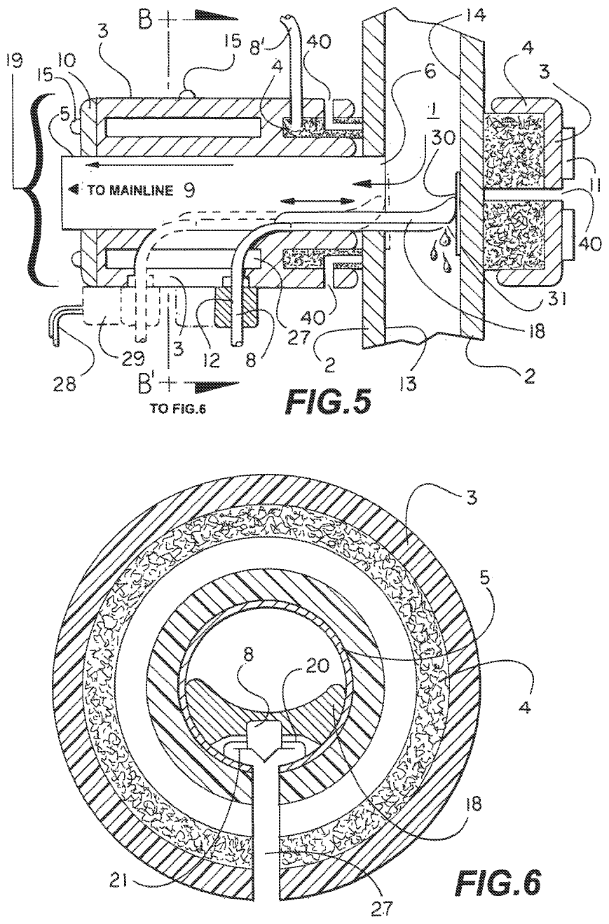

A separate line, smaller in gauge than mainline 9, the sideline 8, is connected to the outlet of a small flat drug reservoir implanted in the pectoral region to serve as the drugline or sideline 8 feeding into the accessory channel, also 8 to make it clear that both the line and accessory channel through the jacket or valve represent the same continuous passageway. Instead of a water jacket, valves for placement along vessels such as those shown in FIGS. 5, 7, and 8 incorporate an ostium obturator 30 which blocks blood from escaping while trepan 6 cuts the plug of tissue to create the side-entry opening, or ostium and once the ostium obturator is brought flush against the near endothelial surface 13 of lumen 1.

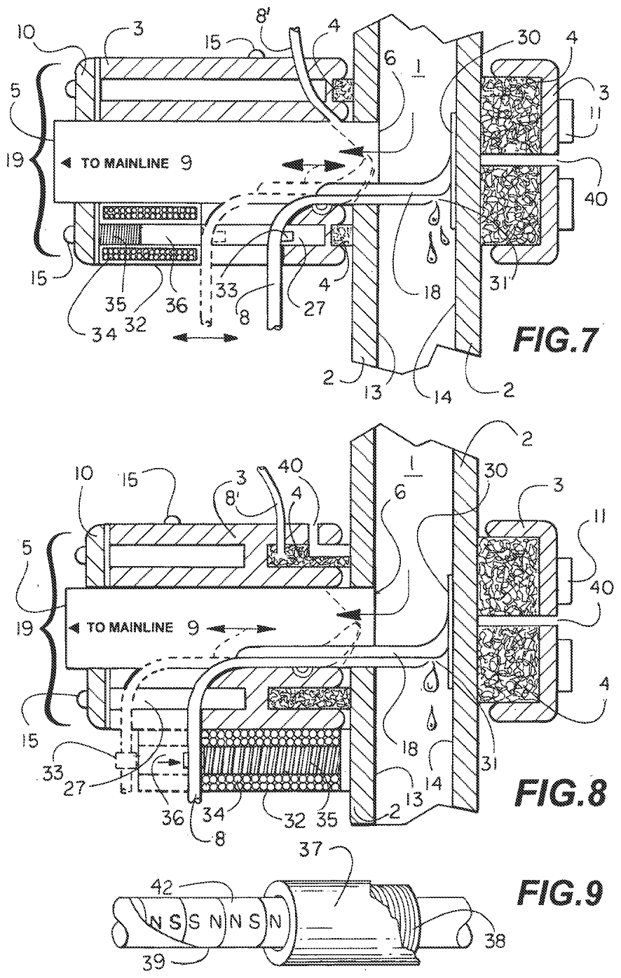

In a sudden switch organ transplant driven by plunger solenoids such as those shown in FIGS. 7 and 8, the ostium obturators are abruptly moved through ostium to rest flush against the endothelial lining on the far side 14 of the substrate vessel. In a vascular servovalve, the ostium obturator is initially positioned flush against the near wall 13 of lumen 1 by the operator through the use of a direct manual fine control mechanism such as that shown in FIGS. 10A thru 10C before electrical control is initiated, whereupon the microcontroller or control microprocessor registers this positional reference datum. Once this position is entered, the balance of the transplantation procedure and attendant administration of drugs as necessary is entrusted to the implanted microcontroller or microprocessor for execution under automatic control.

Adjustable, or controllable, diverters allow switching from nondiverted flow, that is, normal ureteral through-flow into the bladder or diverted flow into a bag, for example. This allows switching between the periodic release of medication into the bladder and wetting down the walls of the neoureters with an antimicrobial or crystal solvent, for example. Both nonadjustable and adjustable diversion jackets requiring the diversion chute to be slid at least forward, the sliding base plate at the bottom of the diversion chute and the sliding track or rail along which it rides, seen in the cross sections that follow are included in all diversion jackets.

Once the bladder has healed, the neoureters are endoscopically removed and the diversion chutes left in place to allow the continued release of medication directly into the ureters. Then the diversion jackets should be bilateral. Part number 10 is a lock nut to fix trepan tube 5 in position, 11 are spring hinges that urge the jacket into a closed position to stably cinch about the substrate ureter 12 is the accessory channel stabilizer, 13 is the urothelial lining of the operator-proximal luminal wall, 14 the urothelial lining of the operator-distal luminal wall, 15 suture loop to pass through suture if the ureter with jacket should be held away from neighboring tissue, 16 the sliding positive (male, eminent) detent, and 17 the receiving, or female, detent to retain diversion chute 18 in position unless the operator exerts sufficient force to move it.

Detents are unsuitable for use in servovalves such as shown in FIG. 10A, which are continuously adjustable and were it necessary, can be programmed to hold fast. Only non-servovalves, that is, bistable valves, which are manually set in shunt or bypass prostheses in the urinary tract such as shown in FIG. 2 and solenoid-driven valves such as shown in FIGS. 7 an 8, also suitable for nominally one-time setting of prostheses as irreversible, of which the use may be unavoidable for an emergency sudden bypass, or switch, organ transplant requiring the simultaneous energization of numerous such valves incorporate detents.

If necessary, such detents can be overridden through the application of somewhat greater than usual force. The reason servovalves are preferred for transplantation is the mid- and postprocedural versatility of these vis a vis nominally one-time solenoid driven valves. Servovalves, however, require a prescription-program for the implanted controlling microprocessor adapted for the specific patient, which requires lead-time that an emergency precludes. A solenoid-driven transplant can respond to a prescription-program limited to the direct pipe-targeting of drugs but not valve adjustments responsive to physiological indicia.

Where the use of solenoid-driven valves is unavoidable, greater vigilance of the patient and the manual administration of drugs, for example, is necessary. Restriction to the clinic rather than the automatic release of drugs in an ambulatory patient is in itself a deterrent. Except in a patient considered too frail to undergo revision, solenoid-driven valves should eventually be replaced with servovalves operating under a personalized prescription-program. Awareness of this eventuality is justified only when the need for immediate action does not allow adequate time for preparation.

Indicated by the absence of an ostium obturator, the diversion jacket shown in FIG. 2 is a ureteral or vascular takeoff jacket for placement at the inlet to a shunt or bypass, here a neoureter, where it will remain permanently as an nonadjustable full flow diversion prosthesis. Remote controlled adjustability of a takeoff jacket by means of push/pull, or Bowden cable or an electromechanical actuator renders it a valve, which not limited to full flow diversion, must incorporate an ostium obturator to prevent diversion, or outflow when the diversion chute is fully retracted, and assists the diversion chute in splitting the flow when partially extended.

The operator pushes accessory channel stabilization collar 12 forward until an audible click indicates the engagement of positive sliding detent or eminence 16 in stationary receiving detent or depression 17. The detent is not so resistant to disengagement that the operator is unable to retract it should the need arise. When placed endoscopically, this action is expedited through the use of a Bowden cable of the same kind as shown below in another embodiment whereby the wearer can adjust the diversion chute by turning a small dial on a small body surface port with urine outflow opening hole at the center. When used to aid placement, the cable is disconnected before closing.

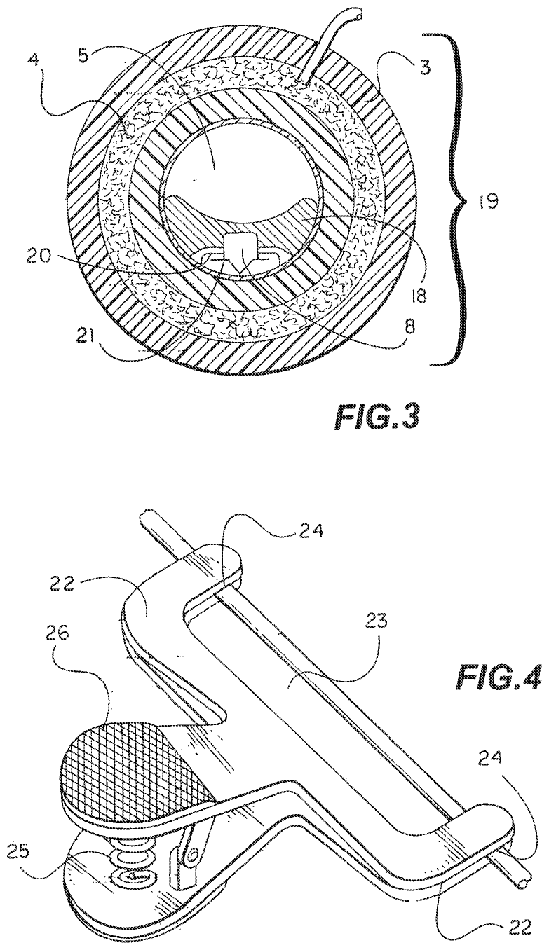

FIG. 3 is a cross-section taken along line A-A′ in FIG. 2 through side-stem 19 of the diversion jacket shown in FIG. 2 to provide greater clarity as to the structure of the accessory channel 8 at the bottom center of diversion flow chute 18, and that of sliding baseplate 20 which rides over slideway, or raceway, track, or rail 21.

Accordingly, as shown in FIGS. 3 and 6, the chute 18 with accessory channel 8 travel along a raceway or slideway comprised of horizontal extensions or wings 21 which to assure smooth and properly aligned movement ride within guide restraints. Over the segment of the proximal groove past the tubular accessory channel and extending toward the ureteral lumen, or distally, to the level half way into the ureteral lumen with the chute deployed, the groove is lined with thin inverted V stock stainless steel with a horizontal side wing to either side impressed by bending the V stock longitudinally along its length with a press brake or draw press.

The wings ride along the bottom of the terminal segment of the mainline with trepan leading edge beneath guides made of slippery gear-grade implantable nylon or polyvinyl chloride comprising an alignment track (raceway, slideway). In addition to smooth and properly aligned advancement of the chute into the ureteral lumen, the stainless steel (inox) liner supports the chute to prevent downward prolapse into the ureter and imparts the stiff relation to assure positive engagement with an audible click of the detents that signals full deployment. Both trepan and chute can be moved back and forth.

FIG. 4 shows a side-entry jacket or valve jacket placement clamp (straddle-clamp, forked vessel segment-straddling spring-loaded double clamp, forked double clamp). Most ductus can be clamped briefly, exceptions being the coronary and carotid arteries, which are never to be clamped however briefly. Unlike the basic side-entry jacket shown in FIG. 1 wherein the path through the lumen of trepan tube is clear of any obstruction that might pose interference to the extraction of the side-entry tissue plug into which trepan tube 5 must be inserted, a valve jacket which incorporates a diversion chute with ostium obturator at its distal tip, or nose, inside trepan tube 5, thus obstructing the trepan tube lumen as the passageway through which the plug must be extracted, necessitates an alternative method for removing the plug to clear the way for insertion of the trepan tube.

An advantage of the straddle-clamp or separate small clamps where that downstream is place first is that this facilitates use of a trepan tube. Elimination of the trepan tube necessitates the use of an ostium obturator greater in pliancy and resiliency than needed in a valve with a trepan tube. A straddle-clamp, that is, a forked ductus segment-straddling spring double clamp or side-entry or valve jacket placement clamp, provides a simplified means for plug extraction past diversion chute 18. The straddle-clamp shown in FIG. 4 is one of a set including clamps permanently positioned at different distances from one another.

Alternatively, a double clamp can be provided such that one handle controls two clamps which can be adjusted in separation and/or the rotational angle of each. Where the segment is small so that little blood would be lost, two separate clamps can be used, allowing placement of the first downstream to increase the pressure in the segment, and that upstream placed immediately afterwards, thus slightly dilating the segment (as well as the upstream artery), making insertion of the trepan tube easier.

A ureteral takeoff jacket or valve can be placed with the aid of a clamp briefly placed upstream to interrupt continued kidney outflow, but never during a ureteral jet; extravasation analogous to that possible with a vessel is not applicable (Baker, S. M. and Middleton, W. D. 1992. “Color Doppler Sonography of Ureteral Jets in Normal Volunteers: Importance of the Relative Specific Gravity of Urine in the Ureter and Bladder,” American Journal of Roentgenology 159(4):773-775; Jequier, S., Paltiel, H., and Lafortune, M. 1990. “Ureterovesical Jets in Infants and Children: Duplex and Color Doppler US [ultrasound] Studies,” Radiology 175(2):349-353.

The forked double clamp is intended to suddenly stop the flow of blood through a segment of a vessel for valve placement. When clamped, the blood in the segment between the clamp arms is trapped, maintaining the segment at its noncollapsed diameter, so that when the valve trepan with suction pump attached to the trepan tube is used to extract the plug, only the small amount of blood that had been trapped runs out (bleeds, extravasates). With the aid of the clamp, to insert the trepan into the side-entry opening, or ostium created, and lock the jacket in place onto the vessel or ureter is accomplished in seconds. The same procedure can be used in placing any basic side-entry jacket where the extraction of the tissue plug might prove problematic. The momentary interruption in blood flow is innocuous.

Blood in the trepan tube is readily washed out with heparin. Such a clamp can be used to expedite the placement of any type jacket about any type ductus. At least one sideline leading to what serves as the water jacket during placement of a basic ductus side-entry jacket and thereafter used as an accessory channel for the delivery of drugs is attached to the side-entry jacket before placement. Differing only in functional distinction at different steps during placement as clarified by the context and structurally continuous, a water jacket, sideline, and accessory channel are all designated by part number 8. A forked double clamp is structurally little different in structure than an office paper pinch clamps; however, the differences are important.

The differences are that the return force must be no greater than is essential to stop flow through the substrate vessel, the contact surfaces on the vascular adventitia must be cushioned using biocompatible materials, and rather than continuous from side to side, such clamps have a central portion removed to create two arms spaced apart by the length of the segment to be bounded. For such use, the clamp is either coated or made of spring stainless steel. Discouraged is a clamp with adjustable spring return force to allow the use of a single clamp with vessels of different diameters; too little compression will prevent neither partial flow nor continuous extravasation, and too great a force can result in intimal injury.

Instead, the clamps are made in different sizes with different spring return forces, thus precluding complications due to human errors in adjusting the return force as might result in intimal injury. Instead, each size clamp exerts a nonadjustable return or clamping force determined by the inherent and worked factors of the spring steel, spring stainless steel, titanium, or polymer used. FIG. 3 shows the simplest of many possible embodiments incorporating torsion, tension, or compression springs and made of metal or plastic.

In FIG. 4, 22 are the clamping arms to either side of straddle-clamp, 23 the space separating clamp arms 22, which defines the segment between clamp arms 22 for the entrapment of blood inside the segment to facilitate the removal of a side-entry tissue plug from the wall of the vessel to be jacketed and placement of the diversion valve. Parts numbered 24 are the adventitial contact area cushions, 25 the extension spring-backed hinge that provides the clamp spring return force, and 26 the thumb and index finger pads the operator presses together to place, or set, and remove, or release, the clamp. Provided it is coated The suction pump hose as temporary mainline 9 can be used to pull through the plug, and if successful, can then be detached for replacement with a catheteric mainline through which blood will outflow and exit or drugs enter and inflow.

Otherwise, the suction hose removed, the straddle-clamp is left in place just long enough to allow the use of a small dental pick or similar hand tool to extract the plug. The permanent catheteric mainline through which blood will flow out or a drug flow through can then be attached and the clamp removed. A stopper affixed to the nose of the diversion chute, or ostium obturator, which serves to prevent outflow through the trepan tube when diversion is not in use, appears in the diversion valves and servovalves to be described.

The ostium obturator then prevents outflow long enough for the opposite end of the mainline to be attached. The absence of an ostium obturator indicates that outflow during placement and thereafter is not a problem. The same use of straddle-clamps can be applied to the placement of any basic side-entry jacket where the extraction of the tissue plug might encounter interference, the brief interruption in blood flow innocuous.

FIG. 5 shows a manually operated urinary flow diversion valve wherein flow diversion chute 18 is driven by a push/pull control cable between the fully closed chute extended full flow diversion and fully open chute retracted normal flow through positions. The fully extended position is suitable for use during the night to avoid being awakened by the need to void, whereas the fully retracted position is suitable for use during the day to void normally. Other applications include preventing an interruption during a public performance to the extent that urge sensation is never felt.

With the embodiment shown in FIG. 5, an upstream basic side entry jacket can be placed to supplement incorporated accessory channel 8 and 8′ in periodically targeting medication to the downstream intima as an automatic drip controlled by the implanted microcontroller or microprocessor. Incorporated accessory channel 8′ allows the diffusion through open cell viscoelastic polyurethane foam 4 of medication to target the adventitia. Incorporated accessory channel 8 is available to drip medication downstream of diversion chute 18.

When accessory channel 8′ outlets in front of collapsed diversion chute 18, diversion chute 18 sweeps the medication over the small ostial trepan wound and delivers medication into substrate native lumen 1 when ostium obturator is not set flush against the proximal intima 13. When accessory channel 8′ outlets into the open cell polyurethane protective foam 4, medication continues to diffuse through foam 4 to medicate the adventitia, the extent to which the medication reaches circumferentially determined by the frequency and volume of the drip.

In any adjustable embodiment such as this, an ostium obturator 30, shown here fully extended for full diversion, is essential to prevent outflow through trepan tube 5 when diversion chute 18 is retracted to allow normal ureter-to-bladder drainage. In FIGS. 5, 7, and 8, the ostium obturator, effectively a tiny version of an especially elastic and compliant rubbery polymeric tub drain stopper, is shown both fully extended and ‘ghosted’ to show it prior to deployment when it is retracted in the side-stem outside lumen wall 2. Accessory channel 8 for the delivery of drugs into the substrate ureter to flow thence into the bladder outlets at diversion chute 18 nose underside just within the ostium obturator 30 through opening 31.

A jacket of this type can be provided with a diversion chute detent-fixed in position with or without an accessory channel as shown in FIG. 1 or can be switched between chute 18 fully open or fully closed positions. When switchable thus, the diversion jacket is a valve capable of continuously adjustable control but degrees of extension between fully open and closed are not pertinent for the wearer who usually wishes full diversion during the night and when performing before an audience. However, this does not preclude the use of a mid-way position to delay voiding either normal or through the neobladder shown below. Accordingly, such a manually controlled valve is unsuited for use along the vascular tree where an implanted micro master controller can continuously apply fine adjustment to the valve in response to sensor data continuously fed to the controller.

FIG. 6 shows a cross-sectional view of the diversion jacket shown in FIG. 5 along line B-B′ to show diversion chute 18 handle or accessory channel 8 and diversion chute 18 reciprocal adjustment clearance slot 27 through which diversion chute 18 passes when driven by push/pull cable 28, cable 28 connected to stabilization collar 12 by coupling block 29. The rail device upon which chute 18 rides when advanced into or retracted from the substrate native lumen 1 is the same as that shown in FIG. 3 with the difference, however, that section line A-A′ indicated in FIG. 2 is not through the part of the valve where slot 27 must be cut out for passage of the accessory channel 8 as is the case along section line B-B′ in FIG. 6

To eliminate the need for direct manual contact of the operator with stabilization collar 12 and thus expedite endoscopic placement of a nonadjustable urine flow diversion jacket with optional but usually preferred accessory channel 8 for the direct delivery of drugs into the ureter 1 and bladder, cable 28 can be adjusted by means of a mechanical foot pedal control similar to that on a lawn tractor, then—retraction of chute 18 in this static prosthesis thereafter unnecessary—removed.

FIG. 7 shows a diversion jacket with flow diversion chute 18 deployed by a highly damped nonsparking miniature plunger solenoid. This allows the deployment of multiple chutes at the same instant by pressing a single switch, as is necessary in performing a sudden switch heart transplantation, for example. The device is intended to shift diversion chute 18 suddenly to the fully extended position, there to be left in place. Bistable, that is fully retracted or extended but unable to be positioned at a point intervening between these, the valve is actually less adjustable than is one cable driven, but allows remote control over multiple such devices, essential in a sudden switch solid organ transplant, for example.

With the embodiment shown in FIG. 7, as with any jacket-integral, or incorporated accessory channel, an upstream basic side entry jacket can be placed to periodically target medication to the downstream intima as an automatic drip controlled by the implanted microcontroller or microprocessor. Incorporated accessory channel 8′ allows the diffusion through open cell viscoelastic polyurethane foam 4 of medication to target the adventitia. When incorporated accessory channel 8′ outlets into the open cell polyurethane protective foam 4, medication continues to diffuse through foam 4 to medicate the adventitia, the extent to which the medication reaches circumferentially determined by the frequency of the drip.

When incorporated accessory channel 8′ outlets just ahead of ostium obturator 30 while ostium obturator 30 remains collapsed at the distal tip of diversion chute 30 prior to being deployed, ostium obturator 30 sweeps the medication, typically an anti-inflammatory, over the edges of the small ostial wound just cut. Where accessory channel 8′ with outlet into foam 4 remains effective indefinitely, positioning accessory channel 8′ to outlet just ahead of ostium obturator 30 prior to its deployment results in a reduction in its effectiveness at delivering medication into lumen 1. At all times, incorporated accessory channel 8 remains available to release medication through outlet pore 31 into the downstream flow.

Simultaneous actuation is by the operator or an assistant by direct wire connection, the wires thereafter removed and the jacket with accessory channel or channels left in place. In certain locations, the anatomy will better accommodate a valve of this configuration rather than that shorter shown in FIG. 8. If needed, Suture loops 15 at the abaxial or butt end of the valve side stem allow suture to be run through for connection to nearby connective tissue to aid in defraying the weight of the valve. Unneeded suture loops 15 are easily trimmed away with a scalpel.

Bistable control thus is distinct from control gained with a servomotor-driven diversion valve such as those shown below, wherewith continuously and valve to valve variable control is exerted by an implanted microcontroller fed pertinent data from implanted sensors and applied by the controller prescription-program. In a patient requiring the simultaneous surveillance and treatment of comorbidities, an implanted microprocessor serves as the master controller over a hierarchical control system programmed to seek out and apply that multimodal therapeutic response most likely to achieve the optimal homeostatic result across all comorbidities during and following a transplantation procedure, for example. The control of comorbidities can critically affect the sustainability of an organ transplant.

More complex control thus requires multiple command-execution channels, which unless posing a risk of strangulating tissue, are delivered along implanted wires. Otherwise, wireless such as radio remote control is used, each actuator singled out through the use of a unique carrier frequency. Part numbers in FIG. 7 are the same as in FIG. 5 with highly damped miniature nonsparking plunger solenoid plunger solenoid 32 replacing the cable drive, 33 the connector of the solenoid plunger (thrust rod, slider, shaft) to accessory channel 8, 34 the solenoid electromagnetic coil or winding, 35 the solenoid compression spring, and 36 the solenoid plunger.

Also in FIG. 7, parts numbered 40 are the ‘breathing’ apertures, which can be slits, or round holes, for example, to allow contact with the outer body cavity of the adventitia, such communication long established as necessary to prevent atherosclerotic degeneration at the intima due to compression of the nervelets, or vasa nervora, and fine blood vessels, or vasa vasora of the adventitia. Such perforations or apertures are numerous, most off the plane of section depicted in the drawings.

In FIG. 8, highly damped miniature nonsparking plunger solenoid 32 has been positioned in reverse beneath rather than in front of accessory channel feedline 8 beneath side-stem 19 to pull rather than push accessory channel feedline 8 used as the diversion chute handle. Part numbers in FIG. 8 are consistent with those in the other drawing figures. This arrangement allows the valve to be shorter expediting miniaturization for use in an infant, for example. In addition to the use of silver wire in the windings and the use of lightweight plastics, a significant reduction in size and weight for use in a small child is the use of servovalves which incorporate 2-way or bidirectional diversion chutes such as shown in FIGS. 11 and 12.

Accessory channel 8′ allows an automatically released drip to diffuse through the open cell viscoelastic polyurethane adventitia protective foam 4 to wet the adventitia. Were accessory channel 8′ continued through servovalve jacket shell 3 to outlet into the trepan tube 5 just in front of ostium obturator 30 before ostium obturator 30 is advanced into substrate ductus lumen 2, the medication released would coat the ostium about to be cut by trepan 6 at the front end of trepan tube 5 but would then become ineffective for further treatment of the intima. However, both positionings can be incorporated into the valve jacket, just as both can be incorporated into the ductus side-entry servovalves shown in FIGS. 5, 7. 9, and 10.

FIG. 9 shows one of several servomotor types suitable for use in a vascular servovalve wherein it is positioned within the valve jacket as are the plunger solenoids shown in FIGS. 7 and 8. The criteria for a linear servoactuator include precision performance, and suitability for miniaturization to achieve a size and weight least likely to cause discomfort. Unacceptable is any motor design that significantly deviates from cylindricality, such as a motor mounted to extend away from the central or long axis.

Linear actuators include those which combine a rotary motor with planetary gearing and a machine screw drive to convert rotary to linear motion and those intrinsically linear. Of the two, intrinsically linear designs are direct-drive, eliminating cogging and backlash, and better lend themselves to a tighter cylindricality at the driver end, small size, and low weight. Of the various types of linear motors—iron core, U-channel, tubular linear, miniaturized versions of linear induction machines—one that lends itself to miniaturization and a compact cylindrical shape and provides the level of precise control suitable for use in vascular valves is the pulse tubular linear shaft motor.

Such a motor is brushless, of high flux density with a conformation that affords maximum magnetic efficiency, zero cogging, highly reliable, and precise, as well as direct drive. Void of mechanical linkages, hence, backlash-free, the motor requires no maintenance, is virtually instantaneous in response, and from the standpoint of compactness, is scarcely deviant from a straight tubular conformation, making it suitable simple to adapt for use in infants. As shown for plunger solenoid diversion jackets in FIGS. 7 and 8 respectively, the linear or other form of servomotor can be positioned within the valve with connector 33 to push or pull accessory channel 8, without the limitation in performance of full displacement from one end of the stroke or the other.

In FIG. 9, adapted from illustrations published by Nippon Pulse America, Radford, Va., such a pulse linear shaft motor comprises forcer, here adapted with cylindrical housing 37, enclosing adjacent circular electromagnet coils 38 in circumferential and surrounding relation to the motor shaft 39 which incorporates successive pairs of cylindrical neodymium iron boron permanent magnets 42. The magnets are arranged along the shaft so that successive pairs consist of a forward magnet with south pole at the leading end, for example, and north pole at the trailing end, the following magnet in the pair reversing the poles so that each pair has facing north poles where the members of the pair come together and adjacent south poles where the successive pairs come together.

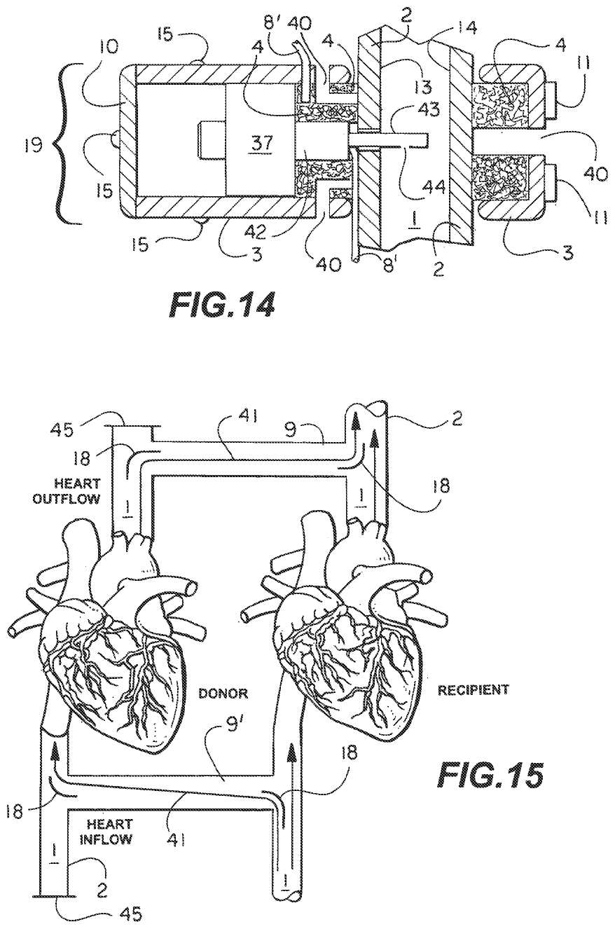

Shown in FIG. 10 depicting a vascular servovalve with direct manual fine control attachment and FIG. 14 of a servochoke, miniaturized versions of such a tubular linear shaft pulse servomotor are suitable for use in any side-entry valve or servochoke. At the sizes required, most tiny, the neodymium magnets should not present an absolute sum weight such that the normal alignment provided by the jacket with foam lining, and if necessary, the aid of suture run through a suture loop 15 and wound about the substrate ductus upstream will exert a levering force to compress or flex the substrate ductus. Accessory channels in the vascular servovalve shown in FIG. 10 can include any or all of those described in conjunction with the valves shown in FIGS. 2, 5, 7, and 8 bearing part number 8 or 8′.

In placing a valve along a carotid or coronary, it is important that no interruption occur in the flow of blood. Accordingly, the use, however momentary, of a clamp, one such shown in FIG. 4 is disallowed. In entering such an artery, first the suction pump applied to the mainline is used to pull the wall of the artery past the trepan to cut a plug of tissue. The pump is reversible and equipped with a limit switch, so that the instant resistance to the vacuum disappears, the pump shuts off. The plug may have adhered within the circle incised, but more likely has been extracted, but blocked by the diversion chute and the undeployed, therefore collapsed, ostium obturator, has become lodged at the front of the trepan tube. The plug must be removed with no interruption in the flow of blood as could induce a cerebral or myocardial infarction.

Now 1. The plug must be removed, and 2. Once the plug has been removed and the diversion chute passed into the substrate ostium, the ostium obturator must fit flush over the ostium to prevent blood from leaking.

Removal of the plug is by drawing the trepan tube from the ostium where the operator has wetted his glove with a heparin and positioned the thumb adjacent to the opening. The instant the trepan is removed, the operator slides a thumb over the opening, preventing blood from escaping and an assistant reverses the vacuum pump to expel one or more forceful discharges of air, driving the plug out the front of the trepan tube. If as improbable, following one or more strong puffs, or ‘blasts,’ of air the plug still adheres at the front of the trepan tube, an assistant quickly retrieves it with a tweezers or dental pick. The trepan tube is reintroduced into the opening simultaneously with the sliding away of the thumb and the valve jacket allowed to close about the artery.

Because the walls of larger arteries incorporate elastic laminae and smooth muscle, and because the ostium obturator must comprehend elasticity and resilience consistent with the need to fully deploy from a collapsed condition, despite the use of the vacuum pump or controller in an attempt to retract the obturator into a flush fit against the inside of the side-entry opening, or ostium, some rebound will usually occur which only precise manual control can overcome. Shown in FIGS. 10A thru 10E and 25 is direct manual fine control attachment device to a vascular servovalve. The attachment is used only during attachment of the valve and removed before closing. It is then sterilized by autoclave or ethylene oxide gas for use with another valve of the same type and size.

Attachment of the direct manual fine control to the servovalve is accomplished preoperatively and does not add to the duration of the operation. Adjustment of the ostium obturator 30 to fit flush against the ostium can only be accomplished by the operator mid-procedurally once the diversion chute has been moved into the lumen of the substrate native vessel. Attachment of the direct manual fine control device can serve as a precaution should a sudden vacuum force exerted on the ostium obturation by the suction pump or the automatic program itself not seat the obturator flush against the ostium. FIG. 25A illustrates the prefinal step in placement in a bypass to both the internal and external carotids, and FIG. 25B the same where only the internal carotid must be bypassed just before the attachments, each matched in size to its valve, are removed.

To minimize the weight of the servomotor, coil 38 is wound with silver wire. To this end, the longitudinal extension of the valve and consequent levering moments of force, is kept as small as possible. The pulling rather than pushing driver configuration shown in FIG. 8 is shown adapted in FIG. 10 to allow shortening the valve, reducing its moments of force, and therewith any tendency to lever under gravity, imposing a bending force on the substrate vessel.

FIG. 10A shows a puller-configured double outlet tubular pulse linear shaft motor-driven vascular side-entry diversion servovalve of the kind shown in FIG. 9 with the addition of a detachable direct manual fine control device positioned beneath servovalve side-stem 19 shown in FIGS. 10A thru 10E suitable for performing a carotid endarterectomy, for example, where one valve on the common carotid bypasses blood into the internal of the internal and external carotids. As shown in FIG. 25B, where the external carotid is unaffected, only a bypass to the internal carotid is needed. In both, the diversion chute of the valve on the common carotid must remain partially retracted to allow blood to pass through the common carotid into the facial, lingual, and superior thyroid arteries, as well as numerous arterial ‘twigs.’

Attached to the servomotor housing before use and detached after, the manual fine control, or override, device is used to manually initialize the precise horizontal position of the servomotor shaft if necessary before valve placement along the substrate vessel. The manual fine control device is housed in a separate enclosure which is attached to the motor housing during use and thereafter removed. The direct manual control device assures the correct alignment of the motor shaft for its attachment and allows the operator to apply small adjustments in the position of the servomotor shaft, hence, correct starting position of the ostium obturator to assure a flush nonleaking fit over the ostium.

The double outlet implements use of the valve a carotid endarterectomy bypass such as that shown in FIG. 25A, which can be used intraoperatively or, where the carotids are heavily diseased or must be resected to remove tumors, as a permanent prosthesis. The direct manual fine control device allows the operator to move motor shaft 39 without snap-back during a condition of no current flow. That is, the direct manual fine control device allows the operator to apply small adjustments in extension or retraction into the substrate native lumen 1 of the ostium obturator at the distal tip of diversion chute to fit flush against the ostium when the implanted control microcontroller or microprocessor has not been additionally programmed to respond to a handheld remote control to allow off-line tiny shifts of the diversion chute and the deployed diversion chute 30 is not flush against proximal intima 13 to prevent any blood from escaping.

### Overall Structure or the Direct Manual Fine Control Device

Part numbers not unique to the direct manual fine control device shown in FIG. 10A are consistent with those shown in the preceding figures. The device enclosure is made of a tough clear transparent polymer such as polyethylene terephthalate, the internal parts rendered radiopaque with contrast, such as Danfoss Tantalum Technologies Danfoss Coating®, thus allowing the operator to directly observe its internal operation whether the valve is applied to the substrate native lumen with side-stem 19 directed to the left as depicted in FIG. 10A or to the right.

To fully deploy, that is, expand from its collapsed position while remaining within trepan tube 5, ostium stopper, or obturator, 30 must enter substrate native lumen 1 to a point slightly beyond a fully flush relation, possibly allowing a slight emission of blood into trepan tube 5. Trepan tube 5 blocks the blood from fouling either the protective 4 or the ‘breathing’ apertures 40. However, the actual size of the largest valves for use along the aorta is many times smaller than the valves have been shown for clarity in the drawing figures, so that unless an anticoagulant such as heparin, is released from an upstream jacket or through accessory channel 8, even a small amount of blood could prove a hindrance. However, an anticoagulant should be avoided in some patients, making an alternative approach necessary. Were blood to foul the protective foam separating the valve from the substrate vessel, the valve is supplied with replacement foam.

If used, the anticoagulant is released just before ostium obturator 30 is advanced into the native lumen 1. The advancement of ostium obturator 30 then sweeps the anticoagulant forward to wet the cut edges of the ostium. Ostium obturator thus serves three functions:

1. In advance of actuating diversion chute 18 it squeegees an anticoagulant or any other drug in fluid form through the ostium, thus immediately medicating the tiny perforating wound created.
2. While held against the proximal intima of the substrate native lumen, ostium obturator 30 prevents blood from leaking into trepan tube 5, and
3. It defines by assisting to separate into separate flow columns, hence, the relative proportion, of blood to be diverted through trepan tube 5 and that to continue antegrade past it.

When fully automated, the servomotor may be programmed to retract ostium obturator 30 immediately upon insertion of ostium obturator 30 through the ostium just enough to assure that ostium obturator 30 rests flush against the proximal internal surface of the substrate native ductus. Alternatively, the section pump can be used to generate a vacuum that pulls the obturator flush over the ostium. Normally, the process can proceed without interruption. If for any reason, however, the operator sees that a completely flush fit has not been accomplished, the direct manual fine control device, used with the power turned off, can be used to prevent even a negligible amount of blood from escaping. Precautions notwithstanding, should blood enter the foam in an amount that could significantly foul its permeability, the foam affected is quickly replaced with small pieces thereof supplied with the valve.

To allow the operator to switch between right and left hand sides for direct observation of the direct manual fine control device mechanism from either side as essential to direct the side stem 19 to the right or left, for example, the device housing is transparent, and an engagement arm consisting of arms 77 and 77′ with end receivers 78 and 78′ makes it possible to alternately engage and disengage lead nut 72 so that rather than functioning as a screw, it rotates lead nut 72 connected to a locking mechanism in the form more or less of a capital letter Y, where either arm allows such engagement each on its respective side. Engagement arm piece 88 is made in one piece of die cut stainless steel or titanium alloy sheet stock, is burnished, and projects left and right-hand arms 77 and 77′ upwards.

### Internal Structure of the Direct Manual Fine Control Device

As shown in FIG. 10B. left hand arm 77 and right hand arm 77′ further diverge toward their respective sides as these rise, and at their upper ends, engagement arm piece 88 connector 76 catches 78, a triangular socket, on left arm 77 and 78′ on the right arm 77′, are poised so that much as a small socket wrench wraps about a bolt head, rotation of the engagement arm piece 88 as shown to the right causes left handed arm triangular socket, or catch, 78 to engage the left side projection of stationary engagement arm piece connector 76. To not be deflected when engaged, the engagement arm piece 88 must be inflexible, to which end it is preferably die cut from stainless steel or titanium alloy sheet stock.

To securely retain the projection of the engagement arm piece connector 76 within triangular sockets, or catches, 78 and 78′ the sides of these are wide enough to form reentrants too deep to allow engagement arm piece connector 76 to slip out when driven. Turned clockwise with moderate force to prevent accidental rotation, control knob 71 overcomes its central detent (unshown as behind knob 71) to move engagement arm piece 88 until an audible click indicates that the right-hand detent has engaged.

Turning now to FIG. 10C, to rotate engagement arm piece 88, lead nut 72 must be locked to control knob 71, while to rotate leadscrew 73 so that lead nut 72 can move along leadscrew 73, adjusting the proximal end of motor shaft 39 and therefore diversion chute 18 without at the same time rotating engagement arm piece 88 requires that lead nut 72 be unlocked from control knob 71. That is, leadscrew 73 either rotates together with lead nut 72 and engagement arm piece 88, or lead nut 72 is disengaged from the engagement arm piece, allowing engagement arm piece 88 to move along leadscrew 73 to extend diversion chute 18 into the substrate native lumen or to retract diversion chute 18.

FIG. 10C shows push/pull control cable knob 79 at the center of screw rotation control knob 71 when depressed overcomes the restorative force of small compression spring 81 to drive a Bowden cable-like flexible push-pull control wire forward so that its distal end is inserted thru leadscrew 73, then curves upwards into control wire receiver 75 locking knob 71, leadscrew 73, and lead nut 72 so as to rotate the engagement arm piece 88 as a single part.

To allow its removal once ostium obturator 30 has been correctly positioned flush against the proximal intima of the substrate vessel, thus covering over the ostium, the direct manual fine control is housed in a separate enclosure 91 attached to the bottom of enclosure 92 housing servomotor 37. Attachment of enclosure 91 to enclosure 92 is by insertion of pawl 93 into the bottom of a complementary receiving channel at the junction of left and right-hand engagement arm piece arms 77 and 77′. From FIG. 25, it will be clear that characterization of the enclosures as that upper or lower pertains when attachment to the substrate ductus is not inverted as are valves 89 and 90.

To allow the direct manual fine control device to be quickly and easily attached to and detached from the upper enclosure 92 housing servomotor 37 at a single point, engagement arm piece 88 must be positioned firmly enough to allow the insertion and withdrawal of intromitting pawl 93. To this end, the intromittent pawl receiving opening, or pawl receiver, at the bottom center of engagement arm piece 88 is tapered, or flared, wider at the bottom and curves upward to guide intromitting pawl 93 into a close enough relation to the opening that the operator need not direct undue attention or engage in an effort of precision to insert it. Shown in FIGS. 10C and 10E, the intromittent pawl receiving opening at the bottom center of engagement arm piece 88 then rises upwards into a friction fitting upper section of pawl receiver 99.

To meet these requirements, engagement arm piece 88 is suspended in its starting and ending disconnected and centered position shown in FIG. 10B by a miniaturized version of a miniature endoscopic-type grasper indicated in FIG. 10B with more detail provided in FIG. 10E and implemented as described below. The engagement arm piece must also be free to move from side to side to engage engagement arm piece connector 76 from either side as well as move with lead screw 72.

Accordingly, miniature endoscopic-type grasper 96 stably maintains engagement arm piece 88 in position, allowing the operator to insert intromittent pawl 93 into the bottom of engagement arm piece 88 and withdraw it once the adjustment in the horizontal position required has been completed, whereupon pulling down on enclosure 91 withdraws pawl 93 from the pawl receiver at the bottom center of engagement arm piece 88 for the procedure to proceed.

Grasper 96 is mounted to the side of manual control device housing 91, its shaft push/pull knob outside and shaft and grasper clamping pads, or jaws 142 inside housing 91 by a grommet-like surrounding ring, or torus, of stainless steel or titanium. The internal surface of the ring is broad enough to assure fitting about the grasper shaft as to disallow any play of grasper clamping pads 142 due to off-axis displacement at the ring when loaded and along with the shaft, is polished for nonresistant sliding to allow the clamping about and retreating of grasper clamping pads 142 from engagement arm piece 88.

Grasper shaft movement forward into manual fine control device housing 91 to stabilize engagement arm piece 88 by seizing it between grasper clamping pads 142 of grasper 96 before use so that insertion and removal of intromittent pawl 93 into the engagement arm receiver at its bottom center and will be quick and easy is limited to 5.0-7.0 millimeters by detents 94 outside enclosure, or housing 92 just in front of the grasper push/pull control knob 97 at the proximal end of grasper shaft 98, the other detent 95 along grasper shaft 98 inside housing 92. As indicated above, prior to use before engagement, arm piece connector 76 is shown in FIG. 10B ready for engagement either by engagement arm piece connector triangular socket, or catch, 78 on left arm 77 and catch 78′ on the right arm 77″before and after use in FIG. 10B.

Grasper 96 requires only one degree of freedom, that reciprocal to move its grasper clamping pads 142 to and away from engagement arm piece 88, positioned along the axis of movement at the displacement where the ostium obturator 30 will most often fit flush over the side-entry wound, or ostium, so that engagement arm piece 88 remains fixed in position for connection of the direct manual fine control device by soundly grasping engagement arm piece 88 between its grasper clamping pads 142. Requiring only reciprocal movement, grasper 96 is rigidly constrained to eliminate radial deflection.

Insertion and withdrawal of intromitting pawl 93 into the engagement arm receiver at its bottom center is manual. Thus, attaching and detaching the separably housed manual control device from engagement arm piece 88 is accomplished by the same action that connects engagement arm piece 88 for use. Grasper clamping pads 142 have miniature high coefficient of friction rubbery-lined rectangularly shaped grasper clamping pads 142 where the pad lining material can include vacuities to increase retention through the suction created when compressed and are dimensioned to interface with most of the engagement arm piece arm 77 at triangular socket, or catch, 78 or 77′ at catch 78′.

Engagement arm piece 88 is held rigidly in place while clamped by between the grasp grasper clamping pads 142 by a spring powerful for its size, the type spring, located at the junction of the linkage at grasper clamping pads 142, noncritical. To facilitate its quick location and use later by the operator or an assistant, grasper shaft push\pull control knob 97 extends out from the side of servomotor housing 92 to remain ready for later reuse when the shaft is retracted and the manual fine control device, no longer needed, is pulled off from the servomotor housing. No separate control to open and close grasper clamping pads 142 as might hinder the user are required at grasper knob 97.

Instead, a small rod inside the grasper shaft is connected to a linkage familiar to those in the art which opens grasper clamping pads 142 as the edge of engagement arm 77 is approached, and when contacted, a small spring-loaded bar at the junction of grasper clamping pads 142 is depressed to release grasper clamping pads 142 under the restorative force of the spring thus tripped. The unilateral grasper is used when attaching manual fine control deice housing 91 to motor housing 92 before use while outside the body and is easily retracted after use when inside the body by nudging up and slipping a finger the valve to retract grasper clamping pads 142. The linkage is so devised that rather than closing against the sides of engagement arm 77 in scissors fashion as would result in an uneven clamping force, grasper clamping pads 142 remain in parallel relation.

Withdrawal of the grasper by pulling out its push/pull control knob reverses this action, that is, opens grasper clamping pads 142, releasing engagement arm 88. FIG. 10C shows that in order to retract control wire 80 from lead nut 72, thus disconnecting lead nut 72 from engagement arm piece 88, freeing lead nut 72 to pass along leadscrew 73, push/pull control cable knob 79 must be pulled out as far as push/pull control cable knob 79 will allow, thereby retracting control wire 80 entirely from wire receiver 75 connecting engagement arm piece 88 to lead nut 72 as well as lead nut 72. This allows rotation control knob to rotate lead screw 73 rather than rotate lead screw 72 with engagement arm piece 88.

FIGS. 10A thru 10E show a vascular servovalve with direct manual fine control device attached and double outlet suitable for allowing a carotid endarterectomy free of complications due to ischemia, adverse fluctuations in blood pressure, as well as the risks of cerebral or myocardial infarction. In FIG. 10A, part number 82 is the male and 83 the female components of the bypass-line connector from the right common carotid to the internal carotid, and 84 and 85 respectively, those for connection of the bypass line to the external carotid. Part number 86 is the line to the internal carotid, and 87 that to the external carotid. The connections of the bypass lines from the common to the internal and external carotids is shown in FIG. 25.

To facilitate use of control knob 71 when the small button at its center must be depressed to lock lead nut 72 to engagement arm piece 88, rotation control knob 71 has projections 74 about its periphery. Accordingly, rotation of control knob 71 while depressing its central push/pull control cable knob 79 to the right as shown in FIG. 10B thus rotates engagement arm piece 88 to the right causing triangular socket, or catch, 78 to engage engagement arm piece connector 76 on the left, while rotation of engagement arm piece 88 to the left causes triangular socket, or catch, 78′ to engage the engagement arm piece connector 76 on the right side of engagement arm piece connector 76.

Rotation control knob 71 is fixed at its center position by a detent comprising a depression and complementary projection sufficiently retentive to preclude its inadvertent rotation. Accordingly, while push/pull control cable knob 79 is not depressed, rotation control knob 71 cannot rotate engagement arm piece 88 shown in FIG. 10B connected to leadscrew nut 72 in order to rotate engagement arm piece 88 with it but rather rotates leadscrew 73.

That is, when, rotation control knob 71 is rotated clockwise while small button 79 is not depressed, rotation control knob 71 rotates leadscrew 73 to drive lead nut 72 and therewith, accessory channel-feeding drugline 8 forward, hence diversion chute 18 further into lumen 1 of the substrate ductus, typically an artery. Turning knob 71 counterclockwise while push/pull control cable knob 79 is not depressed does the opposite, that is, retracts diversion chute 18.

FIG. 11 provides a schematic representation of solenoid flow diversion jackets or vascular servovalves as fully retracted, or open, on the left to allow nondiverted flow, and on the right, fully extended, or closed, to divert all flow from the substrate to the other vessel. With solenoids movement is limited to full extension or retraction to remedy a transposition of the great arteries when the defect can be addressed without cardiac entry or to accomplish a sudden switch transplant. The part numbers consistent among the drawing figures, 8 are the accessory channels, 9 and 9′ the directionally distinguished mainlines, and 18 the diversion chutes. In FIGS. 11, 12, and 13, 41 and 41′ points to directionally distinguished stream or flow lines.

To best facilitate application, jackets with the aid of a straddle clamp as shown in FIG. 4 or incorporating an ostium obturator 30 and servovalves can be positioned along their respective ductus before or after connection of the jackets or valves to their mainlines 9 and 9′. The arrows at the center denote that the action is reversible. When used for reciprocal cross-circulation during a switch transplant, this reversibility allows the immediate truncation, or ‘bailout’, of the operation by fully extending the flow diversion valves if a solenoid-driven sudden switch, or a variable retraction is a servo-driven metered switch, usually, with ensuing resumption.

Differing from plunger solenoid driven diversion jackets in allowing instantaneously continuous adjustability between full extension and retraction, the applicability of vascular servovalves extends to metered switch transplantation whereby an implanted microcontroller dynamically adjusts the valves to the achieve optimal acceptance by the recipient based upon the data provided to it by implanted physiological sensors.

Mainlines 9 should be kept as short as possible by positioning the donor and recipient to best accommodate the chirality only when the difference in distance is significant. Accordingly, whether mainlines 9 and 9′ crisscross or decussate in bias depends upon how the subjects are disposed in relation to one another. The directions of accessory channels 8 serves pictorial clarity; in fact, these are routed as least disturbs neighboring tissue.

FIG. 12 shows the use of bidirectional side-entry diversion servovalves to take the place of separate valves as shown in FIG. 11 where, in particular with neonates and infants, space to accommodate separate valves to perform a metered switch transplant is at a premium. Whereas FIG. 11 shows the separate valves switched from fully retracted from the substrate lumen, or closed on the left, thus playing no part in the normal flow of blood through the substrate lumen, and fully extended, or open for full flow diversion on the right, FIG. 12 shows the bidirectional valves at the half-way setting to allow half the flow to be exchanged between recipient and donor and half to continue the flow of blood native to the subject, the extent of mixture throughout either circulatory system contingent upon the duration of exchange.

When the defect does not reach down into the roots of the aorta and pulmonary artery, the same arrangement using solenoid-driven diversion jackets is suitable for the extracardiac correction of transposition of the great arteries in a neonate or infant. The half-way position denotes servovalve rather than solenoid control, this position exemplary for what is actually the continuous variability essential for metered switch transplantation. The use of bidirectional rather than separate servo-driven valves indicates that depicted is a moment in a metered transplant in a neonate or infant.

The valves controlled in a complementary manner, a complete shutoff or lack of outflow equates to the concurrent lack of inflow; otherwise, the volume of blood outflow equals the volume of inflow. Were the valves used in a neonate or infant to perform an extracardiac transposition of the great arteries, an adverse immune reaction would not be involved, so that to adjust and monitor the response would not be necessary. Bidirectional diversion chutes can also be incorporated into highly damped nonsparking solenoid-driven diversion jackets where the space-saving attribute facilitates the placement of duplicate jackets.

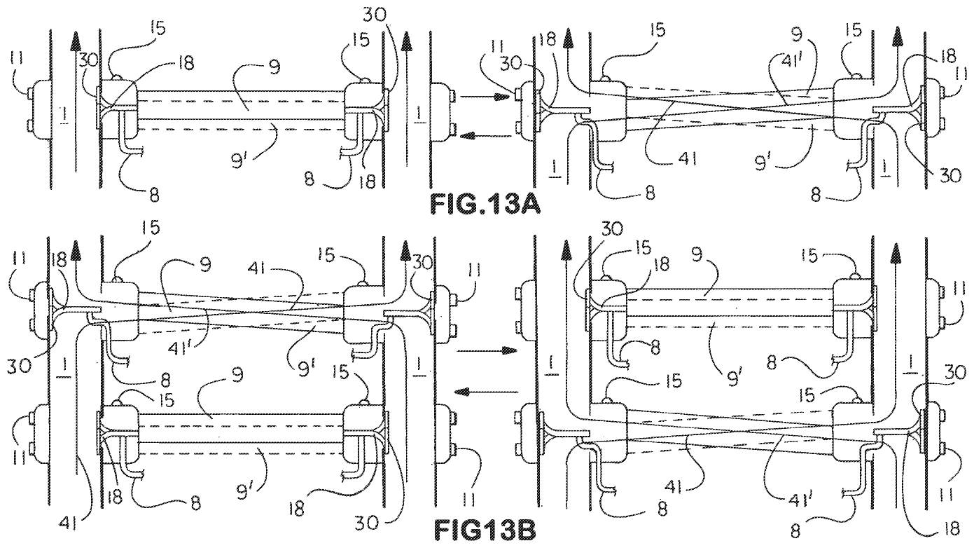

For example, shown in FIG. 13B, used to exchange flow between the great arteries when permanent placement to correct a transposition is needed, any irritation is avoided by periodically switching the exchange of flow between upper and lower pairs of jackets. If retained in fully extended or retracted position by detents, the push-pull solenoids require a momentary surge of current to overcome. Irritation and displacement are countered because:

1. The jackets are lined with a relatively thick layer of viscoelastic polyurethane foam.
2. Perforations through the jacket shells 3 and foam 4 expose sufficient surface area of the substrate ductus adventitia to the internal environment to avert the otherwise inevitable and quick atherosclerotic degeneration associated with complete enclosure.
3 In larger jackets that afford adequate clearance to allow the delivery to the adventitia of medication, accessory channels can directly release a steroid, for example.
4. The jacket spring-hinges are specified to comply with minimal resistance to the pulse of the substrate artery while applying sufficient cinching force to avert displacement, or migration, along it.
5. The suture loops at the abaxial or butt end of the valve side stem and at other points about the outer shell 3 allow connection to nearby minimally or noninnervated connective tissue to stabilize the jacket from levering with the pulse.

For these reasons, irritation, if it occurs at all, should arise only after long intervals, at which time the remedial measures are noninvasive. Lacking the versatility of servovalves, solenoid-driven diversion jackets still allow an exchange of blood within one or between two bodies to be stopped immediately. To accommodate the quick pace of growth at the outset of life, jackets are lined with somewhat thicker highly compliant viscoelastic polyurethane foam 4 and sprung hinges 11, and mainlines 9 made of highly elastic and usually accordioned or convoluted tubing. At the small diameters of the mainlines as bloodlines, the use of valves with accessory channels to directly target heparin, for example, is necessary.

FIG. 13A shows an arrangement of either bidirectional diversion jackets for the extracardiac correction of a transposition of the great arteries, or of diversion servovalves to perform a metered organ transplant, such as of the heart where space is limited. FIG. 13B shows the doubling of this arrangement at an interval along the arteries so that flow can be switched between the upper (superior, cephalad, craniad) and lower (inferior, caudad) pairs of jackets in a transposition repair or valves in a metered switch transplantation as one of several measures enumerated just above in this section to counter irritation of the substrate artery. Part numbers among the various drawings are consistent.

FIG. 14 shows a vascular servochoke. The miniature tubular pulse motor shown in FIG. 9 comprises two major outwardly evident components, shaft 42 and forcer 37. To prevent irritation to neighboring tissue, the corners and edges of the device housing (enclosure, case) are rounded. The adventitia is contacted entirely around only by felt lining 4, and rather than fully enclosed as would cause intimal degeneration, exposed to the surrounding cavity through numerous perforations 40 placed all about the jacket. As in all side-entry devices, outer shell 3 is never allowed into contact with the adventitia. Various means for providing a drugline to deliver mediation directly to the adventitia are addressed above under Background of the Invention.

In FIG. 14, part number 43 is the chokeplate (choke plate, obstruction plate, obstructor) and 10 a lock nut cap that gives access to the motor. Accessory channel 8 is shown entering with outlet into open cell viscoelastic polyurethane protective foam 4, releasing medication for diffusion to the adventitia. Accessory channel 8′ is shown entering obstruction plate 43 from below, releasing medication to flow through and out of obstruction plate 43 bottom pore 44. Dishing out, that is, creating a depression or concavity on the underside of obstruction plate 43 increases the impedance to flow, which can be used, for example, to increase uptake of the drug released through bottom pore 44, the impedance variable according to the depth and conformation of the depressions.

Flowing against gravity through a capillary tube-gauged catheter serving as accessory channel 8′ requires not only pressure equalization but increased rate and pressure of delivery by the outlet pump of the small drug reservoir implanted in the pectoral region. One function of a servochoke to increase the pressure of the oncoming flow, assuming flow is upward (cephalad, craniad) through native lumen 1, chokeplate bottom pore 44 can be used to release a nanoparticle carrier-bound drug which the choking action will pressurize for facilitated uptake in the intima. Where uptake is still inadequate, a magnetized perivascular jacket, described and illustrated in copending application Ser. No. 15/932,172, entitled Integrated System for the Infixion and Retrieval of Implants, is positioned along the segment to be penetrated, the nanoparticulate then superparamagnetic.

Radiation shielded delivery lines are described and illustrated in copending application Ser. No. 14/998,495, entitled Nonjacketing Side-entry Connectors and Prosthetic Disorder Response Systems, and Ser. No. 15/998,002, entitled Ductus Side-entry Jackets and Prosthetic Disorder Response Systems. Allowing the irradiation of ductus lining epithelia is highly exceptional and essentially limited to short segments of vessels intimately associated with malignant tissue. Such application is rarely other than endoscopic or long term. Obstruction plate 43 is introduced into the substrate native lumen 1 through a stab wound, eliminating the need for a trepan tube and thus allowing a significant reduction in size.

The action can be accomplished so quickly as to minimize if not eliminate any seepage of blood or the need for a straddle-clamp, one configuration thereof shown in FIG. 4, to define the segment for entry. Radially symmetrical, hence, neutral as to antegrade or retrograde flow, a servo chokevalve can be positioned along any vessel without regard to the direction of flow through the substrate vessel. Not requiring a well cut round entry aperture, the vascular servochoke shown in FIG. 14, chokeplate 43 is inserted through substrate lumen wall 2 through a stab wound which the operator should be able to place so quickly and with as little loss of blood as to not even require the aid of a straddle-clamp.

On the unseen backside of the servo-driven chokevalve shown in FIG. 14, a drugline enters servochoke side-stem 19 through the outer jacket 3 and foam 4 to inlet chokeplate 43. Passage through chokeplate 43 as an accessory channel is through a micromachine milled channel created when chokeplate 43 is bonded together from an upper and a lower layer as two complementary plies. The accessory channel outlet is beneath chokeplate 43 at 44.

The fluid drug and electrical lines having entered on the unseen or far side of the chokevalve, accessory channel outlet 44 drips drugs which the disruption in streamline flow backscatters for uptake into the endothelium 13 and 14. Other part numbers are consistent with those in the preceding drawings. Vascular servochokes (choke servovalves, chokevalves, choke-valves) are used primarily to raise the local blood pressure to increase the upstream uptake of drugs released into the vessel, usually a vein with lower blood pressure, a moment earlier.

A perivascular magnet jacket shown in FIGS. 2 thru 7 and described in copending application Ser. No. 15/932,172, entitled Integrated System for the Infixion and Retrieval of Implants, allows increased uptake of superparamagnetic nanoparticle-carried drugs, the more so when combined with a chokevalve. The drug may be one systemically circulated where higher uptake is wanted along the upstream segment. Release of the drug is through an accessory channel with outlet beneath the chokeplate or from an accessory channel in an upstream jacket or valve, larger volumes of an agent delivered through the its mainline. Another type of choke servovalve is the sphincteric, shown in FIGS. 11 and 12 and described in copending application Ser. No. 15/998,002 entitled Ductus Side-entry Jackets and Prosthetic Disorder Response Systems.

Chokevalves are always driven by a servomotor automatically controlled by an implant microcontroller, or in comorbid disease, a microprocessor, serving as the master controller in a hierarchical control system in accordance with physiological data fed up through the hierarchy to the master controller which executes a prescription-program devised for the specific patient. The microcontroller or microprocessor controls a small peristaltic pump at the outlet pump of a small flat drug reservoir implanted subcutaneously in the pectoral region.

### Valve Applications—FIGS. 15 Thru 17 and 22 Thru 25

FIG. 15 is a simplified schematic representation of a switch transplantation, in which blood flow through the actual vessels of the native solid organ—here the heart—have been schematically summarized to make clear how flow is switched from the native to the donor organ. Depicted as applied to the heart, the same process pertains to a kidney, liver, lung, gland, or any other graft organ. Heart outflow is through the aorta and pulmonary artery (pulmonary trunk, main pulmonary artery) and inflow through the venae cavae. In an orthotopic switch transplantation, once all blood flow is through the donor organ, the native organ is removed. Alternatively, blood flow can be continued through both native and donor organs indefinitely, thus adding the donor organ as supernumerary in a heterotopic transplant.

Shown is the compound bypass transfer of blood flow from the native to the donor organ at the half-way point, 45 representing the transections at the ends of the stumps of the donor graft organ. The chutes have been depicted at an intervening point in extension instantly traversed in a sudden switch transplant but the midpoint intervening between complete retraction with the organs independently perfused and complete extension during which both organs are supplied blood by both the donor on life support and the recipient to blend the blood in order to effect a measure of immune tolerance induction.

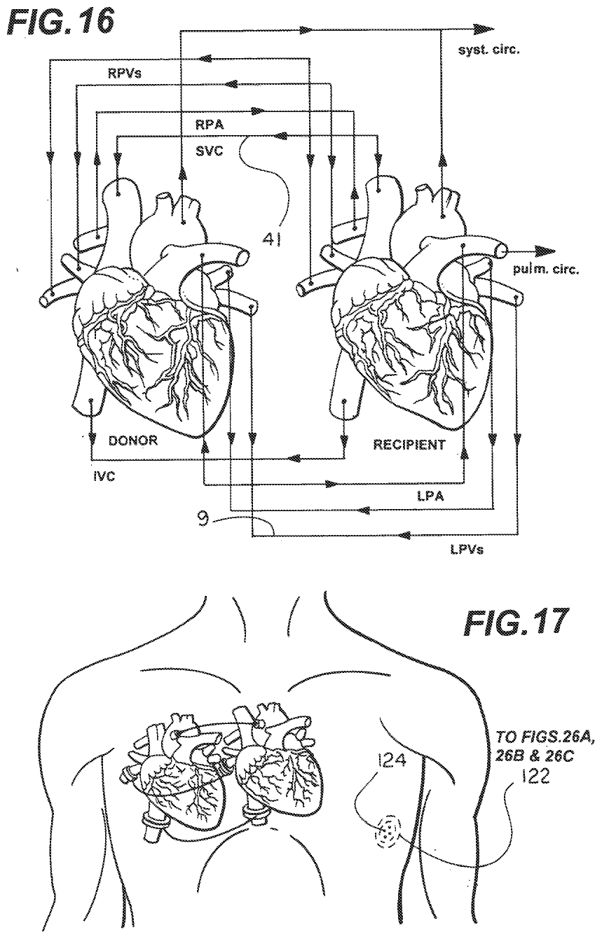

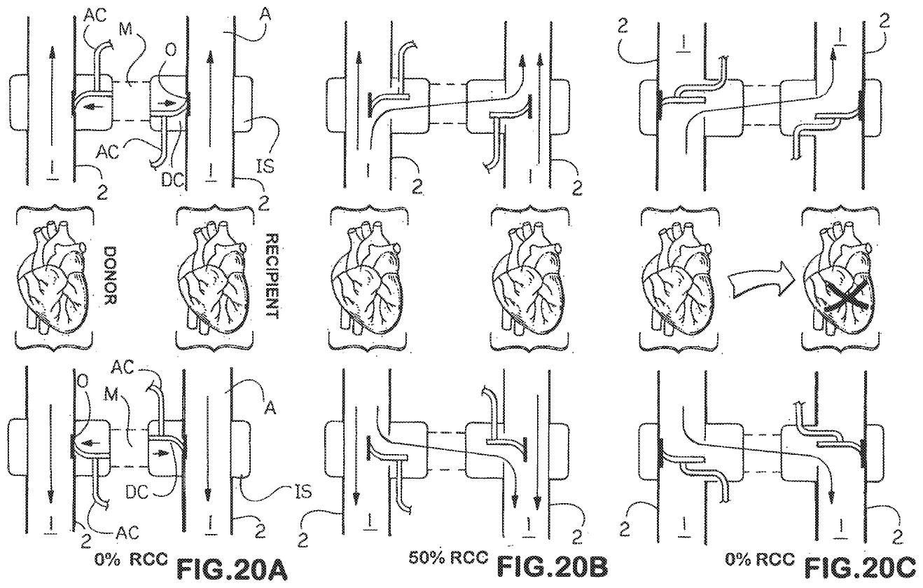

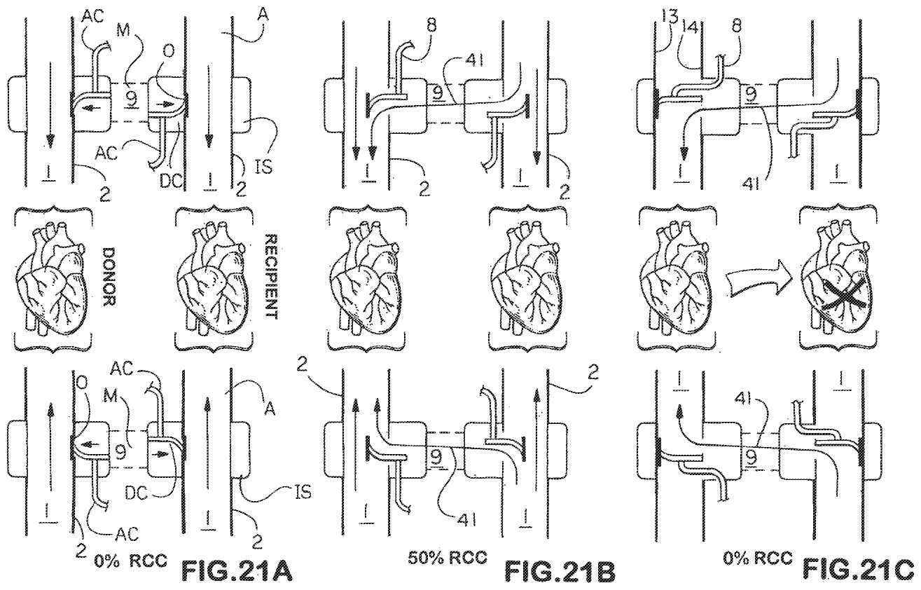

In a metered switch transplant, this intervening point in chute extension may be temporary, the chutes partially retracted or further extended from it, or can represent the final setpoint, adjusted for a mismatch in the relative size, consistent with the absolute stroke volume, or ejection fraction if either heart. This midway point interposed between donor-recipient circulatory independence and blending is shown for arterial flow in FIG. 20B and for venous flow in FIG. 21B. The vessels involved are identified in FIG. 16. The extent to which the blood is blended is controlled by the duration the diversion chutes are positioned at such an intervening extent.

In an orthotopic transplant, excision of the donor organ is not performed until blood flow has been passed to it exclusively. That the figure represents the heart having been excised with the vascular servovalve diversion chutes at the half-way point to equally apportion flow through both indicates that this is a switch heterotopic, or double heart transplant as shown in FIGS. 16 and 17, and that while no such size matching is necessary for this procedure increasing the pool of replacement hearts, the added heart is similar in size to that native.

This continuity of circulation while the donor is kept past death on life support in the same center eliminates storage, and therewith, the endothelial degeneration and cellular breakdown products which contribute to early hyperacute rejection and late term vasculopathy. That neither native nor donor organ are incised, the recipient placed under regional, not general, anesthesia without cardiopulmonary machine support, and can remain conscious throughout the procedure likewise offer benefits over conventional transplantation. In a metered switch transplant, where the transfer of circulation from the native to the donor organ is gradual, a choice can be made at this intermediate stage as to whether the donor organ should orthotopically replace or heterotopically supplement the native organ.

Part numbers are consistent with those identified previously. If supplementation is chosen, circulation remains divided between the native and the graft organ. An hypoxic condition of either organ is detected and signaled by implanted sensors to the implanted microcontroller which then toggles the diversion chutes to alternately apportion a nearly full complement of blood to either. In contrast to metered switch transplantation, which also allows immune tolerance induction concomitant with organ transfer, a sudden switch transplant transfers circulation from the native to the donor organ abruptly and entirely without the ability to stop at an intervening point or reverse the diversion chutes, so that immune tolerance induction therapy must be pre- and postprocedural.

FIG. 16 identifies each of the nine actual vessels summarized in FIG. 15. In FIG. 16, the letter A stands for the aortae; LPA stands for the left pulmonary arteries; LPVs for the left pulmonary veins; IVC for the inferior venae cavae; SVC for the superior venae cavae; and RPVs for the right pulmonary veins. Arrowheads directed to the right denote outflow, those to the left inflow. The double heart transplant having been completed as depicted, flow through the vascular diversion servovalves have been brought to the half way retracted or withdrawn from the lumina to pass half the blood of the donor through the recipient and donor hearts.

At eighteen, the number of valves required gives emphasis to the importance of achieving the lowest weight and greatest miniaturization of each, obtained primarily through microfabrication and the use of light plastic and metal parts and electrical windings of silver. For visual clarity FIG. 16 omits flow lines through the accessory channels, or druglines, associated with each valve. All lines fluid and electrical must be routed to avoid strangulating tissue. To implement this, accessory channels can enter into the valves at a number of places such as along circumferentially running perforations 40 or transversely to sidestem 19 and intotrepan tube 5 or foam 4. If direct routing will not allow this, then fluid and electrical lines are rerouted. When clearly safer for the patient, hard wires are replaced by remote radio channels such as ‘Bluetooth.’

If valve obtrusiveness A metered switch solid organ transplant requires the use of vascular servovalve with direct manual fine control such as that shown in FIGS. 10A thru 10C so that the operator can make minor adjustments in diversion chute position during the condition of no current flow before the automated process of organ transfer is initiated. Were the hearts different in size than as shown, this valve setting would have been adjusted to either side of the half way point to accommodate the size difference in absolute stoke volume, or stoke volume, of either heart. In FIG. 16, the ‘corners’ at the connections of the mainlines, here bloodlines 9 to each of the vessels is the side-entry vascular servovalves by which the connection is made.

Since these valves not only mediate circulation postoperatively, but the transfer of circulation between the two in transferring the donor heart into the circulatory system of the recipient, only fully operational servovalves are used, disallowing the use of passive, or nonadjustable diversion jackets. This ability to apportion blood flow through either heart in accordance with the relative size or stroke volume of each heart eliminates the complications normally associated with hearts mismatched in size in heterotopic transplantation of a smaller donor heart, the use thereof making it possible to retain more of the right lung as preferred.

While the vessels can be anastomosed and bloodlines 9 removed, the jackets with druglines 8 attached and continuous with the valve accessory channels remain to deliver drugs detected as necessary by implanted sensors so that the master controller is signaled of the need for direct pipe-targeting of a medicinal or electrostimulatory therapy as necessary. FIG. 15 also represents the half-way point in an orthotopic metered switch heart transplant before the valves are adjusted to direct all blood flow through the donor heart. To perform a switch orthotopic heart transplant, the native heart is then removed and the graft organ set in its place.

Leaving the vascular diversion servovalves at the half-extended position or somewhat aside thereof to accommodate a graft organ of different size, allows the native heart to remain and the donor heart to be positioned beside it, for example, some volume of the right lung removed to accomplish this. Whether such a metered switch heterotopic for double heart transplant places the added heart in the abdominal cavity or nestled in the iliac fossa, for example, it is noteworthy that the flow of blood through all the significant vessels of both the native and donor hearts is that assigned by nature.

Downward directed arrowheads denote venous return, upward directed arrowheads ejection, and the arrowheads along the bloodlines indicate the direction of flow. The same connections apply regardless of where the donor heart is positioned in the recipient; this configuration provides a distribution of the blood-moving load proper for the chambers and the pattern of flow normal for the vessels. Preservation thus is best with the donor heart positioned as shown, but where unavoidably, placement must be lower in the body, function will still be much closer to normal than were connections made to local vessels. Direct connection to the inferior vena cava and abdominal aorta, for example, results in much flow past the offline transplant, and therewith, disuse atrophy.

The added heart functioning as an adjunct or reinforcer of the native heart, only the native heart is directly connected to the rest of the circulatory system. With the servovalves connecting the bloodlines set to favor both equally at the half way point, each heart contributes half to the overall volume of blood ejected. In fact, the valve setpoints are shifted to favor the hearts according to the competency, that is, the ejection fraction, or absolute stroke volume, of each. Thus, when the hearts are equal in this regard, the servovalves connecting the mainlines are set at about the half way point and can be intermittently shifted between an almost entirely open and closed position to allow either heart to receive the full rather than half the normal volume of blood.

Venous return is from the native to the added heart, while ejection is from the added heart to and through the native heart, so that with the hearts positioned as shown, the overall flow of blood is clockwise. The metered switch pattern of flow shown was initiated after the valve jackets and communicating lines were connected to the donor heart on the left while still in the donor on life support. This reciprocal cross-circulation was continued long enough to accomplish a measure of immune tolerance induction as integral in the process of gradually transferring the heart from the donor to the recipient.

Because unlike other organs, the heart cannot be taken from a living patient, this process can continue over a longer duration. The donor heart is then harvested, the vascular stumps sealed with suture and a fibrin sealant, and placed in the recipient without disconnecting the lines or valves. Connected thus and alternated as indicated, the ejection and venous return of two hearts add with a time lag smallest when adjacent in the chest as shown and increased as the donor heart is positioned farther down in the body.

In FIG. 17, the native or earlier orthotopically transplanted heart on the right is undisturbed in its connections to the rest of the circulatory system, the diversion servovalves extracardiac, placed on its in- and outflow vessels. The bypass, or metered switch, transplanted heart on the left in the figure communicates with the circulatory system by connection to and through the vessels of the native heart shown on the right. That flow through the graft is ‘natural’ is intended to eliminate disuse atrophy as occurs in the direct connection of a redundant heart to the inferior vena cava and abdominal aorta, for example, as has often been done in immunological research, for example.

The connection of the added to the native heart is accomplished by truncating a metered switch orthotopic heart replacement at about the half way point, then clamping, harvesting, and implanting the donor heart without disconnecting the blood diverting lines. A double heart transplant is always metered, never sudden switched. In a double heart transplant, the vascular servovalves—those of the recipient at the right end of the connecting bloodlines and those of the donor at the left end—placed while the donor on life support remains intact—mediate the compound bypass of the native organ and thereafter are not removed but rather remain to channel the blood as shown and allow the direct pipe-targeting of drugs through any valve.

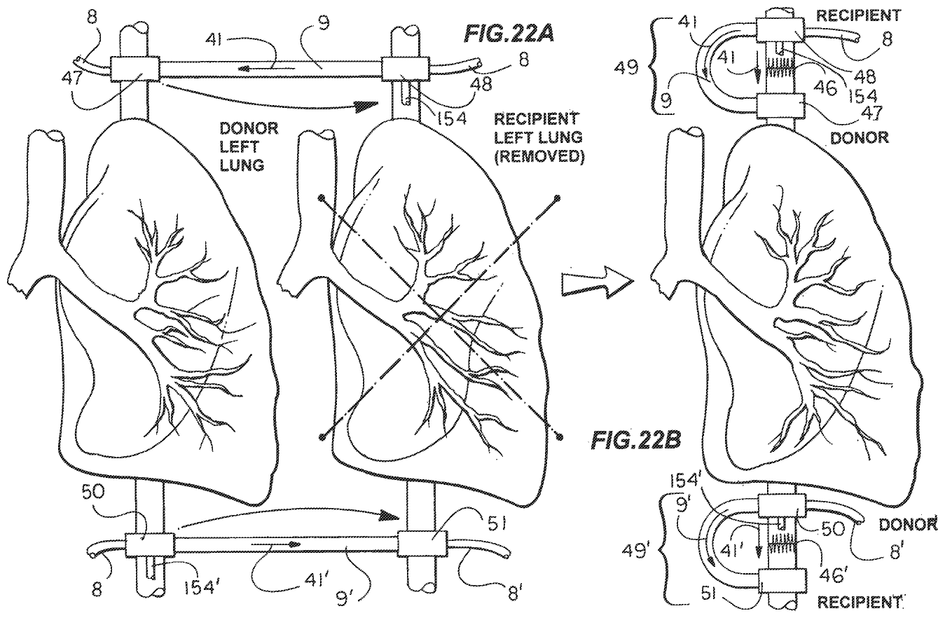

For visual clarity, connecting bloodlines in the chest view of FIG. 17 were limited to three—the aortae, venae cavae, and right inferior pulmonary veins. As shown in FIG. 16, there are, however, actually nine major incurrent and excurrent major vessels, hence, nine mainlines, or bloodlines, eighteen servovalves, and 18 druglines, each routed to an accessory channel in one of the valves. In contrast to the lung transplant depicted in FIG. 22, the stumps are distant and not anastomosed, the severed ends sealed with a fibrin sealant and suture. Thus, whereas in a switch double heart transplant the 18 valves are best left in place rather than replaced with simpler side-entry jackets before closing, in an orthotopic switch heart transplant, donor and recipient stumps can be anastomosed allowing 9 of the valves to be removed.

More specifically, in a double heart transplant, retention of the valves allows maximum flexibility in automatic flow adjustments, continued maintenance and drug delivery on an indefinite basis. For these reasons, the midprocedural number of valves, rather than replaced by basic side-entry jackets, for example, are best retained after the operation has been completed. However, this separation may actually ameliorate irritation associated with direct contact, and the immediate support of directly targeted drug delivery should overcome any drawback this separation might otherwise impose.

The need for 18 valves demonstrates the importance of achieving small size, light weight, and minimal cost in valves for use in such a double heart transplant, of which the advantages, enumerated herein, are numerous, substantial, and basic With the diversion valves set at the halfway point, when both hearts beat simultaneously, the hearts divide the normal volume of blood equally. The servovalves can divide the full volume of venous return between the two hearts in any proportion, or the entire volume to either heart at a given moment, but cannot double the volume available to pass a full volume of blood to both simultaneously. To allow an almost full volume of blood to be processed by each in turn, the valves are switched to favor the one, then the other in alternation.

Cardiac hypovolemia is detected by oximeter implants in either heart and prevented by automatically initiated toggling of the servovalves to alternately favor either heart at the optimal rate the microprocessor is programmed to hunt for and set. Should a dysthythmia arise, it is detected by implant sensors using circuitry much the same as that used in an implantable cardioverter-defibrillator and the disruption electrically resynchronized, or reversed through the application of shocks initiated at a level calculated to avoid injury, which is stepped up until successful.

Thus, to prevent the inducement of a dysrhythmia that could lead to a sudden arrest, and because for the heart to beat when empty can be injurious, sufficient blood is always provided to both to avoid these eventualities. Because this arrangement allows venous return and ejection to be apportioned between the hearts, the added heart can be and is best somewhat smaller in size to allow placement with the least removal of lung tissue. Lung tissue regenerates not by increasing in mass but rather by increasing the number of alveoli per cubic centimeter, making it doubtful that the patient will experience even a temporary postoperative shortness of breath worthy of note. The graft organ should not interfere with the restoration of the ribcage to very nearly if not exactly its normal conformation before closing.

Drug delivery responsive to implanted sensor input, all of the servovalves incorporate drug-delivery, or accessory channels, through which the control system can target drugs through catheters connected to each valve from small flat reservoirs placed subdermally in the pectoral region. Drug replenishment is by injection into the reservoirs through a body surface port, of which one type is shown herein.

FIG. 17 shows the positions of the hearts following a double heart switch transplant, the added heart when possible having been placed in the chest as preferred for several reasons, to include protection by the rib cage as close to the natural position intended therefor, reducing the need to work against gravity, and for other physiological reasons. Positioning thus is overridden only due to deformity or a crushing injury to the right side of the chest where an urgent need for a heart outweighs the additional time it would take to reconstruct the damage. The vessels having already been identified in FIG. 16, to avoid clutter, only three recipient-donor connections have been shown.

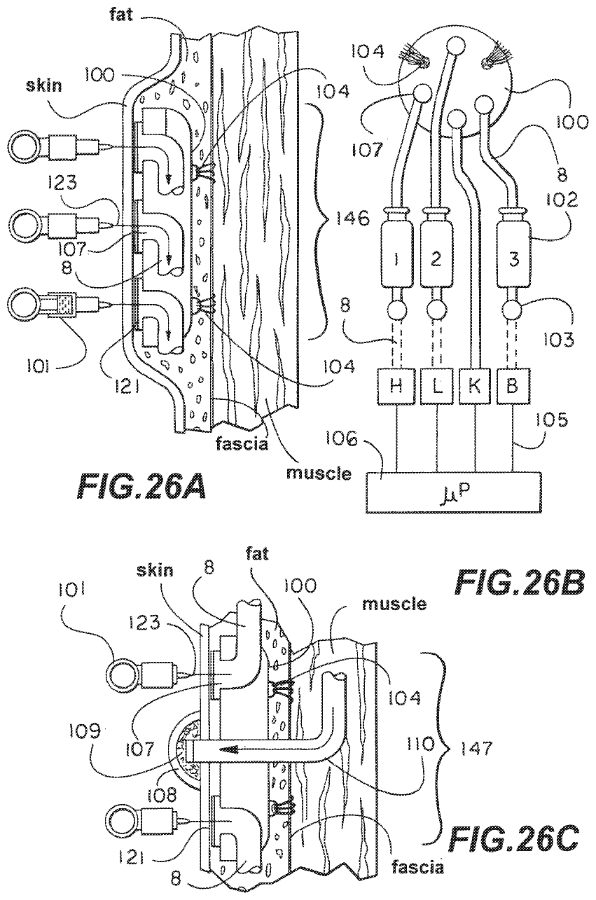

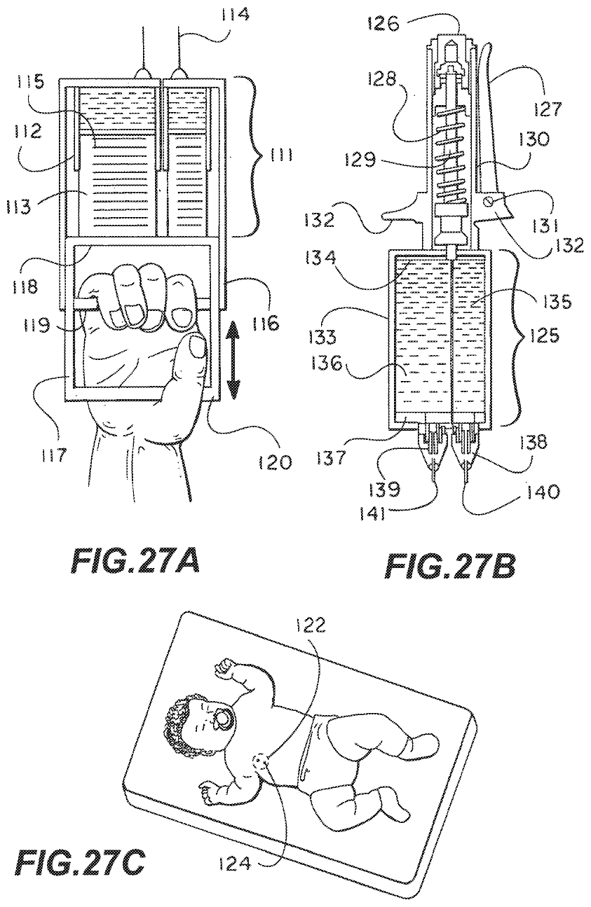

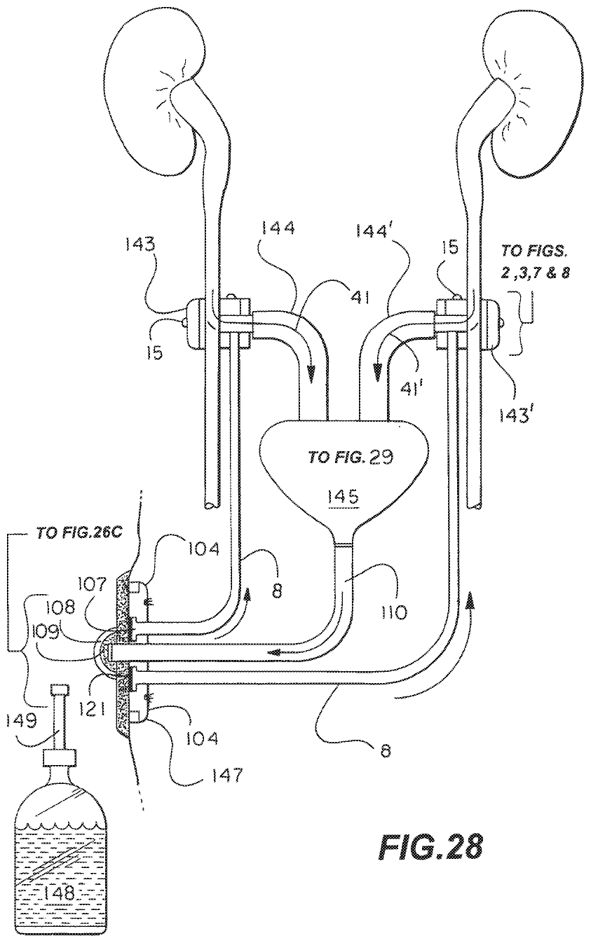

Shown in FIGS. 17 and 27C, subcutaneously implanted multiple injection point port 122 of the portacath or mediport type corresponding to port 146 in FIG. 26A with on-the-skin, or cutaneous injection point indicating marks 124 allows the replenishment of implanted drug reservoirs such as shown in FIG. 26B by multiple injection means such as those shown in FIGS. 27A and 27B through tattooed injection point indicators 124. Although to be certain that the multiple injector is exactly positioned to replenish each drug through its intended injection opening only one injection point need be identified on the skin, here out of an abundance of caution, two injection points 124 are shown as preferred. The multiple injectors shown in FIGS. 27A and 27B comprise four injectors of which the injection openings 107 are shown in FIG. 26B, for which two of the injection points 124 are marked on the skin as shown in FIG. 27C.

While a port with drain 110 to the exterior such as shown in FIG. 26C for the outflow of urine would ordinarily be positioned to a side of the mons pubis where no pump is needed and the wearer has a clear view of it, where the frequent insertion of cabled diagnostic and/or therapeutic devices at a higher level is required, the same kind of port, shown as 147 in FIG. 26C, would normally be positioned in the pectoral region. Thus, depending upon the application, both ports with an opening to the exterior such as port type 147 shown in FIG. 26C and those fully subcutaneous such as port type 146 shown in FIGS. 17, 26A 26B, and 27 can be positioned at different levels.

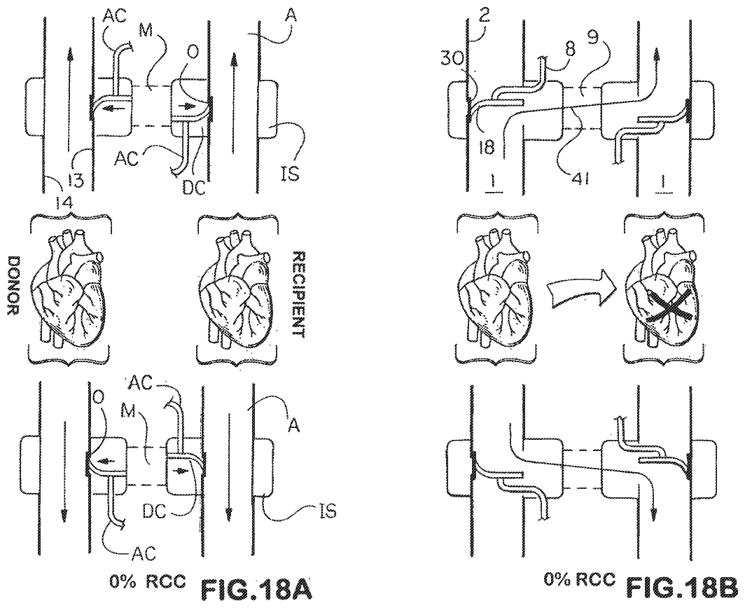

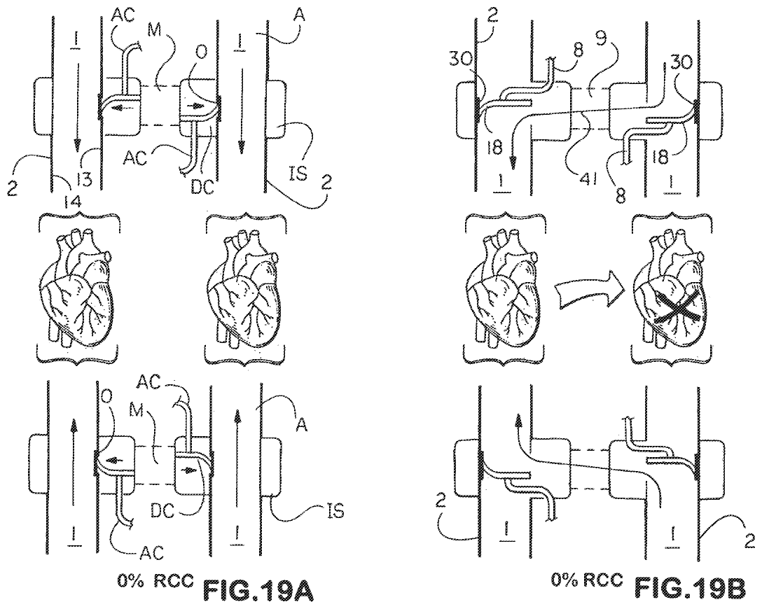

FIG. 18 depicts the flow through one of the arteries shown in FIG. 15 in a sudden switch heart transplant. In FIG. 17A. highly damped nonsparking solenoid-driven flow diversion chutes are fully retracted, ostium obturator 30 covering over the side-entry opening along near luminal wall 13, thus blocking the outflow of blood through the ostium. When the operator or an assistant depresses a switch to send current to the solenoids at the donor and recipient ends of the mainline, or bloodline, connecting each of the vessels shown in FIG. 15, all of the blood flow diversion chutes simultaneously snap from full retraction against near luminal wall 13 to full extension, whereby all flow is diverted from the recipient to the donor graft organ, here the heart, the ostium obturator now pressed flush out of the flow path against far luminal wall 14.

In FIG. 18A, arterial flow from the donor heart before the diversion chutes are deployed is summarized, in that all arteries are depicted as one on the left, and all flow from the diseased heart of the recipient is summarized on the right. Arterial flow after the chutes have been deployed is shown in FIG. 18B, where the X strike-out indicates that with the donor organ now incorporated into the circulatory system of the recipient, the innate organ has been bypassed and removed. FIGS. 5, 7, and 8 show the structure of intravascular diversion valves and servovalves. The sizes of the jackets vary according to those of the respective substrate vessels, the rotational orientation chosen to least encroach upon neighboring tissue.

FIG. 19 depicts the same action as shown for the arterial chutes in FIG. 16 as applied to the veins. More specifically, FIG. 19A is a schematic summarization of venous flow to the donor heart on the left and the innate heart of the recipient on the right, before venous switching in FIG. 19A, and after switching in FIG. 19B. In FIG. 19B, the X strike-out indicates that circulation through the donor heart having been switched from the donor into the circulatory system of the recipient, the native heart has been removed.

FIG. 20 depicts the same starting and ending points of the blood diversion chutes at the ends of any one of the arterial bloodlines shown in FIG. 15, that is, full retraction and full extension, shown in FIGS. 18 and 19 where an intervening position at the center indicates that this is a metered rather than a sudden switch transplantation. Instead of suddenly shifting from the fully retracted to the fully extended positions, movement of the blood diversion chutes between these endpoints is gradual, implanted sensors alerting the implanted controller as to the extent of antibody release. The chute driving servomotors such as that shown in FIG. 9 can advance or retract the chutes at any rate to any point along the trajectory separating complete retraction to complete extension.

In this way, the process effectively ‘feels’ and ‘inches’ its way to full extension at which blood flow is entirely through the donor organ. Were this a metered double heart transplant, the endpoint would be closer to if not at the state depicted in FIG. 20B at which the blood of donor and recipient would eventually represent a 50/50 mix. The extent to which the process and therefore the proportion of donor blood in the mix depends when the chutes can remain at the more central endpoint with no more than manageable disturbance induced. In both sudden and metered switch transplants, the implanted disorder response system, druglines, and jacket accessory channels into which these feed remain for postoperative therapy. If donor and recipient stumps are anastomosed, the bloodlines or mainlines can be removed.

FIG. 21 shows the same three stages in a metered switch organ transplant, here the heart, for each of the veins shown in FIG. 16 as FIG. 20 shows for the arteries.

FIG. 22 shows the disposition of the bloodlines connecting the donor and the recipient organ to be replaced before the graft organ has been harvested in FIG. 22A and after it has been placed in the recipient in FIG. 22B. Transplantation of a lung requires connection of the pulmonary arteries and pulmonary veins, schematically represented here as summarized. Lungs are orthotopically, not heterotopically transplanted. The practicability much less necessity for the heterotopic transplantation of a lung are the least of any major organ.

In FIGS. 22A and 22B, part numbers are consistent with those in the other drawing figures, so that 8 is a schematically depicted arterial drugline, while 8′ is a venous drugline, all druglines passing into an accessory channel inside the valve jackets. Part number 9 represents a single bloodline as a schematic summarization of the pulmonary blood supply normally consisting of three arteries of the bronchial circulation which supply oxygenated blood and the pulmonary arteries which supply deoxygenated blood, 9′ an equivalent summarization of the venous drainage, and 41 is a flow or stream line showing the direction of flow. The condition depicted in FIG. 22B offers different options in addition to allowing the condition shown to remain so that blood flow can be switched between the native vessels and either or both arterial bypass 49 or venous bypass 49′.

Determined on the basis of clinical judgment, anastomosis of either or both stump junctions along suture lines that arterial 46 and that venous 46′ can be omitted and the bypass left in place indefinitely as a prosthesis. One consideration in this decision is that a later transluminal, or transcatheteric, procedure will have to pass through the prosthetic bypass rather than through the native structure. If the drugline or internal configuration of the diversion jacket or valve prohibits transluminal treatment, the procedure is endoscopic. Because all basic side-entry jackets, diversion jackets, and valves incorporate at least one accessory channel to directly pipe-target drugs to the treatment site, the need for a later transluminal intervention is slight.

Hypothetically then, should the donor heart in a heterotopic double heart switch transplant require the removal of obstructive plaque, for example, a coronary artery bypass graft, preferably performed endoscopically rather than a percutaneous transluminal, or transcatheteric, angioplasty is performed, the surgical procedure long established as more durable than that transluminal. When donor and recipient stumps are anastomosed as shown at 46 and 46′ respectively, either or both arterial bypass 49 and venous bypass 49′ can be removed. The valves and bloodlines will continue circulation whether donor and recipient stumps are anastomosed before closing or separately terminated with a fibrin sealant and suture.

Ordinarily, the stumps are anastomosed and only recipient arterial diversion valve 48 and donor venous diversion valve 50 left in place in order to retain their respective accessory channels upstream to anastomotic suture lines 46 for continued therapy as necessary. Periodic imaging should supplement implanted sensor feedback to the drug regulating implanted microcontroller, or more likely microprocessor, which controls the drug reservoir outlet pumps. Accordingly, every case unique, different vessels differently affected, arterial and venous bypasses would normally be treated alike, but to accommodate different conditions of disease or injury, either can deviate in any of the foregoing particulars.

Rather than to cover these over, the valves are set off at a slight distance from and positioned to directly target immunosuppressive, anti-inflammatory, and antimicrobial medication, for example, to the anastomoses. The anastomoses are therefore neither cut off from the surrounding milieu nor hidden from sight or endoscopic access. Targeting thus spares nontargeted tissue the complications that inevitably arise with immunosuppressive and chemotherapeutic drugs, for example.

While the use of permanent magnet perivascular jackets, or impasse-jackets, as described in copending application Ser. No. 15/932,172, entitled Integrated System for the Infixion and Retrieval of Implants, can achieve targeting by drawing superparamagnetic nanoparticle or microparticle carrier bound drugs into the intima, these do so at the level where they are positioned, whereas fluid drug delivery through the valve accessory channels allows the intact fluid to run down across the anastomoses. However, projecting no part into the native lumen, impasse-jackets do not pose an obstacle to transluminal passage with a cabled device or angioplasty balloon, for example, whereas valves do in situations where to retract the diversion chutes may not be practicable.

Bloodlines, or mainlines, summarized as part numbers 9 and 9′, must span the distance between the donor and the recipient over a length that may result in these being too long to position in the recipient so as not to encroach upon neighboring tissue, as well as necessitate more pressure to traverse these spans. Once the graft organ has been placed in the recipient, mainlines, here bloodlines 9 and 9′ and druglines 8 continuous with the accessory channels which course through the valves, are reduced in length to that appropriate in any of a number of ways. These include:

1. The use of highly elastic accordion-configured tubing that spontaneously shortens as this distance is reduced.
2. Additional shortening is achieved by running a bead of surgical cyanoacrylate cement adherent to the tubing plastic along the apices of the pleats of the accordion tubing to run down into the reentrants between the pleats thence to the underside of the pleat reentrants. The operator or an assistant then further shortens bloodlines 9 and 9′ and druglines 8 by pressing the tubing shorter so that successive accordion bellows-like pleats are glued together side to side. The height of the pleats sets the degree to which the tubing can be shortened. The blood passing through the tubing little diverted into the pockets posed by the entries into the pleats from the lumen, the internal diameter of the tubing is little affected, and the accumulation of clot these would normally promote is dispelled by using druglines 8 to release an intermittent low dose heparin drip.
3. Compatible with the foregoing is the use of heat shrinkable elastic accordioned tubing. In some cases the use of straight-walled, or non-accordioned, cylindrical heat shrinkable tubing will be sufficient.
4. Where the distance separating donor and recipient allows, tubing that incorporates successive thicker and thinner walled sections allows involuting (telescoping, invaginating) the thinner into the thicker sections. This reduces the internal diameter of bloodlines 9 and 9′ and druglines 8, which if necessary, is easily avoided by compensating for the reduction through the use of tubing equally larger in internal diameter at the outset. Use of this method assumes that the successive telescoped sections are sufficiently pliant as not to irritate neighboring tissue.
5. Bloodlines 9 and 9′ and druglines 8, are interposed by abridge section connected at donor and recipient ends by diversion jackets. Once the graft organ has been placed in the recipient, facing ends of bloodlines 9 and 9′ and druglines 8 are ‘anastomosed’ with strongly adherent surgical grade tape and the diversion jackets with bypass tubing removed.

While a protective lung cage and the removal of an adequate length of gut would allow positioning a lung in the abdominal cavity, air delivered to the lung through a trachea extended with synthetic tubing at the correct pressure would likely require the additional implantation of a small assist air pump, pressure sensor, and oximeter sensors. However, because the blood supply and drainage afforded by switch transplantation assure virtually normal oxygenation and removal of carbon dioxide, hypoxia in any lobe should not arise.

Such an arrangement could be made to work, but the need for it would appear rare, mostly due to chest injuries that irreversibly damaged both lungs where the use of extracorporeal membrane oxygenation, for example, would not sustain the patient for more than a short time. Lung disease and related defects often extending to the trachea and bronchi, when diseased or defective as to justify its replacement, the trachea and much of the bronchus, ordinarily not included in the transplant, is included as shown.

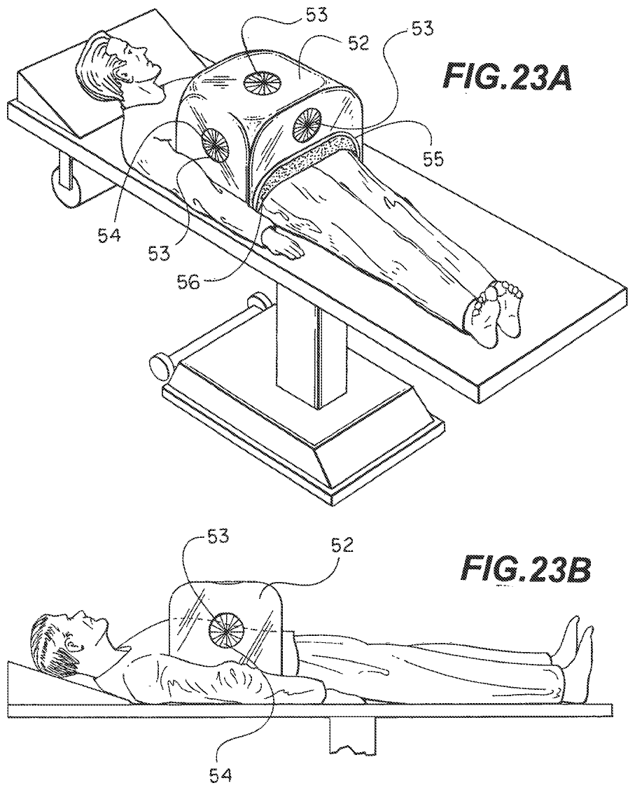

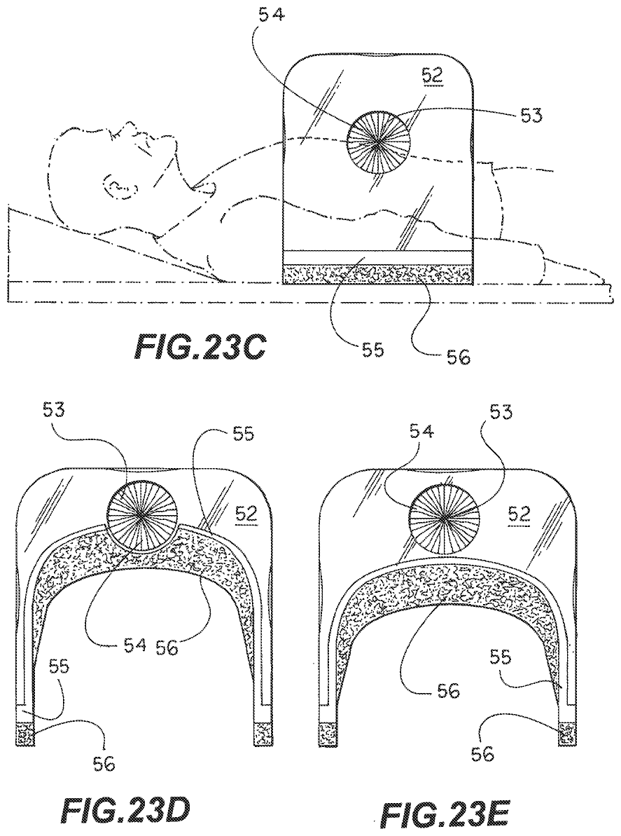

FIG. 23 provides in FIG. 23A an overall perspectival view of a surgical chestdome, an overall side view in FIG. 23B, a closer side view showing wraparound cast iron weight 55 and foam cushion 56 running around the bottom, in FIG. 23C, the upper, or head-facing (superior, headward or cephalad, craniad), surface thereof in FIG. 23D, and the lower, or foot-facing (inferior, caudad. footward), surface in FIG. 23E. A surgical chestdome serves to equalize the pressure surrounding the surgically breached chest with that intrapulmonary, thereby averting a surgical pneumothorax. The weak vacuum inside the chestdome will usually require the introduction of oxygen or if necessary, the use of mechanical assistance, preferably through a nasal tube, to allow spontaneous breathing.

Surgical chestdome 52 is applicable to any surgical procedure that requires entry into the chest but was devised to allow spontaneous ventilation, or normal breathing where cardiopulmonary support or extracorporeal membrane oxygenation is not used, under regional if not local anesthesia as preferable in a switch transplant. Cardiopulmonary support and/or general anesthesia often leave the patient with cognitive deficits, and a mechanical ventilator operated by anyone not expert in its use can do serious damage to the lungs.

If air enters the dome, the response of the vacuum pump with limit switch is to instantaneously equalize the pressure within to that outside the dome When the graft organ, such as a heart, is to be transplanted, the organ is harvested and placed within the dome with bloodlines and druglines attached before the chest of the patient is opened and dome 52 placed over the patient. Alternatively, a larger rectangular glove opening is provided to allow such a larger object to be passed through. In FIG. 23A surgical chestdome 52 is made of a clear transparent plastic such as polycarbonate. Glove openings 53 on each side of dome 52 give the operator and assistants easy access to the operative field. Glove openings 53 are filled by stiff bristles several layers deep 54 which brush against the sides of the gloves and any objects introduced or removed to leave virtually no space through which air might enter.

Should air enter nevertheless, the vacuum pump with limit switch will immediately reinstate the rarified condition inside dome 52. Further to assure an airtight fit of dome 52 to the surface of the operating table and/or any bedding or covering, weight 55 runs around the dome 52 just above foam cushion 56 along the bottom periphery. For rust resistance, cast iron dome surround 55 toward the bottom of the dome is galvanized with 0.001 to 0.003 inch thick inorganic zinc anti rust spray, then topcoated with a rust resistant epoxy, phenolic, acrylic, or silicone paint, for example. When the graft organ is placed beside or on the patient before dome 52 is set down, small segments of foam lining 56 if not sufficiently compliant as not to compress the bloodlines are replaced with a more compliant foam.

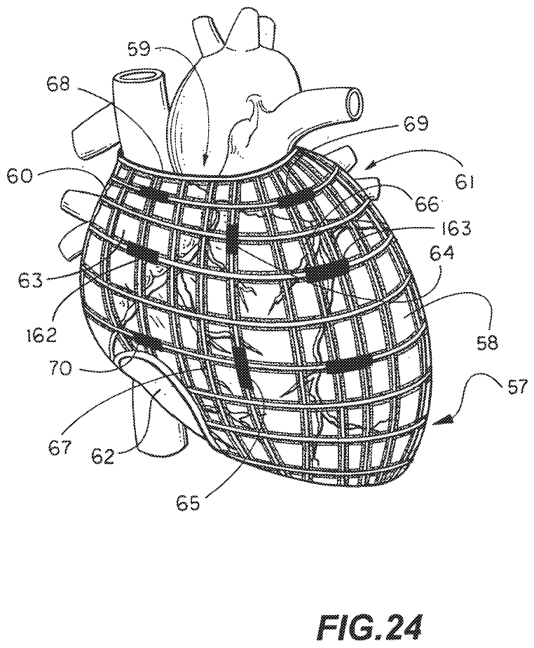

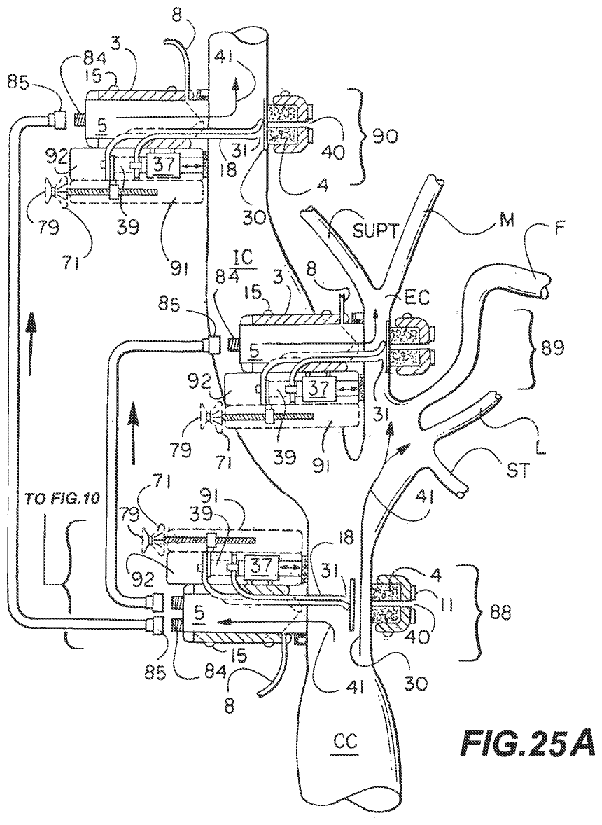

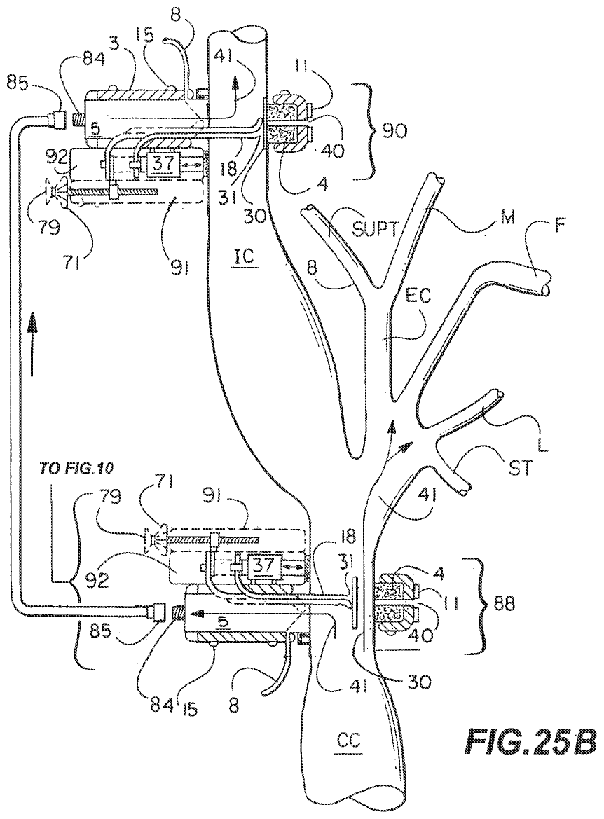

FIG. 24 shows a heart cage (grid, grating) for surrounding a heterotopically bypass, or switch, transplanted heart when placed in the lower abdomen, for example, where adequate protection by surrounding bone is lacking. To minimize its weight, the cage is fabricated from narrow hollow titanium or stainless steel tubing. FIG. 24 provides an anterior (frontal, face-on) view of a heart cage. The cage, fabricated by electrical resistance (‘spot,’ electrowelding), comprises two complementary sides, each conformant to the shape (profile, contour) on its respective side of the heart, always asymmetrical, to be enveloped and complementary to the periphery of the opposite half. The halves are hinged at the back by electrically welded hinges (not shown) made of the same titanium or stainless steel as the grid.

For strength and to avoid current flow, the weld metal is the same as the grid. The halves come flush together at the front, where spring-loaded upper latch-type lock fastener 64 and that lower 65, likewise attached to the grid frame and made of the same material, allow the cage to be opened should a follow-up procedure, such as examination by a transluminally passed intravascular ultrasound probe. or a coronary artery bypass graft become necessary. All of the hinges to include those along the back, or posterior, junction where the sides come flush together or seam and spring-loaded latch fastened locks similarly positioned along the front, or anterior, seam are made of the same titanium or stainless steel, each electrowelded to the grid about a third of the distance from the top and bottom.

The cage comprises gridwork 57 of hollow titanium or stainless steel tubing 3/16 inch or ½ centimeter in outer diameter, with a tubing wall thickness of 1/16 inch and grid apertures (holes, openings) 58 of 1 inch or 2½ centimeters, following the same outer shape (profile, contour) as the contained heart itself. The grid stands off at a distance of about ½ inch or 1¼ centimeters from the heart per se, affording clearance for the interposition of a protective layer of perforated viscoelastic polyurethane foam, bonded along the interior surface of the cage grid tubing and provides an opening at the top 59 to allow the superior vena cava, aortic arch, and its vessels with any perivascular fat, vasa vasora, and vasa nervora to pass through without contacting the sides of the foam-lined top opening.

The grid also has a side opening 60 at the upper right and one at the upper left 61 to allow the pulmonary arteries and veins with supportive nervelets and fine vessels pass through. Toward the bottom on the right is another opening 62 to pass through the inferior vena cava with sufficient clearance for its supportive nervelets and fine vessels to disallow encroachment by the margins of the opening. Applied after reciprocal cross-circulation through the mainlines connecting the donor and recipient to effect the metered bypass, or switch, has already been instituted, which must not be interrupted, the heart cage requires not only permanent openings 59, 60, 61, and 62 but must continue with openable flap sections respective of each, opening flap 63, spring-loaded hinged at 68, lift flap 66, spring-loaded hinged at 69, and flap 67, spring-loaded hinged at 70.

Opening flaps 63, 66, and 67 lock in the closed position are preferably kept closed as indicated by spring loaded hinges but can also use the same type spring-loaded latch engaging fasteners as those at the anterior midline to remain secure when not lifted to allow this clearance. Such an optional fastener is shown on the heart right side as part number 162 and on the heart left side as part number 163. The grid tubing must be able to withstand deformation as the result of a direct blow and at the same time, allow the operator to intentionally introduce bends as might be necessary to fit a less than ideal graft heart that had disproportionately adapted to its impairment as to present an anomalous contour. This is accomplished by heating the grid where the bends are needed with a small hand-held propane blow torch, for example, rendering the areas to be altered pliant.

Accompanied and assisted by another heart, supported by medication immediately dispensed automatically upon detection of the need therefor, and removed from the toxic condition in its host, the donor as well as the native heart should experience a significant measure of relief and recovery, even if that native had undergone heart failure. Only a significant alteration in contour after placement warrants its removal for remolding. The heart cage incorporates a sufficient number of flexible points to facilitate its removal if needed.

In use, the cage is opened, and the donor heart, having been mobilized, that is, freed of any attachments or adhesions, especially to its rear, with diversion valves, drug, and bloodlines attached to the vascular stumps, is set down on the foam lining the grid. Care is given to assure that vessel stumps rising upward clear top opening 59 without contact other than lightly against the foam lining opening 59 and pulmonary vessel stumps at the upper sides pass through side openings 60 with associated clearance flap opening 63 and opening 61 with associated clearance flap 66, and opening 62 with associated clearance flap 67 without metal contact. The cage is then closed around the heart and locked closed by means of spring-loaded latch fasteners along the front seam where right and left halves come together.

FIG. 25 shows a bypass device to allow an ischemia-free carotid endarterectomy, which in the rare circumstance that either or both the native structures have been irreparably damaged by disease or the need to resect tumors, for example, can be left in place permanently as a prosthesis, with the immediate diagnosis and treatment of erratic fluctuations in blood pressure, blood gases, and blood glucose applied by the implanted disorder response system. These mid- and postprocedural problems associated with the loss due to surgical resection or trauma of the carotid bulbs where such a prosthesis without the support of an implanted automatic disorder response system is not used are briefly reviewed above in Background of the Invention.

In FIG. 25, of the branches of the right external carotid artery included in the figure, ST is the superior thyroid artery, L the lingual artery, F the facial artery, M the maxillary artery, and SupT the superior temporal artery. Part number 88 is the servovalve attached to the common carotid, 89 that attached to the external carotid, and 90 that connected to the internal carotid. The orientation of the three valves usually required to perform a carotid endarterectomy or the two in the infrequent circumstance that the external carotid is unaffected, is that least encroaching upon neighboring tissue and minimally if at all presenting a cosmetically conspicuous bulging of the neck.

The edges and corners of the valves are rounded to eliminate abrasive or gouging contact, and the valves can be wrapped within a soft sock to eliminate any irritation. Accordingly, the three valves shown in FIG. 25 have been drawn in the plane of the figure for visual clarity and must not suggest such a disposition is prescriptive. Each servovalve is shown with a direct manual fine control assist device detachably attached beneath it in a separate enclosure which will be removed before the patient is closed. The structure of the direct manual fine control device is delineated above and shown in FIGS. 10A thru 10C. Valve 89 is positioned just above the bifurcation of the external carotid and facial arteries to minimize is not eliminate the blind pocket, or cecum, wherein clot would accumulate were it positioned at a higher level.

Valve 88 on the common carotid must remain sufficiently open, or retracted, to allow blood to pass into the facial, lingual and superior thyroid arteries. While it might appear that the internal carotid has been shown disproportionately wider than the external carotid, the illustrations provided in the popular anatomy textbooks such as Cunningham, Morris, Gray, Grant, and Sobotta, were drawn from cadaver preparations, not surgical patients in whom the blood under pressure produces distention (dilatation, dilation) such as that shorn. The common and internal carotids are large enough in caliber that in order to minimize their size and weight, the lumina of the vascular servovalves connected to these are made smaller. The rationale for this reduction without adverse sequelae is provided above under Background of the Invention.

### Ports

This application concerned with vascular valves and servovalves, only a condensed summary and simplified description of body surface (on-skin), subsurface (subcutaneous subdermal), and ports which combine opening on and beneath the skin for use with an implanted automatic disorder response system can be reviewed. Suffice it to say, such ports can incorporate power button cells, which stored above-skin can be replaced, or beneath skin recharged transdermally; or transcutaneously, mechanical control knobs such as to extend or retract a push/pull, or Bowden, cable, for example, in urological applications as will be delineated below; electrical button switches to actuate tiny lamps for illumination and implanted electrostimulators when not triggered automatically by sensor feedback to the system control microprocessor; and locked system override switches which the clinician can unlock if for any reason such becomes necessary.

Subsurface (subcutaneous, subdermal) body ports for use with an implanted automatic disorder response system are portacaths, or mediports, such as those in common use, which have been modified to incorporate multiple entry openings to allow the simultaneous replenishment of subcutaneously, or subdermally, positioned small flat reservoirs storing drugs in fluid form as well as any of the manual controls listed above. As is true with a conventional single entry portacath, when drug delivery passive due to gravity and the opening is above-skin, that is, at the exterior rather than subcutaneous, the way is clear for diagnostic use, such as the drawing of blood or delivery directly to the pipe-targeted organ, gland, or nidus, for example, of fine transluminal diagnostic devices, such as an angioscope, and therapeutic devices such as an excimer laser.

In summary, such ports fall into three groups, those nonurological which are usually positioned subcutaneously in the pectoral region, those urological which are usually positioned to a side of the mons or mons veneris, and those mixed which incorporate both, wherein the medicinal openings are still subcutaneous. Both for reasons of comfort and cosmetic, in all cases, a central object is to achieve a small shape factor with minimal weight. When drug delivery is not through a passive gravity drip but rather directed by the microcontroller—or in more complex comorbid disease, a microprocessor—the reservoir and its outlet pump obstruct such passage.

For this reason, the attempt is made to always include a gravity fed line. In general, only urological ports provide an opening to the exterior for connection of a tube to pass ursine into a collection bag. Such ports will often include electrical switches to control and one or more button cell batteries to power electrical components, and control knobs mechanical Bowden cable or electrical, for example, in addition to subcutaneously positioned inlet openings for injecting drugs. Otherwise urine effluent line 110 from the bladder or neobladder is always free of obstructions.

As shown in FIG. 26B drugline K to either or both kidneys, such a drugline omits components, here the drug reservoir 102 and the drug outlet pump 103 which would block the path of the cabled device to be employed. Accordingly, nonurological ports incorporate an above-skin opening and omit the reservoir 102 and pump 103 only when diagnostic devices will be used or biopsy samples drawn, other openings for the delivery of medication located beneath the skin.

A port preferably positioned to a side of mons pubis having an above-skin central outlet pipe to pass urine into a urine collection bag tethered about the ipsilateral thigh and surrounding subcutaneous drug injection openings is shown in FIG. 26C. In a patient with retained urge and urinary tract sensation, the system used is of the type shown in FIG. 30 where a port of the kind shown in FIG. 26C provides controls for the user to switch between unilateral or bilateral partial or total automatic or voluntary evacuation, hence, the rate of collection.

The line from the bladder or neobladder can be used to pass through an endoscope to diagnose or laser to treat, for example, the interior of the surfaces in the lower urinary tract. Cabled devices best passed through a passageway that is sterile when urine effluent (outflow, emptying, egress) line 110 may become contaminated with urine containing bacteria, fungi, or other pathogen where the patient is affected by both urinary incontinence and frequent urination—the two common in infections of the lower urinary tract—are accommodated by providing an additional opening leading into an obstruction-free 8 line such as that shown as K (leading to a kidney) in FIG. 26B.

When frequent urination is not a problem, bladder effluent line 110 from the bladder or neobladder can be sanitized by releasing an antimicrobial through the accessory channel of the nonjacketing side-entry connected positioned along the bottom of the bladder or ductus side-entry jacket on the ureter and/or an antimicrobial swab run up through bladder effluent line 110 just before use. Any type port can incorporate such above-skin openings and/or components, but for better protection against infection, drug injection openings are best subcutaneous. Subcutaneous drugline entry openings also eliminate the need to remove a cap before injection, thus facilitating quick drug replenishment when all drugline openings can be accessed simultaneously with a multiple needle or needle free disposable cartridge jet injector nozzle injection head such as those depicted in FIG. 27.

That urine outlet bladder effluent pipe 110 opening and any other above-skin opening to the exterior—always covered over by a protective cap and structured to retain antimicrobials—can be used in the reverse direction to pass diagnostic tools for examining the interior of the bladder, ureters, and renal pelves on a permanent basis without the need for a ‘keyhole’ incision each time or a bandaged over, readily infected wound to allow periodic reexamination should be evident, as should the option to remove the device once a temporary condition clears.

FIG. 26A shows a vertical left side sectional view of a nonurological subcutaneous port with multiple openings for injecting drugs, FIG. 26B providing a schematized depiction of the same port in the context of an implanted automatic disorder response system. If few, the drugs are injected sequentially by hypodermic syringes or alternative means of injection 101. To minimize the duration and irritation of injection, more than two drugs are preferably injected simultaneously with a multiple needle or multiple nozzle needle free disposable cartridge jet injector head such as that depicted in FIG. 27.

Covering a 2 centimeter or so circular area of skin prepared by electrolytically removing the hair over the area, urological ports must provide an underlying surface treatment such that the interface with the skin will not result in infection or irritation such as itching. The port removable properly only by a trained clinician, the wearer can accomplished sanitizing and inflammation reversal without removing the port by using a eye dropper to drip a back pad of gauze placed before the port is attached with an antimicrobial such as an alcohol or hydrogen peroxide and/or an anti-inflammatory such as dilute prednisolone or a fluid preparation of triamcinolone provided in a vial small enough to prevent misapplication.

New materials which incorporate antimicrobials and anti-inflammatories are briefly addressed above under the section entitled Background of the Invention. Drug injection openings 107 in medicinal ports serve as the points of entry into the sidelines, thence through accessory channels 8 of the side-entry connectors, jackets, and valves at the target tissue or vessel and to prevent their disconnection are fused to the drugline 8 into which each flows. Multiple self-sealing membranes, or septa, 121 are shown as covering each port opening separately but can comprise one continuous membrane, or septum. Self-sealing membranes 121 are absorbent and wetted with an antimicrobial at the same time with a syringe introduced into injection openings 107 by inserting the Huber injection needle to just enter openings 107.

Druglines 8 are subcutaneously tunneled to the level of the target organ, gland, nidus, or tissue and then routed along the course to the target least like to strangulate intervening tissue. In FIG. 26C, the urological port is positioned to a side of the mons pubis or mons veneris so that druglines 8 must move upwards. Accordingly, in the lower position of FIG. 26C, drugline 8 would actually proceed craniad or as nearly so as would preclude kinking upon bodily movement, for example, directly upon exiting from drug injection opening 107.

As shown in FIG. 26C, were it necessary to move downward, would turn around while subcutaneous into the upward, or craniad, direction. Highly dilute (low viscosity, watery) injectants can be delivered directly to the target tissue by gravity. In FIG. 26B, where to allow direct gravity feed, the drug delivery line (drugline) 8 leading into an accessory channel of a nonjacketing side-entry connector on the organ fibrosa or into that of a side-entry perivascular jacket or servovalve on the renal artery, for example, to the kidney, denoted by the K, is continuous, that is, omits a drug storage reservoir 102 and reservoir outlet pump 103. If the line is ordinarily used for higher viscosity substances, a reservoir 101 and pump 102 are present and actuated as needed manually.

Higher viscosity drugs are injected into reservoirs such as those marked H for the heart, L for the liver, and B for the brain. The size of any component in FIG. 26B, such as the drug reservoirs 1, 2, and 3 generically identified as 102 or drug reservoir outlet pumps 103 will vary with the dose and dose release schedule of the substance injected, and the length of druglines 8 will vary based upon the distance from the port 100 to the target side-entry component. Replenishment of the flat vial storage reservoirs 102 for the independent release of each drug to a different organ, gland, or nidus to treat comorbid disease under the morbidity-distinguished hierarchical control channels or branches executed and coordinated by the system controlling microcontroller or microprocessor according to its prescription-program.

As in a conventional portacath, or mediport, migration is prevented by passing suture though small loops as shown or holes 104 or about the periphery of the port, and migration of the lines feeding into the accessory channels 8 by omitting joints with the potential to become disconnected. Shown in FIG. 26B, the pumps at the outlets of drug reservoirs 1, 2, and 3 are controlled through wires 105 by the fully implanted automatic control system microcontroller or microprocessor 106. If for any reason hard wiring should be avoided, the commands are issued by controller 106 by radio remote control, typically Bluetooth, the frequencies employed cautiously chosen to avoid extrinsic actuation by garage door openers, for example.

FIG. 26C shows a vertical cross sectional side view of a port which combines an above-skin urine outlet 110 from the bladder or neobladder and subcutaneous drug injection openings 107. In contrast to nonurological ports which are positioned subcutaneously in the pectoral region, urological ports are preferably positioned to a side of the mons pubis or mons veneris where these are easily seen and used by the patient, whether to connect a urine drain hose to or disconnect it from the urine outlet opening, adjust a ureteral valve, or make less awkward the syringe-injection of a drug into an above skin drugline opening by the patient.

Accordingly urological ports must be provided with a. Covering components to prevent microbial intrusion, and b. Situated inferior to the target tissue where passive gravity feed is not possible, a small pump, typically peristaltic. To assure freedom from infection, urine outlet, or effluent, line, or tube 110 is closed off by airtight screw-on or press-to-engage detent cap 108, and the cap filled with a small gauze wad 109 wetted with a potent antimicrobial.

Thus, should it become contaminated, the opening into urine outlet, or effluent, line 110 is routinely sterilized. The diversity of implanted components that might be controlled from such a port is considerable, and as commented upon above, includes neuromodulatory devices such as electrostimulators, control knobs the wearer uses to switch urine outflow from that normal into a proper receptacle to effluence internally through the port into a collection bag, prepositioned diagnostic viewing devices, and manual switches to release drugs by the wearer in response to predictable sources of irritation.

FIG. 27 shows the two main means for simultaneously replenishing the drug reservoirs implanted with an automatic disorder response system. The main object in simultaneous replenishment is to expedite this process with patients having multiple comorbidities who whether because of cognitive impairment due to dementia or infancy are prone to become frustrated and unruly when several injections are needed. Another advantage is that the individual syringes or needle free disposable cartridge jet injectors are prepared and confirmed as correct according to the prescription load list well before the procedure, thus effectively eliminating human error. Autoinjectors such an epinephrine pen are discounted as facilitating use by the patient, which is discouraged; replenishment should last for a period long enough that return to the clinic is not so frequent as to provoke noncompliance.

FIG. 27A shows a metal or plastic manual syringe simultaneous injector which allows multiple substantially conventional individual injection syringes 101 in FIGS. 26A and 26C collectively 111, to be used simultaneously. The drawing figures comprehend four syringes, but any number that will allow injection into such a port with multiple openings, typically 2 to 4 centimeter in diameter, and will fit into the frame can be used. Each syringe 111 will usually contain a single drug; however, drugs otherwise mixable can be combined in any one. Pending the development of switchable, such as rotatable turret mounted port openings or druglines, each opening is immutably continuous with a certain drugline, so that the destination as to organ, tissue, gland, or nidus, for example, must determine the contents of each syringe 101 in the set thereof 111.

For example, with the openings and druglines permanently associated, unless more than one drugline is led to the same destination, to inject a large volume of a drug intended for the same destination will require the use of an additional syringe to sequentially inject the same opening, and if the same drug is to be pipe-targeted to different organs, glands, tissues or nidi, these must be separated into different syringes, each aligned to the respective port opening for the destination intended. Alternatively, a confluence path between tips of adjacent syringe barrels can be used to allow two or more syringes to empty into the same reservoir. Using syringes having barrels different in diameter and loading different concentrations of the drugs are two ways to adjust the dose.

Otherwise, the set 111 of individual syringes 101 are unconventional in lacking barrel 112 or plunger 113 flanges, and in situating the hypodermic needles 114 with syringe tips (Luer, slip) as close to the periphery of each syringe as necessary so that each needle will align with its respective opening in the port. Hypodermic needles 123 can be of the Huber noncoring type with tips curved but no more than is essential to avoid coring. The barrels are conventionally calibrated with volume markings 115 in cubic centimeters or milliliters. The manual syringe simultaneous injector is comprised of outer rectangular frame 116 made of plastic or metal fabricated of channel, or U-cross section stock, through which reciprocally slidable complementary n-section internal rectangular frame 117, made of the same or a different material, can slide reciprocally as a track.

The upper member of internal frame 117 must have bonded to its upper side a floor plate 118 upon which the syringes rest covering an area that will assure the stably vertical alignment of the set 111 of individual syringes 101. As illustrated, simultaneous injection of the set of syringes 111 is accomplished by using the fingers to pull down the lower member, or crosspiece 119, of outer stationary rectangular frame 116 toward the lower reciprocally sliding member, or crosspiece 120, of internal frame 117. This drives floor 118 bonded atop the lower member 119 of outer rectangular frame 116 upward, thus forcing plungers 113 upward to eject their contents. Injection is into multiple openings 107 of subcutaneous port 146 in the sectional view of FIGS. 26A and 122 in the extracorporeal view of FIGS. 17 and 27C with backside view in FIG. 26B. Injection point indicating tattoo marks on the skin are shown as 124 in FIGS. 17 and 27C.

The markings consist of two tiny tattoo dots, which barely noticeable, can be different in color and lased away if and when the port is removed. The same markings pertain whether the device of injection is that using hypodermic needles as depicted in FIG. 27A or jet injection micronozzles as shown in FIG. 27B. Showing two of the four injection points is sufficient to align all four or more of the hypodermic needle tips or orifices of the hex nozzles of the jet injector. Front views, FIG. 27A conceals two additional hypodermic needles, and FIG. 27B two additional jet injection fill chambers to the rear. In either, provided the outlets are aligned to the injection openings in the subcutaneously implanted port, the drug fill chambers of the injectors to the rear can be of any diameter.

More specifically, the relative drug holding capacities of the syringe and fill chambers to the rear may be the same, the reverse, or different than either of those seen at the front. Nonmanual injection using ports of the Bard PowerPort® (division of Becton Dickinson-C. R. Bard, Covington, Ga.) type allow the subcutaneously implanted drug reservoirs to be replenished at the rate of 5 milliliters per/second at 300 pounds per square inch, further reducing patient annoyance. Unlike an embodiment comprising disposable cartridge jet injectors, the multiple jet injector shown in FIG. 27B is analogous to the hypodermic needle embodiment shown in FIG. 27A in applying a common driving means to the ejection plural plungers.

Means for the injection of drugs essential to support an implanted automatic disorder response system, FIG. 27B omits tiny seals, washers, valves, and screws, for example, the showing of which would be superfluous for those familiar with such devices. In FIG. 27B, 125 is the head assembly; 126 is the injection thumb actuated release button; 127 the spring release or cocking lever; 128 the main power spring, 129 the common shaft for the drug fill chambers; 130 the cocking lever spring that primes main spring 128; 131 the cocking lever hinge; 132 index and middle finger rests; 133 the injector body; 134 the upper seals of the two front fill chambers 135 and 136; 137 the lower seals of the two front fill chambers; 138 and 139 the two front fill chamber 135 and 136 ejection nozzles; and 140 and 141 the ejection outlet orifices.

FIG. 27C shows an entirely subcutaneous implanted multiport in a baby where the purpose thereof will almost always be other than to support a double heart switch, or bypass transplant as shown in FIG. 17. The entirely subcutaneous port is of the type shown in FIGS. 26A and 26B as 146 and shown as 122 in the extracorporeal view of FIG. 27C, where the on-the-skin, or cutaneous injection point indicator marks appear as 124.

FIG. 28 shows a prosthesis for the automatic elimination of urine in a patient with a missing or severely defective lower urinary tract into a synthetic neobladder for drainage into a paracorporeal collection bag usually cinched about a thigh, or directly into the collection bag. Both vascular valve implemented urinary prostheses and assist devices can be unilateral, bilateral, or switchable between the two. As a prosthesis, the system requires no voluntary controls for the wearer to turn on or off automatic collection and voiding, for example, as does a partial dependency system, or urinary assist device for a patient with intact urge and urinary tract pain sensation.

Gut not adapted to conduct urine and prone to metaplastic degeneration which can lead to malignancy, the surgical reconstruction of the urinary bladder or a stoma with harvested gut is discouraged. Because it reveals an inherent proclivity toward malignancy, this is pertinent to a lower tract missing following evisceration due to a malignancy that had metastasized to neighboring tissue. A prosthesis, the system uses nonadjustable, that is, permanently set, diversion jackets, which nonadjustable are not properly referred to as valves, such as those shown in FIGS. 2 and 3 or solenoid-driven valves such as shown in FIGS. 7 and 8.

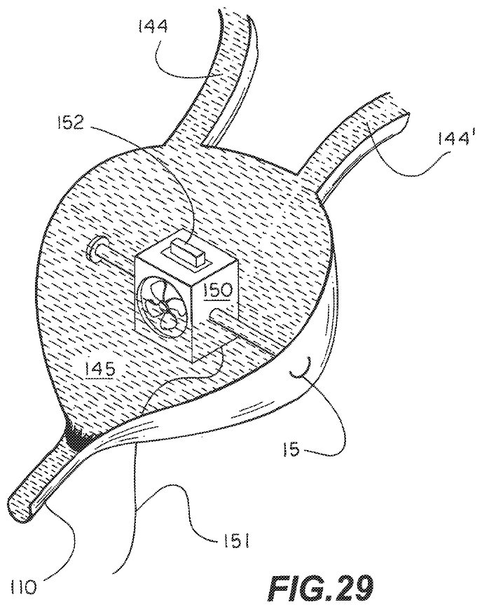

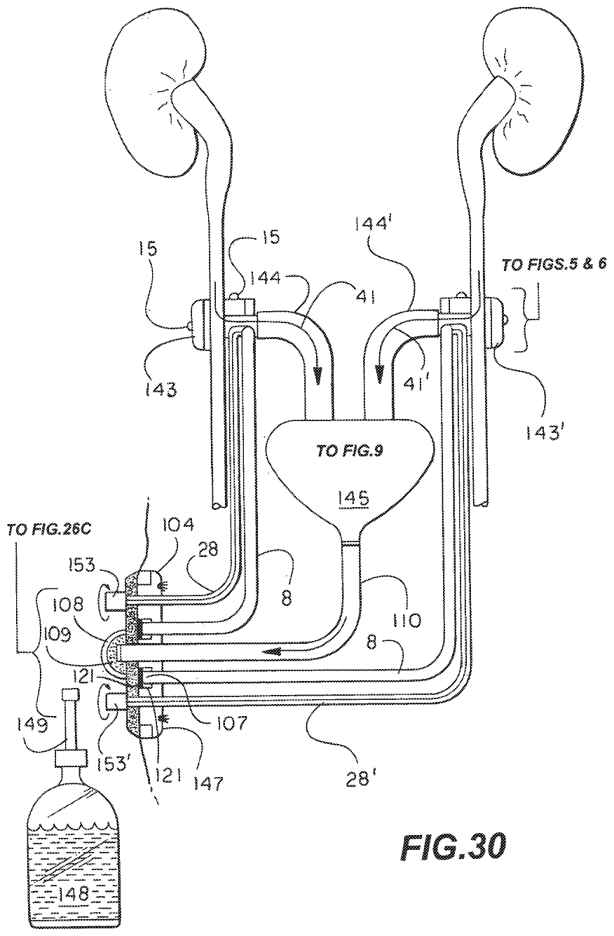

While nonadjustable by the patient, post-implantation adjustment by a clinician can be readily accomplished by endoscopic access through a ‘keyhole’ incision. In FIG. 28, diversion jackets 143 and 143′ are of the nonadjustable nonvalve type shown in FIGS. 2, 3, 7, and 8. Nonadjustable in this regard denotes that the patient, who due to the absence or abnormality of the lower urinary tract lacks urge and pain sensation, requires full automatic control over urinary voiding. Diversion jackets 143 and 143′ tap off, divert, and pass urine, of which 41 and 41′ represent the flowlines, into synthetic prosthetic ureters, or neoureters, 144 and 144,′ which drain into synthetic neobladder, or neoureter confluence chamber 145, shown in greater detail in FIG. 29.

While the patient is erect, neobladder 145 drains into collection bag 148 under gravity. In this urinary prosthesis where urinary tract sensation and/or self-control is missing, control over voiding when the patient is recumbent must be automatic. To this end, as neoureter confluence chamber, or synthetic neobladder 145 fills, a small bar which presses down on a strain gauge (not shown) eventually imposes the threshold level of force for initiating evacuation (emptying), passing current through a small box fan-configured impeller 150 on the floor, or as shown in FIG. 29, suspended at the center of polymeric confluence chamber 145 to drive the urine out through urine effluent line, or outlet pipe 110 which exits the body through the center of combined above-skin, or cutaneous, outlet, with surrounding subcutaneous drug inlet openings such as those shown in the body surface port 147 depicted in FIG. 26C, thence through collection bag connecting hose 149 into collection bag 148.

FIG. 29 provides a more detailed view of the neobladder or neoureter confluence chamber 145 shown in FIGS. 28 and 30. In FIG. 29, small impeller 150, configured much as a box fan, is positioned at the bottom center, or as shown, suspended at the center of confluence chamber 145. Confluence chamber 145 is molded to the same shape as a native bladder and to allow the longest collection time, as large in capacity as does not encroach on neighboring tissue or present a weight when filled that induces the sensation of a foreign object. Storage in confluence chamber, or neobladder 145 causes less discomfort than loading collection bag 148 at a quicker rate.

Impeller 150 draws electrical power though wire 151 from one or more button cells positioned about the central bladder effluent pipe 110 outlet in the body surface ports as are the drug injection openings 107 shown in FIGS. 26B, 26C, and 28 and the push/pull cable control knobs shown in FIG. 30. Confluence chamber 145 is made of a light weight polymer such as polyethylene terephthalate, polypropylene, or polyether ether ketone. As shown in FIG. 28, the prosthetic lower urinary tract is not switchable to normal voiding during the daytime or when the user is before the public, for example.

Accordingly, an antiseptic delivered into the ureters through valves 143 and 143′ will be effective through the entire length of the synthetic system and passed into collection bag 148 so that native tissue is not exposed to it, making disinfection through the use of more concentrated antiseptic unobjectionable. Unless the reservoir and outlet pump or pumps are positioned within the pelvis, so that the injection openings in the surface port can be incorporated into a port positioned to a side of the mons pubis, an antiseptic, anti-inflammatory, or analgesic, for example, must be injected through a port superior to, that is, above the level of side-entry diversion valves 143 and 143′ to arrive by passive gravity without the need for intrapelvic componentry.

Ordinarily, this will be through a port positioned in the pectoral region as are conventional portacaths or mediports. The user-controllable system shown in FIG. 30 uses drugs in the same manner as the prosthesis shown in FIG. 28 only when the user switches it to urinary diversion. Switching diversion off, then, allows medication introduced through the drug openings, or injection points 107 in the surface valve treats the entire native lower urinary tract. In FIG. 29, small bar 152 positioned atop impeller 150 pushes down a small rod at the center of a strain gauge (unshown). In a standalone urinary application, the strain gauge sends its output directly to the implanted system microcontroller.

Representation of the impeller as centered within the confluence chamber and the strain gauge at the tope center thereof is exemplary. Either can be located together or separately at a number of positions. That is, while shown positioned on top of impeller 150, small bar 152 to push against rod and strain gauge can be a button of any shape and situated in any location along the interior surface of confluence chamber 145 except at the top thereof. To assure comfort and allow the confluence chamber 145 to be produced in no more than a few sizes, the controller is programmed to initiate voiding, into a collection bag 148, for example, when the signal strength received from the strain gauge is just short of that at which the user experiences discomfort.

In a system to treat comorbid disease whereof the urinary voiding subsystem constitutes but one level requiring therapeutic monitoring and coordinated response by a hierarchical control system in the treatment of multiple conditions, the strain gauge sends its output through the local or lowest level control channel, meaning that dedicated to the urinary subsystem, whence the signal rise up through and is processed by increasingly higher levels of control in the hierarchy to the multiple implant system master controller, here, a microprocessor.

FIG. 30 shows a urinary assist, or voiding control device, which allows the patient with intractable nocturia, urinary incontinence, or frequent urination, or one who must participate in an activity which does not allow for interruption, such as public appearance or performance, to switch between voidance into confluence chamber 145, with automatic emptying into collection bag 148, or into a bathroom receptacle. Following a surgical procedure on or involving the lower urinary tract, such a system is useful on a temporary basis for relieving the lower tract of constant exposure to urine by bypassing the healing tissue, thus expediting healing of the tract.

To best expedite healing, postprocedural bypass can be continuous or intermittent and automatically coordinated with the automatic pipe-targeted release of a drug, during which diversion is not used, or during prosthesis disinfection or crystal dissolution, during which diversion is not used. When administering drugs, the controller switches off the bypass feature, thus gaining access to the native tissue. When administering system maintenance substances, the controller switches on the bypass feature, thus causing the substance or substances to flow through the synthetic device. Use thus is possible whether the urinary diversion system is implanted for the procedure or was already in place.

Such use is similar to the use of a carotid prostheses such as shown in FIGS. 25A and 25B following a carotid endarterectomy, for example, where bypass to expedite healing and expose or bypass the native tissue as required can likewise be continuous or intermittent. No less pertinent for expediting healing is the capability to directly pipe-target drugs such as anti-inflammatory, antimicrobial, and/or analgesic to the treatment site, thus precluding exposure to potentially harmful substances of tissue outside the urinary tract, thus avoiding adverse side effects and the risk of complications, while at the same time allowing the use of dose levels in the treatment of the lower urinary tract not restricted due to this consideration.

If explanted, leaving in place the body surface port, controller, druglines, and accessory channels of lower urinary tract and carotid prostheses by replacing the valves with basic ductus side-entry jackets allows continued automatic targeted treatment, if necessary, to the end of life even in a younger patient. Such extended use is made possible because the system controller can schedule the periodic release of substances to maintain the system itself, such as anticoagulants, thrombolytics, and crystal dissolvents on the same basis as drugs.

Except in cases of megaureter where servomotor-driven diversion chutes controlled by potentiometers in place of mechanical control knobs 153 and 153′ positioned around urine effluent line 110 (which in turn is connected to urine effluent hose 149 for drainage into urine collection bag 148) at the center of surface port 104 positioned to a side of the mons pubis or mons veneris, the diversion chutes in valves 143 and 143′ need not advance and retract more than 5 millimeters. This small excursion allows a simple in-line direct drive mechanical embodiment providing continuous adjustability, not bistable as would a solenoid, to be produced without the expense of servomotors.

In the front schematic view of such a mechanically controllable embodiment shown in FIG. 30, push/pull cable rotating knob 153; controlling side-entry diversion valves of the kind shown in FIGS. 5 and 6 on the right hand side of the patient and valve 153′ controlling that on the left rotate proximal twisted strip segments next to be described (behind control knobs 153 and 153′ and unseen in FIG. 30) at the proximal end of push/pull control cables 28 and 28′, also shown in FIG. 5 depicting the type valve used. So that one half turn of knobs 153 and 153′ clockwise fully extends push/pull cables 28 and 28′ respectively, while one half counterclockwise rotation fully retracts each, the turns ratio of the knobs to chute displacement is best one to two; that is, so that the half way rotation of the knobs moves the diversion chutes from one end to the other, not just half way.

Requiring to control movement over a distance no more than millimetric, the push/pull control cable extension and retraction mechanism inside port 147 in FIG. 30 providing control knobs positioned as would injection openings or button cells about urine effluent line 110 as shown in FIG. 26C can be made small enough to allow the one body surface port to include subdermal injection openings and button cells as well as control knobs. The twisted strip segment of each control which is separate between the sides consists of two turns covering an overall length one millimeter greater than the equivalent excursion sought of the diversion chutes and is distinct from the proximal ends of the push/pull cables fused to the rear of the ferrules.

In the control for each side, the forward or distal of the two turns of the twisted strip is contained inside, and the near (rear, proximal) turn outside its respective ferrule. The ferrule has a rectangular opening at the front end so that rotation of the twisted strip clockwise causes the outer turn to drive the ferrule, and therewith, the push/pull cable ending at the valve continuous with it forward over the distance equivalent to the entire displacement required of the diversion chutes. The outside turn drives the cables forward and the chutes deeper into the ureter, and the inside turn pulls the cable backward, withdrawing the chutes from the ureter.

Along with the clockwise or forward detent of the control knobs, contact of the rear end of the inside turn against the rear wall of the ferrules sets the limit to movement of cables 28 and 28′ in the forward or chute deployment direction. Along with the counterclockwise or backward detent of the control knobs, the limit of backward (chute withdrawal equivalent, retractive, proximal) chute movement is set by contact of the turn in front of the ferrule with the rear of the control knob.

Accordingly, the rear ends of the ferrules fused to and continuous with the solid cables, clockwise rotation of knobs 153 and 153′ causes the respective twisted strips to drive the ferrules and thus the cables and diversion chutes continuous with these forward, or distally, deeper into the lumen of the substrate ductus, while rotation of the knobs counterclockwise draws the ferrules proximally and therewith, the cables and the chutes retracted from the lumen of the substrate ductus. The small distance for the cables to move thus allows not only the avoidance of the cost for servovalves but the need for a lever mechanism likely to catch on clothing or cause an occasional scrape.

### Anastomosis Coupling or Flow Diverter

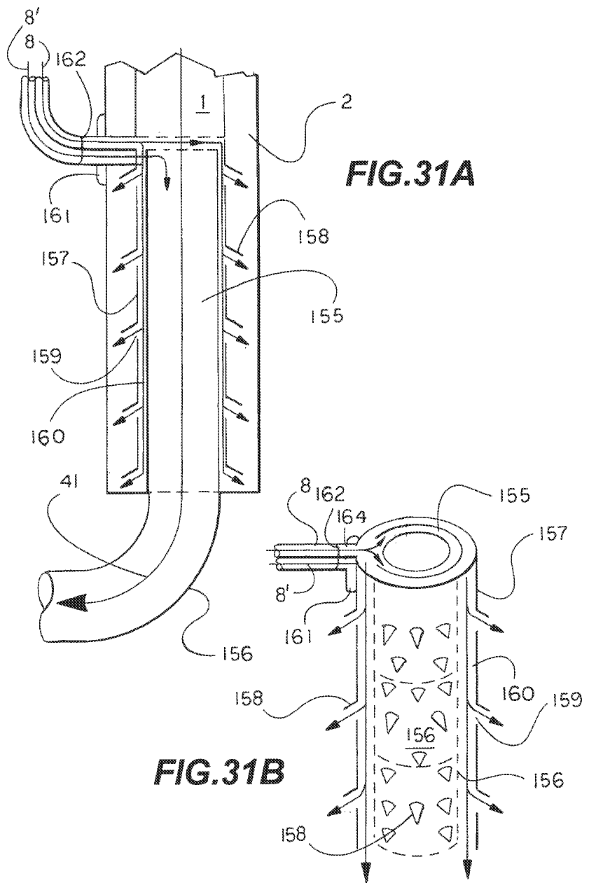

The functionality and functions of endoluminal anastomosis coupling or flow diverters in comparison with that of side-entry diversion jackets is addressed above in section 2. Background of the Invention, which briefly describes their structure. In FIG. 31A, part number 155 designates the endoluminal flow diverter in its entirety. Two accessory channels are provided, one, part number 8, delivers drugs into the intertube space, or the passageway between inner tube 156 and outer tube 157 so that under the slight to moderate pressure of the implanted drug reservoir outlet pump, these flow around and down intertube space 160 to emerge through apertures 159, one each created by the die punching outward of a pointed retention tine (barb, prong) 158.

Drugs such as anti-inflammatories delivered through accessory channel 8′ flow over the urothelium, tunica mucosa, or endothelium of the substrate ductus for absorption and uptake, not just at apertures 159, while accessory channel 8′ delivers an anticoagulant, for example, into the native lumen in vessels and a crystal solvent, for example, in the ureters. Double, or dual, lumen tubular extension 164, which protrudes radially outward from the adventitia no more than is needed to securely friction fit druglines and accessory channels 8 and 8′, opens adaxially into intertube space 160 separating inner tube 156 from outer tube 157. To avoid the need for a second puncture wound through luminal wall 2, the druglines connected to tubular extension 164 arrive through double lumen catheters, or the lines flowing into accessory channels 8 and 8′ can be separate and run in parallel from the drug sources, normally, small flat drug reservoirs implanted in the pectoral region.

Grommet 161 then encloses the single perforation. To prevent incising the tunica mucosa, urothelium, or endothelium when the endoluminal anastomosis coupling or diverter is inserted, double lumen tubular extension 164 has a smoothly burnished end 162, and along with antegrade inclined retention tines (barbs, prongs) 158, is blanketed over or with a coating of a readily absorbable substance such as a sugar or chilled butter, which can be quickly dispersed by passing water or enzymes respectively through apertures 159. Flowline 41 indicates the direction of flow through inner tube 156.

Slightly but palpably protrusive, extension 164 is easily spotted at the surface of the adventitia as a slight protuberance, showing the operator the point to remove a plug of tissue no larger than is needed to allow double lumen extension 164 to protrude out through the side of the adventitia. Grommet 161, incorporating or coated with an anti-inflammatory and antimicrobial polymer, is then placed in surrounding relation to double lumen extension 164 against the adventitia to completely enclose this junction and prevent microbial intrusion.

When inserted into the cut end of the native ductus, the tube within a tube flow diverter or adventitia coupling as shown does not allow switching flow between the diversion path and that normal. For this reason, it is functionally distinct from a side-entry diversion jacket, which is more versatile in flow control. By the same token, a straight, or nondiverter, embodiment of the device has applications of which the periductal jacket is incapable, such as transluminal placement as a stent or anastomosis coupling. Both allow the diversion of flow through a shunt or bypass, but only the diversion jacket allows the outflow to be divided.

1. A device selectively positionable about a tubular anatomical structure, said structure itself the product of nature and not claimed, for delivering drugs, passing cabled devices into, and extracting luminal contents and biopsy tissue samples from within the lumen of said tubular anatomical structure comprising:
an outer shell of semicylindrical halves joined together along a common edge where these meet by spring loaded hinges, so that said semicylindrical halves when opened and placed to encircle said tubular anatomical structure, the halves grip about said tubular anatomical structure as a stationary collar wherein;
a cushioning layer to protect the small nerves and vessels that enter and depart from the outer surface of said tubular anatomical structure lines the internal surface of each said shell half;
perforations which pass entirely through said outer shell and said internal cushion lining are placed to give access to the internal environment of said tubular anatomical structure;
an opening in the side of said collar into which a side tube with trepan front edge can be inserted, said side tube rotatable around and reciprocable along its longitudinal axis, allowing a plug of tissue to be excised from the wall of said tubular anatomical structure so that the lumen of said side tube will be continuous with the lumen of said tubular anatomical structure; said side tube fixable in rotational angle and depth of penetration into the side of said tubular anatomical structure;
a self-locking screw down cap with internal expansion bushing that fits onto an external thread at the base of said side tube allows said side tube to be fixed in rotational angle and depth of insertion into said side opening.
a projectable and retractable tongue-shaped diversion chute with upturned distal end mounted within said side tube with trepan front edge wherewith to slide reciprocally to selectively positional depths into said tubular anatomical structure so that the column of bodily fluid antegrade to it is diverted out through said side hole and said trepan tube for continued flow through a synthetic tube, said combination of elements comprising a vascular valve.

2. A valve according to claim 1 where said tongue is driven by a solenoid.

3. A valve according to claim 1 where said tongue is driven by a servomotor.

4. A valve according to claim 1 which incorporates a permanent magnet layer along the internal surface of said outer shell, said magnet layer interposed between said outer shell and said cushion layer and interrupted to accommodate the opening and closing of said collar and the passing through of said perforations, said magnet layer magnetized to exert a tractive force centrally toward and perpendicular to the longitudinal axis of said collar, making possible the detention and extraction of magnetically susceptible luminal contents.

5. A valve according to claim 1 which incorporates a plurality of electromagnets between said outer shell and said cushion layer and interrupted to accommodate the opening and closing of said collar and the passing through of said perforations, said plurality of electromagnets selectively energizable to exert tractive force eccentrically and collectively energizable to exert tractive force centrally toward and perpendicular to the longitudinal axis of said collar, making possible the detention and extraction of magnetically susceptible luminal contents.

6. A valve according to claim 1 which omits said perforations and incorporates a layer of radiation shielding material in concentric relation to said long axis of said collar, said radiation shield layer situated along the internal surface of said outer shell to surround said magnet layer when present, and interposed between said outer shell and said cushion layer when said magnet layer is absent, said radiation shield layer interrupted to accommodate the opening and closing of said collar, said radiation shield layer serving to allow the passage through said collar and the line leading to it of low to moderate radiation dose rate radionuclides and radioactive isotopes without causing radiation injury to surrounding tissue.

7. A valve according to claim 1 wherein said side tube is in turn entered by a catheteric side tube subsidiary to said side tube, said subsidiary side tube allowing the directly targeted delivery into said side tube, collar, and native lumen, hence, treatment site, of fluid drugs, medicinal solutions, and tubing maintenance solutions, such as ureterolith and nephrolith solvents when said collar is applied along the urinary tract and antithrombotic medication when said collar is applied along the vascular tree.

8. A plurality of vascular valves according to claim 1 wherewith at least one pump supplying fluid medicinals to said diversion jackets is controlled according to a prescription program by a microcontroller such that: a plurality of physiological parameter sensors implanted at different locations in the body send outputs as subordinate negative feedback loop nodes in a hierarchical control system to said microcontroller as master node; these outputs represent feedback where each signals to the microcontroller an out of range condition which necessitates the prescribed medication;
the microcontroller responds by causing said pump to index to and release the medication prescribed for that subordinate node in the dose proportional to the out of range feedback signal received; as master node,
the microcontroller governs the discharge of the prescription program to include dispensing the medication through each subsidiary control loop as a subordinate node in a coordinated manner as governed by the prescription program so that
dosing among the nodes is interrelated to attain the highest possible overall homeostasis; such a system overall thus able to treat comorbid conditions affecting different organ systems in a coordinated manner as an automatic homeostasis stabilizer and ambulatory prosthetic disorder response system.

9. The method of solid organ transplantation from a brain-dead organ donor placed on life support prior to and continued following death to an organ recipient through the seamless transfer of the organ blood supply and drainage from the recipient to the donor, the donor organ then harvested and placed in the recipient.

10. The method according to claim 9 whereby different organs are transplanted into an organ recipient through the seamless transfer of the organ blood supplies and drainages from the recipient to the donor.

11. The method according to claim 9 whereby different organs are transplanted into different recipients through the seamless transfer of the organ blood supplies and drainages from the recipients to the donor.

12. The method of transplanting one of a paired solid organ from a living organ donor to an organ recipient through the seamless transfer of the organ blood supply and drainage from the recipient to the donor.

13. The method of transplanting one of a paired solid organ from each of two living organ donors to an organ recipient through the seamless transfer of the organ blood supply and drainage from the recipient to the donor.

14. The method according to claims 9 thru 13 where this procedure is carried out under regional anesthesia without the need for cardiopulmonary bypass.

15. The method according to claims 9 thru 13 whereby said transfer of circulation is administered by an automatic control system comprising connectors to ductus and tissue surfaces, said connectors incorporating access channels for the direct delivery of drugs, synthetic fluid lines that deliver the drugs into the access channels, drug reservoirs to store the drugs,
